(19) United States
(12) Patent Application Publication
(10) Pub. No.: US 2026/0116991 A1
(43) Pub. Date: Apr. 30, 2026

(54) MONOCLONAL ANTIBODY FOR SPECIFICALLY RECOGNIZING GLYPICAN-3, AND APPLICATION THEREOF

(71) Applicant: HAINAN SIMCERE ZAIMING PHARMACEUTICAL CO., LTD., Haikou (CN)

(72) Inventors: Shumei YOU, Shanghai (CN); Yingying HU, Shanghai (CN); Cuiqing YANG, Shanghai (CN); Zhuoxiao CAO, Shanghai (CN); Renhong TANG, Shanghai (CN); Jinsheng REN, Shanghai (CN)

(73) Assignee: HAINAN SIMCERE ZAIMING PHARMACEUTICAL CO., LTD., Haikou (CN)

(21) Appl. No.: 18/275,529

(22) PCT Filed: Jan. 29, 2022
(86) PCT No.: PCT/CN2022/074950

§ 371 Date: Aug. 2, 2023

(30) Foreign Application Priority Data
Feb. 3, 2021 (CN) 202110147290.2

(51) Int. Cl. C07K 16/30

(52) U.S. Cl. CPC C07K 16/303; C07K 2317/24; C07K 2317/33; C07K 2317/92

(57) ABSTRACT

Provided are an isolated monoclonal antibody specifically binding to glypican-3 (GPC3) with high affinity, a nucleic acid molecule encoding a GPC3 antibody, an expression vector, a host cell, a method for preparing a GPC3 antibody, and an immunoconjugate, chimeric antigen receptor, immunocompetent cell, multispecific molecule, and pharmaceutical composition comprising the GPC3 antibody. Further provided are a method for detecting GPC3, and a method for treating GPC3-related diseases comprising hepatocellular carcinoma.

### CROSS-REFERENCE TO RELATED APPLICATIONS

This is a U.S. National Phase application under 35 U.S.C. § 371 of International Application No. PCT/CN2022/074950, titled “MONOCLONAL ANTIBODY FOR SPECIFICALLY RECOGNIZING GLYPICAN-3, AND APPLICATION THEREOF”, filed on Jan. 29, 2022, which claims the priority to Chinese Patent Application No. 202110147290.2, titled “MONOCLONAL ANTIBODY FOR SPECIFICALLY RECOGNIZING GLYPICAN-3, AND APPLICATION THEREOF”, filed on Feb. 3, 2021, with the China National Intellectual Property Administration, which are incorporated herein by reference in their entireties.

### INCORPORATION OF SEQUENCE LISTING

The sequence listing that is contained in the file named “UNIT-P0114US_Corrected Sequence Listing”, which is 247 KB (as measured in Microsoft Windows®) and was created on Feb. 29, 2024, is filed herewith by electronic submission, and is incorporated by reference herein.

### TECHNICAL FIELD

The present invention relates to the field of tumor immunotherapy or diagnosis, in particular to a monoclonal antibody specifically recognizing glypican-3 (GPC3) and use thereof.

### BACKGROUND

Glypican-3 (GPC3) is a heparan sulfate (HS) glycoprotein, belonging to a family of the heparan sulfate proteoglycans. GPC3 is attached to the cell membrane by a glycophosphatidylinositol (GPI) anchor. The GPC3 core protein consists of 580 amino acids and is about 70 kD in size. The protein can be cleaved by furin to produce a 40-kD N-terminal subunit and a 30-kD C-terminal subunit, and the two subunits are linked by disulfide bonds. Two HS side chains of GPC3 are attached near the C-terminal position (Takahiro Nishida, Hiroaki Kataoka. Glypican 3-Targeted Therapy in Hepatocellular Carcinoma, Cancers 2019; 11 (9): 1339).

GPC3 is believed to play a crucial regulatory role in cellular proliferation in embryonic mesodermal tissues since deletion of the GPC3 gene leads to the development of gigantism/overgrowth syndrome known as Simpson-Golabi-Behmel syndrome (SGBS). GPC3 is expressed throughout the fetal period, but is seldom expressed after birth in normal tissues despite the weak expression in placental, mammary, mesothelial, ovarian, lung and kidney tissues. Abnormal expression of GPC3 has been reported in various tumor tissues in adults, such as hepatocellular carcinoma (HCC), lung squamous cell carcinoma, gastric cancer, and ovarian cancer. GPC3 is highly expressed especially in HCC cells, and promotes growth and invasion of HCC cells by enhancing autocrine/paracrine canonical Wnt signaling (Capurro M I, Xiang Y-Y, Lobe C, Filmus J. Glypican-3 promotes the growth of hepatocellular carcinoma by stimulating canonical Wnt signaling. Cancer Res 2005; 65:6245-54). Immunohistochemical staining revealed that high expression of GPC3 protein was observed in tumor tissues of approximately 70% of patients with HCC (Capurro M, Wanless I R, Sherman M, et al. Glypican-3: a novel serum and histochemical marker for hepatocellular carcinoma. Gastroenterology 2003; 125:89-97), and therefore, GPC3 is considered a candidate target for tumor therapy.

As a recombinant humanized monoclonal antibody developed by Chugai Pharmaceutical, codrituzumab (codrituzumab, also known as GC33 antibody) binds to a membrane proximal region of the GPC3 protein. The GC33 antibody targets GPC3-positive HCC cells and can produce antibody-dependent cellular cytotoxicity (ADCC). In a clinical phase I trial, codrituzumab showed good immune tolerance and produced anti-tumor effects in patients with HCC (Ikeda M, Ohkawa S, Okusaka T, et al. Japanese phase I study of GC33, a humanized antibody against glypican-3 for advanced hepatocellular carcinoma. Cancer Sci. 2014, 105, 455-462). However, in a phase II clinical trial that enrolled 185 patients with advanced hepatocellular carcinoma, codrituzumab was less effective compared with controls, and investigators concluded that patient outcomes could be improved either by using high doses of codrituzumab or by selecting patients expressing higher levels of GPC3 or CD16 (Abou-Alfa G. K, Puig O, Daniele B, et al. Randomized phase II placebo controlled study of codrituzumab in previously treated patients with advanced hepatocellular carcinoma. J. Hepatol. 2016, 65, 289-295). In conclusion, clinical application of this antibody remains to be discussed.

In 1975, Kohler and Milstein discovered that hybrid cells formed by fusing mouse myeloma cells with splenocytes from mice immunized with sheep red blood cells could produce antibodies and immortalize, thereby creating a monoclonal antibody hybridoma technique (Köhler G, Milstein C: Continuous cultures of fused cells secreting antibody of predefined specificity. Nature. 1975, 256, 495-497). A traditional hybridoma technique involves fusion of splenocytes from an immunized animal with myeloma cells of a cogenetic animal under the induction of polyethylene glycol (PEG). The method is simple to operate but low in fusion efficiency. In recent years, electrofusion of cells has been rapidly developed and applied in the field of preparation of monoclonal antibodies, and hybridomas secreting monoclonal antibodies can be more effectively obtained due to high fusion frequency of electrofusion.

### SUMMARY

In response to the problems in the prior art, the present invention provides an isolated monoclonal antibody binding with high affinity to GPC3 protein. The present invention further provides an immunoconjugate comprising the antibody, a chimeric antigen receptor, an immunocompetent cell, a multispecific molecule, a nucleic acid molecule, an expression vector, a host cell, a pharmaceutical composition, a preparation method, and use. Various GPC3-targeted hybridoma monoclonal antibodies are obtained in the present invention by electrofusion, providing support for development of GPC3-targeted therapeutic antibodies. According to a first aspect, the present invention provides an anti-glypican-3 (GPC3) antibody or antigen-binding moiety, wherein the antibody or antigen-binding moiety comprises heavy chain CDRs having CDR1-VH, CDR2-VH and CDR3-VH, wherein the CDR1-VH, the CDR2-VH and the CDR3-VH have any sequence selected from the following or a sequence combination having 1, 2, 3 or more amino acid insertions, deletions and/or substitutions compared with the sequence, and preferably, the substitutions are conservative amino acid substitutions:
(1) the CDR1-VH may be selected from SEQ ID NO: 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168, 171, 174, 177, 180, 183, 186, 189, 192, 195, 198, 201, 204, 207, 210, 213, 216, 219, 222, 225, 228, 231, 234, 237, 240, 243, 246, 249, 252, 255, 258, 261, 264, 267, 270, 273, 276, 279, 282, 285, 288, 291, 294, 297, 300, 303, 306, 309, 312, 315, 318, 321, 324, 327, 330, 333, 336, 339, 342, 345, 348, 351, 354, 357, 360, 363, 366, 369 or 372;
(2) the CDR2-VH may be selected from SEQ ID NO: 70, 73, 76, 79, 82, 85, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, 217, 220, 223, 226, 229, 232, 235, 238, 241, 244, 247, 250, 253, 256, 259, 262, 265, 268, 271, 274, 277, 280, 283, 286, 289, 292, 295, 298, 301, 304, 307, 310, 313, 316, 319, 322, 325, 328, 331, 334, 337, 340, 343, 346, 349, 352, 355, 358, 361, 364, 367, 370, 373 or 604;
(3) the CDR3-VH may be selected from SEQ ID NO: 71, 74, 77, 80, 83, 86, 89, 92, 95, 98, 101, 104, 107, 110, 113, 116, 119, 122, 125, 128, 131, 134, 137, 140, 143, 146, 149, 152, 155, 158, 161, 164, 167, 170, 173, 176, 179, 182, 185, 188, 191, 194, 197, 200, 203, 206, 209, 212, 215, 218, 221, 224, 227, 230, 233, 236, 239, 242, 245, 248, 251, 254, 257, 260, 263, 266, 269, 272, 275, 278, 281, 284, 287, 290, 293, 296, 299, 302, 305, 308, 311, 314, 317, 320, 323, 326, 329, 332, 335, 338, 341, 344, 347, 350, 353, 356, 359, 362, 365, 368, 371 or 374;
and/or, light chain CDRs having CDR1-VL, CDR2-VL and CDR3-VL, wherein the CDR1-VL, the CDR2-VL and the CDR3-VL have any sequence selected from the following or a sequence combination having 1, 2, 3 or more amino acid insertions, deletions and/or substitutions compared with the sequence, and preferably, the substitutions are conservative amino acid substitutions:
(4) the CDR1-VL may be selected from SEQ ID NO: 375, 378, 381, 384, 387, 390, 393, 396, 399, 402, 405, 408, 411, 414, 417, 420, 423, 426, 429, 432, 435, 438, 441, 444, 447, 450, 453, 456, 459, 462, 465, 468, 471, 474, 477, 480, 483, 486, 489, 492, 495, 498, 501, 504, 507, 510, 513, 516, 519, 522, 525, 528, 531, 534, 537, 540, 543, 546, 549, 552, 555, 558, 561, 564, 567, 570, 573, 576, 602, 603, 613, 614, 623, 624, 636, 637, 638, 639, 640, 652, 653, 654, 655 or 656;
(5) the CDR2-VL may be selected from SEQ ID NO: 376, 379, 382, 385, 388, 391, 394, 397, 400, 403, 406, 409, 412, 415, 418, 421, 424, 427, 430, 433, 436, 439, 442, 445, 448, 451, 454, 457, 460, 463, 466, 469, 472, 475, 478, 481, 484, 487, 490, 493, 496, 499, 502, 505, 508, 511, 514, 517, 520, 523, 526, 529, 532, 535, 538, 541, 544, 547, 550, 553, 556, 559, 562, 565, 568, 571, 574 or 577; and
(6) the CDR3-VL may be selected from SEQ ID NO: 377, 380, 383, 386, 389, 392, 395, 398, 401, 404, 407, 410, 413, 416, 419, 422, 425, 428, 431, 434, 437, 440, 443, 446, 449, 452, 455, 458, 461, 464, 467, 470, 473, 476, 479, 482, 485, 488, 491, 494, 497, 500, 503, 506, 509, 512, 515, 518, 521, 524, 527, 530, 533, 536, 539, 542, 545, 548, 551, 554, 557, 560, 563, 566, 569, 572, 575 or 578.

In some embodiments, the CDR1-VH, the CDR2-VH and the CDR3-VH of the antibody or antigen-binding moiety of the present invention are selected from any sequence combination of the following VH1-VH102 or sequence combinations having 1, 2, 3 or more amino acid insertions, deletions and/or substitutions compared with the sequence combination, and preferably, the substitutions are conservative amino acid substitutions:

| | SEQ ID NO | | |
|---|---|---|---|
| No. | CDR1-VH | CDR2-VH | CDR3-VH |
| VH1 | 69 | 70 | 71 |
| VH2 | 72 | 73 | 74 |
| VH3 | 75 | 76 | 77 |
| VH4 | 78 | 79 | 80 |
| VH5 | 81 | 82 | 83 |
| VH6 | 84 | 85 | 86 |
| VH7 | 87 | 88 | 89 |
| VH8 | 90 | 91 | 92 |
| VH9 | 93 | 94 | 95 |
| VH10 | 96 | 97 | 98 |
| VH11 | 99 | 100 | 101 |
| VH12 | 102 | 103 | 104 |
| VH13 | 105 | 106 | 107 |
| VH14 | 108 | 109 | 110 |
| VH15 | 111 | 112 | 113 |
| VH16 | 114 | 115 | 116 |
| VH17 | 117 | 118 | 119 |
| VH18 | 120 | 121 | 122 |
| VH19 | 123 | 124 | 125 |
| VH20 | 126 | 127 | 128 |
| VH21 | 129 | 130 | 131 |
| VH22 | 132 | 133 | 134 |
| VH23 | 135 | 136 | 137 |
| VH24 | 138 | 139 | 140 |
| VH25 | 141 | 142 | 143 |
| VH26 | 144 | 145 | 146 |
| VH27 | 147 | 148 | 149 |
| VH28 | 150 | 151 | 152 |
| VH29 | 153 | 154 | 155 |
| VH30 | 156 | 157 | 158 |
| VH31 | 159 | 160 | 161 |
| VH32 | 162 | 163 | 164 |
| VH33 | 165 | 166 | 167 |
| VH34 | 168 | 169 | 170 |
| VH35 | 171 | 172 | 173 |
| VH36 | 174 | 175 | 176 |
| VH37 | 177 | 178 | 179 |
| VH38 | 180 | 181 | 182 |
| VH39 | 183 | 184 | 185 |
| VH40 | 186 | 187 | 188 |
| VH41 | 189 | 190 | 191 |
| VH42 | 192 | 193 | 194 |
| VH43 | 195 | 196 | 197 |
| VH44 | 198 | 199 | 200 |
| VH45 | 201 | 202 | 203 |
| VH46 | 204 | 205 | 206 |
| VH47 | 207 | 208 | 209 |
| VH48 | 210 | 211 | 212 |
| VH49 | 213 | 214 | 215 |
| VH50 | 216 | 217 | 218 |
| VH51 | 219 | 220 | 221 |
| VH52 | 222 | 223 | 224 |
| VH53 | 225 | 226 | 227 |
| VH54 | 228 | 229 | 230 |
| VH55 | 231 | 232 | 233 |
| VH56 | 234 | 235 | 236 |
| VH57 | 237 | 238 | 239 |
| VH58 | 240 | 241 | 242 |
| VH59 | 243 | 244 | 245 |
| VH60 | 246 | 247 | 248 |
| VH61 | 249 | 250 | 251 |
| VH62 | 252 | 253 | 254 |
| VH63 | 255 | 256 | 257 |
| VH64 | 258 | 259 | 260 |
| VH65 | 261 | 262 | 263 |
| VH66 | 264 | 265 | 266 |
| VH67 | 267 | 268 | 269 |
| VH68 | 270 | 271 | 272 |
| VH69 | 273 | 274 | 275 |
| VH70 | 276 | 277 | 278 |
| VH71 | 279 | 280 | 281 |
| VH72 | 282 | 283 | 284 |
| VH73 | 285 | 286 | 287 |
| VH74 | 288 | 289 | 290 |
| VH75 | 291 | 292 | 293 |
| VH76 | 294 | 295 | 296 |
| VH77 | 297 | 298 | 299 |
| VH78 | 300 | 301 | 302 |
| VH79 | 303 | 304 | 305 |
| VH80 | 306 | 307 | 308 |
| VH81 | 309 | 310 | 311 |
| VH82 | 312 | 313 | 314 |
| VH83 | 315 | 316 | 317 |
| VH84 | 318 | 319 | 320 |
| VH85 | 321 | 322 | 323 |
| VH86 | 324 | 325 | 326 |
| VH87 | 327 | 328 | 329 |
| VH88 | 330 | 331 | 332 |
| VH89 | 333 | 334 | 335 |
| VH90 | 336 | 337 | 338 |
| VH91 | 339 | 340 | 341 |
| VH92 | 342 | 343 | 344 |
| VH93 | 345 | 346 | 347 |
| VH94 | 348 | 349 | 350 |
| VH95 | 351 | 352 | 353 |
| VH96 | 354 | 355 | 356 |
| VH97 | 357 | 358 | 359 |
| VH98 | 360 | 361 | 362 |
| VH99 | 363 | 364 | 365 |
| VH100 | 366 | 367 | 368 |
| VH101 | 369 | 370 | 371 |
| VH102 | 372 | 373 | 374 |
| VH103 | 267 | 604 | 269 |

The CDR1-VL, the CDR2-VL and the CDR3-VL are selected from any sequence combination of the following VL1-VL68 or sequence combinations having 1, 2, 3 or more amino acid insertions, deletions and/or substitutions compared with the sequence combination, and preferably, the substitutions are conservative amino acid substitutions:

| | SEQ ID NO | | |
|---|---|---|---|
| No. | CDR1-VL | CDR2-VL | CDR3-VL |
| VL1 | 375 | 376 | 377 |
| VL2 | 378 | 379 | 380 |
| VL3 | 381 | 382 | 383 |
| VL4 | 384 | 385 | 386 |
| VL5 | 387 | 388 | 389 |
| VL6 | 390 | 391 | 392 |
| VL7 | 393 | 394 | 395 |
| VL8 | 396 | 397 | 398 |
| VL9 | 399 | 400 | 401 |
| VL10 | 402 | 403 | 404 |
| VL11 | 405 | 406 | 407 |
| VL12 | 408 | 409 | 410 |
| VL13 | 411 | 412 | 413 |
| VL14 | 414 | 415 | 416 |
| VL15 | 417 | 418 | 419 |
| VL16 | 420 | 421 | 422 |
| VL17 | 423 | 424 | 425 |
| VL18 | 426 | 427 | 428 |
| VL19 | 429 | 430 | 431 |
| VL20 | 432 | 433 | 434 |
| VL21 | 435 | 436 | 437 |
| VL22 | 438 | 439 | 440 |
| VL23 | 441 | 442 | 443 |
| VL24 | 444 | 445 | 446 |
| VL25 | 447 | 448 | 449 |
| VL26 | 450 | 451 | 452 |
| VL27 | 453 | 454 | 455 |
| VL28 | 456 | 457 | 458 |
| VL29 | 459 | 460 | 461 |
| VL30 | 462 | 463 | 464 |
| VL31 | 465 | 466 | 467 |
| VL32 | 468 | 469 | 470 |
| VL33 | 471 | 472 | 473 |
| VL34 | 474 | 475 | 476 |
| VL35 | 477 | 478 | 479 |
| VL36 | 480 | 481 | 482 |
| VL37 | 483 | 484 | 485 |
| VL38 | 486 | 487 | 488 |
| VL39 | 489 | 490 | 491 |
| VL40 | 492 | 493 | 494 |
| VL41 | 495 | 496 | 497 |
| VL42 | 498 | 499 | 500 |
| VL43 | 501 | 502 | 503 |
| VL44 | 504 | 505 | 506 |
| VL45 | 507 | 508 | 509 |
| VL46 | 510 | 511 | 512 |
| VL47 | 513 | 514 | 515 |
| VL48 | 516 | 517 | 518 |
| VL49 | 519 | 520 | 521 |
| VL50 | 522 | 523 | 524 |
| VL51 | 525 | 526 | 527 |
| VL52 | 528 | 529 | 530 |
| VL53 | 531 | 532 | 533 |
| VL54 | 534 | 535 | 536 |
| VL55 | 537 | 538 | 539 |
| VL56 | 540 | 541 | 542 |
| VL57 | 543 | 544 | 545 |
| VL58 | 546 | 547 | 548 |
| VL59 | 549 | 550 | 551 |
| VL60 | 552 | 553 | 554 |
| VL61 | 555 | 556 | 557 |
| VL62 | 558 | 559 | 560 |
| VL63 | 561 | 562 | 563 |
| VL64 | 564 | 565 | 566 |
| VL65 | 567 | 568 | 569 |
| VL66 | 570 | 571 | 572 |
| VL67 | 573 | 574 | 575 |
| VL68 | 576 | 577 | 578 |
| VL69 | 602 | 508 | 509 |
| VL70 | 603 | 508 | 509 |
| VL71 | 613 | 502 | 503 |
| VL72 | 614 | 502 | 503 |
| VL73 | 623 | 538 | 539 |
| VL74 | 624 | 538 | 539 |
| VL75 | 636 | 532 | 533 |
| VL76 | 637 | 532 | 533 |
| VL77 | 638 | 532 | 533 |
| VL78 | 639 | 532 | 533 |
| VL79 | 640 | 532 | 533 |
| VL80 | 652 | 544 | 545 |
| VL81 | 653 | 544 | 545 |
| VL82 | 654 | 544 | 545 |
| VL83 | 655 | 544 | 545 |
| VL84 | 656 | 544 | 545 |

In some embodiments, the antibody or antigen-binding moiety of the present invention comprises a combination of heavy chain CDRs and light chain CDRs selected from: VH1+VL1, VH2+VL1, VH3+VL2, VH4+VL3, VH5+VL3, VH6+VL4, VH7+VL5, VH8+VL5, VH9+VL6, VH10+VL7, VH11+VL7, VH12+VL8, VH13+VL9, VH14+VL9, VH15+VL10, VH16+VL11, VH17+VL11, VH18+VL12, VH19+VL13, VH20+VL13, VH21+VL14, VH22+VL15, VH23+VL15, VH24+VL16, VH25+VL17, VH26+VL17, VH27+VL18, VH28+VL19, VH29+VL19, VH30+VL20, VH31+VL21, VH32+VL21, VH33+VL22, VH34+VL23, VH35+VL23, VH36+VL24, VH37+VL25, VH38+VL25, VH39+VL26, VH40+VL27, VH41+VL27, VH42+VL28, VH43+VL29, VH44+VL29, VH45+VL30, VH46+VL31, VH47+VL31, VH48+VL32, VH49+VL33, VH50+VL33, VH51+VL34, VH52+VL35, VH53+VL35, VH54+VL36, VH55+VL37, VH56+VL37, VH57+VL38, VH58+VL39, VH59+VL39, VH60+VL40, VH61+VL41, VH62+VL41, VH63+VL42, VH64+VL43, VH65+VL43, VH66+VL44, VH67+VL45, VH68+VL45, VH69+VL46, VH70+VL47, VH71+VL47, VH72+VL48, VH73+VL49, VH74+VL49, VH75+VL50, VH76+VL51, VH77+VL51, VH78+VL52, VH79+VL53, VH80+VL53, VH81+VL54, VH82+VL55, VH83+VL55, VH84+VL56, VH85+VL57, VH86+VL57, VH87+VL58, VH88+VL59, VH89+VL59, VH90+VL60, VH91+VL61, VH92+VL61, VH93+VL62, VH94+VL63, VH95+VL63, VH96+VL64, VH97+VL65, VH98+VL65, VH99+VL66, VH100+VL67, VH101+VL67, VH102+VL68, VH103+VL45, VH103+VL69, VH103+VL70, VH67+VL69, VH67+VL70, VH64+VL71, VH64+VL72, VH82+VL73, VH82+VL74, VH79+VL75, VH79+VL76, VH79+VL77, VH79+VL78, VH79+VL79, VH85+VL80, VH85+VL81, VH85+VL82, VH85+VL83, VH85+VL84, and a combination of CDRs having 1, 2, 3 or more amino acid insertions, deletions and/or substitutions compared with a sequence of the combination of heavy chain CDRs and light chain CDRs, and preferably, the substitutions are conservative amino acid substitutions.

In some embodiments, the antibody or antigen-binding moiety of the present invention comprises a sequence having at least 80%, 85%, 90%, 91%, 92%, 93%, 94%, 95%, 96%, 97%, 98%, 99% or 100% identity to the CDR1, the CDR2, and/or the CDR3, respectively. In some embodiments, the antibody or antigen-binding moiety of the present invention comprises: (1) a heavy chain variable region set forth in any one of SEQ ID NOs: 1-34, 594, 599-601, 605, 610-612, 615, 620-622, 625, 633-635, 641, and 649-651; or a sequence having at least 80%, 85%, 90%, 91%, 92%, 93%, 94%, 95%, 96%, 97%, 98%, 99% or 100% identity to the sequence set forth in any one of SEQ ID NOs: 1-34, 594, 599-601, 605, 610-612, 615, 620-622, 625, 633-635, 641, and 649-651; or a sequence having at most 20, 19, 18, 17, 16, 15, 14, 13, 12, 11, 10, 9, 8, 7, 6, 5, 4, 3, 2 or 1 mutation compared with the sequence set forth in any one of SEQ ID NOs: 1-34, 594, 599-601, 605, 610-612, 615, 620-622, 625, 633-635, 641, and 649-651; and the mutation may be selected from an insertion, a deletion and/or a substitution, and preferably, the substitution is a conservative amino acid substitution; and/or
(2) a light chain variable region set forth in any one of SEQ ID NOs: 35-68, 595-597, 606-609, 616-619, 626-632, and 642-648; or a sequence having at least 80%, 85%, 90%, 91%, 92%, 93%, 94%, 95%, 96%, 97%, 98%, 99% or 100% identity to the sequence set forth in any one of SEQ ID NOs: 35-68, 595-597, 606-609, 616-619, 626-632, and 642-648; or a sequence having at most 20, 19, 18, 17, 16, 15, 14, 13, 12, 11, 10, 9, 8, 7, 6, 5, 4, 3, 2 or 1 mutation compared with the sequence set forth in any one of SEQ ID NOs: 35-68, 595-597, 606-609, 616-619, 626-632, and 642-648; and the mutation may be selected from an insertion, a deletion and/or a substitution, and preferably, the substitution is a conservative amino acid substitution.

In some embodiments, the antibody or antigen-binding moiety of the present invention comprises:
(1) the heavy chain variable region and the light chain variable region have sequences set forth in SEQ ID NO: 1 and SEQ ID NO: 35, respectively;
(2) the heavy chain variable region and the light chain variable region have sequences set forth in SEQ ID NO: 2 and SEQ ID NO: 36, respectively;
(3) the heavy chain variable region and the light chain variable region have sequences set forth in SEQ ID NO: 3 and SEQ ID NO: 37, respectively;
(4) the heavy chain variable region and the light chain variable region have sequences set forth in SEQ ID NO: 4 and SEQ ID NO: 38, respectively;
(5) the heavy chain variable region and the light chain variable region have sequences set forth in SEQ ID NO: 5 and SEQ ID NO: 39, respectively;
(6) the heavy chain variable region and the light chain variable region have sequences set forth in SEQ ID NO: 6 and SEQ ID NO: 40, respectively;
(7) the heavy chain variable region and the light chain variable region have sequences set forth in SEQ ID NO: 7 and SEQ ID NO: 41, respectively;
(8) the heavy chain variable region and the light chain variable region have sequences set forth in SEQ ID NO: 8 and SEQ ID NO: 42, respectively;
(9) the heavy chain variable region and the light chain variable region have sequences set forth in SEQ ID NO: 9 and SEQ ID NO: 43, respectively;
(10) the heavy chain variable region and the light chain variable region have sequences set forth in SEQ ID NO: 10 and SEQ ID NO: 44, respectively;
(11) the heavy chain variable region and the light chain variable region have sequences set forth in SEQ ID NO: 11 and SEQ ID NO: 45, respectively;
(12) the heavy chain variable region and the light chain variable region have sequences set forth in SEQ ID NO: 12 and SEQ ID NO: 46, respectively;
(13) the heavy chain variable region and the light chain variable region have sequences set forth in SEQ ID NO: 13 and SEQ ID NO: 47, respectively;
(14) the heavy chain variable region and the light chain variable region have sequences set forth in SEQ ID NO: 14 and SEQ ID NO: 48, respectively;
(15) the heavy chain variable region and the light chain variable region have sequences set forth in SEQ ID NO: 15 and SEQ ID NO: 49, respectively;
(16) the heavy chain variable region and the light chain variable region have sequences set forth in SEQ ID NO: 16 and SEQ ID NO: 50, respectively;
(17) the heavy chain variable region and the light chain variable region have sequences set forth in SEQ ID NO: 17 and SEQ ID NO: 51, respectively;
(18) the heavy chain variable region and the light chain variable region have sequences set forth in SEQ ID NO: 18 and SEQ ID NO: 52, respectively;
(19) the heavy chain variable region and the light chain variable region have sequences set forth in SEQ ID NO: 19 and SEQ ID NO: 53, respectively;
(20) the heavy chain variable region and the light chain variable region have sequences set forth in SEQ ID NO: 20 and SEQ ID NO: 54, respectively;
(21) the heavy chain variable region and the light chain variable region have sequences set forth in SEQ ID NO: 21 and SEQ ID NO: 55, respectively;
(22) the heavy chain variable region and the light chain variable region have sequences set forth in SEQ ID NO: 22 and SEQ ID NO: 56, respectively;
(23) the heavy chain variable region and the light chain variable region have sequences set forth in SEQ ID NO: 23 and SEQ ID NO: 57, respectively;
(24) the heavy chain variable region and the light chain variable region have sequences set forth in SEQ ID NO: 24 and SEQ ID NO: 58, respectively;
(25) the heavy chain variable region and the light chain variable region have sequences set forth in SEQ ID NO: 25 and SEQ ID NO: 59, respectively;
(26) the heavy chain variable region and the light chain variable region have sequences set forth in SEQ ID NO: 26 and SEQ ID NO: 60, respectively;
(27) the heavy chain variable region and the light chain variable region have sequences set forth in SEQ ID NO: 27 and SEQ ID NO: 61, respectively;
(28) the heavy chain variable region and the light chain variable region have sequences set forth in SEQ ID NO: 28 and SEQ ID NO: 62, respectively;
(29) the heavy chain variable region and the light chain variable region have sequences set forth in SEQ ID NO: 29 and SEQ ID NO: 63, respectively;
(30) the heavy chain variable region and the light chain variable region have sequences set forth in SEQ ID NO: 30 and SEQ ID NO: 64, respectively;
(31) the heavy chain variable region and the light chain variable region have sequences set forth in SEQ ID NO: 31 and SEQ ID NO: 65, respectively;
(32) the heavy chain variable region and the light chain variable region have sequences set forth in SEQ ID NO: 32 and SEQ ID NO: 66, respectively;
(33) the heavy chain variable region and the light chain variable region have sequences set forth in SEQ ID NO: 33 and SEQ ID NO: 67, respectively;
(34) the heavy chain variable region and the light chain variable region have sequences set forth in SEQ ID NO: 34 and SEQ ID NO: 68, respectively;
(35) the heavy chain variable region and the light chain variable region have sequences set forth in SEQ ID NO: 594 and SEQ ID NO: 595, respectively;
(36) the heavy chain variable region and the light chain variable region have sequences set forth in SEQ ID NO: 599 and SEQ ID NOs: 596-598, respectively;
(37) the heavy chain variable region and the light chain variable region have sequences set forth in SEQ ID NO: 600 and SEQ ID NOs: 596-598, respectively;
(38) the heavy chain variable region and the light chain variable region have sequences set forth in SEQ ID NO: 601 and SEQ ID NOs: 596-598, respectively;
(39) the heavy chain variable region and the light chain variable region have sequences set forth in SEQ ID NO: 605 and SEQ ID NO: 606, respectively;
(40) the heavy chain variable region and the light chain variable region have sequences set forth in SEQ ID NO: 610 and SEQ ID NOs: 607-609, respectively;
(41) the heavy chain variable region and the light chain variable region have sequences set forth in SEQ ID NO: 611 and SEQ ID NOs: 607-609, respectively;
(42) the heavy chain variable region and the light chain variable region have sequences set forth in SEQ ID NO: 612 and SEQ ID NOs: 607-609, respectively;
(43) the heavy chain variable region and the light chain variable region have sequences set forth in SEQ ID NO: 615 and SEQ ID NO: 616, respectively;
(44) the heavy chain variable region and the light chain variable region have sequences set forth in SEQ ID NO: 620 and SEQ ID NOs: 617-619, respectively;
(45) the heavy chain variable region and the light chain variable region have sequences set forth in SEQ ID NO: 621 and SEQ ID NOs: 617-619, respectively;
(46) the heavy chain variable region and the light chain variable region have sequences set forth in SEQ ID NO: 622 and SEQ ID NOs: 617-619, respectively;
(47) the heavy chain variable region and the light chain variable region have sequences set forth in SEQ ID NO: 625 and SEQ ID NO: 626, respectively;
(48) the heavy chain variable region and the light chain variable region have sequences set forth in SEQ ID NO: 633 and SEQ ID NOs: 627-629, respectively;
(49) the heavy chain variable region and the light chain variable region have sequences set forth in SEQ ID NO: 634 and SEQ ID NOs: 627-629, respectively;
(50) the heavy chain variable region and the light chain variable region have sequences set forth in SEQ ID NO: 635 and SEQ ID NOs: 627-632, respectively;
(51) the heavy chain variable region and the light chain variable region have sequences set forth in SEQ ID NO: 641 and SEQ ID NO: 642, respectively;
(52) the heavy chain variable region and the light chain variable region have sequences set forth in SEQ ID NO: 649 and SEQ ID NOs: 643-648, respectively;
(53) the heavy chain variable region and the light chain variable region have sequences set forth in SEQ ID NO: 650 and SEQ ID NOs: 643-645, respectively;
(54) the heavy chain variable region and the light chain variable region have sequences set forth in SEQ ID NO: 651 and SEQ ID NOs: 643-645, respectively;
(55) the heavy chain variable region and the light chain variable region have sequences having at least 70%, 75%, 80%, 85%, 90%, 95%, 96%, 97%, 98%, 99% or more identity to the sequences set forth in (1) to (54) above, respectively.

In some embodiments, the antibody or antigen-binding moiety of the present invention specifically binds to a human, monkey, and/or murine GPC3 protein; and preferably, the antibody or antigen-binding moiety binds to human, monkey, and/or murine GPC3 with a dissociation constant (KD) not greater than 1.00E-7 M, 1.00E-8 M, 2.00E-8 M, 3.00E-8 M, 4.00E-8 M, 5.00E-8 M, 6.00E-8 M, 7.00E-8 M, 8.00E-8 M, 9.00E-8 M, 1.00E-9 M, 2.00E-9 M, 3.00E-9 M, 4.00E-9 M, 5.00E-9 M, 6.00E-9 M, 7.00E-9 M, 8.00E-9 M, 9.00E-9 M or 1.00E-10 M.

In some embodiments, the antibody or antigen-binding moiety of the present invention is selected from full-length antibodies, VH single domain antibodies, Fab fragments, Fab′ fragments, F(ab)′2 fragments, Fd fragments, Fv fragments, complementarity determining region (CDR) fragments, single chain variable fragments (scFv), scFv2, disulfide stabilized variable fragments (dsFv), domain antibodies, bivalent single chain antibodies, single chain phage antibodies, bispecific diabodies, triabodies, tetrabodies, and minimal recognition units of antibodies.

In some embodiments, the antibody or antigen-binding moiety of the present invention is a murine antibody, a humanized antibody, a fully human antibody, or a chimeric antibody. In some embodiments, the antibody or antigen-binding moiety of the present invention is capable of specifically binding to a peptide having a sequence of amino acid residues 524-563 of glypican-3.

According to another aspect, the present invention provides an immunoconjugate comprising any one of the foregoing antibodies or antigen-binding moieties and an effector molecule; and preferably, the effector molecule is linked to the antibody or antigen-binding moiety.

In some embodiments, the effector molecule comprises a therapeutic agent or a marker; preferably, the therapeutic agent is selected from a drug, a toxin, a radioisotope, a chemotherapeutic drug, and an immunomodulator, and the marker is selected from an isotope, a fluorescent compound, a chemiluminescent compound, an enzyme, a metal ion, a radiocontrast medium, a paramagnetic ion, an ultrasound contrast agent, and a photosensitizer; and more preferably, the drug is vinblastine or daunomycin, and the toxin is Pseudomonas exotoxin, diphtheria toxin, alkaloids, methotrexate, anthracyclines (doxorubicin), taxanes, or a toxin compound.

In some embodiments, the immunoconjugate further comprises a linker for conjugating the effector molecule to the antibody or antigen-binding moiety, and the linker includes but is not limited to hydrazones, thioethers, esters, disulfides, and peptide-containing linkers. According to another aspect, the present invention provides a chimeric antigen receptor (CAR) comprising an extracellular antigen-binding domain, a transmembrane domain, and an intracellular signaling domain, and the extracellular antigen-binding domain comprises the antibody or antigen-binding moiety according to any one of the foregoing implementations.

According to another aspect, the present invention provides an immunocompetent cell, the immunocompetent cell expresses any one of the foregoing chimeric antigen receptors or comprises a nucleic acid molecule encoding any one of the foregoing chimeric antigen receptors; and preferably, the immunocompetent cell is selected from: a T cell, a natural killer cell (NK cell), a natural killer T cell (NKT cell), a double negative T cell (DNT cell), a monocyte, a macrophage, a dendritic cell, and a mast cell, and the T cell is preferably selected from a cytotoxic T cell, a regulatory T cell, and a helper T cell.

According to another aspect, the present invention provides a multispecific molecule comprising any one of the foregoing antibodies or antigen-binding moieties; and preferably, the multispecific molecule further comprises an antibody or antigen-binding moiety specifically binding to an antigen other than GPC3 or binding to a GPC3 epitope different from any one of the foregoing antibodies or antigen-binding fragments.

In some embodiments, the antigen other than GPC3 is an antigen on surface of a T cell, a B cell, a natural killer cell, a dendritic cell, a macrophage, a monocyte, or a neutrophil; and preferably, the antigen other than GPC3 is selected from: CD3, CD3γ, CD3δ, CD3ε, CD3ζ, CD16, CD16A, CD32B, PD-1, PD-2, PD-L1, VEGF, NKG2D, CD19, CD20, CD40, CD47, 4-1BB, CD137, EGFR, EGFRvIII, TNF-alpha, CD33, HER2, HER3, HAS, CD5, CD27, EphA2, EpCAM, MUC1, MUC16, CEA, Claudin18.2, a folate receptor, Claudin6, WT1, NY-ESO-1, MAGE3, ASGPR1, and CDH16.

In some embodiments, the multispecific molecule is a tandem scFv, a bifunctional antibody (Db), a single chain bifunctional antibody (scDb), a dual affinity retargeting (DART) antibody, F(ab′)2, a dual variable domain (DVD) antibody, a knobs-into-holes (KiH) antibody, a dock-and-lock (DNL) antibody, a chemically cross-linked antibody, a heteropolyantibody or a heteroconjugate antibody.

According to another aspect, the present invention provides an isolated nucleic acid molecule encoding any one of the foregoing antibodies or antigen-binding moieties, any one of the foregoing chimeric antigen receptors, and any one of the foregoing multispecific molecules.

According to another aspect, the present invention provides a vector comprising the nucleic acid molecule.

According to another aspect, the present invention provides a host cell comprising the nucleic acid molecule or the expression vector, and preferably, the host cell is a prokaryotic cell or a eukaryotic cell, comprising a bacteria (E. coli), a fungus (yeast), an insect cell, or a mammalian cell (CHO cell line or 293T cell line).

According to another aspect, the present invention provides a method for preparing any one of the foregoing antibodies or antigen-binding moieties, or multispecific molecules, comprising: culturing the host cell, and isolating an antibody or antigen-binding moiety expressed by the cell, or isolating a multispecific molecule expressed by the cell.

According to another aspect, the present invention provides a method for preparing the immunocompetent cell, comprising: introducing a nucleic acid fragment encoding any one of the foregoing chimeric antigen receptors into the immunocompetent cell, and optionally, the method further comprises initiating expression of any one of the foregoing chimeric antigen receptors by the immunocompetent cell.

According to another aspect, the present invention provides a pharmaceutical composition comprising a therapeutically effective amount of one or a combination of: any one of the foregoing antibodies or antigen-binding moieties; any one of the foregoing immunoconjugates; any one of the foregoing immunocompetent cells; any one of the foregoing multispecific molecules; any one of the foregoing nucleic acid molecules, expression vectors or host cells; or the product prepared by any one of the foregoing methods, and a pharmaceutically acceptable carrier.

According to another aspect, the present invention further discloses use of any one of the foregoing antibodies or antigen-binding moieties, the immunoconjugate, the immunocompetent cell, the multispecific molecule, the nucleic acid molecule, the expression vector, the product prepared by the method, or the pharmaceutical composition in preparation of a drug for treatment of a GPC3-mediated tumor; wherein the tumor is preferably selected from diseases such as hepatocellular carcinoma, melanoma, ovarian clear cell carcinoma, hepatoblastoma, neuroblastoma, Wilms' tumor, small cell lung cancer, lung adenocarcinoma, gastric cancer, colon cancer, rectal cancer, cervical cancer, breast cancer, ovarian cancer, skin cancer, lymphoma, prostate cancer, pancreatic cancer, renal cancer, esophageal cancer, thyroid cancer, testicular cancer, bladder cancer, bronchogenic carcinoma, nasopharyngeal cancer, head and neck cancer, endometrial cancer, brain cancer, bone cancer, leukemia, malignant mesothelioma, and liposarcoma; and preferably, hepatocellular carcinoma.

According to another aspect, the present invention further provides a method for treating a subject suffering from a GPC3-mediated tumor, comprising: selecting a subject suffering from a cancer expressing GPC3, and administering to the subject a therapeutically effective amount of any one of the foregoing antibodies or antigen-binding moieties, the immunoconjugate, the immunocompetent cell, the multispecific molecule, the nucleic acid molecule, the expression vector, the product prepared by the method, or the pharmaceutical composition; wherein the tumor is preferably selected from diseases such as hepatocellular carcinoma, melanoma, ovarian clear cell carcinoma, hepatoblastoma, neuroblastoma, Wilms' tumor, small cell lung cancer, lung adenocarcinoma, gastric cancer, colon cancer, rectal cancer, cervical cancer, breast cancer, ovarian cancer, skin cancer, lymphoma, prostate cancer, pancreatic cancer, renal cancer, esophageal cancer, thyroid cancer, testicular cancer, bladder cancer, bronchogenic carcinoma, nasopharyngeal cancer, head and neck cancer, endometrial cancer, brain cancer, bone cancer, leukemia, malignant mesothelioma, and liposarcoma; and preferably, hepatocellular carcinoma.

According to another aspect, the present invention further provides use of any one of the foregoing antibodies or antigen-binding moieties, the immunoconjugate, the immunocompetent cell, the multispecific molecule, the nucleic acid molecule, the expression vector, the product prepared by the method, or the pharmaceutical composition in treatment of a GPC3-positive tumor or cancer; wherein the tumor is preferably selected from diseases such as hepatocellular carcinoma, melanoma, ovarian clear cell carcinoma, hepatoblastoma, neuroblastoma, Wilms' tumor, small cell lung cancer, lung adenocarcinoma, gastric cancer, colon cancer, rectal cancer, cervical cancer, breast cancer, ovarian cancer, skin cancer, lymphoma, prostate cancer, pancreatic cancer, renal cancer, esophageal cancer, thyroid cancer, testicular cancer, bladder cancer, bronchogenic carcinoma, nasopharyngeal cancer, head and neck cancer, endometrial cancer, brain cancer, bone cancer, leukemia, malignant mesothelioma, and liposarcoma; and preferably, hepatocellular carcinoma.

According to another aspect, the present invention further provides a kit, comprising any one of the foregoing antibodies or antigen-binding moieties, any one of the foregoing immunoconjugates, the foregoing immunocompetent cell, any one of the foregoing multispecific molecules, the foregoing nucleic acid molecule, the foregoing expression vector, the product prepared by any one of the foregoing methods, or the pharmaceutical composition.

According to another aspect, the present invention further provides use of any one of the foregoing antibodies or antigen-binding moieties in preparation of a reagent for detection or diagnosis of a tumor with a high expression of GPC3.

According to another aspect, the present invention further provides a method for detecting GPC3 expression in a biological sample, comprising: exposing a sample from a subject to any one of the foregoing antibodies or antigen-binding moieties, and detecting binding of the antibody or antigen-binding moiety to the sample.

### TERMINOLOGY AND DEFINITIONS

To make the present invention easier to understand, the selected terms are defined below.

The term “antibody” used herein generally refers to all antigen compound binding fragments or proteins containing antigen compound binding fragments, including polyclonal antibodies and monoclonal antibodies, as well as antigen compound binding fragments of such antibodies. Examples of such antibodies include full-length antibodies, VH single domain antibodies, Fab fragments, Fab′ fragments, F(ab)′2 fragments, Fd fragments, Fv fragments, complementarity determining region (CDR) fragments, single chain variable fragments (scFv), scFv2, disulfide stabilized variable fragments (dsFv), domain antibodies, bivalent single chain antibodies, single chain phage antibodies, bispecific diabodies, triabodies, tetrabodies, or minimal recognition units of antibodies. The type of the antibody can be selected from IgG1, IgG2, IgG3, IgG4, IgA, IgM, IgE and IgD; the same class of Ig can be divided into different subclasses according to differences in amino acid composition of hinge regions and differences in quantity and positions of heavy chain disulfide bonds, for example, IgG can be divided into IgG1, IgG2, IgG3 and IgG4, and IgA can be divided into IgA1 and IgA2. Light chains are divided into κ or λ chains according to differences in the constant regions. Each of the five classes of Ig may have a κ chain or a λ chain. In addition, “antibodies” include naturally occurring antibodies and non-naturally occurring antibodies, including, for example, chimeric antibodies, bifunctional antibodies, humanized antibodies, fully human antibodies, and related synthetic isoforms.

“Antibody” herein may be derived from any animal, including but not limited to human and non-human animals which may be selected from primates, mammals, rodents, and vertebrates, such as Camelidae species, Lama glama, Lama guanicoe, Vicugna pacos, sheep, rabbits, mice, rats, or Chondrichthyes (e.g., shark).

An antibody includes a glycoprotein or an antigen-binding moiety thereof having at least two heavy (H) chains and two light (L) chains that are linked to each other by disulfide bonds. Each heavy chain includes a heavy chain variable region (VH) and a heavy chain constant region (CH). The heavy chain constant region includes three domains: CH1, CH2, and CH3. Each light chain includes a light chain variable region (VL) and a light chain constant region. The light chain constant region includes a domain, i.e., CL. The VH region and the VL region can be further subdivided into hypervariable regions called complementarity determining regions (CDRs), and the CDRs partition the variable regions into framework regions (FRs). Each VH and VL include three CDRs and four FRs, which are arranged from an N-terminus to a C-terminus in the following order: FR1, CDR1, FR2, CDR2, FR3, CDR3, and FR4. These CDRs form a circular structure and are spatially close to each other through a β-sheet formed by the FRs therebetween, and CDRs on the heavy chain and CDRs on the corresponding light chain constitute an antigen-binding site of the antibody, while amino acid sequences of the FRs are relatively conserved and are not directly involved in a binding reaction. Constant regions of the antibody may mediate binding of immunoglobulin to a host tissue or factor, and the host tissue or factor includes various cells (e.g., effector cells) of an immune system and a first component (C1q) of a classical complement system.

The term “CDR” used herein may be labeled and defined in a way well known in the art, including but not limited to, the Kabat numbering scheme, the Chothia numbering scheme, or the IMGT numbering scheme: the tool sites used include but not limited to, AbRSA site (http://cao.labshare.cn/AbRSA/cdrs.php), abYsis site (www.abysis.org/abysis/sequence_input/key_annotation/key_annotation.cgi), and IMGT site (http://www.imgt.org/3Dstructure-DB/cgi/DomainGapAlign.cgi#results). The CDR herein includes overlaps and subsets of amino acid residues defined in different ways.

The term “Kabat numbering scheme” used herein generally refers to the immunoglobulin alignment and numbering scheme proposed by Elvin A. Kabat (see, e.g., Kabat et al., Sequences of Proteins of Immunological Interest, 5th Ed. Public Health Service, National Institutes of Health, Bethesda, Md., 1991).

The term “Chothia numbering scheme” used herein generally refers to the immunoglobulin numbering scheme proposed by Chothia et al., which is a classical rule for identifying CDR region boundaries based on the position of structural loop regions (see, e.g., Chothia & Lesk (1987) J. Mol. Biol. 196:901-917; Chothia et al., (1989) Nature 342:878-883).

The term “IMGT numbering scheme” used herein generally refers to a numbering scheme based on the international ImMunoGeneTics information system (IMGT) initiated by Lefranc et al., see Lefranc et al., Dev. Comparat. Immunol. 27:55-77, 2003.

The term “antigen-binding moiety” used herein generally refers to all proteins/protein fragments including CDRs. Such fragments have a length, for example, between about 8 and about 1500 amino acids, properly between about 8 and about 745 amino acids, properly about 8 to about 300 amino acids, for example, about 8 to about 200 amino acids, or about 10 to about 50 or 100 amino acids. It has been shown that an antigen-binding function of an antibody can be performed by fragments of a full-length antibody.

The term “monoclonal antibody” used herein refers to an antibody obtained from a substantially homogeneous population in which individual antibodies are the same except for a few possible naturally occurring mutations. The modifier “monoclonal” indicates only a property of an antibody and is derived from a substantially homogeneous population of antibodies, which is not to be construed as requiring any particular method for producing the antibody.

The term “chimeric antibody” used herein refers to an antibody having framework residues from one species (such as human) and CDRs (which generally attribute antigen binding) from another species.

The term “fully human antibody” used herein refers to an antibody in which framework regions and CDRs of variable regions are derived from human germline immunoglobulin sequences. Furthermore, if the antibody contains constant regions, the constant regions are also derived from human germline immunoglobulin sequences.

The term “humanized antibody” used herein refers to an antibody in which CDR sequences derived from the germline of another mammalian species (such as mice) have been grafted onto human framework sequences. Other modifications to framework regions can be made within the human framework sequences.

The term “epitope” used herein refers to an antigenic determinant. These are specific chemical groups or peptide sequences on molecules that are antigenic (i.e., induce a specific immune response). The antibody specifically binds to a specific epitope on a polypeptide (e.g., GPC3).

The term “isolated” used herein means that a protein, polypeptide, or nucleic acid is not in its natural medium or in its natural form. Therefore, the term “isolated” refers to a molecule that is substantially free from its natural environment. For example, an isolated protein is substantially free of cellular materials or other proteins from a cell or tissue source from which it was derived. The term “isolated” also refers to a formulation in which an isolated protein is pure enough to be administered as a pharmaceutical composition, or at least 70-80% (w/w) pure, more preferably at least 80-90% (w/w) pure, even more preferably 90-95% pure, and most preferably at least 95%, 96%, 97%, 98%, 99% or 100% (w/w) pure. When associated with a nucleic acid, the term “isolated” or “purified” indicates, for example, that the nucleic acid is not in its natural genomic context (e.g., in a vector, as an expression cassette, linked to a promoter, or artificially introduced into a heterologous host cell).

The term “specific binding” used herein means that an antigen-binding molecule (e.g., an antibody) specifically binds to an antigen and substantially identical antigens, generally with high affinity, but does not bind with high affinity to unrelated antigens. Affinity is generally reflected in an equilibrium dissociation constant (KD), where a low KD indicates a high affinity.

The term “Ka” used herein refers to an association rate of a particular antibody-antigen interaction, while the term “Kd” used herein refers to a dissociation rate of a particular antibody-antigen interaction. The term “KD” used herein refers to a dissociation constant, which is obtained from a ratio of KD to Ka (i.e., KD/Ka) and is expressed as a molar concentration (M). A KD value of an antibody can be determined by using a method established in the art. A preferred method for determining KD of an antibody is surface plasmon resonance (e.g., Biacore), preferably a biosensor system.

The term “high affinity” used herein refers to specific binding of an antibody to a target protein, e.g., a human, monkey, and/or murine GPC3 protein, and the antibody specifically binds to human, monkey, and/or murine GPC3 with a dissociation constant (KD) not greater than 1.00E-7 M, 1.00E-8 M, 2.00E-8 M, 3.00E-8 M, 4.00E-8 M, 5.00E-8 M, 6.00E-8 M, 7.00E-8 M, 8.00E-8 M, 9.00E-8 M, 1.00E-9 M, 2.00E-9 M, 3.00E-9 M, 4.00E-9 M, 5.00E-9 M, 6.00E-9 M, 7.00E-9 M, 8.00E-9 M, 9.00E-9 M or 1.00E-10 M.

The term “conservative amino acid” used herein generally refers to amino acids that belong to the same class or have similar characteristics (e.g., charge, side chain size, hydrophobicity, hydrophilicity, backbone conformation, and rigidity). For example, the amino acids in each of the following groups are conservative amino acid residues of each other, and substitutions of amino acid residues within the groups are conservative amino acid substitutions:
(1) acidic amino acids: Asp (D) and Glu (E);
(2) basic amino acids: Lys (K), Arg (R), and His (H);
(3) hydrophilic uncharged amino acids: Ser(S), Thr (T), Asn (N), and Gln (Q);
(4) aliphatic uncharged amino acids: Gly (G), Ala (A), Val (V), Leu (L), and Ile (I);
(5) non-polar uncharged amino acids: Cys (C), Met (M), and Pro (P); and
(6) aromatic amino acids: Phe (F), Tyr (Y), and Trp (W).

The terms “identity” and “sequence . . . identity” used herein are interchangeable and are obtained by calculating as follows: to determine the percent “identity” of two amino acid sequences or two nucleic acid sequences, the sequences are aligned for optimal comparison purposes (e.g., for optimal alignment, gaps can be introduced in one or both of the first and second amino acid sequences or nucleic acid sequences, or non-homologous sequences can be discarded for comparison). Amino acid residues or nucleotides at corresponding amino acid positions or nucleotide positions are then compared. When a position in the first sequence is occupied by the same amino acid residue or nucleotide at the corresponding position in the second sequence, the molecules are identical at this position.

The term “immunoconjugate” used herein refers to a polypeptide molecule containing at least one effector molecule and at least one antibody or a functional fragment thereof.

The term “effector molecule” used herein is a part of an immunoconjugate, which is intended to have a desired effect on cells targeted by the immunoconjugate. An effector molecule is also referred to as an effector moiety (EM), a therapeutic agent, a diagnostic agent, a tracer, or a similar term.

The term “chimeric antigen receptor (CAR)” used herein refers to an artificial cell surface receptor engineered to be expressed on an immunocompetent cell and specifically binds to an antigen, which comprises at least (1) an extracellular antigen-binding domain, e.g., a variable heavy chain or light chain of an antibody, (2) a transmembrane domain that anchors the CAR into the immunocompetent cell, and (3) an intracellular signaling domain. The CAR is capable of redirecting T cells and other immunocompetent cells to a selected target, e.g., a cancer cell, in a non-MHC-restricted manner using the extracellular antigen-binding domain.

The term “multispecific molecule” used herein refers to a molecule having at least two antigen-binding sites, each of the at least two antigen-binding sites binds to a different epitope of the same antigen or a different epitope of a different antigen. Thus, the terms such as “bispecific”, “trispecific”, and “tetraspecific” refer to the number of different epitopes to which an antibody/antigen-binding molecule can bind.

The term “immunocompetent cell” used herein refers to a cell that performs immune functions in an organism. Examples of immunocompetent cells include: lymphocyte cells such as T cells, natural killer cells (NK cells), and B cells: antigen-presenting cells such as monocytes, macrophages, and dendritic cells; and granulocytes such as neutrophils, eosinophils, basophils, and mast cells. Specifically, T cells or NK cells from mammals such as human, dogs, cats, pigs, and mice are preferably enumerated, and preferably T cells or NK cells from human. In addition, T cells can be isolated and purified from body fluids such as blood and marrow fluids, from tissues such as spleen, thymus, and lymph nodes, or from immunocompetent cells infiltrating cancer tissues such as primary tumors, metastatic tumors, and malignant ascites. T cells made from ES cells and iPS cells can also be used. Examples of the T cells include α-β T cells, γ-δ T cells, CD8⁺ T cells, CD4⁺ T cells, tumor infiltrating T cells, memory T cells, naive T cells, and NKT cells. It should be noted that a source of immunocompetent cells and a subject to be administered may be the same or different. Then, when the subject to be administered is human, autologous cells derived from a patient as the subject to be administered, or allogeneic cells derived from another person may be used as the immunocompetent cells. That is, the donor and the receptor may or may not be identical, and preferably are identical.

The term “vector” used herein refers to a nucleic acid molecule that is introduced into a host cell to produce a transformed host cell. The vector may contain a nucleic acid sequence (e.g., an origin of replication) that allows replication in the host cell. The vector may also contain one or more selectable marker genes and other genetic elements known in the art.

The term “host cell” used herein refers to a cell in which a vector can proliferate and its DNA can be expressed, and the cell may be a prokaryotic cell or a eukaryotic cell. The term also includes any progeny of the subject host cell. It should be understood that not all progeny are identical to a parent cell, because mutations may occur during replication, such progeny are included.

The term “pharmaceutically acceptable carrier” used herein includes any and all physiologically compatible solvents, dispersion media, coatings, antibacterial agents, antifungal agents, isotonic agents, absorption retardants, and the like. Generally, the nature of the carrier depends on a specific method of administration used. For example, a parenteral formulation typically contains an injectable fluid as a vehicle, and the injectable fluid contains a pharmaceutically and physiologically acceptable fluid such as water, physiological saline, a balanced salt solution, aqueous dextrose, and glycerol. For solid compositions (e.g., in powder, pill, tablet, or capsule forms), conventional non-toxic solid carriers may include, for example, pharmaceutical mannitol, lactose, starch, or magnesium stearate. In addition to biologically neutral carriers, the pharmaceutical composition to be administered may also contain small amounts of non-toxic auxiliary substances such as a wetting agent or an emulsifier, a preservative, and a pH buffer, for example, sodium acetate or dehydrated sorbitan monolaurate.

The term “therapeutically effective amount” used herein refers to an amount of the anti-GPC3 antibody or composition as disclosed herein that is effective in “treating” a disease or disorder of an individual. In the case of a cancer, a therapeutically effective amount of the anti-GPC3 antibody or composition as disclosed herein can reduce the number of cancer cells: reduce tumor size or weight: inhibit (i.e., slow down to some extent and preferably prevent) infiltration of cancer cells into surrounding organs: inhibit (i.e., slow down to some extent and preferably prevent) tumor metastasis: inhibit tumor growth to some extent; and/or alleviate, to some extent, one or more symptoms associated with the cancer. To the extent that the anti-GPC3 antibody or composition as disclosed herein can prevent growth and/or kill existing cancer cells, the anti-GPC3 antibody or composition may be cytostatic and/or cytotoxic. In some embodiments, the therapeutically effective amount is a growth inhibiting amount. In some embodiments, the therapeutically effective amount is an amount that prolongs survival of a patient. In some embodiments, the therapeutically effective amount is an amount that improves progression-free survival of a patient.

The term “treatment” used herein refers to a method for obtaining beneficial or desired outcomes (including clinical outcomes). For purposes of the present invention, beneficial or desired clinical outcomes include (but are not limited to) one or more of: alleviating one or more symptoms caused by the disease, attenuating the extent of the disease, stabilizing the disease (e.g., preventing or delaying disease progression), preventing or delaying disease spread (e.g., metastasis), preventing or delaying disease recurrence, delaying or slowing down disease progression, improving disease status, enabling disease remission (partial or complete), reducing the dose of one or more other drugs required to treat the disease, delaying disease progression, improving the quality of life, increasing weight gain, and/or prolonging survival. “Treatment” also encompasses a reduction in pathological outcomes (e.g., tumor volume) of a cancer. The method provided in the present invention encompasses any one or more of these therapeutic aspects.

The term “subject” used herein refers to an organism that receives treatment for a particular disease or disorder described herein. Examples of subjects and patients include mammals, such as human, primates (e.g., monkey), or non-primate mammals, that receive treatment for a disease or disorder.

The term “diagnosis” used herein refers to identification of the presence or nature of a pathological disorder, including but not limited to, liver cancer, ovarian cancer, melanoma, or lung cancer. Diagnostic methods vary in sensitivity and specificity. The “sensitivity” of a diagnostic assay is the percentage (percentage of true positives) of individual patients who are tested to be positive. The “specificity” of a diagnostic assay is 1 minus a false positive rate, where the false positive rate is defined as the proportion of those individuals who do not suffer from a disease but are tested to be positive. Although a particular diagnostic method may not provide a definitive diagnosis for a disorder, it is sufficient that the method can provide a positive indication to assist in diagnosis.

The term “GPC3” or “glypican-3” used herein is a member of the glypican family of heparan sulfate (HS) proteoglycans, which is attached to the cell surface by a glycosylphosphatidylinositol anchor. Human GPC3 has four known subtypes (subtypes 1-4) whose nucleic acid and amino acid sequences are known, including GenBank IDs: NM_001164617 and NP_001158089 (subtype 1): NM_004484 and NP_004475 (subtype 2): NM_001164618 and NP_001158090 (subtype 3); and NM_001164619 and NP_001158091 (subtype 4). In some embodiments disclosed herein, the antibodies disclosed herein may bind to one or more of the four GPC3 subtypes, or conservative variants thereof.

The term “hepatocellular carcinoma (HCC)” used herein refers to a primary malignant liver tumor that typically occurs in patients with inflammatory livers caused by viral hepatitis, hepatotoxins, or cirrhosis (often caused by alcoholism). HCC is also referred to as malignant hepatoma.

When there is an inconsistency between the sequences described in the specification of the present invention and the sequences in the sequence listing, the sequences described in the specification shall prevail.

### BRIEF DESCRIPTION OF THE DRAWINGS

Unless otherwise defined herein, scientific and technical terms used in correlation with the present invention shall have the meanings that are commonly understood by those skilled in the art.

FIG. 1A shows assay results for binding reactions of control antibodies with a human GPC3-His protein by ELISA;

FIG. 1B shows assay results for binding reactions of control antibodies with a monkey GPC3-His protein by ELISA;

FIG. 1C shows assay results for binding reactions of control antibodies with a murine GPC3-His protein by ELISA;

FIG. 2 shows assay results for binding reactions of a polypeptide GC3pep protein with control antibodies by ELISA;

FIG. 3 shows assay results for expression levels of GPC3 in HepG2 cells using antibody Y035 and antibody T2-23 by FACS;

FIG. 4 shows assay results for expression levels of GPC3 in CHO-K1-human GPC3 cells using antibody Y035 by FACS;

FIG. 5 shows assay results for expression levels of GPC3 in HEK293T-monkey GPC3 cells using antibody Y035 by FACS;

FIG. 6A shows assay results for binding of mouse serum antibodies immunized with a human GPC3-hFc protein to a human GPC3-his protein by ELISA;

FIG. 6B shows assay results for binding of mouse serum antibodies immunized with a human GPC3-his protein to a human GPC3-his protein by ELISA;

FIG. 6C shows assay results for binding of mouse serum antibodies immunized with a GC3pep polypeptide to a human GPC3-his protein by ELISA;

FIG. 7A shows assay results for binding of mouse serum antibodies immunized with a human GPC3-hFc protein to a GC3pep polypeptide by ELISA;

FIG. 7B shows assay results for binding of mouse serum antibodies immunized with a human GPC3-his protein to a GC3pep polypeptide by ELISA;

FIG. 7C shows assay results for binding of mouse serum antibodies immunized with a GC3pep polypeptide to a GC3pep polypeptide by ELISA;

FIG. 8A shows assay results for binding of mouse serum antibodies immunized with a human GPC3-hFc protein to a murine GPC3 protein by ELISA;

FIG. 8B shows assay results for binding of mouse serum antibodies immunized with a human GPC3-his protein to a murine GPC3 protein by ELISA;

FIG. 8C shows assay results for binding of mouse serum antibodies immunized with a GC3pep polypeptide to a murine GPC3 protein by ELISA;

FIG. 9A shows assay results for binding of mouse serum antibodies immunized with a human GPC3-hFc protein to a monkey GPC3 protein by ELISA;

FIG. 9B shows assay results for binding of mouse serum antibodies immunized with a human GPC3-his protein to a monkey GPC3 protein by ELISA;

FIG. 9C shows assay results for binding of mouse serum antibodies immunized with a GC3pep polypeptide to a monkey GPC3 protein by ELISA;

FIG. 10A shows assay results for binding of mouse serum antibodies immunized with a human GPC3-hFc protein to HepG2 cells by FACS;

FIG. 10B shows assay results for binding of mouse serum antibodies immunized with a human GPC3-his protein to HepG2 cells by FACS;

FIG. 10C shows assay results for binding of mouse serum antibodies immunized with a GC3pep polypeptide to HepG2 cells by FACS;

FIG. 11 shows assay results for binding reactions of chimeric antibodies with a human GPC3-his protein by ELISA;

FIG. 12A shows assay results for binding reactions of chimeric antibodies with CHO-K1-human GPC3 cells by FACS;

FIG. 12B shows assay results for binding reactions of chimeric antibodies with CHO-K1 cells by FACS;

FIG. 13A shows assay results for binding reactions of chimeric antibodies with HepG2 tumor cells by FACS;

FIG. 13B shows assay results for binding reactions of chimeric antibodies with A431 tumor cells by FACS;

FIG. 14 shows assay results for binding reactions of chimeric antibodies with a murine GPC3-his protein by ELISA;

FIG. 15 shows assay results for binding reactions of chimeric antibodies with a monkey GPC3-His protein by ELISA;

FIG. 16A shows assay results for binding reactions of chimeric antibodies with HEK293T-monkey GPC3 cells by FACS;

FIG. 16B shows assay results for binding reactions of chimeric antibodies with HEK293T cells by FACS;

FIG. 17 shows assay results for binding reactions of chimeric antibodies with a GC3pep polypeptide protein by ELISA;

FIG. 18A to FIG. 18G show assay results for binding reactions of humanized antibodies with a human GPC3-his protein by ELISA;

FIG. 19A to FIG. 19G show assay results for binding reactions of humanized antibodies with CHO-K1-human GPC3 cells by FACS;

FIG. 20A to FIG. 20C show assay results for binding reactions of humanized antibodies with HepG2 tumor cells by FACS;

FIG. 21 shows assay results for binding reactions of humanized antibodies with a mouse GPC3-his protein by ELISA;

FIG. 22A to FIG. 22G show assay results for binding reactions of humanized antibodies with a monkey GPC3-his protein by ELISA; and

FIG. 23A to FIG. 23G show assay results for binding reactions of humanized antibodies with HEK293T-monkey GPC3 cells by FACS.

### DETAILED DESCRIPTION

The present invention relates to isolated antibodies or antigen-binding moieties that specifically bind with high affinity to glypican-3.

### Anti-GPC3 Antibody

The antibodies provided in the present invention specifically bind to GPC3. Preferably, the antibodies provided in the present invention bind with high affinity (e.g., with a KD of 1×10⁻⁷M or lower) to GPC3. The GPC3 antibody provided in the present invention preferably exhibits one or more of the following properties:
(a) binding with a KD of 1×10⁻⁷M or lower to GPC3; and
(b) binding with cells expressing GPC3 (e.g., HepG2 or CHO cells expressing a human GPC3 protein).

In some embodiments, the cells are cancer cells. In some embodiments, the cancer cells are present in solid tumors (e.g., liver cancer, for example, HCC). In some embodiments, the cancer cells are metastatic cancer cells (e.g., metastatic HCC).

In some embodiments, the anti-GPC3 antibody can cross-react with GPC3 from a species other than human.

In some embodiments, the antibody may be prepared by preparing a peptide containing an amino acid sequence of a target region based on an amino acid sequence of human glypican-3 and using the peptide as an immunogen.

CDRs of the antibodies provided in the present invention may include two, three, four, five or all six CDRs provided herein; and preferably, the antibodies include heavy chain CDR3 or light chain CDR3 provided herein. The CDRs of sequences for heavy chain and light chain variable regions of the antibodies provided in the present invention are analyzed by using Kabat, Chothia and IMGT software, respectively, and corresponding sequence information is shown in Table 1 and Table 2. Table 1 shows VH and VL sequences of the anti-GPC3 antibody, and Table 2 shows results of the VH and VL sequences of the anti-GPC3 antibody analyzed by Kabat, Chothia and IMGT.

TABLE 1
VH and VL sequences of anti-GPC3 antibody

| | | |
|---|---|---|
| Antibody | SEQ ID | |
| ID | NO: | Sequence |

Anti-GPC3 antibody: VH

| | | |
|---|---|---|
| F1.2.14 | 1 | RVQLQQSGAELVKPGASVRMSCKASGYTFTAYSIEWMKQN |
| | | LGKSLEWIGNFRPYNDDTKYNENFKGRATLTVEKSSATVY |
| | | LELSRLTSDDSAVYYCARSGPTDYFDYWGQGTTLIVSS |
| F1.21.8 | 2 | EVQLQQSGPELVKPGASVKISCKTSGFLFTGHYMHWVKSS |
| | | HGNILDWIGYIYPYNAISFYNQKFKGKATLTVDKSSNTAY |
| | | MELRSLTSEDSAVYYCARGDYGNYVTAYWGQGTLVTVSA |
| F1.41.17 | 3 | EVOLVESGGDSVKPGGSLKLSCAASGFTFSSYGMSWVRQT |
| | | PDKRLEWVATINSGGSYTYYQDSVKGRFTISRDNAKNTLY |
| | | LQMSSLKSEDTAIYYCVRHHYGSSYDYFDYWGQGTTLTVS |
| | | S |
| F1.42.8 | 4 | HVQLQQPGAELVKPGASVKLSCKASGYTFTSYWMHWVRQR |
| | | PGQGLEWIEMIHPNSGSTNYNEKFKSKATLTVDKSSSTAY |
| | | MQLSSLTSEDSAVYYCARGNYNSSLYAMDYWGQGTSVTVS |
| | | S |
| F1.57.21 | 5 | QVQLQQSGAELARPGASVKLSCKASGYTFTSYGISWVKQI |
| | | TGQGLEWIGEIYPRSESTYYNEKFKGKATLTADKSSSTAY |
| | | MELRSLTSEDSAVYFCARDYGNIYSFAYWGQGTLVTVSA |
| F1.73.9 | 6 | QVQLKQSGAELVRPGASVKLSCKASGYTFTDYYINWVKQR |
| | | PGQGLEWIARIYPGSGNTYYNEKFKGKATLTAEKSSSTAY |
| | | MQFSSLTSEDSAVYFCACSTYYTMDYWGQGTSVTVSS |
| F1.78.24 | 7 | QVQLQQSGAELVKPGASVKMSCKASGYTFTTYPIEWMKQN |
| | | HGKSLEWIGNFHPYNDDTNYNVKFKGKATLTVEKSSSTVY |
| | | LELSRLTSDDSALYYCARRREYYGSFDVWGTGTTVTVSS |
| F1.83.6 | 8 | QVQLQQSGPELVKPGASVKISCKASGYAFSSSWMNWVKQR |
| | | PGKGLEWIGRIYPGDGDTNYNGRFKGKATLTVDKSSSTAY |
| | | MQLSSLTSEDSAVYFCARGGVFTMALDHWGQGTTLTVSS |
| F1.92.17 | 9 | EVQLQQSGPELVKPGASVKMSCKASGYTFTDYNMHWVRQS |
| | | HGKSLEWIGYINPNNGDTIYNQNFKGKATLTVNKSSSTAY |
| | | MELRSLTSEDSAVYYCSRTITYYYSMDYWGPGTSVTVSS |
| F1.110.24 | 10 | EVKLVESGGGLVQPGGSLKLSCAASGFTFSDFYMYWVRQT |
| | | PEKRLEWVAFISNGGGSTYYPDTVKGRFTISRDNVKNTLY |
| | | LQMSRLKSEDTAMYYCARPDTSWFAYWGQGSLVTVSA |
| F1.111.11 | 11 | EVQLQQSGPELVKPGASVKISCKASGYTFTDYYMNWVKQS |
| | | HGKSLEWIGDIYPNNGDTTYNQRFKGKATLTVDKSSSTAY |
| | | MELRSLASEDSAVYYCACPYNNPFGYWGQGTLVTVSA |
| F1.115.8 | 12 | EVQLQQSGPELVKPGASVKMSCKASGYTFTEYYMHWVKQS |
| | | HGKSLEWIGYIYPNNGGNGYNQKFKGKATLTVDKSSSTAY |
| | | MGLRSLTSEDSAVYYCVRGYYSNWYFDVWGTGTTVTVSS |
| F1.118.4 | 13 | QVQLQQSGAELAKPGASVKLSCKASGYSFTSYWIHWVKQR |
| | | PGQGLEWIGYINPSSGYTKYNQKFKDKATLTADKSSSTAY |
| | | MQLSSLTYEDSAVYYCARFGSSSFYFDYWGQGTTLTVSS |
| F1.120.16 | 14 | EFQLQQSGPELVKPGASVKISCKASGYSFTDYNMNWVKQS |
| | | NGKSLEWIGLIHPKFSVTSYNRKFKGKATLTVDQSSSTAY |
| | | MQLNSLTSEDSAVYYCARWAGDYFDYWGQGTTLTVSS |
| F1.136.7 | 15 | EVQLQQSGPVMVKPGPSVKISCQTSGFTFTDYYIHWVKQS |
| | | HGKSLEWIGLVFPYDGGTNYNQKFKDKATLTLDTSSSTAY |
| | | MELNSLTSEDSAVYYCARAGGFSYALNYWGQGTSVTVSS |
| F1.145.22 | 16 | QVQLQQSGTDLARPGASVKLSCKASGYTFTSFGISWVKQR |
| | | TGQGLEWIGEIYPRSGSPYYNEKFTGKATLTADKSSSTAY |
| | | MELRSLTSEDSAVYFCAKDFGSSYVFDYWGQGTALTVSS |
| F1.3.9 | 17 | QVQLQQPGTELVKPGASVKLSCKASGYTFTSYWIHWVKQR |
| | | PGQGLEWIGNINPPNAGTNYNEKFKSKATLTVDKSSSTAY |
| | | MQLSSLTSEDSAVYYCARGRWLLHDYWGQGTTLTVSS |
| F1.9.19 | 18 | DVQLQESGPGLVKPSQSLSLTCSVTGYSITSGYYWNWIRQ |
| | | FPGNRLEWMAYISYDGINNYNPSLKNRISIARDTSTNQFF |
| | | LKLNSVTSEDTATYYCATTTEVALFDHWGQGTTLTVSS |
| F1.62.9 | 19 | QVQLQQSDAELVKPGASVKISCKVSGYIFTDHTIHWMKQR |
| | | PEQGLEWIGYIYPRDGSIKYNENFKGKATLTADKSSSATY |
| | | MQLNSLTSEDSAVYFCARPIYYDYAYYFDYWGQGTTLTVS |
| | | S |
| F1.153.1 | 20 | QIQLVQSGPELKKPGETVKISCKASGYTFTTAGMQWVQKM |
| | | PGKGFKWIGWINTHSGEPQYAEDFKGRFAFSLETSASTAY |
| | | LQISNLKNEDTATYFCARSFYYHGSSFFYFDYWGQGTTLT |
| | | VSS |
| F2.55.11 | 21 | EVKLVESGGGLVKPGGSLKLSCAASGLTFSTCTMSWVRQT |
| | | PAKRLEWVATISSGGGNTYYPDSVKGRFTISRDNARNTLY |
| | | LQMSSLRSEDTAMYYCVRLYYDGSPYAMDYWGQGTSVTVS |
| | | S |
| F2.154.1 | 22 | QVQLQQSGAELVRPGASVKLSCKASGYRFTDYEMHWVKQT |
| | | PVHGLEWIGTIDPETGNTAYTQKFMDKATLTADKSSSTAY |
| | | MELRSLTSEDSAVYYCTRGYSFTYWGQGTLATVSA |
| F2.169.2 | 23 | EVQLVETGGGLVQPKGSLKLSCAASGFSFNTNAMNWVRQA |
| | | PGKGLEWVARIRSKSNNYATYYADSVKDRFTISRDDSQSM |
| | | LYLQMNNLKTEDTAMYYCVRDGYYVLFAYWGQGTLVTVSA |
| F2.39.2 | 24 | QVQLQQSGAELVRPGASVKVSCKASGYTFTDYEMHWMKQT |
| | | PVHGLEWIGANEPETGRSAYDQKFKGKATLTADKSSSTAY |
| | | MELRSLTSEDSAVYYCTRFYSFAYWGQGTLVTVSA |
| F2.92.1 | 25 | QVQLQQSGAELVRPGASVKLSCKASGYIFSDYEIHWVKQT |
| | | PVHGLECIGAVDPETGGTAYNQKFKGKATLTADKSSSTAY |
| | | MELRSLTSEDSAVYFCTRFYSFAYWGQGTLVTVST |
| F2.152.3 | 26 | EVQLQQSGPELVKPGASVKMSCKASGYTFTDYYMTWVKQS |
| | | HGKSLEWIGRVNPRNGDTTYNQKFEGRATLTLDKSLSTAF |
| | | MQLNSLTSEDSAVYYCINSKGHFDYWGQGTTLTVSS |
| F3.38.7 | 27 | QVQLQQSGAELVRPGASVKLSCKASGYTFTDYEIHWVKQT |
| | | PVLGLEWIGAIDPKTGNTAYNQKFMGKATLTADKSSTTAY |
| | | MDLRSLTSEDSAVYYCTRYFSFAYWGQGTLVTVSA |
| F3.54.12 | 28 | QVQLQQSGAELVRPGASVKLSCKASGYTFTDYEMHWVKQT |
| | | PVHGLEWIGAIDPETGNTAYIQKFKGKATLTTDKSSSTAY |
| | | MELRSLTSEDSAVYYCTRFYSYSHWGQGTTLTVSS |
| F3.81.19 | 29 | QVQLQQSGAELVRPGASVKLSCKASGYTFTDYEMHWVKQT |
| | | PVHGLEWIGTIDPETGNTAYNQKFKGKATLTADKSSSTAY |
| | | MELRSLTSEDSAVYYCTRPYSLTYWGQGTLVTVSA |
| F4.13.7 | 30 | QVQLQQPGAELVKPGASVKLSCKASGYTFTNNWVHWVKQR |
| | | PGQGLEWTGMIHPDSGSTIYNEKFQSKATLTVDKSSSTAY |
| | | MQLSSLTSEDSAVYYCARGYYDSSSYYAMDYWGQGTSVTV |
| | | SS |
| F4.18.4 | 31 | QVQLHQSGAEMAKPGASVNLSCKTSGFTFTDYGISWVKQR |
| | | TRQGLEWIGDIYPRHINIYYNEKFKDKATLTADRSSSTAY |
| | | MELRSMTSEDSAVYFCARRGTTGRGALDYWGQGTSLTVSS |
| F4.21.16 | 32 | DVQLQESGPGLVKPSQSLSLTCSVTGYSITSGYYWNWIRQ |
| | | FPGNKLEWMGYISYDVTVNYNPSLKNRISITRDTSKNQFF |
| | | LKLNSVTTEDTATYYCATITSVASMDHWGQGTSVTVSS |
| F4.26.1 | 33 | QVQLKQSGPGLVQPSQSLSITCTVSGFSLTSYGVHWVRQS |
| | | PGKGLEWLGVIWSSGNTDYNAVLISRLSIKKDDSKSQVFF |
| | | KMNSPQVDDSAIYYCGRNGGWLLRGFMDFWGQGTSVTVSS |
| F2.23.8 | 34 | QVQLQQSGAELVRPGASVKLSCKASGYTFTDYEMHWVKQT |
| | | PVHGLEWFGAIDPETGGTAYNQKFRAKATLTADKSSSTAY |
| | | MELRSLTSEDSAVYYCTRFYSFAYWGQGTLVTVSA |

Anti-GPC3 antibody: VL

| | | |
|---|---|---|
| F1.2.14 | 35 | DIQMTQSPASLSASVGETVTITCRASDNIYSYLAWYQQKQ |
| | | GQSPQLLVYNAKTLAEGVPSRFSGSGSVTQFSLKIISLQP |
| | | EDFGTYYCQHHFGTPRTFGGGTKLEIK |
| F1.21.8 | 36 | DVLMTQTPLSLPVSLGDQASISCRSSQSIVHSSGNTYLEW |
| | | YLQKPGQSPKLLIYKVSNRFSGVPDRFSGSGSGTDFTLKI |
| | | SRVEAEDLGVYYCFQGSHLPWTFGGGTKLDIK |
| F1.41.17 | 37 | DIVMTQAAPSVPVMPGESVSISCRSSKSLLHSNGNTHLYW |
| | | FLQRPGQSPQLLIYRMSNLASGVPDRFSGSGSGTAFTLRI |
| | | SRVEAEDVGVYYCMQHLEYPLTFGAGTKLELK |
| F1.42.8 | 38 | DIQMTQTTSSLSASLGDRVTISCSASQGISNYLNWYQQKP |
| | | DGTVKLLIYYASNLHSGVPSRFSGSGSGTAYSLTISNLEP |
| | | EDIATYYCQQYSILPYTFGGGTKLEIK |
| F1.57.21 | 39 | DIQMTQSPASLSASVGETVTITCRTSENIYSYLAWYQQKQ |
| | | GKSPQLLVYNAKTLAEGVPSRFSGSGSGTQFSLKINSLQP |
| | | EDFGSYYCQHHYGTPLTFGAGTKLELK |
| F1.73.9 | 40 | QIVLTQSPAIMSASLGERVTMTCTASSGVSSSYLHWYQQK |
| | | PGSSPKLWIYTTSNLASGVPARFSGSGSGTSYSLTISSME |
| | | AEDAATYYCHQYHRSPYTFGGGTKLEIK |
| F1.78.24 | 41 | DIVMTQSPSSLAMSVGQKVTMSCKSSQSLLNRRNQKNYLA |
| | | WYQQKPGQSPKLLVYFASTRESGVPDRFIGSGSGTDFTLT |
| | | ISSVQAADLADYFCQQHYSTPYTFGGRTKLEIK |
| F1.83.6 | 42 | DIQMTQSPSSLSASLGGKVTITCKASQDINKYIAWYQHKP |
| | | GKGPRLLIHYTSTLQPGIPSRFSGSGSGRDYSFSISNLEP |
| | | EDIATYYCLQYDNLFTFGGGTKLEIK |
| F1.92.17 | 43 | QIVLTQSPALMSASPGEKVTMTCSASSSVSYMYWYQQKPR |
| | | SSPKPWIYLTSNLASGVPARFSGSGSGTSYSLTISSMEAE |
| | | DAATYYCQQWSSNPLTFGAGTKLELK |
| F1.110.24 | 44 | DIQMTQTTSSLSASLGDRVTISCRASQDIDNYLNWYQQKP |
| | | DGTVKVLISYTSRLHSGVPSRFSGSGSGTDYSLTISNLEQ |
| | | EDIATYFCQQGNTLPWTFGGGTKLEIK |
| F1.111.11 | 45 | DVVMTQTPLSLPVSLGDQASISCRSSQSLVHSNGNTYLHW |
| | | YLQKPGQSPKLLIYKVSNRFSGVPDRFSGSGSGTDFTLKI |
| | | SRVEAEDLGVYFCSQSTHVPWTFGGGTKLEIK |
| F1.115.8 | 46 | DVLMTQTPLSLPVSLGDQASISCRSSQSIVHSNGKTYLEW |
| | | YLQKPGQSPKLLIYKVSNRFSGVPDRFSGSGSGTDFTLKI |
| | | SRVEAEDLGVYYCFQGSHVPFTFGSGTKLEIK |
| F1.118.4 | 47 | DIVLTQSPASLAVSLGQRATISCRASQSVSSSSYSYMHWY |
| | | QQKPGQPPKLLIKYASNLDSGVPARFSGSGSGTDFTLNIH |
| | | PVEEEDTATYYCQHSWEIPAFGGGTKLEIK |
| F1.120.16 | 48 | DIVLTQSPATLSVTPGDSVSLSCRASQSISNNLHWYQQKS |
| | | HESPRLLIKYASQSISGIPSRFSGSGSGTDFTLTINSVET |
| | | EDFGMYFCQQSNSWPQTFGGGTKLEIK |
| F1.136.7 | 49 | DIQMTQSPSSLSASLGGKVTITCKASQDINNYIAWYQHKP |
| | | GKGPRLLIHFTSTLQPGIPSRFSGSGSGRDYSFSISNLEP |
| | | EDIATYYCLQYDNLLLTFGAGTKLELK |
| F1.145.22 | 50 | DIQMTQSPASLSASVGETVTITCRASENIYSYLTWYQQKQ |
| | | GKSPQLLVYNAKTLAEGVPSRFSGSGSGTQFSLKINSLQP |
| | | EDFGTYYCQHHYGTPPTFGTGTKLDLK |
| F1.3.9 | 51 | DIQMTQSPASLSISVGETVTITCRASENIYSNLAWYQQKQ |
| | | GKSPQLLVYGTKNLAEGVPSRFSGSGSGTQYSLKINSLQS |
| | | EDFGNYYCQHFWGTPYTFGGGTKLEIL |
| F1.9.19 | 52 | DVLLTQTPLSLPVSLGDQASISCRSSQNIVHGNGNTYLEW |
| | | FLQKPGQSPKLLIYKVSNRFSGVPDRFSGSGSGTDFTLKI |
| | | SRVEAEDLGLYYCFQASHVPWTFGGGTKLEIK |
| F1.62.9 | 53 | DVVMTQSPLSLPVSLGDQASISCRSRQSLVHSNGNTYLHW |
| | | YLQKPGQSPKVLIYKVSNRFSGVPDRFSGSGSGTDFTLKI |
| | | SRVEAEDLGVYFCSQSTHVPYTFGGGTKLEIK |
| F1.153.1 | 54 | DIVMTQSQKFMSTTVGDRVSITCKASQNVGTAVAWYQQKP |
| | | GQSPKLLIYSASNRYTGVPDRFTGSGSGADFTLTISNMQS |
| | | EDLADYFCQQYSRYPLTFGAGTKLELK |
| F2.55.11 | 55 | DIQMTQTTSSLSASLGDRVTISCRASQDISNHLNWYQQKP |
| | | DGTVKLLIYYTSRLHSGVPSRFSGSGSGTDYSLTISNLEQ |
| | | EDIATYFCQQDSKPPPTFGSGTKLEIK |
| F2.154.1 | 56 | DVLMTQTPLSLPVSLGDQASISCRSSQNIVHSNGNTYLQW |
| | | YLQKPGQSPKLLIFKVSNRFSGVPDRFSGSGSGTDFTLKI |
| | | SRVEAEDLGVYYCFQGSHVPFTFGTGTKLEIK |
| F2.169.2 | 57 | DVLMTQTPLSLPVSLGDQASISCRFSQSIVNSNGNTYLEW |
| | | FLQKPGQSPKLLIYKVSNRFSGVPDRFSGSGSGTDFTLKI |
| | | SRVEAEDLGVYYCFQGSHVPFTFGTGTKLEIK |
| F2.39.2 | 58 | DVVMTQTPLSLPVSLGDQASISCRSSQGLVHNNGNTYLHW |
| | | YLQKPGQSPKLLIYKVSNRFSGVPDRFSGSGSGTDFTLKI |
| | | SRVEAEDLGVYFCSQNTHVPPTFGGGTKLEIK |
| F2.92.1 | 59 | DVVMTQTPLSLPVSLGDQGSISCRSSQSLVHSNGNTYLQW |
| | | YLQKPGQSPKLLIYKVSNRFSGVPDRFSGSGSGTDFTLKI |
| | | SRVEAEDLGVYFCCQSTHVLPTFGGGTKLEIK |
| F2.152.3 | 60 | DVLMTQTPLSLPVSLGDQASISCRSSQSIVHSNGNTYLEW |
| | | FLQKPGQSPKSLIYKVSNRFSGVPDRESGSGSGTDFTLKI |
| | | SRVEAEDLGVYYCFQGSHVPPTFGAGTKLELK |
| F3.38.7 | 61 | DVLMTQTPLSLPVSLGDQASISCRSSQTFVHSNGNTYLQW |
| | | YLQKPGQSPKLLIYKVSNRFSGVPDRFSGSGSGTDFTLKI |
| | | SRVEAEDLGVYYCFQGSHVPYTFGSGTKLEIK |
| F3.54.12 | 62 | DVVMTQTPLSLPVSLGDQASISCRSSQSLVHSNGNTYLHW |
| | | YLQKPGQSPKLLIYKVSNRFSGVPDRFSGSSSGTDFTLKI |
| | | SRVEAEDLGVYFCSQNTHVPPTFGGGTKLEIK |
| F3.81.19 | 63 | DVLLTQTPLSLPVSLGDQASISCRSSQSFVHSNGDTYLQW |
| | | YLQKPGQSPKLLIYKVSNRFSGVPDRFSGSGSGTDFTLKI |
| | | SRVEAEDLGVYYCFQGSHVPYTFGSGTKLEIK |
| F4.13.7 | 64 | NIQMTQTTSSLSASLGDRVTISCSASQGIFNYLNWYQQKP |
| | | DGTVKLLIYYTSSLHSGVPSRFSGSGSGTDYSLTISNLEP |
| | | EDIATYYCQQYSKLPWTFGGGTKLEIK |
| F4.18.4 | 65 | DILLTQSPAILSVSPGERVSFSCRASQSIGTSIHWYQQRT |
| | | NGSPRLLIKYAFESISGIPSRFGGSGSRTDFTLTINSVES |
| | | EDIADYYCQQTYNWPYTFGGGTKLEIK |
| F4.21.16 | 66 | DILMTQTPLSLPVSLGDQASISCRSSQNIVHRNGNTYLEW |
| | | YLQKPGQSPKLLLYKVSNRFSGVPDRFSGSGSGTDFTLKI |
| | | SRVEAEDLGIYYCFQGSHFPFTFGSGTKLEIK |
| F4.26.1 | 67 | DVVMTQTPLSLPVSLGDQASISCRSSQSLLHSNGNTYLHW |
| | | YLQRSGQSPNLLIYKVSNRFSGVPDRFSGGGSGTDFTLKI |
| | | SRVEAEDLGIYFCSQSTHVPWTFGGGTKLEIK |
| F2.23.8 | 68 | DVVMTQTPLSLPVSLGDQASISCRSSQSLVHSNGNTYLQW |
| | | YLQKPGQSPKLLIYKVSNRFSGVPDRFSGSGSGTDFTLKI |
| | | TRVEAEDLGVYFCSQSTHVPYTFGSGTKLEIK |

TABLE 2
Kabat, Chothia and IMGT analysis results of VH and VL sequences of
anti-GPC3 antibody

| Heavy chain/ | | | | |
|---|---|---|---|---|
| light chain | | | | |
| variable | Definition | | | |
| regions | method | CDR1 | CDR2 | CDR3 |
| SEQ ID NO: 1 | Kabat | AYSIE | NFRPYNDDTKYNENFKG | SGPTDYFDY |
| | | SEQ ID NO: 69 | SEQ ID NO: 70 | SEQ ID NO: 71 |
| | Chothia | GYTFTAY | RPYNDD | SGPTDYFDY |
| | | SEQ ID NO: 72 | SEQ ID NO: 73 | SEQ ID NO: 74 |
| | IMGT | GYTFTAYS | FRPYNDDT | ARSGPTDYFDY |
| | | SEQ ID NO: 75 | SEQ ID NO: 76 | SEQ ID NO: 77 |
| SEQ ID NO: 2 | Kabat | GHYMH | YIYPYNAISFYNQKFKG | GDYGNYVTAY |
| | | SEQ ID NO: 78 | SEQ ID NO: 79 | SEQ ID NO: 80 |
| | Chothia | GFLFTGH | YPYNAI | GDYGNYVTAY |
| | | SEQ ID NO: 81 | SEQ ID NO: 82 | SEQ ID NO: 83 |
| | IMGT | GFLFTGHY | IYPYNAIS | ARGDYGNYVTAY |
| | | SEQ ID NO: 84 | SEQ ID NO: 85 | SEQ ID NO: 86 |
| SEQ ID NO: 3 | Kabat | SYGMS | TINSGGSYTYYQDSVKG | HHYGSSYDYFDY |
| | | SEQ ID NO: 87 | SEQ ID NO: 88 | SEQ ID NO: 89 |
| | Chothia | GFTFSSY | NSGGSY | HHYGSSYDYFDY |
| | | SEQ ID NO: 90 | SEQ ID NO: 91 | SEQ ID NO: 92 |
| | IMGT | GFTFSSYG | INSGGSYT | VRHHYGSSYDYF |
| | | SEQ ID NO: 93 | SEQ ID NO: 94 | DY |
| | | | | SEQ ID NO: 95 |
| SEQ ID NO: 4 | Kabat | SYWMH | MIHPNSGSTNYNEKFKS | GNYNSSLYAMDY |
| | | SEQ ID NO: 96 | SEQ ID NO: 97 | SEQ ID NO: 98 |
| | Chothia | GYTFTSY | HPNSGS | GNYNSSLYAMDY |
| | | SEQ ID NO: 99 | SEQ ID NO: 100 | SEQ ID NO: 101 |
| | IMGT | GYTFTSYW | IHPNSGST | ARGNYNSSLYAM |
| | | SEQ ID NO: 102 | SEQ ID NO: 103 | DY |
| | | | | SEQ ID NO: 104 |
| SEQ ID NO: 5 | Kabat | SYGIS | EIYPRSESTYYNEKFKG | DYGNIYSFAY |
| | | SEQ ID NO: 105 | SEQ ID NO: 106 | SEQ ID NO: 107 |
| | Chothia | GYTFTSY | YPRSES | DYGNIYSFAY |
| | | SEQ ID NO: 108 | SEQ ID NO: 109 | SEQ ID NO: 110 |
| | IMGT | GYTFTSYG | IYPRSEST | ARDYGNIYSFAY |
| | | SEQ ID NO: 111 | SEQ ID NO: 112 | SEQ ID NO:113 |
| SEQ ID NO: 6 | Kabat | DYYIN | RIYPGSGNTYYNEKFKG | STYYTMDY |
| | | SEQ ID NO: 114 | SEQ ID NO: 115 | SEQ ID NO: 116 |
| | Chothia | GYTFTDY | YPGSGN | STYYTMDY |
| | | SEQ ID NO: 117 | SEQ ID NO: 118 | SEQ ID NO: 119 |
| | IMGT | GYTFTDYY | IYPGSGNT | ACSTYYTMDY |
| | | SEQ ID NO: 120 | SEQ ID NO: 121 | SEQ ID NO: 122 |
| SEQ ID NO: 7 | Kabat | TYPIE | NFHPYNDDTNYNVKFKG | RREYYGSFDV |
| | | SEQ ID NO: 123 | SEQ ID NO: 124 | SEQ ID NO: 125 |
| | Chothia | GYTFTTY | HPYNDD | RREYYGSFDV |
| | | SEQ ID NO: 126 | SEQ ID NO: 127 | SEQ ID NO: 128 |
| | IMGT | GYTFTTYP | FHPYNDDT | ARRREYYGSFDV |
| | | SEQ ID NO: 129 | SEQ ID NO: 130 | SEQ ID NO: 131 |
| SEQ ID NO: 8 | Kabat | SSWMN | RIYPGDGDTNYNGREKG | GGVFTMALDH |
| | | SEQ ID NO: 132 | SEQ ID NO: 133 | SEQ ID NO: 134 |
| | Chothia | GYAFSSS | YPGDGD | GGVFTMALDH |
| | | SEQ ID NO: 135 | SEQ ID NO: 136 | SEQ ID NO: 137 |
| | IMGT | GYAFSSSW | IYPGDGDT | ARGGVFTMALD |
| | | SEQ ID NO: 138 | SEQ ID NO: 139 | H |
| | | | | SEQ ID NO: 140 |
| SEQ ID NO: 9 | Kabat | DYNMH | YINPNNGDTIYNQNFKG | TITYYYSMDY |
| | | SEQ ID NO: 141 | SEQ ID NO: 142 | SEQ ID NO: 143 |
| | Chothia | GYTFTDY | NPNNGD | TITYYYSMDY |
| | | SEQ ID NO: 144 | SEQ ID NO: 145 | SEQ ID NO: 146 |
| | IMGT | GYTFTDYN | IGYTFTDYT | SRTITYYYSMDY |
| | | SEQ ID NO: 147 | SEQ ID NO: 148 | SEQ ID NO: 149 |
| SEQ ID NO: | Kabat | DFYMY | FISNGGGSTYYPDTVKG | PDTSWFAY |
| 10 | | SEQ ID NO: 150 | SEQ ID NO: 151 | SEQ ID NO: 152 |
| | Chothia | GFTFSDF | SNGGGS | PDTSWFAY |
| | | SEQ ID NO: 153 | SEQ ID NO: 154 | SEQ ID NO: 155 |
| | IMGT | GFTFSDFY | ISNGGGST | ARPDTSWFAY |
| | | SEQ ID NO: 156 | SEQ ID NO: 157 | SEQ ID NO: 158 |
| SEQ ID NO: | Kabat | DYYMN | DIYPNNGDTTYNQRFKG | PYNNPFGY |
| 11 | | SEQ ID NO: 159 | SEQ ID NO: 160 | SEQ ID NO: 161 |
| | Chothia | GYTFTDY | YPNNGD | PYNNPFGY |
| | | SEQ ID NO: 162 | SEQ ID NO: 163 | SEQ ID NO: 164 |
| | IMGT | GYTFTDYY | IYPNNGDT | ACPYNNPFGY |
| | | SEQ ID NO: 165 | SEQ ID NO: 166 | SEQ ID NO: 167 |
| SEQ ID NO: | Kabat | EYYMH | YIYPNNGGNGYNQKFKG | GYYSNWYFDV |
| 12 | | SEQ ID NO: 168 | SEQ ID NO: 169 | SEQ ID NO: 170 |
| | Chothia | GYTFTEY | YPNNGG | GYYSNWYFDV |
| | | SEQ ID NO: 171 | SEQ ID NO: 172 | SEQ ID NO: 173 |
| | IMGT | GYTFTEYY | IYPNNGGN | VRGYYSNWYFD |
| | | SEQ ID NO: 174 | | V |
| | | | SEQ ID NO: 175 | SEQ ID NO: 176 |
| SEQ ID NO: | Kabat | SYWIH | YINPSSGYTKYNQKFKD | FGSSSFYFDY |
| | | SEQ ID NO: 177 | SEQ ID NO: 178 | SEQ ID NO: 179 |
| 13 | Chothia | GYSFTSY | NPSSGY | FGSSSFYFDY |
| | | SEQ ID NO: 180 | SEQ ID NO: 181 | SEQ ID NO: 182 |
| | IMGT | GYSFTSYW | INPSSGYT | ARFGSSSFYFDY |
| | | SEQ ID NO: 183 | SEQ ID NO: 184 | SEQ ID NO: 185 |
| SEQ ID NO: | Kabat | DYNMN | LIHPKFSVTSYNRKFKG | WAGDYFDY |
| 14 | | SEQ ID NO: 186 | SEQ ID NO: 187 | SEQ ID NO: 188 |
| | Chothia | GYSFTDY | HPKFSV | WAGDYFDY |
| | | SEQ ID NO: 189 | SEQ ID NO: 190 | SEQ ID NO: 191 |
| | IMGT | GYSFTDYN | IHPKFSVT | ARWAGDYFDY |
| | | SEQ ID NO: 192 | SEQ ID NO: 193 | SEQ ID NO: 194 |
| SEQ ID NO: | Kabat | DYYIH | LVFPYDGGTNYNQKFKD | AGGFSYALNY |
| 15 | | SEQ ID NO: 195 | SEQ ID NO: 196 | SEQ ID NO: 197 |
| | Chothia | GFTFTDY | FPYDGG | AGGFSYALNY |
| | | SEQ ID NO: 198 | SEQ ID NO: 199 | SEQ ID NO: 200 |
| | IMGT | GFTFTDYY | VFPYDGGT | ARAGGFSYALNY |
| | | SEQ ID NO: 201 | SEQ ID NO: 202 | SEQ ID NO: 203 |
| SEQ ID NO: | Kabat | SFGIS | EIYPRSGSPYYNEKFTG | DFGSSYVFDY |
| 16 | | SEQ ID NO: 204 | SEQ ID NO: 205 | SEQ ID NO: 206 |
| | Chothia | GYTFTSF | YPRSGS | DFGSSYVFDY |
| | | SEQ ID NO: 207 | SEQ ID NO: 208 | SEQ ID NO: 209 |
| | IMGT | GYTFTSFG | IYPRSGSP | AKDFGSSYVFDY |
| | | SEQ ID NO: 210 | SEQ ID NO: 211 | SEQ ID NO: 212 |
| SEQ ID NO: | Kabat | SYWIH | NINPPNAGTNYNEKFKS | GRWLLHDY |
| 17 | | SEQ ID NO: 213 | SEQ ID NO: 214 | SEQ ID NO: 215 |
| | Chothia | GYTFTSY | NPPNAG | GRWLLHDY |
| | | SEQ ID NO: 216 | SEQ ID NO: 217 | SEQ ID NO: 218 |
| | IMGT | GYTFTSYW | INPPNAGT | ARGRWLLHDY |
| | | SEQ ID NO: 219 | SEQ ID NO: 220 | SEQ ID NO: 221 |
| SEQ ID NO: | Kabat | SGYYWN | YISYDGINNYNPSLKN | TTEVALFDH |
| 18 | | SEQ ID NO: 222 | SEQ ID NO: 223 | SEQ ID NO: 224 |
| | Chothia | GYSITSGY | SYDGI | TTEVALFDH |
| | | SEQ ID NO: 225 | SEQ ID NO: 226 | SEQ ID NO: 227 |
| | IMGT | GYSITSGYY | ISYDGIN | ATTTEVALFDH |
| | | SEQ ID NO: 228 | SEQ ID NO: 229 | SEQ ID NO: 230 |
| SEQ ID NO: | Kabat | DHTIH | YIYPRDGSIKYNENFKG | PIYYDYAYYFDY |
| 19 | | SEQ ID NO: 231 | SEQ ID NO: 232 | SEQ ID NO: 233 |
| | Chothia | GYIFTDH | YPRDGS | PIYYDYAYYFDY |
| | | SEQ ID NO: 234 | SEQ ID NO: 235 | SEQ ID NO: 236 |
| | IMGT | GYIFTDHT | IYPRDGSI | ARPIYYDYAYYF |
| | | SEQ ID NO: 237 | SEQ ID NO: 238 | DY |
| | | | | SEQ ID NO: 239 |
| SEQ ID NO: | Kabat | TAGMQ | WINTHSGEPQYAEDFKG | SFYYHGSSFFYFD |
| 20 | | SEQ ID NO: 240 | SEQ ID NO: 241 | Y |
| | | | | SEQ ID NO: 242 |
| | Chothia | GYTFTTA | NTHSGE | SFYYHGSSFFYFD |
| | | SEQ ID NO: 243 | SEQ ID NO: 244 | Y |
| | | | | SEQ ID NO: 245 |
| | IMGT | GYTFTTAG | INTHSGEP | ARSFYYHGSSFFY |
| | | SEQ ID NO: 246 | SEQ ID NO: 247 | FDY |
| | | | | SEQ ID NO: 248 |
| SEQ ID NO: | Kabat | TCTMS | TISSGGGNTYYPDSVKG | LYYDGSPYAMDY |
| 21 | | SEQ ID NO: 249 | SEQ ID NO: 250 | SEQ ID NO: 251 |
| | Chothia | GLTFSTC | SSGGGN | LYYDGSPYAMDY |
| | | SEQ ID NO: 252 | SEQ ID NO: 253 | SEQ ID NO: 254 |
| | IMGT | GLTFSTCT | ISSGGGNT | VRLYYDGSPYAM |
| | | SEQ ID NO: 255 | SEQ ID NO: 256 | DY |
| | | | | SEQ ID NO: 257 |
| SEQ ID NO: | Kabat | DYEMH | TIDPETGNTAYTQKFMD | GYSFTY |
| 22 | | SEQ ID NO: 258 | SEQ ID NO: 259 | SEQ ID NO: 260 |
| | Chothia | GYRFTDY | DPETGN | GYSFTY |
| | | SEQ ID NO: 261 | SEQ ID NO: 262 | SEQ ID NO: 263 |
| | IMGT | GYRFTDYE | IDPETGNT | TRGYSFTY |
| | | SEQ ID NO: 264 | SEQ ID NO: 265 | SEQ ID NO: 266 |
| SEQ ID NO: | Kabat | TNAMN | RIRSKSNNYATYYADSVKD | DGYYVLFAY |
| 23 | | SEQ ID NO: 267 | SEQ ID NO: 268 | SEQ ID NO: 269 |
| | Chothia | GFSFNTN | RSKSNNYA | DGYYVLFAY |
| | | SEQ ID NO: 270 | SEQ ID NO: 271 | SEQ ID NO: 272 |
| | IMGT | GFSFNTNA | IRSKSNNYAT | VRDGYYVLFAY |
| | | SEQ ID NO: 273 | SEQ ID NO: 274 | SEQ ID NO: 275 |
| SEQ ID NO: | Kabat | DYEMH | ANEPETGRSAYDQKFKG | FYSFAY |
| 24 | | SEQ ID NO: 276 | SEQ ID NO: 277 | SEQ ID NO: 278 |
| | Chothia | GYTFTDY | EPETGR | FYSFAY |
| | | SEQ ID NO: 279 | SEQ ID NO: 280 | SEQ ID NO: 281 |
| | IMGT | GYTFTDYE | NEPETGRS | TRFYSFAY |
| | | SEQ ID NO: 282 | SEQ ID NO: 283 | SEQ ID NO: 284 |
| SEQ ID NO: | Kabat | DYEIH | AVDPETGGTAYNQKFKG | FYSFAY |
| 25 | | SEQ ID NO: 285 | SEQ ID NO: 286 | SEQ ID NO: 287 |
| | Chothia | GYIFSDY | DPETGG | FYSFAY |
| | | SEQ ID NO: 288 | SEQ ID NO: 289 | SEQ ID NO: 290 |
| | IMGT | GYIFSDYE | VDPETGGT | TRFYSFAY |
| | | SEQ ID NO: 291 | SEQ ID NO: 292 | SEQ ID NO: 293 |
| SEQ ID NO: | Kabat | DYYMT | RVNPRNGDTTYNQKFEG | SKGHFDY |
| 26 | | SEQ ID NO: 294 | SEQ ID NO: 295 | SEQ ID NO: 296 |
| | Chothia | GYTFTDY | NPRNGD | SKGHFDY |
| | | SEQ ID NO: 297 | SEQ ID NO: 298 | SEQ ID NO: 299 |
| | IMGT | GYTFTDYY | VNPRNGDT | INSKGHFDY |
| | | SEQ ID NO: 300 | SEQ ID NO: 301 | SEQ ID NO: 302 |
| SEQ ID NO: | Kabat | DYEIH | AIDPKTGNTAYNQKFMG | YFSFAY |
| 27 | | SEQ ID NO: 303 | SEQ ID NO: 304 | SEQ ID NO: 305 |
| | Chothia | GYTFTDY | DPKTGN | YFSFAY |
| | | SEQ ID NO: 306 | SEQ ID NO: 307 | SEQ ID NO: 308 |
| | IMGT | GYTFTDYE | IDPKTGNT | TRYFSFAY |
| | | SEQ ID NO: 309 | SEQ ID NO: 310 | SEQ ID NO: 311 |
| SEQ ID NO: | Kabat | DYEMH | AIDPETGNTAYIQKFKG | FYSYSH |
| 28 | | SEQ ID NO: 312 | SEQ ID NO: 313 | SEQ ID NO: 314 |
| | Chothia | GYTFTDY | DPETGN | FYSYSH |
| | | SEQ ID NO: 315 | SEQ ID NO: 316 | SEQ ID NO: 317 |
| | IMGT | GYTFTDYE | IDPETGNT | TRFYSYSH |
| | | SEQ ID NO: 318 | SEQ ID NO: 319 | SEQ ID NO: 320 |
| SEQ ID NO: | Kabat | DYEMH | TIDPETGNTAYNQKFKG | PYSLTY |
| 29 | | SEQ ID NO: 321 | SEQ ID NO: 322 | SEQ ID NO: 323 |
| | Chothia | GYTFTDY | DPETGN | PYSLTY |
| | | SEQ ID NO: 324 | SEQ ID NO: 325 | SEQ ID NO: 326 |
| | IMGT | GYTFTDYE | IDPETGNT | TRPYSLTY |
| | | SEQ ID NO: 327 | SEQ ID NO: 328 | SEQ ID NO: 329 |
| SEQ ID NO: | Kabat | NNWVH | MIHPDSGSTIYNEKFQS | GYYDSSSYYAMD |
| 30 | | SEQ ID NO: 330 | SEQ ID NO: 331 | Y |
| | | | | SEQ ID NO: 332 |
| | Chothia | GYTFTNN | HPDSGS | GYYDSSSYYAMD |
| | | SEQ ID NO: 333 | SEQ ID NO: 334 | Y |
| | | | | SEQ ID NO: 335 |
| | IMGT | GYTFTNNW | IHPDSGST | ARGYYDSSSYYA |
| | | SEQ ID NO: 336 | SEQ ID NO: 337 | MDY |
| | | | | SEQ ID NO: 338 |
| SEQ ID NO: | Kabat | DYGIS | DIYPRHINIYYNEKFKD | RGTTGRGALDY |
| 31 | | SEQ ID NO: 339 | SEQ ID NO: 340 | SEQ ID NO: 341 |
| | Chothia | GFTFTDY | YPRHIN | RGTTGRGALDY |
| | | SEQ ID NO: 342 | SEQ ID NO: 343 | SEQ ID NO: 344 |
| | IMGT | GFTFTDYG | IYPRHINI | ARRGTTGRGALD |
| | | SEQ ID NO: 345 | SEQ ID NO: 346 | Y |
| | | | | SEQ ID NO: 347 |
| SEQ ID NO: | Kabat | SGYYWN | YISYDVTVNYNPSLKN | ITSVASMDH |
| 32 | | SEQ ID NO: 348 | SEQ ID NO: 349 | SEQ ID NO: 350 |
| | Chothia | GYSITSGY | SYDVT | ITSVASMDH |
| | | SEQ ID NO: 351 | SEQ ID NO: 352 | SEQ ID NO: 353 |
| | IMGT | GYSITSGYY | ISYDVTV | ATITSVASMDH |
| | | SEQ ID NO: 354 | SEQ ID NO: 355 | SEQ ID NO: 356 |
| SEQ ID NO: | Kabat | SYGVH | VIWSSGNTDYNAVLIS | NGGWLLRGFMD |
| 33 | | SEQ ID NO: 357 | SEQ ID NO: 358 | F |
| | | | | SEQ ID NO: 359 |
| | Chothia | GFSLTSY | WSSGN | NGGWLLRGFMD |
| | | SEQ ID NO: 360 | SEQ ID NO: 361 | F |
| | | | | SEQ ID NO: 362 |
| | IMGT | GFSLTSYG | IWSSGNT | GRNGGWLLRGF |
| | | SEQ ID NO: 363 | SEQ ID NO: 364 | MDF |
| | | | | SEQ ID NO: 365 |
| SEQ ID NO: | Kabat | DYEMH | AIDPETGGTAYNQKFRA | FYSFAY |
| 34 | | SEQ ID NO: 366 | SEQ ID NO: 367 | SEQ ID NO: 368 |
| | Chothia | GYTFTDY | DPETGG | FYSFAY |
| | | SEQ ID NO: 369 | SEQ ID NO: 370 | SEQ ID NO: 371 |
| | IMGT | GYTFTDYE | IDPETGGT | TRFYSFAY |
| | | SEQ ID NO: 372 | SEQ ID NO: 373 | SEQ ID NO: 374 |
| SEQ ID NO: | Kabat/ | RASDNIYSYLA | NAKTLAE | QHHFGTPRT |
| 35 | Clothia | SEQ ID NO: 375 | SEQ ID NO: 376 | SEQ ID NO: 377 |
| | IMGT | DNIYSY | NAK | QHHFGTPRT |
| | | SEQ ID NO: 378 | SEQ ID NO: 379 | SEQ ID NO: 380 |
| SEQ ID NO: | Kabat/ | RSSQSIVHSSGNT | KVSNRFS | FQGSHLPWT |
| 36 | Clothia | YLE | SEQ ID NO: 382 | SEQ ID NO: 383 |
| | | SEQ ID NO: 381 | | |
| | IMGT | QSIVHSSGNTY | KVS | FQGSHLPWT |
| | | SEQ ID NO: 384 | SEQ ID NO: 385 | SEQ ID NO: 386 |
| SEQ ID NO: | Kabat/ | RSSKSLLHSNGNT | RMSNLAS | MQHLEYPLT |
| 37 | Clothia | HLY | SEQ ID NO: 388 | SEQ ID NO: 389 |
| | | SEQ ID NO: 387 | | |
| | IMGT | KSLLHSNGNTH | RMS | MQHLEYPLT |
| | | SEQ ID NO: 390 | SEQ ID NO: 391 | SEQ ID NO: 392 |
| SEQ ID NO: | Kabat/ | SASQGISNYLN | YASNLHS | QQYSILPYT |
| 38 | Clothia | SEQ ID NO: 393 | SEQ ID NO: 394 | SEQ ID NO: 395 |
| | IMGT | QGISNY | YAS | QQYSILPYT |
| | | SEQ ID NO: 396 | SEQ ID NO: 397 | SEQ ID NO: 398 |
| SEQ ID NO: | Kabat/ | RTSENIYSYLA | NAKTLAE | QHHYGTPLT |
| 39 | Clothia | SEQ ID NO: 399 | SEQ ID NO: 400 | SEQ ID NO: 401 |
| | IMGT | ENIYSY | NAK | QHHYGTPLT |
| | | SEQ ID NO: 402 | SEQ ID NO: 403 | SEQ ID NO: 404 |
| SEQ ID NO: | Kabat/ | TASSGVSSSYLH | TTSNLAS | HQYHRSPYT |
| 40 | Clothia | SEQ ID NO: 405 | SEQ ID NO: 406 | SEQ ID NO: 407 |
| | IMGT | SGVSSSY | TTS | HQYHRSPYT |
| | | SEQ ID NO: 408 | SEQ ID NO: 409 | SEQ ID NO: 410 |
| SEQ ID NO: | Kabat/ | KSSQSLLNRRNQK | FASTRES | QQHYSTPYT |
| 41 | Clothia | NYLA | SEQ ID NO: 412 | SEQ ID NO: 413 |
| | | SEQ ID NO: 411 | | |
| | IMGT | QSLLNRRNQKNY | FAS | QQHYSTPYT |
| | | SEQ ID NO: 414 | SEQ ID NO: 415 | SEQ ID NO: 416 |
| SEQ ID NO: | Kabat/ | KASQDINKYIA | YTSTLQP | LQYDNLFT |
| 42 | Clothia | SEQ ID NO: 417 | SEQ ID NO: 418 | SEQ ID NO: 419 |
| | IMGT | QDINKY | YTS | LQYDNLFT |
| | | SEQ ID NO: 420 | SEQ ID NO: 421 | SEQ ID NO: 422 |
| SEQ ID NO: | Kabat/ | SASSSVSYMY | LTSNLAS | QQWSSNPLT |
| 43 | Clothia | SEQ ID NO: 423 | SEQ ID NO: 424 | SEQ ID NO: 425 |
| | IMGT | SSVSY | LTS | QQWSSNPLT |
| | | SEQ ID NO: 426 | SEQ ID NO: 427 | SEQ ID NO: 428 |
| SEQ ID NO: | Kabat/ | RASQDIDNYLN | YTSRLHS | QQGNTLPWT |
| 44 | Clothia | SEQ ID NO: 429 | SEQ ID NO: 430 | SEQ ID NO: 431 |
| | IMGT | QDIDNY | YTS | QQGNTLPWT |
| | | SEQ ID NO: 432 | SEQ ID NO: 433 | SEQ ID NO: 434 |
| SEQ ID NO: | Kabat/ | RSSQSLVHSNGNT | KVSNRFS | SQSTHVPWT |
| 45 | Clothia | YLH | SEQ ID NO: 436 | SEQ ID NO: 437 |
| | | SEQ ID NO: 435 | | |
| | IMGT | QSLVHSNGNTY | KVS | SQSTHVPWT |
| | | SEQ ID NO: 438 | SEQ ID NO: 439 | SEQ ID NO: 440 |
| SEQ ID NO: | Kabat/ | RSSQSIVHSNGKT | KVSNRFS | FQGSHVPFT |
| | | YLE | | |
| 46 | Clothia | SEQ ID NO: 441 | SEQ ID NO: 442 | SEQ ID NO: 443 |
| | IMGT | QSIVHSNGKTY | KVS | FQGSHVPFT |
| | | SEQ ID NO: 444 | SEQ ID NO: 445 | SEQ ID NO: 446 |
| SEQ ID NO: | Kabat/ | RASQSVSSSSYSY | YASNLDS | QHSWEIPA |
| 47 | Clothia | MH | SEQ ID NO: 448 | SEQ ID NO: 449 |
| | | SEQ ID NO: 447 | | |
| | IMGT | QSVSSSSYSY | YAS | QHSWEIPA |
| | | SEQ ID NO: 450 | SEQ ID NO: 451 | SEQ ID NO: 452 |
| SEQ ID NO: | Kabat/ | RASQSISNNLH | YASQSIS | QQSNSWPQT |
| 48 | Clothia | SEQ ID NO: 453 | SEQ ID NO: 454 | SEQ ID NO: 455 |
| | IMGT | QSISNN | YAS | QQSNSWPQT |
| | | SEQ ID NO: 456 | SEQ ID NO: 457 | SEQ ID NO: 458 |
| SEQ ID NO: | Kabat/ | KASQDINNYIA | FTSTLQP | LQYDNLLLT |
| 49 | Clothia | SEQ ID NO: 459 | SEQ ID NO: 460 | SEQ ID NO: 461 |
| | IMGT | QDINNY | FTS | LQYDNLLLT |
| | | SEQ ID NO: 462 | SEQ ID NO: 463 | SEQ ID NO: 464 |
| SEQ ID NO: | Kabat/ | RASENIYSYLT | NAKTLAE | QHHYGTPPT |
| 50 | Clothia | SEQ ID NO: 465 | SEQ ID NO: 466 | SEQ ID NO: 467 |
| | IMGT | ENIYSY | NAK | QHHYGTPPT |
| | | SEQ ID NO: 468 | SEQ ID NO: 469 | SEQ ID NO: 470 |
| SEQ ID NO: | Kabat/ | RASENIYSNLA | GTKNLAE | QHFWGTPYT |
| 51 | Clothia | SEQ ID NO: 471 | SEQ ID NO: 472 | SEQ ID NO: 473 |
| | IMGT | ENIYSN | GTK | QHFWGTPYT |
| | | SEQ ID NO: 474 | SEQ ID NO: 475 | SEQ ID NO: 476 |
| SEQ ID NO: | Kabat/ | RSSQNIVHGNGNT | KVSNRFS | FQASHVPWT |
| 52 | Clothia | YLE | SEQ ID NO: 478 | SEQ ID NO: 479 |
| | | SEQ ID NO: 477 | | |
| | IMGT | QNIVHGNGNTY | KVS | FQASHVPWT |
| | | SEQ ID NO: 480 | SEQ ID NO: 481 | SEQ ID NO: 482 |
| SEQ ID NO: | Kabat/ | RSRQSLVHSNGNT | KVSNRFS | SQSTHVPYT |
| 53 | Clothia | YLH | SEQ ID NO: 484 | SEQ ID NO: 485 |
| | | SEQ ID NO: 483 | | |
| | IMGT | QSLVHSNGNTY | KVS | SQSTHVPYT |
| | | SEQ ID NO: 486 | SEQ ID NO: 487 | SEQ ID NO: 488 |
| SEQ ID NO: | Kabat/ | KASQNVGTAVA | SASNRYT | QQYSRYPLT |
| 54 | Clothia | SEQ ID NO: 489 | SEQ ID NO: 490 | SEQ ID NO: 491 |
| | IMGT | QNVGTA | SAS | QQYSRYPLT |
| | | SEQ ID NO: 492 | SEQ ID NO: 493 | SEQ ID NO: 494 |
| SEQ ID NO: | Kabat/ | RASQDISNHLN | YTSRLHS | QQDSKPPPT |
| 55 | Clothia | SEQ ID NO: 495 | SEQ ID NO: 496 | SEQ ID NO: 497 |
| | IMGT | QDISNH | YTS | QQDSKPPPT |
| | | SEQ ID NO: 498 | SEQ ID NO: 499 | SEQ ID NO: 500 |
| SEQ ID NO: | Kabat/ | RSSQNIVHSNGNT | KVSNRFS | FQGSHVPFT |
| 56 | Clothia | YLQ | SEQ ID NO: 502 | SEQ ID NO: 503 |
| | | SEQ ID NO: 501 | | |
| | IMGT | QNIVHSNGNTY | KVS | FQGSHVPFT |
| | | SEQ ID NO: 504 | SEQ ID NO: 505 | SEQ ID NO: 506 |
| SEQ ID NO: | Kabat/ | RFSQSIVNSNGNT | KVSNRFS | FQGSHVPFT |
| 57 | Clothia | YLE | SEQ ID NO: 508 | SEQ ID NO: 509 |
| | | SEQ ID NO: 507 | | |
| | IMGT | QSIVNSNGNTY | KVS | FQGSHVPFT |
| | | SEQ ID NO: 510 | SEQ ID NO: 511 | SEQ ID NO: 512 |
| SEQ ID NO: | Kabat/ | RSSQGLVHNNGN | KVSNRFS | SQNTHVPPT |
| 58 | Clothia | TYLH | SEQ ID NO: 514 | SEQ ID NO: 515 |
| | | SEQ ID NO: 513 | | |
| | IMGT | QGLVHNNGNTY | KVS | SQNTHVPPT |
| | | SEQ ID NO: 516 | SEQ ID NO: 517 | SEQ ID NO: 518 |
| SEQ ID NO: | Kabat/ | RSSQSLVHSNGNT | KVSNRFS | CQSTHVLPT |
| 59 | Clothia | YLQ | SEQ ID NO: 520 | SEQ ID NO: 521 |
| | | SEQ ID NO: 519 | | |
| | IMGT | QSLVHSNGNTY | KVS | CQSTHVLPT |
| | | SEQ ID NO: 522 | SEQ ID NO: 523 | SEQ ID NO: 524 |
| SEQ ID NO: | Kabat/ | RSSQSIVHSNGNT | KVSNRFS | FQGSHVPPT |
| 60 | Clothia | YLE | SEQ ID NO: 526 | SEQ ID NO: 527 |
| | | SEQ ID NO: 525 | | |
| | IMGT | QSIVHSNGNTY | KVS | FQGSHVPPT |
| | | SEQ ID NO: 528 | SEQ ID NO: 529 | SEQ ID NO: 530 |
| SEQ ID NO: | Kabat/ | RSSQTFVHSNGNT | KVSNRFS | FQGSHVPYT |
| 61 | Clothia | YLQ | SEQ ID NO: 532 | SEQ ID NO: 533 |
| | | SEQ ID NO: 531 | | |
| | IMGT | QTFVHSNGNTY | KVS | FQGSHVPYT |
| | | SEQ ID NO: 534 | SEQ ID NO: 535 | SEQ ID NO: 536 |
| SEQ ID NO: | Kabat/ | RSSQSLVHSNGNT | KVSNRFS | SQNTHVPPT |
| 62 | Clothia | YLH | SEQ ID NO: 538 | SEQ ID NO: 539 |
| | | SEQ ID NO: 537 | | |
| | IMGT | QSLVHSNGNTY | KVS | SQNTHVPPT |
| | | SEQ ID NO: 540 | SEQ ID NO: 541 | SEQ ID NO: 542 |
| SEQ ID NO: | Kabat/ | RSSQSFVHSNGDT | KVSNRFS | FQGSHVPYT |
| 63 | Clothia | YLQ | SEQ ID NO: 544 | SEQ ID NO: 545 |
| | | SEQ ID NO: 543 | | |
| | IMGT | QSFVHSNGDTY | KVS | FQGSHVPYT |
| | | SEQ ID NO: 546 | SEQ ID NO: 547 | SEQ ID NO: 548 |
| SEQ ID NO: | Kabat/ | SASQGIFNYLN | YTSSLHS | QQYSKLPWT |
| 64 | Clothia | SEQ ID NO: 549 | SEQ ID NO: 550 | SEQ ID NO: 551 |
| | IMGT | QGIFNY | YTS | QQYSKLPWT |
| | | SEQ ID NO: 552 | SEQ ID NO: 553 | SEQ ID NO: 554 |
| SEQ ID NO: | Kabat/ | RASQSIGTSIH | YAFESIS | QQTYNWPYT |
| 65 | Clothia | SEQ ID NO: 555 | SEQ ID NO: 556 | SEQ ID NO: 557 |
| | IMGT | QSIGTS | YAF | QQTYNWPYT |
| | | SEQ ID NO: 558 | SEQ ID NO: 559 | SEQ ID NO: 560 |
| SEQ ID NO: | Kabat/ | RSSQNIVHRNGNT | KVSNRF | FQGSHFPFT |
| 66 | Clothia | YLE | SEQ ID NO: 562 | SEQ ID NO: 563 |
| | | SEQ ID NO: 561 | | |
| | IMGT | QNIVHRNGNTY | KVS | FQGSHFPFT |
| | | SEQ ID NO: 564 | SEQ ID NO: 565 | SEQ ID NO: 566 |
| SEQ ID NO: | Kabat/ | RSSQSLLHSNGNT | KVSNRFS | SQSTHVPWT |
| 67 | Clothia | YLH | SEQ ID NO: 568 | SEQ ID NO: 569 |
| | | SEQ ID NO: 567 | | |
| | IMGT | QSLLHSNGNTY | KVS | SQSTHVPWT |
| | | SEQ ID NO: 570 | SEQ ID NO: 571 | SEQ ID NO: 572 |
| SEQ ID NO: | Kabat/ | RSSQSLVHSNGNT | KVSNRFS | SQSTHVPYT |
| 68 | Clothia | YLQ | SEQ ID NO: 574 | SEQ ID NO: 575 |
| | | SEQ ID NO: 573 | | |
| | IMGT | QSLVHSNGNTY | KVS | SQSTHVPYT |
| | | SEQ ID NO: 576 | SEQ ID NO: 577 | SEQ ID NO: 578 |

### Immunoconjugates

The present invention provides immunoconjugates. In some embodiments, the immunoconjugates provided in the present invention include the antibodies or antigen-binding moieties provided in the present invention, and therapeutic agents (also referred to herein as “antibody-drug conjugates” or “ADCs”). The choice of a particular therapeutic agent depends on particular target molecules or cells, and desired biological effects. Thus, for example, the therapeutic agents may be cytotoxins used to cause death of particular target cells (e.g., tumor cells). Conversely, where it is desired to induce a non-lethal biological response, the therapeutic agents can be conjugated to non-lethal agents or liposomes containing non-lethal agents.

In some other embodiments, the immunoconjugates provided in the present invention include the antibodies or antigen-binding moieties provided in the present invention, and detectable markers. The detectable markers can directly or indirectly produce detectable signals, and thus in some embodiments, these immunoconjugates may be used for research or diagnostic applications, such as in vivo cancer detection. For example, the markers may be radiopaque isotopes or radioisotopes, such as ³H, actinium-225, astatine-211, bismuth-212, carbon-14, chromium-51, chlorine-36, cobalt-57, cobalt-58, and copper-67; fluorescent (fluorophore) or chemiluminescent (chromophore) compounds, such as fluorescent isothiocyanates, rhodamine, and fluorescein; enzymes, such as alkaline phosphatase, β-galactosidase, or horseradish peroxidase; or metal ions, such as chromium (III), manganese (II), iron (III), and iron (II); and radiocontrast medium.

Effector molecules may be linked to target antibodies by using any number of means known to those skilled in the art. In some embodiments, covalent and non-covalent linking methods may be used. Methods for linking effector molecules to antibodies vary depending on chemical structures of the effector molecules. Antibodies may be derivatized to expose or link reactive functional groups, and the derivatization may include linking any of known linker molecules. A linker may be any molecule used to link an antibody to an effector molecule. The linker is capable of forming a covalent bond with both the antibody and the effector molecule.

In some cases, an effector molecule needs to be released from an antibody when an immunoconjugate has reached its target site. Thus, in some embodiments, the immunoconjugate includes cleavable bonds near the target site. Cleavage of the linker to release the effector molecule from the antibody may be induced by enzymatic activity, or by a condition in which the immunoconjugate is located within the target cell or near the target site.

### Chimeric Antigen Receptors and Immunocompetent Cell

The present invention provides chimeric antigen receptors (CARs), which are collectively referred to as GPC3-CAR. This chimeric antigen receptor is expressed on the surface of an immunocompetent cell, so that the immunocompetent cell has highly specific cytotoxic effect on tumor cells expressing GPC3.

### Multispecific Molecules

The present invention further provides multispecific molecules. In some embodiments, a multispecific (e.g., bispecific) anti-GPC3 molecule is provided, including a) an anti-GPC3 antibody moiety that specifically recognizes target GPC3, and b) a second antibody moiety that specifically recognizes a second antigen. In some embodiments, the anti-GPC3 antibody moiety specifically recognizes GPC3 protein or GPC3 binding to the cell surface. In some embodiments, the second antibody moiety specifically recognizes a different GPC3 antigenic determinant as compared to the GPC3 antibody moiety. In some embodiments, the second antibody moiety specifically recognizes a different form of GPC3 as compared to the GPC3 antibody moiety.

### Pharmaceutical Composition

The present invention further provides a pharmaceutical composition that can be used for treatment and/or prevention of diseases (e.g., cancers) associated with cell proliferation, and is particularly useful for treatment and/or prevention of liver cancer.

In some embodiments, when the antibodies provided in the present invention are used as the pharmaceutical composition, the antibodies can be formulated into dosage forms by methods well known to those skilled in the art. For example, the antibodies can be used by injection as sterile solutions or suspensions in water or other pharmaceutical solutions. For example, the antibodies can be properly mixed with pharmaceutically acceptable solvents such as sterile water, physiological saline, vegetable oils, emulsifiers, suspensions, surfactants, stabilizers, perfumes, excipients, carriers, preservatives, and binders to prepare, in the form of lyophilized products or aqueous solutions, into formulations in desired unit dosage forms. “Pharmaceutically acceptable” means that a molecule, a molecular fragment or a composition, when properly administered to an animal or human, does not produce adverse, allergic or other adverse reactions.

In some embodiments, the composition provided in the present invention is formulated for parenteral administration, including but not limited to intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intrasternal, and intratumoral injection and infusion.

In some embodiments, the composition provided in the present invention may also contain one or more active compounds other than the anti-GPC3 antibody, preferably those having complementary activities that have no harmful effects on each other, as needed for a particular indication to be treated. For example, it may be required to further provide an anti-neoplastic agent, a growth inhibitor, a cytotoxic agent, or a chemotherapeutic agent in addition to the anti-GPC3 antibody. Such compounds are desirably present in combinations in amounts effective for intended purposes. The effective amount depends on the following factors: an amount of the anti-GPC3 antibody present in the formulation, the type of disease or disorder or treatment, and other factors as described above.

### Therapeutic Use

The present invention further provides a method for treating diseases and/or disorders (including, for example, cancers) involving abnormally high expression of GPC3.

In some embodiments, a therapeutically effective amount of the antibody is administered to a subject in an amount that induces the following biological activities in vivo and in vitro: inhibiting the growth, replication or metastasis of cancer cells having a high expression of GPC3: mediating phagocytosis or ADCC of cells having a high expression of GPC3 in the presence of effector cells; or preventing GPC3 ligands from binding to GPC3.

The therapeutically effective amount of the antibodies provided in the present invention will depend on severity of diseases and health status of patients. In addition, the amount to be administered also depends in part on binding affinity of the antibodies to antigens and pharmacokinetic properties of the antibodies in subjects. The dose and method of administration may vary depending on the weight, age and symptoms of patients, and are appropriately selected by those skilled in the art.

In some embodiments, the antibodies provided in the present invention may be used alone or in combination with other therapeutic agents or therapies. Such therapeutic agents include, but are not limited to, antineoplastic agents, such as chemotherapeutic agents, alkylating agents, antimetabolites, natural products, various agents (e.g., platinum coordination complexes), hormones, antagonists, and 1 immunomodulators.

Co-administration can resolve problems caused by development of drug resistance or changes in antigenicity of tumor cells (that would render them unresponsive to the antibodies). In some embodiments, the antibodies may be administered before, after, or in combination with the therapeutic agents, or may be co-administered with other known therapies (e.g., anticancer therapies, such as radiation).

### Detection and Diagnostic Use

The present invention provides a method for detecting or diagnosing a disease such as a tumor. In some embodiments, the detection is a quantitative detection or a non-quantitative detection. The quantitative detection includes determining concentration and content of a GPC3 protein. The non-quantitative detection includes, for example, determining only the presence of the GPC3 protein, determining the presence of a particular amount or more of the GPC3 protein, and determining the amount of the GPC3 protein as compared to the amount of GPC3 in another sample (e.g., a control sample).

Test samples are not particularly limited as long as the samples may contain the GPC3 protein, preferably samples collected from living organisms such as mammals, and more preferably samples collected from human. Specific examples of the test samples may include, for example, blood, interstitial fluid, plasma, extravascular fluid, cerebral fluid, synovial fluid, pleural fluid, serum, lymph, saliva, and preferably blood, serum and plasma. In addition, test samples obtained from, for example, cell culture solutions collected from living organisms are also included in the test samples provided in the present invention.

The GPC3 to be detected is not particularly limited and may be full-length GPC3 or a fragment thereof. When detecting fragments of GPC3, the fragments may be N-terminal fragments or C-terminal fragments. In addition, the GPC3 protein may be GPC3 or GPC3 core protein to which heparan sulfate is added.

The method for detecting the GPC3 protein contained in the test samples is not particularly limited, and is preferably performed by immunological methods using an anti-GPC3 antibody. Examples of the immunological methods include, for example, radioimmunoassay, enzyme-linked immunoassay, fluoroimmunoassay, luminescence immunoassay, immunoprecipitation, and turbidimetric immunoassay, preferably enzyme-linked immunoassay, and even more preferably an ELISA (e.g., indirect ELISA). The foregoing immunological methods may be recognized by means known to those skilled in the art.

The present invention will be further described with reference to specific examples, and the advantages and features of the present invention will become more apparent with the description. Experimental procedures without specified conditions in the examples are conducted according to conventional conditions or conditions recommended by the manufacturer. Reagents or instruments without specified manufacturers used herein are conventional products that are commercially available.

The examples are exemplary only and do not limit the scope of the present invention in any way. It will be understood by those skilled in the art that various changes or substitutions in form and details may be made to the technical solutions of the present invention without departing from the spirit and scope of the present invention, and that these changes and substitutions shall fall within the scope of the present invention.

### Example 1: Preparation of Control Antibodies, Preparation of Human GPC3 Polypeptides, Identification of Endogenous Cells, and Preparation of Cell Strains of Over-Expressed GPC3 Protein

### (A) Preparation of Control Antibodies

Y035, T2-23, and GC33 clones are antibodies that recognize human GPC3 protein, have strong binding affinity to the human GPC3 protein, and can also bind to cell lines with a high expression of GPC3, such as HepG2.

Sequences for heavy chain variable regions and light chain variable regions of the Y035 clone and the T2-23 clone are obtained according to the patent US2019/0046659A1, and sequences for heavy chain variable regions and light chain variable regions of the GC33 clone are obtained according to the patent CN101287492B. VL and VH of Y035 and T2-23, which recognize human GPC3, and human IgG1 Fc were linked in an order from an N-terminus to a C-terminus, respectively, wherein the VH and the VL were linked by 3 GGGGS linkers to form scFv-human IgG1 Fc (scFv-hFc). VH and VL of GC33, which recognizes human GPC3, and human IgG1 Fc were linked in an order from an N-terminus to a C-terminus, respectively, wherein the VH and the VL were linked by 3 GGGGS linkers to form scFv-human IgG1 Fc (scFv-hFc). The GC33 expressed complete IgGs at the same time. Sequences of Y035 VH, Y035 VL, Y035 scFv-hFc, T2-23 VH, T2-23 VL, T2-23 scFv-hFc, GC33 VH, GC33 VL, GC33 scFv-hFc, and GC33 IgG are shown in Table 3, respectively. Nucleotide sequences were cloned into a pTT5 vector (purchased from Youbio) respectively, and plasmids were prepared according to established standard molecular biology methods. See Sambrook, J., Fritsch, E. F., and Maniatis, T. (1989). Molecular Cloning: A Laboratory Manual, Second Edition (Plainview, New York: Cold Spring Harbor Laboratory Press). HEK293E cells (purchased from the Cell Bank of Type Culture Collection of Chinese Academy of Sciences) were transiently transfected with expression vectors according to PEI (purchased from Polysciences) instructions, cultured at 37° C. for 5 consecutive days using FreeStyle™ 293 (Invitrogen), and centrifuged to remove cell components to obtain culture supernatants containing antibodies in the form of scFv-human IgG1 Fc (hFc) or IgG. The culture supernatants were each loaded on a Protein A chromatography column (Protein A packing AT Protein A Diamond and column BXK16/26 were both purchased from Bestchrom), washed with a PBS phosphate buffer (pH 7.4), then washed with 20 mM PB and 1M NaCl (pH 7.2), and finally eluted with a citrate buffer at pH 3.4. An Fc-tagged antibody eluted from the Protein A chromatography column was collected, neutralized with 1/10 volume of 1M Tris at pH 8.0, and dialyzed with PBS at 4° C. overnight. The concentration of the dialyzed antibody was determined using Nanodrop, the purity of the antibody was determined using HPLC-SEC, the endotoxin content of the antibody was determined using an endotoxin assay kit (purchased from Zhanjiang A&C Biological Ltd.), and finally the control antibody was aseptically filtered through a 0.22 μM filter membrane, subpackaged, and stored at −80° C.

TABLE 3
Amino acid sequences of control antibodies

| Sequence | Sequence | |
|---|---|---|
| name | No. | Amino acid sequence |
| Y035 VH | SEQ ID | EVQLVQSGAEVKKPGASVKVSCKASGYTFSDYEMHWVR |
| | NO: 579 | QAPGQGLEWMGAIHPGSGDTAYNQRFKGRVTITADKSTS |
| | | TAYMELSSLRSEDTAVYYCARFYSYAYWGQGTLVTVSA |
| Y035 VL | SEQ ID | DIVMTQTPLSLPVITGEPASISCRSSQSLVHSNGNTYLQWY |
| | NO: 580 | LQKPGQSPQLLIYKVSNRFSGVPDRFSGSGSGTDFTLKISR |
| | | VEAEDVGVYYCSQSIYVPYTFGQGTKLEIKR |
| Y035 | SEQ ID | DIVMTQTPLSLPVITGEPASISCRSSQSLVHSNGNTYLQWY |
| scFv- | NO: 581 | LQKPGQSPQLLIYKVSNRFSGVPDRFSGSGSGTDFTLKISR |
| hFc | | VEAEDVGVYYCSQSIYVPYTFGQGTKLEIKRGGGGSGGG |
| | | GSGGGGSEVQLVQSGAEVKKPGASVKVSCKASGYTFSDY |
| | | EMHWVRQAPGQGLEWMGAIHPGSGDTAYNQRFKGRVTI |
| | | TADKSTSTAYMELSSLRSEDTAVYYCARFYSYAYWGQGT |
| | | LVTVSAEPKSADKTHTCPPCPAPELLGGPSVFLFPPKPKDT |
| | | LMISRTPEVTCVVVDVSHEDPEVKFNWYVDGVEVHNAK |
| | | TKPREEQYNSTYRVVSVLTVLHQDWLNGKEYKCKVSNK |
| | | ALPAPIEKTISKAKGQPREPQVYTLPPSREEMTKNQVSLTC |
| | | LVKGFYPSDIAVEWESNGQPENNYKTTPPVLDSDGSFFLY |
| | | SKLTVDKSRWQQGNVFSCSVMHEALHNHYTQKSLSLSPG |
| | | K |
| T2-23 | SEQ ID | QVQLQESGGGLVQPGRSLRLSCAASGFTFSSYAMHWVRQ |
| VH | NO: 582 | APGKGLEWVSAISSSGRSTYYADSVEGRFTISRDNSKNTL |
| | | YLQMNSLRAEDTAVYYCAKDRRGSHADALNVWGQGTL |
| | | VTVSS |
| T2-23 | SEQ ID | QSALTQPPSASGSPGQSVTISCTGTSSDVGGYNYVSWYQQ |
| VL | NO: 583 | YPGKAPKLLIYGNSNRPSGVPDRESGSKSGTSASLAITGLQ |
| | | AEDGADYYCQSYDSSLRVVFGGGTKVTVLG |
| T2-23 | SEQ ID | QSALTQPPSASGSPGQSVTISCTGTSSDVGGYNYVSWYQQ |
| scFv- | NO: 584 | YPGKAPKLLIYGNSNRPSGVPDRESGSKSGTSASLAITGLQ |
| hFc | | AEDGADYYCQSYDSSLRVVFGGGTKVTVLGGGGGSGGG |
| | | GSGGGGSQVQLQESGGGLVQPGRSLRLSCAASGFTFSSYA |
| | | MHWVRQAPGKGLEWVSAISSSGRSTYYADSVEGRFTISR |
| | | DNSKNTLYLQMNSLRAEDTAVYYCAKDRRGSHADALNV |
| | | WGQGTLVTVSSEPKSADKTHTCPPCPAPELLGGPSVFLFP |
| | | PKPKDTLMISRTPEVTCVVVDVSHEDPEVKFNWYVDGVE |
| | | VHNAKTKPREEQYNSTYRVVSVLTVLHQDWLNGKEYKC |
| | | KVSNKALPAPIEKTISKAKGQPREPQVYTLPPSREEMTKN |
| | | QVSLTCLVKGFYPSDIAVEWESNGQPENNYKTTPPVLDSD |
| | | GSFFLYSKLTVDKSRWQQGNVFSCSVMHEALHNHYTQK |
| | | SLSLSPGK |
| GC33 | SEQ ID | QVQLVQSGAEVKKPGASVKVSCKASGYTFTDYEMHWVR |
| VH | NO: 589 | QAPGQGLEWMGALDPKTGDTAYSQKFKGRVTLTADKST |
| | | STAYMELSSLTSEDTAVYYCTRFYSYTYWGQGTLVTVSS |
| GC33 VL | SEQ ID | DVVMTQSPLSLPVTPGEPASISCRSSQSLVHSNRNTYLHW |
| | NO: 590 | YLQKPGQSPQLLIYKVSNRFSGVPDRFSGSGSGTDFTLKIS |
| | | RVEAEDVGVYYCSQNTHVPPTFGQGTKLEIK |
| GC33 | SEQ ID | QVQLVQSGAEVKKPGASVKVSCKASGYTFTDYEMHWVR |
| scFv-hFc | NO: 591 | QAPGQGLEWMGALDPKTGDTAYSQKFKGRVTLTADKST |
| | | STAYMELSSLTSEDTAVYYCTRFYSYTYWGQGTLVTVSS |
| | | GGGGSGGGGSGGGGSDVVMTQSPLSLPVTPGEPASISCRS |
| | | SQSLVHSNRNTYLHWYLQKPGQSPQLLIYKVSNRFSGVP |
| | | DRFSGSGSGTDFTLKISRVEAEDVGVYYCSQNTHVPPTFG |
| | | QGTKLEIKEPKSSDKTHTCPPCPAPELLGGPSVFLFPPKPK |
| | | DTLMISRTPEVTCVVVDVSHEDPEVKFNWYVDGVEVHN |
| | | AKTKPREEQYNSTYRVVSVLTVLHQDWLNGKEYKCKVS |
| | | NKALPAPIEKTISKAKGQPREPQVYTLPPSREEMTKNQVS |
| | | LTCLVKGFYPSDIAVEWESNGQPENNYKTTPPVLDSDGSF |
| | | FLYSKLTVDKSRWQQGNVFSCSVMHEALHNHYTQKSLSL |
| | | SPGK |
| GC33 | SEQ ID | QVQLVQSGAEVKKPGASVKVSCKASGYTFTDYEMHWVR |
| IgG HC | NO: 592 | QAPGQGLEWMGALDPKTGDTAYSQKFKGRVTLTADKST |
| | | STAYMELSSLTSEDTAVYYCTRFYSYTYWGQGTLVTVSS |
| | | ASTKGPSVFPLAPSSKSTSGGTAALGCLVKDYFPEPVTVS |
| | | WNSGALTSGVHTFPAVLQSSGLYSLSSVVTVPSSSLGTQT |
| | | YICNVNHKPSNTKVDKKVEPKSCDKTHTCPPCPAPELLGG |
| | | PSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALPAPIEKTISKAKGQPREPQVYTLPPSR |
| | | EEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| GC33 | SEQ ID | DVVMTQSPLSLPVTPGEPASISCRSSQSLVHSNRNTYLHW |
| IgG | NO: 593 | YLQKPGQSPQLLIYKVSNRFSGVPDRFSGSGSGTDFTLKIS |
| LC | | RVEAEDVGVYYCSQNTHVPPTFGQGTKLEIKRTVAAPSV |
| | | FIFPPSDEQLKSGTASVVCLLNNFYPREAKVQWKVDNAL |
| | | QSGNSQESVTEQDSKDSTYSLSSTLTLSKADYEKHKVYAC |
| | | EVTHQGLSSPVTKSFNRGEC |

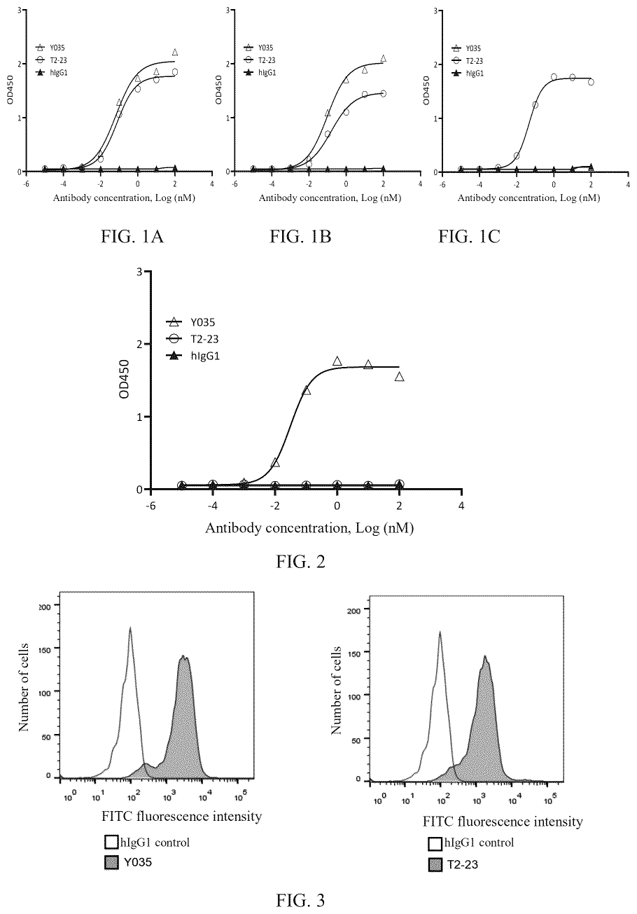

The binding activity of Y035 and T2-23 to a human GPC3-His protein (purchased from Acro, Cat. No. GP3-H52H4), a monkey GPC3-His protein (purchased from Acro, Cat. No. GP3-C5225), and a murine GPC3-His protein (purchased from Sino Biological, Cat. No. 50989-M08B) were assayed by ELISA. The specific method is described as follows: The antigen protein was diluted with PBS to a final concentration of 1 μg/mL, and the diluent was added to a 96-well ELISA plate at 50 μL/well. The plate was sealed with a plastic film and incubated at 4° C. overnight, washed twice with PBS the next day, and a blocking solution [PBS+2% (w/w) BSA] was added for blocking at room temperature for 2 h. The blocking solution was discarded, and 100 nM gradient-diluted control antibody or a negative control antibody was added at 50 μL/well. After incubation at 37° C. for 2 h, the plate was washed 3 times with PBS. A horseradish peroxidase (HRP)-labeled secondary antibody (purchased from Merck, Cat. No. AP113P) was added for incubation at 37° C. for 1 h, and the plate was washed 5 times with PBS. A TMB substrate was added at 50 μL/well for incubation at room temperature for 10 min, then a stop solution (1.0M HCl) was added at 50 μL/well. An ELISA plate reader (Multimode Plate Reader, EnSight, purchased from Perkin Elmer) was used to read OD_{450 nm} values, with results as shown in Table 4 to Table 6, FIG. 1A, FIG. 1B, and FIG. 1C. The results showed that both the antibodies Y035 and T2-23 had good binding activity with the human GPC3 protein and the monkey GPC3 protein; and the Y035 did not bind to the murine GPC3 protein, and the T2-23 bound well to the murine GPC3 protein. The IgG subtype control was human IgG1.

TABLE 4
Assay results for binding reactions of control
antibodies with human GPC3-his protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | blank |
| Y035 | 2.219 | 1.856 | 1.735 | 1.289 | 0.337 | 0.09 | 0.056 | 0.048 |
| T2-23 | 1.85 | 1.706 | 1.534 | 1.06 | 0.231 | 0.073 | 0.068 | 0.049 |
| hIgG1 | 0.072 | 0.05 | 0.048 | 0.05 | 0.048 | 0.048 | 0.047 | 0.049 |

TABLE 5
Assay results for binding reactions of control antibodies
with monkey GPC3-his protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | blank |
| Y035 | 2.103 | 1.888 | 1.706 | 1.091 | 0.254 | 0.072 | 0.047 | 0.046 |
| T2-23 | 1.446 | 1.431 | 1.103 | 0.694 | 0.131 | 0.057 | 0.044 | 0.046 |
| hIgG1 | 0.056 | 0.047 | 0.044 | 0.045 | 0.044 | 0.044 | 0.048 | 0.046 |

TABLE 6
Assay results for binding reactions of control antibodies
with murine GPC3-his protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | blank |
| Y035 | 0.09 | 0.064 | 0.049 | 0.056 | 0.054 | 0.05 | 0.058 | 0.051 |
| T2-23 | 1.672 | 1.761 | 1.77 | 1.252 | 0.303 | 0.086 | 0.05 | 0.052 |
| hIgG1 | 0.105 | 0.055 | 0.05 | 0.05 | 0.052 | 0.051 | 0.049 | 0.057 |

### (B) Preparation of Human Polypeptide GC3Pep

Polypeptide GC3pep (AELAYDLDVDDAPGNSQQATPKDNEISTFHNLGNVHSPLK, SEQ ID NO: 585) of human GPC3 (NCBI: NM_004484.3, Ala524-Lys563) was produced. The prepared polypeptides were assayed by ELISA as described in Example 1 (A), respectively, with positive control antibodies which recognized different epitopes, with results as shown in Table 7 and FIG. 2. The results showed that the T2-23 could not bind to the polypeptide GC3pep, while the Y035 could bind to the polypeptide GC3pep, indicating that the polypeptides with binding activity have been prepared.

TABLE 7
Assay results for binding reactions of control antibodies
with polypeptide GC3pep protein by ELISA

| OD450 | | | | | | | | |
|---|---|---|---|---|---|---|---|---|
| Antibody | Antibody concentration (nM) | | | | | | | |
| name | 100 | 10 | 1 | 0.1 | 0.01 | 0.001 | 0.0001 | blank |
| Y035 | 1.554 | 1.722 | 1.769 | 1.366 | 0.373 | 0.088 | 0.056 | 0.049 |
| T2-23 | 0.069 | 0.049 | 0.059 | 0.051 | 0.046 | 0.065 | 0.063 | 0.048 |
| hIgG1 | 0.069 | 0.05 | 0.05 | 0.047 | 0.046 | 0.046 | 0.06 | 0.051 |

### (C) Identification of Cell Strains Endogenously Expressing GPC3 Protein

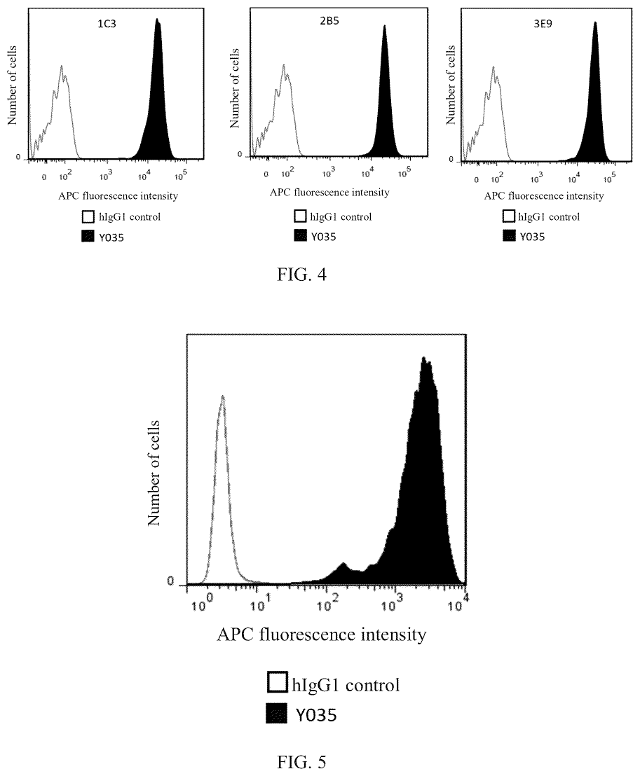

HepG2 cells were expanded to a logarithmic growth phase in a T-75 cell culture flask, the culture supernatant was discarded by centrifugation, and the cell pellet was washed twice with PBS. Assay and analysis were performed by FACS (FACS Canto™, purchased from BD Biosciences) using the antibodies Y035 and T2-23 as primary antibodies and an FITC-labeled secondary antibody (purchased from Invitrogen, Cat. No. A11013), with results as shown in Table 8 and FIG. 3. The results showed that HepG2 cells could bind to both the Y035 and the T2-23.

TABLE 8
Assay results for endogenous cell line HepG2 cells by FACS

| | |
|---|---|
| Antibody | Mean fluorescence intensity of cells |

| No. | name | IgG subtype control | GPC3 antibody |
|---|---|---|---|
| 1 | Y035 | 137 | 3647 |
| 2 | T2-23 | 137 | 2573 |

### (D) Preparation of CHO-K1 Recombinant Cell Strain Expressing Human GPC3 Protein

A nucleotide sequence encoding a full-length amino acid sequence (NCBI: NM_004484.3) of human GPC3 was cloned into a pcDNA3.1 vector (purchased from Clontech), and a plasmid was prepared. After plasmid transfection (Lipofectamine® 3000 Transfection Kit, purchased from Invitrogen, Cat. No. L3000-015), CHO-K1 cell lines (purchased from the Cell Bank of Type Culture Collection of Chinese Academy of Sciences) were subjected to selective culture in a DMEM/F12 medium containing 10% (w/w) fetal bovine serum and containing 10 μg/mL puromycin for 2 weeks, then positive monoclonal cells were sorted onto a 96-well plate on a flow cytometer (FACSAriaII, purchased from BD Biosciences) using an antibody Y035 and a goat anti-human IgG H+L antibody (Jackson, Cat. No. 109605088), and cultured in 5% (v/v) CO₂ at 37° C., and a portion of the monoclonal wells were selected for amplification approximately 2 weeks later. The amplified clones were screened by flow cytometry. Monoclonal cell lines with better growth and higher fluorescence intensity were selected for further expansion and cryopreserved in liquid nitrogen. The results of selection are shown in Table 9 and FIG. 4, in which the IgG subtype control was a human IgG1 control. Table 9 shows that a series of CHO-K1 monoclonal cell lines with positive expression of human GPC3 have been prepared. In FIG. 4, the abscissa represents the fluorescence intensity of the cells, and the ordinate represents the number of the cells. The results indicated that 1C3, 2B5, and 3E9 were cell strains with high-level expression of human GPC3.

TABLE 9
Assay results for CHO-K1 recombinant cell
lines expressing human GPC3 protein by FACS

| | Clone No. of stably | Mean fluorescence intensity of cells | |
|---|---|---|---|
| No. | transfected cell line | IgG subtype control | GPC3 antibody |
| 1 | CHOK1-hGPC3.1C3 | 19.3 | 14300 |
| 2 | CHOK1-hGPC3.2B5 | 19.3 | 18900 |
| 3 | CHOK1-hGPC3.3E9 | 19.3 | 26700 |

### (E) Preparation of Recombinant HEK293T Cell Strains Expressing Monkey GPC3 Protein

A nucleotide sequence encoding a full-length amino acid sequence (NCBI: XP_011739317.1) of monkey GPC3 was cloned into a pcDNA3.1 vector (purchased from Thermofisher scientific), and a plasmid was prepared. After plasmid transfection with FuGENE® HD (Promega, Cat. No. #E2311), HEK293T cell lines were subjected to selective culture in a DMEM medium containing 10% (w/w) fetal bovine serum and containing 10 μg/mL puromycin for 2 weeks, then enriched positive monoclonal cells were sorted onto a 96-well plate on a flow cytometer (FACSAriaII, purchased from BD Biosciences) using an antibody Y035 and a goat anti-human IgG H+L antibody (Jackson, Cat. No. 109605088), cultured in 5% (v/v) CO₂ at 37° C., and amplified about one week later. The amplified cells were assayed by flow cytometry, and cell strains with better growth and higher fluorescence intensity were selected for further expansion and cryopreserved in liquid nitrogen. The results of expression levels are shown in FIG. 5, indicating that HEK293T-monkey-GPC3 subjected to puromycin pressure screening had a single positive peak and could be used for assaying cross-activity of antibodies.

### Example 2 Preparation of Hybridoma Antibody Against GPC3

### (A) Mouse Immunization and Serum Titer Assay

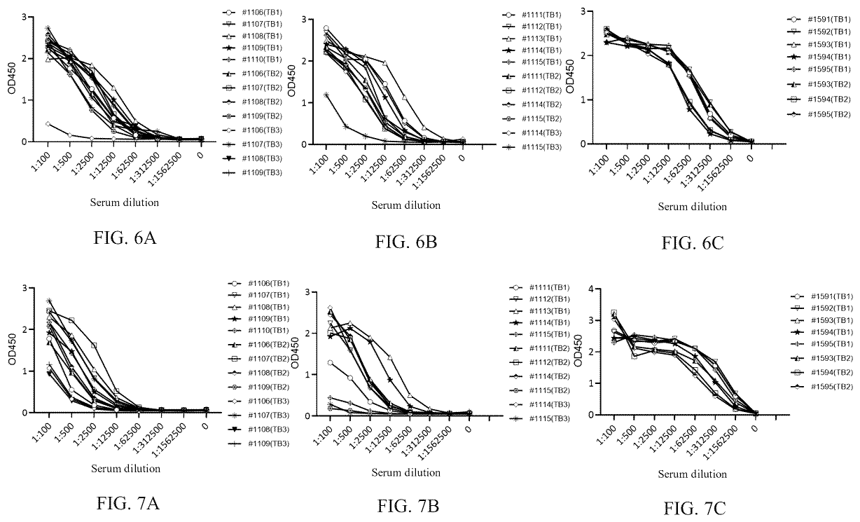

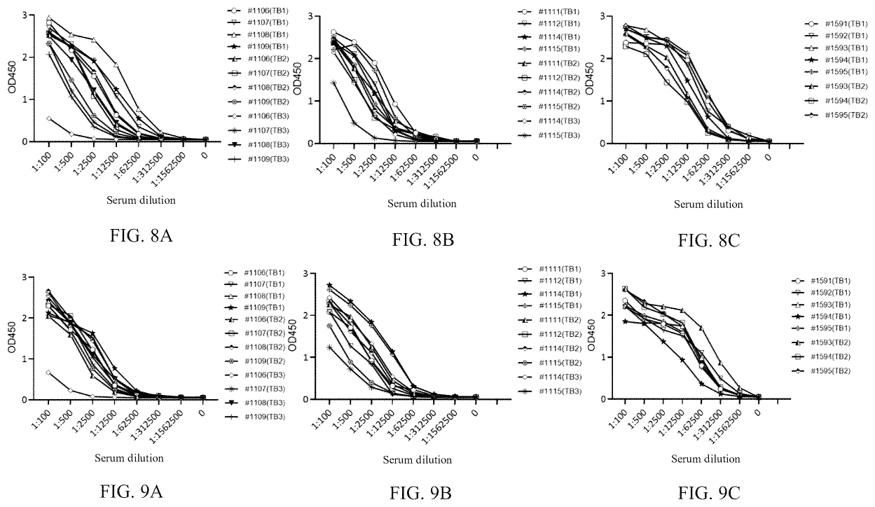

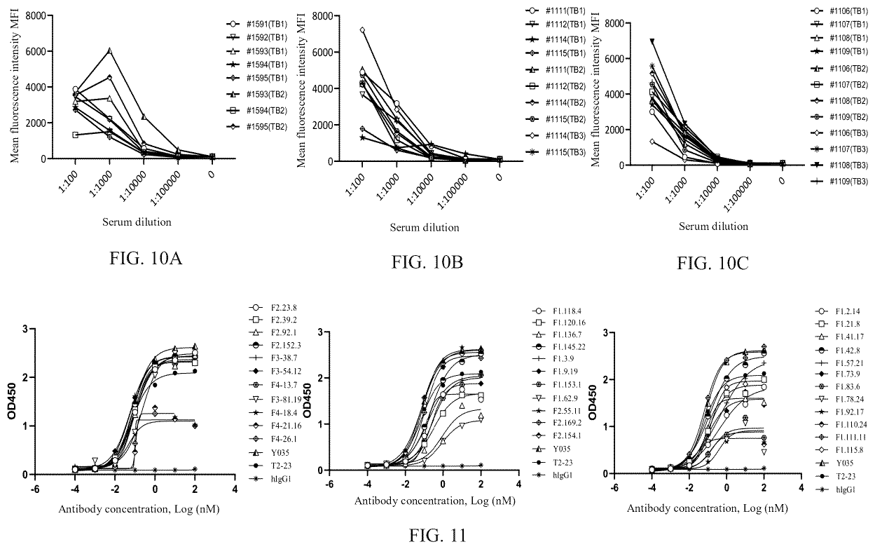

Polypeptide GC3pep coupled to a carrier protein (KLH) was produced, i.e., polypeptide GC3pep-KLH (AELAYDLDVDDAPGNSQQATPKDNEISTFHNLGNVHSPLKC-KLH, SEQ ID NO: 586). For the animal immunization experiment, animals were divided into three groups, the first group was immunized with a human GPC3 (Gln 25-His 559)-hFc protein (purchased from Acro, Cat. No. GP3-H5258), the second group was immunized with a human GPC3 (Met1-His559)-his protein (purchased from Sino Biological, Cat. No. 10088-H08H), and the third group was immunized with polypeptide GC3pep-KLH. The laboratory animals were 6-8 week-old female SJL or MRL/lpr mice (purchased from Shanghai SLAC Laboratory Animal Co., Ltd.) and were housed/kept in an SPF environment. Blood was collected from orbits of the mice before immunization and used as negative serum. During primary immunization, one third of the antigen was co-emulsified with Alum (purchased from Thermo, Cat. No. 77161) and CpG (synthesis entrusted to Sangon Biotech, Cat. No. ODN1826) and injected intraperitoneally (0.1 ml), while the remaining antigen was emulsified with TiterMax (purchased from Sigma, Cat. No. T2684) and injected subcutaneously and intraplantarly at multiple sites, that is, 100 μg of immunogen was injected into each mouse. A booster immunization was performed every other week thereafter, and during the booster immunization, the antigen was emulsified with TiterMax and injected subcutaneously at multiple sites, that is, 50 μg of immunogen was injected into each mouse. Mice in the first two groups received seven booster immunizations, and the immunogen for the seventh booster immunization was polypeptide GC3pep-KLH. Mice in the third group received six booster immunizations. Blood was collected from orbits of the mice after the second, sixth and seventh booster immunizations, respectively, and serum batches were successively named TB1, TB2 and TB3. Binding titers of the antibodies in the mouse serum to the human GPC3-His protein (FIG. 6A, FIG. 6B, FIG. 6C, and Table 10) and binding titers of the antibodies in the mouse serum to polypeptide GC3pep (FIG. 7A, FIG. 7B, FIG. 7C, and Table 11) were assayed by ELISA respectively, and cross-binding activities of the antibodies in the serum to the murine GPC3-His protein and the monkey GPC3-his protein were assayed, with results as shown in FIG. 8A, FIG. 8B, FIG. 8C, and Table 12, as well as FIG. 9A, FIG. 9B, FIG. 9C, and Table 13, respectively. In addition, binding specificity of the antibodies in the serum to HepG2 cells was assayed by FACS, with results as shown in FIG. 10A, FIG. 10B, FIG. 10C, and Table 14. The results showed that the serum of the immunized mice had different degrees of binding to the immunogens, and showed antigen-antibody reactions. The ELISA blank control was 1% (w/w) BSA, and data in the table are OD_{450 nm} values; while the FACS blank control was 2% (w/w) FBS, and data in the table are mean fluorescence intensity MFI.

TABLE 10
Assay results for binding titers of mouse serum antibodies to human GPC3-His protein by ELISA

| | | | Serum dilution | | | | | | | |
|---|---|---|---|---|---|---|---|---|---|---|
| Immunized | | OD450 nm | | | | | | | | Blank |
| animal | Immunogen | Batch | 1:100 | 1:500 | 1:2500 | 1:12500 | 1:62500 | 1:312500 | 1:1562500 | control |
| SJL | GPC3-Fc | #1106(TB1) | 2.27 | 1.76 | 1.27 | 0.57 | 0.16 | 0.08 | 0.05 | 0.05 |
| | (immunized | #1107(TB1) | 2.50 | 2.00 | 1.54 | 0.89 | 0.27 | 0.10 | 0.06 | 0.05 |
| | with GC3pep) | #1108(TB1) | 1.98 | 2.02 | 1.84 | 1.30 | 0.50 | 0.17 | 0.07 | 0.05 |
| | | #1109(TB1) | 2.29 | 2.01 | 1.60 | 1.01 | 0.34 | 0.12 | 0.06 | 0.05 |
| | | #1110(TB1) | 2.43 | 2.21 | 1.58 | 0.86 | 0.24 | 0.09 | 0.05 | 0.05 |
| | | #1106(TB2) | 2.22 | 1.94 | 1.16 | 0.40 | 0.12 | 0.07 | 0.05 | 0.06 |
| | | #1107(TB2) | 2.41 | 2.12 | 1.70 | 0.66 | 0.19 | 0.08 | 0.06 | 0.06 |
| | | #1108(TB2) | 2.58 | 2.00 | 1.53 | 0.61 | 0.19 | 0.09 | 0.07 | 0.07 |
| | | #1109(TB2) | 2.41 | 1.62 | 0.76 | 0.25 | 0.10 | 0.07 | 0.06 | 0.07 |
| | | #1106(TB3) | 0.43 | 0.16 | 0.08 | 0.07 | 0.06 | 0.07 | 0.07 | 0.08 |
| | | #1107(TB3) | 2.73 | 1.89 | 1.06 | 0.49 | 0.30 | 0.25 | 0.07 | 0.07 |
| | | #1108(TB3) | 2.37 | 2.03 | 1.22 | 0.55 | 0.38 | 0.07 | 0.06 | 0.07 |
| | | #1109(TB3) | 2.14 | 1.62 | 0.82 | 0.39 | 0.28 | 0.06 | 0.06 | 0.07 |
| SJL | GPC3-his | #1111(TB1) | 2.79 | 2.27 | 2.04 | 1.45 | 0.57 | 0.16 | 0.07 | 0.05 |
| | (immunized | #1112(TB1) | 2.68 | 2.16 | 1.84 | 0.63 | 0.30 | 0.11 | 0.06 | 0.05 |
| | with GC3pep) | #1113(TB1) | 2.49 | 2.21 | 2.11 | 1.96 | 1.14 | 0.41 | 0.14 | 0.05 |
| | | #1114(TB1) | 2.40 | 2.28 | 1.95 | 1.13 | 0.33 | 0.10 | 0.06 | 0.05 |
| | | #1115(TB1) | 2.62 | 2.05 | 2.03 | 1.36 | 0.51 | 0.15 | 0.07 | 0.07 |
| | | #1111(TB2) | 2.33 | 2.06 | 1.54 | 0.64 | 0.19 | 0.08 | 0.06 | 0.06 |
| | | #1112(TB2) | 2.22 | 1.84 | 1.08 | 0.38 | 0.12 | 0.07 | 0.06 | 0.06 |
| | | #1114(TB2) | 2.22 | 1.92 | 1.32 | 0.45 | 0.14 | 0.07 | 0.06 | 0.06 |
| | | #1115(TB2) | 2.30 | 1.86 | 1.35 | 0.54 | 0.19 | 0.08 | 0.06 | 0.06 |
| | | #1114(TB3) | 2.19 | 1.75 | 1.11 | 0.39 | 0.13 | 0.08 | 0.07 | 0.13 |
| | | #1115(TB3) | 1.19 | 0.43 | 0.20 | 0.08 | 0.06 | 0.05 | 0.06 | 0.08 |
| MRL/lpr | GC3pep | #1591(TB1) | 2.52 | 2.28 | 2.16 | 2.16 | 1.62 | 0.68 | 0.20 | 0.06 |
| | | #1592(TB1) | 2.50 | 2.27 | 2.16 | 2.17 | 1.68 | 0.94 | 0.27 | 0.05 |
| | | #1593(TB1) | 2.54 | 2.27 | 2.24 | 2.11 | 1.58 | 0.61 | 0.16 | 0.05 |
| | | #1594(TB1) | 2.30 | 2.21 | 2.14 | 1.82 | 0.78 | 0.23 | 0.08 | 0.05 |
| | | #1595(TB1) | 2.30 | 2.40 | 2.26 | 2.23 | 1.54 | 0.58 | 0.15 | 0.06 |
| | | #1593(TB2) | 2.48 | 2.36 | 2.26 | 2.08 | 1.59 | 0.89 | 0.28 | 0.05 |
| | | #1594(TB2) | 2.60 | 2.25 | 2.14 | 1.80 | 0.95 | 0.31 | 0.10 | 0.05 |
| | | #1595(TB2) | 2.59 | 2.30 | 2.04 | 1.78 | 0.91 | 0.30 | 0.10 | 0.05 |

TABLE 11
Assay results for binding titers of mouse serum antibodies to polypeptide GC3pep by ELISA

| | | | Serum dilution | | | | | | | |
|---|---|---|---|---|---|---|---|---|---|---|
| Immunized | | OD450 nm | | | | | | | | Blank |
| animal | Immunogen | Batch | 1:100 | 1:500 | 1:2500 | 1:12500 | 1:62500 | 1:312500 | 1:1562500 | control |
| SJL | GPC3-Fc | #1106(TB1) | 1.78 | 0.55 | 0.18 | 0.07 | 0.05 | 0.05 | 0.05 | 0.05 |
| | (immunized | #1107(TB1) | 2.42 | 1.71 | 0.83 | 0.29 | 0.10 | 0.06 | 0.05 | 0.05 |
| | with GC3pep) | #1108(TB1) | 2.30 | 1.86 | 1.04 | 0.36 | 0.11 | 0.06 | 0.05 | 0.05 |
| | | #1109(TB1) | 1.92 | 1.47 | 0.50 | 0.16 | 0.07 | 0.05 | 0.05 | 0.05 |
| | | #1110(TB1) | 2.17 | 1.43 | 0.56 | 0.20 | 0.07 | 0.05 | 0.04 | 0.05 |
| | | #1106(TB2) | 1.69 | 0.96 | 0.29 | 0.10 | 0.06 | 0.05 | 0.05 | 0.08 |
| | | #1107(TB2) | 2.45 | 2.22 | 1.62 | 0.52 | 0.13 | 0.06 | 0.05 | 0.05 |
| | | #1108(TB2) | 2.17 | 1.10 | 0.35 | 0.12 | 0.06 | 0.06 | 0.05 | 0.05 |
| | | #1109(TB2) | 2.08 | 0.99 | 0.31 | 0.11 | 0.06 | 0.05 | 0.05 | 0.05 |
| | | #1106(TB3) | 1.07 | 0.33 | 0.11 | 0.08 | 0.07 | 0.07 | 0.07 | 0.07 |
| | | #1107(TB3) | 2.69 | 1.74 | 0.81 | 0.25 | 0.08 | 0.07 | 0.06 | 0.07 |
| | | #1108(TB3) | 0.93 | 0.29 | 0.09 | 0.05 | 0.04 | 0.06 | 0.06 | 0.07 |
| | | #1109(TB3) | 1.16 | 0.37 | 0.13 | 0.06 | 0.05 | 0.05 | 0.06 | 0.07 |
| SJL | GPC3-his | #1111(TB1) | 1.29 | 0.92 | 0.34 | 0.13 | 0.06 | 0.05 | 0.06 | 0.06 |
| | (immunized | #1112(TB1) | 2.24 | 1.59 | 0.68 | 0.18 | 0.08 | 0.06 | 0.05 | 0.05 |
| | with GC3pep) | #1113(TB1) | 2.13 | 2.25 | 1.90 | 1.42 | 0.50 | 0.17 | 0.07 | 0.05 |
| | | #1114(TB1) | 1.92 | 2.12 | 1.79 | 0.87 | 0.23 | 0.08 | 0.06 | 0.05 |
| | | #1115(TB1) | 0.44 | 0.31 | 0.12 | 0.06 | 0.05 | 0.05 | 0.05 | 0.05 |
| | | #1111(TB2) | 2.53 | 1.85 | 0.89 | 0.31 | 0.10 | 0.06 | 0.05 | 0.05 |
| | | #1112(TB2) | 2.01 | 1.87 | 0.80 | 0.25 | 0.09 | 0.06 | 0.05 | 0.05 |
| | | #1114(TB2) | 2.45 | 1.93 | 0.86 | 0.25 | 0.09 | 0.06 | 0.05 | 0.05 |
| | | #1115(TB2) | 0.18 | 0.13 | 0.06 | 0.05 | 0.05 | 0.05 | 0.05 | 0.08 |
| | | #1114(TB3) | 2.62 | 1.59 | 0.68 | 0.23 | 0.10 | 0.08 | 0.07 | 0.09 |
| | | #1115(TB3) | 0.28 | 0.08 | 0.06 | 0.05 | 0.05 | 0.07 | 0.07 | 0.10 |
| MRL/lpr | GC3pep | #1591(TB1) | 2.68 | 2.37 | 2.29 | 2.34 | 2.11 | 1.57 | 0.60 | 0.06 |
| | | #1592(TB1) | 2.63 | 2.33 | 2.34 | 2.42 | 2.11 | 1.69 | 0.68 | 0.05 |
| | | #1593(TB1) | 2.44 | 2.43 | 2.35 | 2.35 | 2.12 | 1.58 | 0.61 | 0.06 |
| | | #1594(TB1) | 2.44 | 2.50 | 2.37 | 2.26 | 1.86 | 1.03 | 0.26 | 0.05 |
| | | #1595(TB1) | 2.30 | 2.55 | 2.47 | 2.37 | 2.10 | 1.43 | 0.48 | 0.05 |
| | | #1593(TB2) | 3.21 | 2.17 | 2.09 | 2.02 | 1.72 | 1.09 | 0.39 | 0.04 |
| | | #1594(TB2) | 3.26 | 1.86 | 2.05 | 1.96 | 1.43 | 0.69 | 0.19 | 0.05 |
| | | #1595(TB2) | 3.04 | 2.12 | 1.99 | 1.90 | 1.25 | 0.60 | 0.17 | 0.04 |

TABLE 12
Assay results for cross-binding titers of mouse serum antibodies to murine GPC3-His protein by ELISA

| | | | Serum dilution | | | | | | | |
|---|---|---|---|---|---|---|---|---|---|---|
| Immunized | | OD450 nm | | | | | | | | Blank |
| animal | Immunogen | Batch | 1:100 | 1:500 | 1:2500 | 1:12500 | 1:62500 | 1:312500 | 1:1562500 | control |
| SJL | GPC3-Fc | #1106(TB1) | 2.82 | 2.17 | 1.55 | 0.63 | 0.18 | 0.08 | 0.06 | 0.05 |
| | (immunized | #1107(TB1) | 2.70 | 2.31 | 1.92 | 1.08 | 0.35 | 0.11 | 0.06 | 0.05 |
| | with GC3pep) | #1108(TB1) | 2.94 | 2.54 | 2.42 | 1.83 | 0.77 | 0.22 | 0.08 | 0.05 |
| | | #1109(TB1) | 2.59 | 2.25 | 1.91 | 1.24 | 0.55 | 0.12 | 0.06 | 0.05 |
| | | #1106(TB2) | 2.53 | 2.25 | 1.18 | 0.41 | 0.12 | 0.07 | 0.05 | 0.05 |
| | | #1107(TB2) | 2.58 | 2.24 | 1.08 | 0.29 | 0.09 | 0.06 | 0.05 | 0.05 |
| | | #1108(TB2) | 2.54 | 2.24 | 1.66 | 0.67 | 0.20 | 0.08 | 0.06 | 0.05 |
| | | #1109(TB2) | 2.33 | 1.46 | 0.62 | 0.19 | 0.08 | 0.06 | 0.05 | 0.05 |
| | | #1106(TB3) | 0.55 | 0.18 | 0.07 | 0.05 | 0.05 | 0.05 | 0.05 | 0.05 |
| | | #1107(TB3) | 2.32 | 1.20 | 0.46 | 0.13 | 0.06 | 0.05 | 0.05 | 0.05 |
| | | #1108(TB3) | 2.50 | 1.94 | 1.22 | 0.45 | 0.13 | 0.06 | 0.05 | 0.05 |
| | | #1109(TB3) | 2.07 | 1.06 | 0.35 | 0.11 | 0.06 | 0.05 | 0.05 | 0.05 |
| | | #1111(TB1) | 2.63 | 2.39 | 1.90 | 0.93 | 0.25 | 0.10 | 0.06 | 0.05 |
| SJL | GPC3-his | #1112(TB1) | 2.49 | 2.11 | 1.40 | 0.32 | 0.11 | 0.07 | 0.06 | 0.06 |
| | (immunized | #1114(TB1) | 2.37 | 2.07 | 1.19 | 0.30 | 0.10 | 0.06 | 0.06 | 0.06 |
| | with GC3pep) | #1115(TB1) | 2.21 | 2.33 | 1.72 | 0.61 | 0.16 | 0.07 | 0.06 | 0.05 |
| | | #1111(TB2) | 2.39 | 1.77 | 1.19 | 0.53 | 0.29 | 0.13 | 0.05 | 0.05 |
| | | #1112(TB2) | 2.37 | 1.50 | 0.60 | 0.33 | 0.22 | 0.16 | 0.05 | 0.05 |
| | | #1114(TB2) | 2.47 | 1.78 | 0.79 | 0.37 | 0.24 | 0.06 | 0.05 | 0.05 |
| | | #1115(TB2) | 2.46 | 1.85 | 0.93 | 0.35 | 0.21 | 0.06 | 0.05 | 0.06 |
| | | #1114(TB3) | 2.15 | 1.43 | 0.66 | 0.21 | 0.08 | 0.05 | 0.05 | 0.05 |
| | | #1115(TB3) | 1.43 | 0.48 | 0.13 | 0.07 | 0.05 | 0.05 | 0.05 | 0.05 |
| | | #1591(TB1) | 2.38 | 2.36 | 2.33 | 1.96 | 1.00 | 0.30 | 0.12 | 0.06 |
| | | #1592(TB1) | 2.70 | 2.51 | 2.43 | 1.85 | 0.76 | 0.39 | 0.19 | 0.05 |
| MRL/lpr | GC3pep | #1593(TB1) | 2.78 | 2.68 | 2.39 | 2.07 | 1.19 | 0.32 | 0.10 | 0.06 |
| | | #1594(TB1) | 2.70 | 2.49 | 2.30 | 1.48 | 0.63 | 0.12 | 0.06 | 0.05 |
| | | #1595(TB1) | 2.78 | 2.50 | 2.46 | 2.13 | 1.14 | 0.41 | 0.10 | 0.05 |
| | | #1593(TB2) | 2.60 | 2.30 | 2.03 | 1.17 | 0.34 | 0.11 | 0.06 | 0.05 |
| | | #1594(TB2) | 2.29 | 2.11 | 1.44 | 0.98 | 0.25 | 0.09 | 0.06 | 0.05 |
| | | #1595(TB2) | 2.57 | 2.26 | 1.78 | 1.05 | 0.29 | 0.09 | 0.06 | 0.05 |

TABLE 13
Assay results for cross-binding titers of mouse serum antibodies to monkey GPC3-His protein by ELISA

| | | | Serum dilution | | | | | | | |
|---|---|---|---|---|---|---|---|---|---|---|
| Immunized | | OD450 nm | | | | | | | | Blank |
| animal | Immunogen | Batch | 1:100 | 1:500 | 1:2500 | 1:12500 | 1:62500 | 1:312500 | 1:1562500 | control |
| SJL | GPC3-Fc | #1106(TB1) | 2.30 | 1.71 | 1.23 | 0.35 | 0.16 | 0.07 | 0.05 | 0.05 |
| | (immunized | #1107(TB1) | 2.50 | 1.89 | 1.52 | 0.51 | 0.18 | 0.10 | 0.06 | 0.05 |
| | with GC3pep) | #1108(TB1) | 2.39 | 1.87 | 1.53 | 0.51 | 0.17 | 0.11 | 0.07 | 0.05 |
| | | #1109(TB1) | 2.13 | 1.88 | 1.63 | 0.77 | 0.22 | 0.08 | 0.06 | 0.05 |
| | | #1106(TB2) | 2.05 | 1.59 | 0.59 | 0.19 | 0.08 | 0.05 | 0.06 | 0.07 |
| | | #1107(TB2) | 2.32 | 2.06 | 1.20 | 0.35 | 0.10 | 0.06 | 0.05 | 0.05 |
| | | #1108(TB2) | 2.66 | 1.99 | 1.45 | 0.53 | 0.15 | 0.07 | 0.05 | 0.05 |
| | | #1109(TB2) | 2.30 | 1.69 | 0.76 | 0.23 | 0.08 | 0.06 | 0.05 | 0.05 |
| | | #1106(TB3) | 0.67 | 0.23 | 0.08 | 0.06 | 0.05 | 0.05 | 0.05 | 0.05 |
| | | #1107(TB3) | 2.63 | 1.85 | 1.04 | 0.38 | 0.12 | 0.07 | 0.05 | 0.05 |
| | | #1108(TB3) | 2.40 | 1.88 | 1.10 | 0.50 | 0.12 | 0.06 | 0.05 | 0.05 |
| | | #1109(TB3) | 2.06 | 1.90 | 0.87 | 0.27 | 0.09 | 0.06 | 0.05 | 0.05 |
| | | #1111(TB1) | 2.08 | 1.71 | 0.92 | 0.32 | 0.23 | 0.08 | 0.05 | 0.06 |
| SJL | GPC3-his | #1112(TB1) | 2.09 | 1.27 | 0.84 | 0.12 | 0.07 | 0.05 | 0.05 | 0.05 |
| | (immunized | #1114(TB1) | 2.72 | 2.34 | 1.84 | 1.14 | 0.31 | 0.12 | 0.07 | 0.06 |
| | with GC3pep) | #1115(TB1) | 2.61 | 2.24 | 1.76 | 1.07 | 0.31 | 0.12 | 0.07 | 0.06 |
| | | #1111(TB2) | 2.26 | 1.97 | 1.25 | 0.45 | 0.14 | 0.07 | 0.05 | 0.05 |
| | | #1112(TB2) | 2.32 | 1.78 | 0.90 | 0.26 | 0.09 | 0.05 | 0.05 | 0.05 |
| | | #1114(TB2) | 2.30 | 1.62 | 1.11 | 0.36 | 0.11 | 0.06 | 0.05 | 0.05 |
| | | #1115(TB2) | 1.75 | 0.89 | 0.40 | 0.15 | 0.06 | 0.05 | 0.05 | 0.06 |
| | | #1114(TB3) | 2.42 | 1.89 | 1.32 | 0.54 | 0.17 | 0.07 | 0.05 | 0.05 |
| | | #1115(TB3) | 1.24 | 0.72 | 0.29 | 0.14 | 0.07 | 0.05 | 0.04 | 0.05 |
| | | #1591(TB1) | 2.35 | 1.91 | 1.79 | 1.56 | 0.80 | 0.24 | 0.09 | 0.05 |
| | | #1592(TB1) | 2.20 | 1.92 | 1.65 | 1.51 | 1.10 | 0.49 | 0.13 | 0.05 |
| MRL/lpr | GC3pep | #1593(TB1) | 2.22 | 1.82 | 1.80 | 1.74 | 0.98 | 0.23 | 0.09 | 0.05 |
| | | #1594(TB1) | 1.85 | 1.80 | 1.37 | 0.93 | 0.36 | 0.12 | 0.06 | 0.05 |
| | | #1595(TB1) | 2.23 | 1.99 | 1.85 | 1.60 | 0.76 | 0.25 | 0.08 | 0.07 |
| | | #1593(TB2) | 2.63 | 2.28 | 2.21 | 2.12 | 1.70 | 0.87 | 0.27 | 0.04 |
| | | #1594(TB2) | 2.63 | 2.19 | 2.03 | 1.82 | 0.93 | 0.28 | 0.09 | 0.05 |
| | | #1595(TB2) | 2.61 | 2.33 | 2.04 | 1.73 | 0.86 | 0.26 | 0.10 | 0.04 |

TABLE 14
Assay results for binding titers of mouse serum antibodies to HepG2 cells by FACS

| | | Mean | | | | | |
|---|---|---|---|---|---|---|---|
| | | fluorescence | Serum dilution | | | | |
| Immunized | | intensity MFI | | | | | Blank |
| animal | Immunogen | Batch | 1:100 | 1:1000 | 1:10000 | 1:100000 | control |
| SJL | GPC3-Fc | #1106(TB1) | 3004 | 457 | 84 | 41 | 109 |
| | (immunized | #1107(TB1) | 3803 | 1427 | 26 | 66 | 109 |
| | with GC3pep) | #1108(TB1) | 3604 | 1809 | 318 | 63 | 109 |
| | | #1109(TB1) | 3401 | 1623 | 260 | 68 | 109 |
| | | #1106(TB2) | 3699 | 1201 | 195 | 68 | 109 |
| | | #1107(TB2) | 4129 | 2129 | 466 | 99 | 109 |
| | | #1108(TB2) | 5166 | 1732 | 275 | 91 | 109 |
| | | #1109(TB2) | 4538 | 856 | 135 | 48 | 109 |
| | | #1106(TB3) | 1327 | 295 | 119 | 112 | 101 |
| | | #1107(TB3) | 5576 | 1666 | 308 | 122 | 101 |
| | | #1108(TB3) | 6948 | 2340 | 392 | 130 | 101 |
| | | #1109(TB3) | 4628 | 1947 | 339 | 124 | 101 |
| SJL | GPC3-his | #1111(TB1) | 4894 | 3175 | 840 | 140 | 109 |
| | (immunized | #1112(TB1) | 3654 | 2219 | 435 | 88 | 109 |
| | with GC3pep) | #1114(TB1) | 1305 | 742 | 928 | 413 | 109 |
| | | #1115(TB1) | 1781 | 613 | 190 | 24 | 109 |
| | | #1111(TB2) | 5055 | 2319 | 362 | 78 | 109 |
| | | #1112(TB2) | 4255 | 1176 | 163 | 53 | 109 |
| | | #1114(TB2) | 4213 | 1539 | 183 | 51 | 109 |
| | | #1115(TB2) | 4731 | 1654 | 252 | 69 | 109 |
| | | #1114(TB3) | 7216 | 2842 | 420 | 126 | 101 |
| | | #1115(TB3) | 4301 | 733 | 162 | 100 | 101 |
| MRL/lpr | GC3pep | #1591(TB1) | 3874 | 2222 | 571 | 122 | 109 |
| | | #1592(TB1) | 3454 | 2186 | 351 | 106 | 109 |
| | | #1593(TB1) | 3192 | 3371 | 408 | 148 | 109 |
| | | #1594(TB1) | 2860 | 1576 | 336 | 69 | 109 |
| | | #1595(TB1) | 2721 | 1204 | 230 | 75 | 109 |
| | | #1593(TB2) | 3689 | 6043 | 2352 | 490 | 104 |
| | | #1594(TB2) | 1325 | 1504 | 328 | 152 | 104 |
| | | #1595(TB2) | 3545 | 4537 | 841 | 239 | 104 |

Mice with high antibody titers in serum were selected for splenocyte fusion after the second, sixth, and seventh booster immunizations, respectively. Booster immunizations were performed 3 days prior to the splenocyte fusion, and an antigen solution prepared from physiological saline was injected subcutaneously, intraplantarly, and intraperitoneally at 50 μg/mouse.

### (B) Splenocyte Fusion and Hybridoma Screening

ACK Lysing Buffer (purchased from Gibco, Cat. No. A1049201) was added to lyse red blood cells mixed in splenocytes to obtain a splenocyte suspension. The cells were washed 3 times by centrifugation at 1000 rpm with a DMEM (purchased from Gibco, Cat. No. 12800017) basal medium, then mixed with mouse myeloma cells SP2/0 (purchased from ATCC) at a ratio of 2:1 in number of viable cells, and the resulting mixture was subjected to cell fusion by BTX ECM2001+ efficient electrofusion (see METHODS IN ENZYMOLOGY, VOL. 220). The fused cells were diluted into a DMEM medium containing 20% fetal bovine serum (purchased from ExCell Bio, Cat. No. FND500), and 1×HAT (purchased from Sigma, Cat. No. H0262-10VL), wherein the percentage was a percentage by mass. Then the medium was added to a 96-well cell culture plate at 2×10⁴/200 μL/well, and the plate was put into a 37° C. incubator with 5% CO₂, wherein the percentage was a percentage by volume. After 14 days, cell fusion plate supernatants were screened by ELISA, and human GPC3 protein and/or polypeptide GC3pep ELISA-positive clones were expanded into a 24-well plate for expansion in DMEM containing 10% HT (purchased from Sigma, Cat. No. H0137-10VL) fetal bovine serum under 5% CO₂ at 37° C. After 3 days of culture, the expanded culture solution in the 24-well plate was centrifuged, and the supernatant was collected. Then the supernatant was analyzed for antibody subtypes, binding activity was assayed using human GPC3 protein and polypeptide GC3pep by ELISA, and binding activity was assayed using HepG2 cells and CHO-K1-human GPC3 cells 2B5 by FACS.

Based on screening results from the 24-well plate, hybridoma cells from the culture supernatant of hybridoma cells positive for binding with the HepG2 cells in the FACS assay were selected as eligible positive clones, and hybridoma cells from positive wells were subcloned into a 96-well plate by limiting dilution assay, and cultured in a DMEM medium containing 10% FBS under 5% CO₂ at 37° C. After 8 days of subcloning, primary screening was performed by ELISA, and a single positive monoclone was selected and amplified into a 24-well plate for further culture. After 3 days, binding activity was assayed using human GPC3 protein and polypeptide GC3pep by ELISA, binding activity was assayed using cells, HepG2 CHO-K1-human GPC3 cells 2B5 and HEK293T-monkey-GPC3 by FACS, and binding specificity was confirmed using negative cells CHO-K1 and HEK293T cells by FACS.

Based on assay results for the sample from the 24-well plate, the optimal clone was selected and amplified in a DMEM medium containing 10% FBS under 5% CO₂ at 37° C., and the amplified clone was cryopreserved in liquid nitrogen to obtain the hybridoma cell provided in the present invention.

### Example 3 Amino Acid Sequencing for Light and Heavy Chain Variable Regions of Hybridoma-Positive Clones

Hybridoma cells in a logarithmic growth phase were collected, then fully lysed with Trizol (Invitrogen, with Cat. No. of 15596-018) and stored at −80° C. for sequencing. Amino acid sequencing for light and heavy chain variable regions of hybridoma-positive clone was completed. Sequencing results were analyzed by using MOE software, then an evolutionary tree was constructed based on amino acid sequences of proteins encoding variable regions, and 48 clones were screened after eliminating sequences that were close to each other on the evolutionary tree based on sequence similarity, including 21 clones in the F1 series, 7 clones in the F2 series, 7 clones in the F3 series, 10 clones in the F4 series, and 3 clones in the F5 series.

Sequences for heavy chain variable regions of the 48 clones were cloned into an expression vector pcDNA3.4-B1HH1 (a sequence of a heavy chain constant region, ASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKTHTCPPCPA PELLGGPSVFLFPPKPKDTLMISRTPEVTCVVVDVSHEDPEVKFNWYVDGVEVHN AKTKPREEQYNSTYRVVSVLTVLHQDWLNGKEYKCKVSNKALPAPIEKTISKAKG QPREPQVYTLPPSREEMTKNQVSLTCLVKGFYPSDIAVEWESNGQPENNYKTTPPV LDSDGSFFLYSKLTVDKSRWQQGNVFSCSVMHEALHNHYTQKSLSLSPGK, SEQ ID NO: 587) containing a signal peptide and a heavy chain constant region of a murine antibody IgG1, and sequences for light chain variable regions were cloned into an expression vector pcDNA3.4-B1HLK (a sequence of a light chain constant region, RTVAAPSVFIFPPSDEQLKSGTASVVCLLNNFYPREAKVQWKVDNALQSGNSQESV TEQDSKDSTYSLSSTLTLSKADYEKHKVYACEVTHQGLSSPVTKSFNRGEC, SEQ ID NO: 588) containing a signal peptide and a Kappa light chain constant region of a human antibody IgG1 to obtain an expression vector for human-murine chimeric antibodies, and antibodies were prepared according to the method described in Example 1 (A).

### Example 4 Identification of GPC3 Human-Murine Chimeric Antibodies

### (A) Assay for Binding of GPC3 Human-Murine Chimeric Antibodies to Human GPC3 Protein by Enzyme-Linked Immunosorbent Assay (ELISA)

The obtained 48 clones were subjected to ELISA and data analysis according to the method described in Example 1 (A). An ELISA plate reader (Multimode Plate Reader, EnSight, purchased from Perkin Elmer) was used to read OD_{450 nm} values, with results for binding activity of GPC3 human-murine chimeric antibodies with the human GPC3 protein as shown in FIG. 11 and Table 15. The results showed that a total of 34 antibodies bound well to the human GPC3 protein, including 20 antibodies in the F1 series, 7 antibodies in the F2 series, 3 antibodies in the F3 series, and 4 antibodies in the F4 series (Table 1). The IgG control was hIgG1, and data in the table are OD_{450 nm} values.

TABLE 15
Assay results for binding reactions of chimeric
antibodies with human GPC3 protein by ELISA

| | Antibody concentration (nM) | | | | | | | |
|---|---|---|---|---|---|---|---|---|
| Antibody | | | | | | | | Blank |
| name | 100 | 10 | 1 | 0.1 | 0.01 | 0.001 | 0.0001 | control |
| F1.2.14 | 1.84 | 1.47 | 1.24 | 0.61 | 0.17 | 0.10 | 0.08 | 0.09 |
| F1.21.8 | 2.00 | 1.70 | 1.71 | 0.90 | 0.25 | 0.12 | 0.08 | 0.09 |
| F1.41.17 | 1.51 | 1.59 | 1.68 | 0.97 | 0.24 | 0.11 | 0.08 | 0.09 |
| F1.42.8 | 2.56 | 2.31 | 2.02 | 1.22 | 0.30 | 0.13 | 0.10 | 0.09 |
| F1.57.21 | 2.35 | 2.02 | 1.50 | 0.80 | 0.21 | 0.11 | 0.08 | 0.09 |
| F1.73.9 | 1.46 | 1.74 | 1.33 | 0.86 | 0.23 | 0.11 | 0.09 | 0.09 |
| F1.78.24 | 0.45 | 1.07 | 0.72 | 0.66 | 0.19 | 0.12 | 0.09 | 0.09 |
| F1.83.6 | 0.76 | 1.19 | 0.83 | 0.40 | 0.17 | 0.10 | 0.08 | 0.08 |
| F1.92.17 | 0.66 | 1.19 | 0.68 | 0.25 | 0.11 | 0.09 | 0.08 | 0.08 |
| F1.110.24 | 0.62 | 1.18 | 0.77 | 0.42 | 0.13 | 0.09 | 0.08 | 0.09 |
| F1.111.11 | 2.70 | 2.46 | 2.41 | 1.61 | 0.41 | 0.14 | 0.10 | 0.10 |
| F1.115.8 | 2.01 | 1.90 | 1.83 | 0.83 | 0.20 | 0.11 | 0.09 | 0.10 |
| F1.118.4 | 1.53 | 1.75 | 1.64 | 0.74 | 0.19 | 0.12 | 0.10 | 0.09 |
| F1.120.16 | 1.62 | 1.63 | 1.23 | 0.56 | 0.22 | 0.14 | 0.11 | 0.09 |
| F1.136.7 | 1.19 | 1.40 | 0.63 | 0.43 | 0.14 | 0.10 | 0.11 | 0.09 |
| F1.145.22 | 2.53 | 2.35 | 1.95 | 0.94 | 0.22 | 0.12 | 0.09 | 0.10 |
| F1.3.9 | 2.07 | 1.95 | 1.66 | 0.92 | 0.21 | 0.11 | 0.10 | 0.11 |
| F1.9.19 | 1.88 | 1.85 | 1.60 | 0.58 | 0.16 | 0.11 | 0.10 | 0.11 |
| F1.62.9 | 1.09 | 0.96 | 0.63 | 0.20 | 0.10 | 0.09 | 0.11 | 0.09 |
| F1.153.1 | 2.06 | 1.85 | 1.56 | 0.64 | 0.15 | 0.10 | 0.11 | 0.09 |
| F2.55.11 | 2.58 | 2.66 | 2.24 | 1.57 | 0.43 | 0.15 | 0.11 | 0.09 |
| F2.154.1 | 2.61 | 2.52 | 2.31 | 1.57 | 0.38 | 0.13 | 0.09 | 0.09 |
| F2.169.2 | 2.43 | 2.52 | 2.24 | 1.25 | 0.27 | 0.11 | 0.10 | 0.10 |
| F2.23.8 | 2.51 | 2.35 | 2.16 | 1.28 | 0.29 | 0.12 | 0.10 | 0.10 |
| F2.39.2 | 2.30 | 2.34 | 2.21 | 1.33 | 0.34 | 0.12 | 0.10 | 0.10 |
| F2.92.1 | 2.45 | 2.23 | 2.24 | 1.23 | 0.32 | 0.12 | 0.11 | 0.10 |
| F2.152.3 | 2.43 | 2.43 | 2.28 | 1.45 | 0.32 | 0.13 | 0.10 | 0.09 |
| F3-38.7 | 2.48 | 2.44 | 2.18 | 1.62 | 0.38 | 0.13 | 0.09 | 0.09 |
| F3-54.12 | 2.44 | 2.41 | 2.29 | 1.58 | 0.48 | 0.15 | 0.13 | 0.13 |
| F3-81.19 | 2.45 | 2.49 | 2.04 | 0.87 | 0.19 | 0.28 | 0.10 | 0.09 |
| F4-13.7 | 2.30 | 2.27 | 2.28 | 1.27 | 0.30 | 0.12 | 0.10 | 0.09 |
| F4-18.4 | 1.02 | 1.18 | 1.41 | 0.83 | 0.26 | 0.15 | 0.16 | 0.10 |
| F4-21.16 | 0.52 | 1.14 | 1.37 | 0.46 | 0.15 | 0.10 | 0.10 | 0.08 |
| F4-26.1 | 1.00 | 1.43 | 1.25 | 0.64 | 0.18 | 0.11 | 0.09 | 0.09 |
| Y035 | 2.64 | 2.59 | 2.37 | 1.49 | 0.34 | 0.12 | 0.10 | 0.11 |
| T2-23 | 2.13 | 2.09 | 1.84 | 1.32 | 0.33 | 0.13 | 0.10 | 0.10 |
| hIgG1 | 0.11 | 0.09 | 0.09 | 0.09 | 0.09 | 0.09 | 0.09 | 0.09 |

### (B) Assay for Binding of GPC3 Human-Murine Chimeric Antibodies to Cells Expressing GPC3 Protein and Cells not Expressing GPC3 Protein (Negative Cells) by Flow Cytometry (FACS)

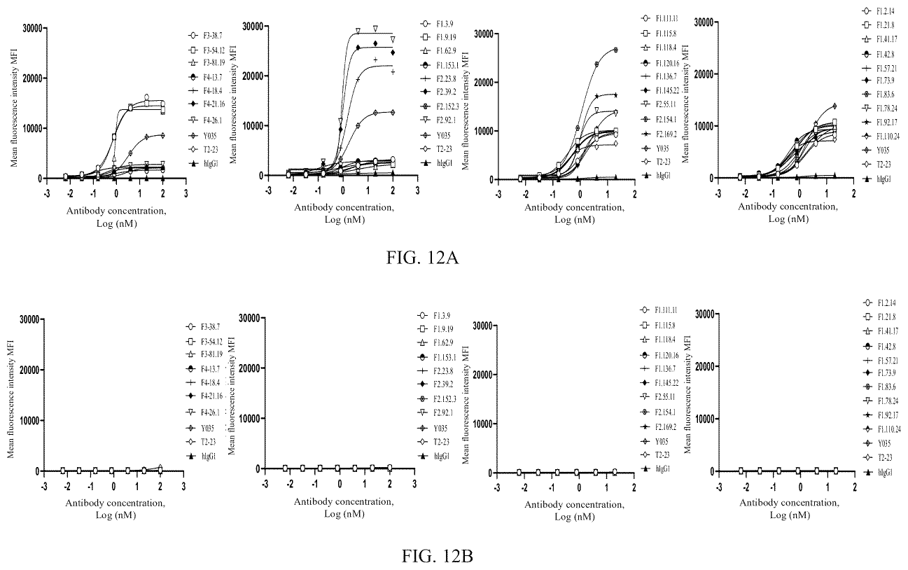

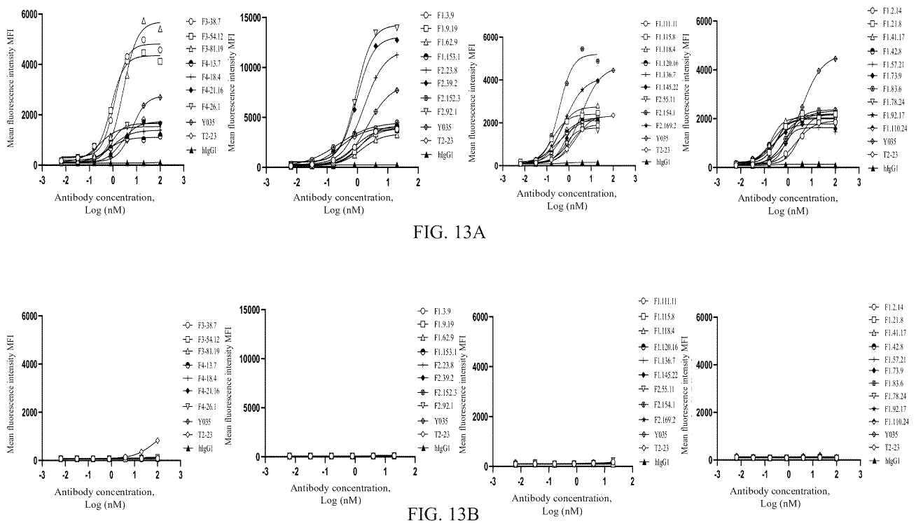

The desired cells were expanded to a logarithmic growth phase in a T-75 cell culture flask, the medium was removed by pipetting, and the cells were washed twice with a PBS buffer, and digested with trypsin. Then a complete medium was added to stop the digestion, and the cells were blown into a single cell suspension. After counting, the cells were centrifuged, and the cell pellet was resuspended with an FACS buffer (PBS+2% fetal bovine serum) to 2×10⁶ cells/mL. The cell resuspension solution was added to a 96-well FACS reaction plate at 50 μl/well, and a chimeric antibody sample to be tested was added at 50 μl/well for incubation at 4° C. for 1 h. After the plate was centrifuged and washed 3 times with a PBS buffer, a goat anti-human IgG H+L antibody (Jackson, Cat. No. 109605088) was added at 50 μl/well for incubation on ice for 1 h. After the plate was centrifuged and washed 3 times with a PBS buffer, 100 μl of the resulting solution was assayed and analyzed by FACS (FACS Canto™, purchased from BD Biosciences). Data analysis was performed by software (FlowJo) to obtain the mean fluorescence intensity (MFI) of the cells. Then, analysis was performed by software (GraphPad Prism8), data were fitted, and EC₅₀ values were calculated. Table 16, FIG. 12A, and FIG. 12B show that the 34 chimeric antibodies could bind to CHO-K1-human GPC3 cells, but did not bind to CHOK1 cells; and Table 17, FIG. 13A, and FIG. 13B show that the 34 chimeric antibodies could bind to HepG2 cells, but did not bind to A431 cells.

TABLE 16
Assay results for binding reactions of chimeric antibodies
with CHO-K1-human GPC3 cells and CHOK1 cells by FACS

| | CHOK1-hGPC3.2B5 | | CHOK1 | |
|---|---|---|---|---|
| | Maximum mean | | Maximum mean | |
| | fluorescence | | fluorescence | |
| Antibody | intensity | Ec50 | intensity | Ec50 |
| name | Max MFI | (nM) | Max MFI | (nM) |
| F1.2.14 | 8281 | 1.67 | 80 | Negative |
| F1.21.8 | 10741 | 0.68 | 82 | Negative |
| F1.41.17 | 9928 | 0.55 | 94 | Negative |
| F1.42.8 | 9418 | 0.40 | 84 | Negative |
| F1.57.21 | 9361 | 1.36 | 88 | Negative |
| F1.73.9 | 8853 | 0.66 | 92 | Negative |
| F1.78.24 | 10397 | 0.76 | 129 | Negative |
| F1.83.6 | 10126 | 1.10 | 87 | Negative |
| F1.92.17 | 9600 | 2.04 | 73 | Negative |
| F1.110.24 | 9366 | 0.90 | 81 | Negative |
| F1.111.11 | 9228 | 1.08 | 422 | Negative |
| F1.115.8 | 9980 | 0.33 | 88 | Negative |
| F1.118.4 | 9636 | 0.53 | 81 | Negative |
| F1.120.16 | 9732 | 0.52 | 106 | Negative |
| F1.136.7 | 9541 | 1.41 | 77 | Negative |
| F1.145.22 | 9698 | 1.18 | 82 | Negative |
| F2.55.11 | 13585 | 0.89 | 80 | Negative |
| F2.154.1 | 26663 | 1.14 | 83 | Negative |
| F2.169.2 | 17317 | 1.06 | 235 | Negative |
| Y035 | 13807 | 2.06 | 82 | Negative |
| T2-23 | 7407 | 0.31 | 189 | Negative |
| hIgG1 | 488 | Negative | 78 | Negative |
| F1.3.9 | 3207 | 2.78 | 237 | Negative |
| F1.9.19 | 2701 | 1.76 | 74 | Negative |
| F1.62.9 | 2157 | 3.49 | 80 | Negative |
| F1.153.1 | 3083 | 0.91 | 75 | Negative |
| F2.23.8 | 23243 | 1.48 | 71 | Negative |
| F2.39.2 | 26519 | 1.00 | 76 | Negative |
| F2.92.1 | 29428 | 0.92 | 92 | Negative |
| F2.152.3 | 2826 | 0.73 | 198 | Negative |
| Y035 | 12626 | 1.72 | 76 | Negative |
| T2-23 | 2914 | 0.10 | 132 | Negative |
| hIgG1 | 568 | Negative | 69 | Negative |
| F3-38.7 | 16184 | 0.85 | 183 | Negative |
| F3-54.12 | 14963 | 0.72 | 118 | Negative |
| F3-81.19 | 14062 | 2.58 | 66 | Negative |
| F4-13.7 | 1707 | 1.102 | 84 | Negative |
| F4-18.4 | 2366 | 0.89 | 86 | Negative |
| F4-21.16 | 2174 | 3.25 | 83 | Negative |
| F4-26.1 | 2811 | 1.21 | 91 | Negative |
| Y035 | 8548 | 3.22 | 74 | Negative |
| T2-23 | 2680 | 0.11 | 766 | Negative |
| hIgG1 | 80 | Negative | 81 | Negative |

TABLE 17
Assay results for binding reactions of chimeric antibodies
with HepG2 cells and A431 cells by FACS

| | HepG2 | | A431 | |
|---|---|---|---|---|
| | Maximum mean | | Maximum mean | |
| | fluorescence | | fluorescence | |
| Antibody | intensity | Ec50 | intensity | Ec50 |
| name | Max MFI | (nM) | Max MFI | (nM) |
| F1.2.14 | 1858 | 2.13 | 91 | Negative |
| F1.21.8 | 2186 | 0.36 | 89 | Negative |
| F1.41.17 | 1990 | 0.20 | 80 | Negative |
| F1.42.8 | 2092 | 0.21 | 83 | Negative |
| F1.57.21 | 1649 | 0.71 | 87 | Negative |
| F1.73.9 | 1881 | 0.25 | 92 | Negative |
| F1.78.24 | 2116 | 0.31 | 82 | Negative |
| F1.83.6 | 2339 | 0.87 | 81 | Negative |
| F1.92.17 | 2295 | 2.90 | 78 | Negative |
| F1.110.24 | 2266 | 0.83 | 79 | Negative |
| F1.111.11 | 1876 | 1.13 | 175 | Negative |
| F1.115.8 | 2462 | 0.18 | 95 | Negative |
| F1.118.4 | 2808 | 0.33 | 99 | Negative |
| F1.120.16 | 2076 | 0.38 | 98 | Negative |
| F1.136.7 | 2238 | 2.00 | 90 | Negative |
| F1.145.22 | 2156 | 0.67 | 89 | Negative |
| F2.55.11 | 1634 | 0.66 | 225 | Negative |
| F2.154.1 | 5450 | 0.37 | 99 | Negative |
| F2.169.2 | 3979 | 0.65 | 145 | Negative |
| Y035 | 4454 | 3.93 | 103 | Negative |
| T2-23 | 2351 | 0.34 | 214 | Negative |
| hIgG1 | 154 | Negative | 92 | Negative |
| F1.3.9 | 3814 | 0.80 | 150 | Negative |
| F1.9.19 | 3784 | 1.12 | 79 | Negative |
| F1.62.9 | 3252 | 1.32 | 206 | Negative |
| F1.153.1 | 4095 | 1.30 | 141 | Negative |
| F2.23.8 | 11252 | 1.64 | 100 | Negative |
| F2.39.2 | 12745 | 0.95 | 96 | Negative |
| F2.92.1 | 13960 | 0.92 | 157 | Negative |
| F2.152.3 | 4494 | 0.23 | 304 | Negative |
| Y035 | 7702 | 2.25 | 83 | Negative |
| T2-23 | 4014 | 0.27 | 159 | Negative |
| hIgG1 | 258 | Negative | 89 | Negative |
| F3-38.7 | 4978 | 1.20 | 85 | Negative |
| F3-54.12 | 4465 | 0.83 | 134 | Negative |
| F3-81.19 | 5736 | 2.72 | 77 | Negative |
| F4-13.7 | 1151 | 0.58 | 70 | Negative |
| F4-18.4 | 1391 | 0.92 | 72 | Negative |
| F4-21.16 | 1710 | 2.31 | 76 | Negative |
| F4-26.1 | 1602 | 0.85 | 78 | Negative |
| Y035 | 2693 | 6.36 | 73 | Negative |
| T2-23 | 1816 | 0.26 | 821 | Negative |
| hIgG1 | 110 | Negative | 90 | Negative |

### Example 5 Assay for Cross-Binding Activity of GPC3 Human-Murine Chimeric Antibodies

### (A) Assay for Binding of Chimeric Antibodies to Monkey GPC3 Protein and Murine GPC3 Protein by ELISA

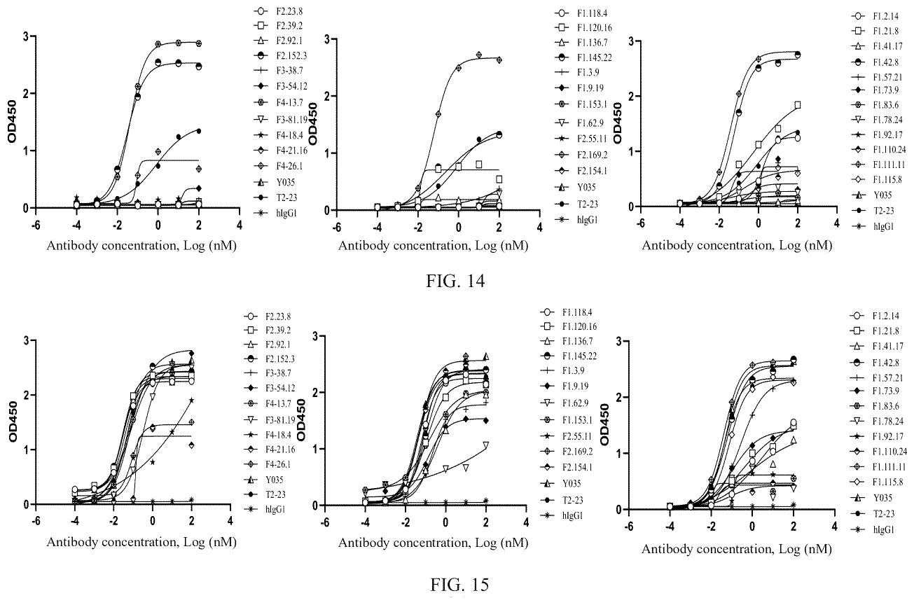

A monkey GPC3-His protein (purchased from Acro, Cat. No. GP3-C5225) and a murine GPC3-his protein (purchased from Sino Biological, Cat. No. 50989-M08B) were subjected to ELISA and data analysis according to the method described in Example 1 (A). The ELISA results for the chimeric antibodies and the murine GPC3 protein are shown in FIG. 14 and Table 18. The results showed that the 8 antibodies F1.2.14, F1.21.8, F1.42.8, F1.111.11, F1.145.22, F2.169.2. F2.152.3, and F4-13.7 bound well to the murine GPC3 protein, the antibody F4-26.1 bound weakly to the murine GPC3 protein, and the remaining antibodies did not bind to the murine GPC3 protein. The IgG control was hIgG1, and data in the table are OD_{450 nm} values.

TABLE 18
Assay results for binding reactions of chimeric
antibodies with murine GPC3 protein by ELISA

| | Antibody concentration (nM) | | | | | | | |
|---|---|---|---|---|---|---|---|---|
| Antibody | | | | | | | | Blank |
| name | 100 | 10 | 1 | 0.1 | 0.01 | 0.001 | 0.0001 | control |
| F1.2.14 | 1.24 | 1.20 | 0.46 | 0.14 | 0.06 | 0.05 | 0.05 | 0.05 |
| F1.21.8 | 1.84 | 1.41 | 1.12 | 0.76 | 0.17 | 0.07 | 0.05 | 0.05 |
| F1.41.17 | 0.11 | 0.08 | 0.07 | 0.06 | 0.05 | 0.05 | 0.06 | 0.05 |
| F1.42.8 | 2.75 | 2.60 | 2.51 | 1.70 | 0.37 | 0.09 | 0.05 | 0.05 |
| F1.57.21 | 0.65 | 0.79 | 0.20 | 0.13 | 0.07 | 0.06 | 0.05 | 0.06 |
| F1.73.9 | 0.52 | 0.86 | 0.41 | 0.58 | 0.06 | 0.06 | 0.05 | 0.05 |
| F1.78.24 | 0.25 | 0.34 | 0.17 | 0.24 | 0.10 | 0.06 | 0.05 | 0.05 |
| F1.83.6 | 0.16 | 0.24 | 0.14 | 0.12 | 0.06 | 0.05 | 0.05 | 0.05 |
| F1.92.17 | 0.13 | 0.22 | 0.11 | 0.06 | 0.05 | 0.05 | 0.05 | 0.05 |
| F1.110.24 | 0.30 | 0.53 | 0.32 | 0.12 | 0.05 | 0.05 | 0.05 | 0.06 |
| F1.111.11 | 3.01 | 2.60 | 2.67 | 2.04 | 0.62 | 0.13 | 0.06 | 0.07 |
| F1.115.8 | 0.60 | 0.70 | 0.40 | 0.34 | 0.07 | 0.05 | 0.05 | 0.06 |
| F1.118.4 | 0.07 | 0.06 | 0.05 | 0.05 | 0.05 | 0.05 | 0.05 | 0.06 |
| F1.120.16 | 0.54 | 0.80 | 0.76 | 0.72 | 0.16 | 0.07 | 0.05 | 0.05 |
| F1.136.7 | 0.17 | 0.91 | 0.17 | 0.22 | 0.06 | 0.05 | 0.05 | 0.05 |
| F1.145.22 | 1.32 | 1.62 | 0.77 | 0.75 | 0.08 | 0.06 | 0.05 | 0.06 |
| F1.3.9 | 0.37 | 0.18 | 0.18 | 0.05 | 0.04 | 0.04 | 0.05 | 0.04 |
| F1.9.19 | 0.07 | 0.05 | 0.05 | 0.05 | 0.05 | 0.04 | 0.05 | 0.04 |
| F1.62.9 | 0.28 | 0.06 | 0.04 | 0.04 | 0.05 | 0.04 | 0.04 | 0.05 |
| F1.153.1 | 0.09 | 0.08 | 0.05 | 0.04 | 0.04 | 0.08 | 0.04 | 0.04 |
| F2.55.11 | 0.16 | 0.07 | 0.05 | 0.05 | 0.05 | 0.05 | 0.05 | 0.06 |
| F2.154.1 | 0.06 | 0.05 | 0.05 | 0.05 | 0.05 | 0.05 | 0.05 | 0.05 |
| F2.169.2 | 2.63 | 2.72 | 2.49 | 1.68 | 0.38 | 0.09 | 0.05 | 0.05 |
| F2.23.8 | 0.06 | 0.05 | 0.06 | 0.04 | 0.07 | 0.06 | 0.05 | 0.04 |
| F2.39.2 | 0.06 | 0.05 | 0.05 | 0.05 | 0.07 | 0.06 | 0.05 | 0.05 |
| F2.92.1 | 0.07 | 0.05 | 0.05 | 0.07 | 0.05 | 0.05 | 0.04 | 0.05 |
| F2.152.3 | 2.47 | 2.53 | 2.54 | 1.94 | 0.67 | 0.12 | 0.05 | 0.04 |
| F3-38.7 | 0.12 | 0.06 | 0.05 | 0.04 | 0.05 | 0.06 | 0.06 | 0.05 |
| F3-54.12 | 0.34 | 0.09 | 0.05 | 0.05 | 0.05 | 0.05 | 0.11 | 0.08 |
| F3-81.19 | 0.10 | 0.05 | 0.05 | 0.04 | 0.04 | 0.05 | 0.05 | 0.07 |
| F4-13.7 | 2.87 | 2.88 | 2.86 | 2.12 | 0.54 | 0.11 | 0.06 | 0.12 |
| F4-18.4 | 0.13 | 0.16 | 0.14 | 0.10 | 0.10 | 0.15 | 0.18 | 0.09 |
| F4-21.16 | 0.08 | 0.05 | 0.05 | 0.09 | 0.04 | 0.05 | 0.05 | 0.05 |
| F4-26.1 | 0.68 | 1.29 | 0.98 | 0.57 | 0.11 | 0.07 | 0.06 | 0.05 |
| Y035 | 0.08 | 0.05 | 0.05 | 0.05 | 0.05 | 0.05 | 0.05 | 0.06 |
| T2-23 | 1.34 | 1.24 | 0.73 | 0.42 | 0.16 | 0.07 | 0.05 | 0.05 |
| hIgG1 | 0.12 | 0.06 | 0.05 | 0.06 | 0.05 | 0.05 | 0.06 | 0.06 |

The ELISA results for the chimeric antibodies and the monkey GPC3 protein are shown in FIG. 15 and Table 19. The results showed that the antibodies F1.78.24. F1.83.6. F1.92.17 and F1.110.24 weakly bound to the monkey GPC3 protein, and the remaining antibodies bound well to the monkey GPC3 protein.

TABLE 19
Assay results for binding reactions of chimeric
antibodies with monkey GPC3 protein by ELISA

| | Antibody concentration (nM) | | | | | | | |
|---|---|---|---|---|---|---|---|---|
| Antibody | | | | | | | | Blank |
| name | 100 | 10 | 1 | 0.1 | 0.01 | 0.001 | 0.0001 | control |
| F1.2.14 | 1.55 | 1.03 | 0.86 | 0.27 | 0.07 | 0.05 | 0.05 | 0.05 |
| F1.21.8 | 1.49 | 1.11 | 1.00 | 0.51 | 0.12 | 0.06 | 0.05 | 0.05 |
| F1.41.17 | 1.24 | 0.81 | 0.83 | 0.46 | 0.11 | 0.06 | 0.05 | 0.05 |
| F1.42.8 | 2.67 | 2.46 | 2.31 | 1.61 | 0.35 | 0.08 | 0.05 | 0.05 |
| F1.57.21 | 2.30 | 2.15 | 1.68 | 0.77 | 0.14 | 0.06 | 0.05 | 0.05 |
| F1.73.9 | 1.47 | 1.27 | 1.14 | 0.73 | 0.18 | 0.06 | 0.05 | 0.05 |
| F1.78.24 | 0.37 | 0.21 | 0.73 | 0.55 | 0.17 | 0.06 | 0.05 | 0.05 |
| F1.83.6 | 0.55 | 0.32 | 0.62 | 0.43 | 0.08 | 0.05 | 0.05 | 0.05 |
| F1.92.17 | 0.58 | 0.62 | 0.64 | 0.15 | 0.06 | 0.05 | 0.05 | 0.05 |
| F1.110.24 | 0.41 | 0.47 | 0.31 | 0.29 | 0.06 | 0.05 | 0.05 | 0.05 |
| F1.111.11 | 2.66 | 2.62 | 2.57 | 1.91 | 0.53 | 0.11 | 0.06 | 0.05 |
| F1.115.8 | 2.26 | 2.36 | 2.14 | 1.33 | 0.27 | 0.07 | 0.05 | 0.05 |
| F1.118.4 | 2.14 | 2.32 | 2.21 | 1.43 | 0.28 | 0.08 | 0.05 | 0.05 |
| F1.120.16 | 2.14 | 2.21 | 1.91 | 1.11 | 0.26 | 0.07 | 0.07 | 0.05 |
| F1.136.7 | 1.96 | 1.96 | 1.33 | 0.62 | 0.10 | 0.05 | 0.05 | 0.05 |
| F1.145.22 | 2.40 | 2.39 | 2.17 | 1.23 | 0.25 | 0.07 | 0.05 | 0.05 |
| F1.3.9 | 1.82 | 1.70 | 1.52 | 0.56 | 0.16 | 0.05 | 0.04 | 0.04 |
| F1.9.19 | 1.50 | 1.53 | 1.39 | 0.72 | 0.30 | 0.25 | 0.04 | 0.04 |
| F1.62.9 | 1.06 | 0.66 | 0.64 | 0.56 | 0.39 | 0.36 | 0.21 | 0.04 |
| F1.153.1 | 2.00 | 1.98 | 1.61 | 1.06 | 0.49 | 0.36 | 0.27 | 0.04 |
| F2.55.11 | 2.23 | 2.54 | 2.30 | 1.76 | 0.49 | 0.10 | 0.05 | 0.05 |
| F2.154.1 | 2.24 | 2.49 | 2.38 | 1.69 | 0.46 | 0.10 | 0.05 | 0.05 |
| F2.169.2 | 1.99 | 2.64 | 2.27 | 1.36 | 0.27 | 0.07 | 0.05 | 0.05 |
| F2.23.8 | 2.25 | 2.24 | 2.22 | 1.80 | 0.58 | 0.28 | 0.27 | 0.04 |
| F2.39.2 | 2.35 | 2.28 | 2.36 | 1.98 | 0.66 | 0.34 | 0.21 | 0.04 |
| F2.92.1 | 2.34 | 2.24 | 2.30 | 1.90 | 0.65 | 0.27 | 0.23 | 0.04 |
| F2.152.3 | 2.41 | 2.31 | 2.52 | 1.92 | 0.69 | 0.31 | 0.24 | 0.04 |
| F3-38.7 | 3.01 | 2.58 | 2.37 | 1.69 | 0.42 | 0.10 | 0.06 | 0.04 |
| F3-54.12 | 2.76 | 2.32 | 2.21 | 1.57 | 0.41 | 0.10 | 0.08 | 0.06 |
| F3-81.19 | 2.49 | 2.55 | 1.96 | 0.62 | 0.16 | 0.06 | 0.05 | 0.04 |
| F4-13.7 | 2.44 | 2.40 | 2.34 | 1.55 | 0.36 | 0.08 | 0.05 | 0.06 |
| F4-18.4 | 1.90 | 1.32 | 0.77 | 0.85 | 0.14 | 0.12 | 0.14 | 0.09 |
| F4-21.16 | 1.08 | 1.65 | 1.41 | 0.10 | 0.05 | 0.06 | 0.04 | 0.06 |
| F4-26.1 | 1.50 | 1.88 | 1.38 | 0.89 | 0.18 | 0.07 | 0.27 | 0.05 |
| Y035 | 2.64 | 2.60 | 2.32 | 1.79 | 0.35 | 0.08 | 0.05 | 0.05 |
| T2-23 | 2.28 | 2.45 | 2.19 | 1.68 | 0.41 | 0.10 | 0.05 | 0.05 |
| hIgG1 | 0.08 | 0.05 | 0.05 | 0.05 | 0.05 | 0.05 | 0.05 | 0.05 |

### (B) Assay for Binding of Chimeric Antibodies to Cells Expressing Monkey GPC3 Protein by FACS

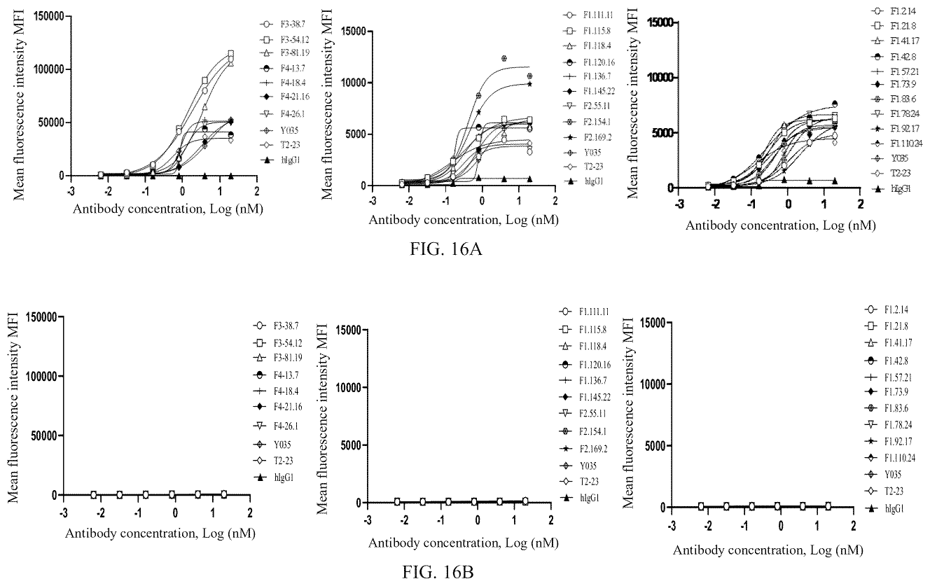

HEK293T-monkey GPC3 cells were subjected to FACS assay and data analysis according to the method described in Example 4 (B). The analysis results are shown in Table 20, FIG. 16A and FIG. 16B. The results showed that all chimeric antibodies bound to HEK293T-monkey GPC3 cells, but did not bind to HEK293T cells.

TABLE 20
Assay results for binding reactions of chimeric antibodies
with HEK293T-monkey GPC3 cells and HEK293T cells by FACS

| | 293T-cynoGPC3 | | 293T | |
|---|---|---|---|---|
| | Maximum mean | | Maximum mean | |
| | fluorescence | | fluorescence | |
| Antibody | intensity | Ec50 | intensity | Ec50 |
| name | Max MFI | (nM) | Max MFI | (nM) |
| F1.2.14 | 4738 | 1.10 | 89 | Negative |
| F1.21.8 | 6317 | 0.29 | 128 | Negative |
| F1.41.17 | 6213 | 0.23 | 68 | Negative |
| F1.42.8 | 7624 | 0.27 | 62 | Negative |
| F1.57.21 | 5382 | 0.77 | 61 | Negative |
| F1.73.9 | 5700 | 0.32 | 59 | Negative |
| F1.78.24 | 6582 | 0.33 | 90 | Negative |
| F1.83.6 | 5878 | 0.53 | 87 | Negative |
| F1.92.17 | 5525 | 2.26 | 71 | Negative |
| F1.110.24 | 5559 | 0.42 | 137 | Negative |
| F1.111.11 | 4453 | 0.42 | 120 | Negative |
| F1.115.8 | 6388 | 0.33 | 80 | Negative |
| F1.118.4 | 6224 | 0.32 | 144 | Negative |
| F1.120.16 | 5539 | 0.25 | 129 | Negative |
| F1.136.7 | 6475 | 1.94 | 111 | Negative |
| F1.145.22 | 6047 | 0.69 | 91 | Negative |
| F2.55.11 | 3530 | 0.31 | 79 | Negative |
| F2.154.1 | 12377 | 0.41 | 128 | Negative |
| F2.169.2 | 9881 | 0.51 | 99 | Negative |
| Y035 | 6281 | 1.14 | 73 | Negative |
| T2-23 | 4938 | 0.44 | 84 | Negative |
| hIgG1 | 680 | Negative | 144 | Negative |
| F3-38.7 | 109799 | 0.85 | 666 | Negative |
| F3-54.12 | 114974 | 0.72 | 556 | Negative |
| F3-81.19 | 106177 | 2.58 | 761 | Negative |
| F4-13.7 | 44123 | 1.10 | 154 | Negative |
| F4-18.4 | 52556 | 0.89 | 208 | Negative |
| F4-21.16 | 50922 | 3.25 | 170 | Negative |
| F4-26.1 | 50462 | 1.21 | 259 | Negative |
| Y035 | 52715 | 5.24 | 388 | Negative |
| T2-23 | 36715 | 0.56 | 490 | Negative |
| hIgG1 | 95 | Negative | 57 | Negative |

### Example 6 Assay for Affinity of GPC3 Human-Murine Chimeric Antibodies

### (A) Assay for Affinity of Chimeric Antibodies to Human GPC3 Protein

Anti-GPC3 human-murine chimeric antibodies were captured using a Protein A chip (GE Healthcare; 29-127-558). The sample and running buffer was HBS-EP+ (10 mM HEPES, 150 mM NaCl, 3 mM EDTA, 0.05% surfactant P20) (GE Healthcare; BR-1006-69). The flow cell was set at 25° C. The sample block was set at 16° C. Both were pretreated with the running buffer. In each cycle, first, the antibody to be tested was captured using the Protein A chip, and then a single concentration of GPC3 antigen protein was injected. The association and dissociation processes of the antibody with the antigen protein were recorded, and finally, the chip was regenerated using Glycine pH 1.5 (GE Healthcare; BR-1003-54). The association was determined by injecting different concentrations of human GPC3-His in the solution and maintaining for 240 s, wherein the flow rate was 30 μL/min, and the protein was diluted at a 1:1 dilution ratio from 200 nM to obtain 5 concentrations in total. The dissociation phase was monitored for up to 600 s and triggered by switching from the sample solution to the running buffer. The surface was regenerated by washing with 10 mM glycine solution (pH 1.5) at a flow rate of 30 μL/min for 30 s. The difference in bulk refractive index was corrected by subtracting the responses obtained from the goat anti-human Fc surface. Blank injections (double reference) were also subtracted. To calculate the apparent KD and other kinetic parameters, the Langmuir 1:1 model was used. The association rate (Kₐ), dissociation rate (K_{d}), and binding affinity (KD) of chimeric antibodies with the human GPC3 protein are shown in the table, in which the antibody Y035 was used as a control. As shown in Table 21, except four antibodies binding with poor fitting to the human GPC3 protein, the affinity of the remaining antibodies to the human GPC3 protein was greater than 1E-7 M.

TABLE 21
Binding affinity of chimeric antibodies to human GPC3 protein

| Antibody name | Ka (1/Ms) | Kd (1/s) | KD (M) |
|---|---|---|---|
| F1.2.14 | 7.16E+04 | 1.70E−04 | 2.37E−09 |
| F1.21.8 | 2.55E+05 | 6.91E−05 | 2.71E−10 |
| F1.41.17 | 2.65E+05 | 2.10E−03 | 7.91E−09 |
| F1.42.8 | Binding with poor fitting | | |
| F1.57.21 | 1.31E+05 | 2.78E−03 | 2.13E−08 |
| F1.73.9 | Binding with poor fitting | | |
| F1.78.24 | 1.06E+05 | 7.50E−04 | 7.06E−09 |
| F1.83.6 | 8.39E+04 | 4.93E−04 | 5.88E−09 |
| F1.92.17 | 2.28E+04 | 1.70E−04 | 7.46E−09 |
| F1.110.24 | 1.45E+05 | 3.62E−04 | 2.50E−09 |
| F1.111.11 | 6.58E+04 | 4.45E−04 | 6.75E−09 |
| F1.115.8 | 8.20E+05 | 1.15E−03 | 1.40E−09 |
| F1.118.4 | 2.51E+05 | 1.18E−04 | 4.72E−10 |
| F1.120.16 | 7.84E+04 | 6.29E−04 | 8.03E−09 |
| F1.136.7 | 8.41E+04 | 7.03E−05 | 8.37E−10 |
| F1.145.22 | 2.18E+05 | 1.64E−04 | 7.54E−10 |
| F1.3.9 | 2.87E+04 | 7.28E−05 | 2.54E−09 |
| F1.9.19 | 3.47E+04 | 3.14E−04 | 9.04E−09 |
| F1.62.9 | 2.80E+04 | 1.29E−03 | 4.60E−08 |
| F1.153.1 | 6.76E+04 | 6.22E−05 | 9.20E−10 |
| F2.55.11 | 5.70E+04 | 4.43E−04 | 7.77E−09 |
| F2.154.1 | 4.66E+05 | 5.63E−04 | 1.21E−09 |
| F2.169.2 | Binding with poor fitting | | |
| F2.23.8 | 1.61E+05 | 3.27E−03 | 2.04E−08 |
| F2.39.2 | 3.02E+05 | 2.10E−03 | 6.97E−09 |
| F2.92.1 | 4.04E+05 | 2.42E−04 | 6.00E−10 |
| F2.152.3 | Binding with poor fitting | | |
| F3-38.7 | 3.62E+05 | 3.61E−04 | 9.97E−10 |
| F3-54.12 | 2.75E+05 | 1.12E−03 | 4.09E−09 |
| F3-81.19 | 4.24E+05 | 2.31E−04 | 5.45E−10 |
| F4-13.7 | 4.84E+04 | 1.56E−03 | 3.23E−08 |
| F4-18.4 | 8.71E+04 | 4.43E−04 | 5.08E−09 |
| F4-21.16 | 4.06E+04 | 2.67E−04 | 6.58E−09 |
| F4-26.1 | 1.97E+05 | 4.61E−04 | 2.34E−09 |
| Y035 | 1.55E+05 | 3.65E−04 | 2.36E−09 |

### (B) Assay for Affinity of Chimeric Antibodies to Monkey GPC3-his Protein

The affinity of chimeric antibodies to the monkey GPC3-His protein was assayed according to the method described in Example 6 (A), in which the antibody Y035 was used as a control. As shown in Table 22, except four antibodies binding with poor fitting to the monkey GPC3 protein, the affinity of the remaining antibodies to the monkey GPC3 protein was greater than 1E-7 M.

TABLE 22
Binding affinity of chimeric antibodies to monkey GPC3 protein

| Antibody name | Ka (1/Ms) | Kd (1/s) | KD (M) |
|---|---|---|---|
| F1.2.14 | 1.25E+05 | 1.56E−04 | 1.25E−09 |
| F1.21.8 | 4.64E+05 | 4.65E−05 | 1.00E−10 |
| F1.41.17 | 4.82E+05 | 2.13E−03 | 4.42E−09 |
| F1.42.8 | Binding with poor fitting | | |
| F1.57.21 | 2.36E+05 | 3.06E−03 | 1.30E−08 |
| F1.73.9 | Binding with poor fitting | | |
| F1.78.24 | 1.96E+05 | 6.39E−04 | 3.26E−09 |
| F1.83.6 | 1.48E+05 | 5.03E−04 | 3.40E−09 |
| F1.92.17 | 2.74E+04 | 1.39E−04 | 5.07E−09 |
| F1.110.24 | 2.48E+05 | 3.42E−04 | 1.38E−09 |
| F1.111.11 | 9.58E+04 | 4.47E−04 | 4.67E−09 |
| F1.115.8 | 1.38E+06 | 1.45E−03 | 1.05E−09 |
| F1.118.4 | 4.53E+05 | 1.02E−04 | 2.26E−10 |
| F1.120.16 | 1.44E+05 | 6.39E−04 | 4.44E−09 |
| F1.136.7 | 1.58E+05 | 2.88E−05 | 1.83E−10 |
| F1.145.22 | 3.11E+05 | 1.64E−04 | 5.25E−10 |
| F1.3.9 | 3.82E+04 | 4.74E−05 | 1.24E−09 |
| F1.9.19 | 4.80E+04 | 3.17E−04 | 6.61E−09 |
| F1.62.9 | 3.55E+04 | 1.56E−03 | 4.40E−08 |
| F1.153.1 | 1.03E+05 | 4.27E−05 | 4.14E−10 |
| F2.55.11 | 8.28E+04 | 4.34E−04 | 5.24E−09 |
| F2.154.1 | 7.13E+05 | 5.04E−04 | 7.06E−10 |
| F2.169.2 | Binding with poor fitting | | |
| F2.23.8 | 2.81E+05 | 3.80E−03 | 1.35E−08 |
| F2.39.2 | 5.12E+05 | 2.19E−03 | 4.28E−09 |
| F2.92.1 | 6.28E+05 | 2.21E−04 | 3.53E−10 |
| F2.152.3 | Binding with poor fitting | | |
| F3-38.7 | 5.32E+05 | 3.86E−04 | 7.25E−10 |
| F3-54.12 | 5.41E+05 | 1.17E−03 | 2.17E−09 |
| F3-81.19 | 6.31E+05 | 2.61E−04 | 4.15E−10 |
| F4-13.7 | 8.40E+04 | 2.64E−03 | 3.14E−08 |
| F4-18.4 | 1.19E+05 | 5.52E−04 | 4.65E−09 |
| F4-21.16 | 3.88E+04 | 3.03E−04 | 7.81E−09 |
| F4-26.1 | 4.25E+05 | 5.87E−04 | 1.38E−09 |
| Y035 | 2.09E+05 | 3.65E−04 | 1.75E−09 |

### (C) Assay for Affinity of Chimeric Antibodies to Murine GPC3-his Protein

The affinity of 9 chimeric antibodies that bound to the murine GPC3-His protein to the murine GPC3-His protein was assayed by ELISA according to the method described in Example 6 (A), in which the antibody T2-23 was used as a control. As shown in Table 23, except four antibodies binding with poor fitting to the murine GPC3 protein, the affinity of the remaining 5 antibodies to the murine GPC3 protein was greater than 1E-8 M.

TABLE 23
Binding affinity of chimeric antibodies to murine GPC3 protein

| Antibody name | ka (1/Ms) | kd (1/s) | KD (M) |
|---|---|---|---|
| F1.2.14 | 1.74E+05 | 1.47E−04 | 8.44E−10 |
| F1.21.8 | 6.28E+05 | 4.84E−05 | 7.71E−11 |
| F1.42.8 | Binding with poor fitting | | |
| F1.111.11 | 1.29E+05 | 4.50E−04 | 3.47E−09 |
| F1.145.22 | 2.51E+05 | 1.73E−03 | 6.87E−09 |
| F2.169.2 | Binding with poor fitting | | |
| F2.152.3 | Binding with poor fitting | | |
| F4-26.1 | 4.92E+05 | 7.20E−04 | 1.46E−09 |
| F4-13.7 | Binding with poor fitting | | |
| T2-23 | 5.05E+05 | 6.75E−05 | 1.34E−10 |

### Example 7 Analysis on Antigen-Binding Epitopes of Antibodies (Identification of Antigen-Binding Regions of Antibodies)

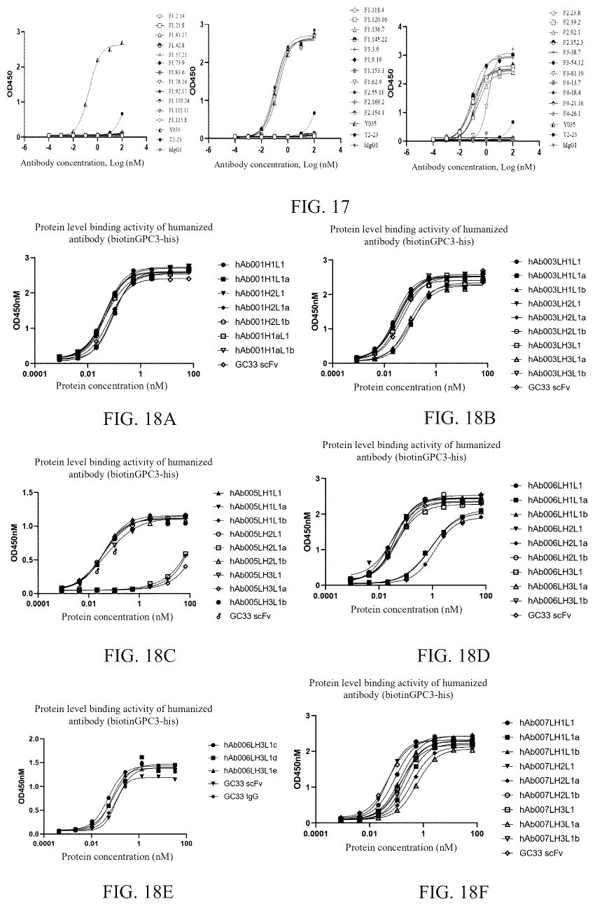

The mature GPC3 protein has a soluble N-terminal peptide of about 40 kD that can enter the blood and a membrane-bound C-terminal peptide of about 30 kD. The antibody Y035 recognizes a region (membrane-proximal region) that is near the cell membrane and that is at the C-terminus of the GPC3 protein, while the antibody T2-23 recognizes non-membrane-proximal regions. To identify whether the antigen-binding epitopes of the chimeric antibodies were located at the membrane proximal end, the chimeric antibodies were subjected to identification of membrane proximal binding by coating the polypeptide GC3pep (membrane proximal end) of human GPC3 according to the ELISA method described in Example 1 (A). As shown in FIG. 17 and Table 24, the 7 antibodies (F2.55.11, F2.154.1, F2.169.2, F2.23.8, F2.39.2, F2.92.1, and F2.152.3) in the F2 series, and the 3 antibodies (F3-38.7, F3-54.12, and F3-81.19) in the F3 series could recognize the membrane proximal polypeptide GC3pep, i.e., C-terminal GPC3 fragments; and none of the remaining 24 antibodies (F1 and F4 series) recognized the membrane proximal polypeptide GC3 pep.

TABLE 24
Assay results for binding reactions of chimeric
antibodies with polypeptide GC3pep by ELISA

| | Antibody concentration (nM) | | | | | | | |
|---|---|---|---|---|---|---|---|---|
| Antibody | | | | | | | | Blank |
| name | 100 | 10 | 1 | 0.1 | 0.01 | 0.001 | 0.0001 | control |
| F1.2.14 | 0.05 | 0.05 | 0.05 | 0.05 | 0.05 | 0.05 | 0.05 | 0.05 |
| F1.21.8 | 0.05 | 0.05 | 0.05 | 0.05 | 0.05 | 0.05 | 0.05 | 0.05 |
| F1.41.17 | 0.06 | 0.05 | 0.05 | 0.05 | 0.05 | 0.05 | 0.05 | 0.05 |
| F1.42.8 | 0.06 | 0.05 | 0.05 | 0.05 | 0.05 | 0.05 | 0.05 | 0.05 |
| F1.57.21 | 0.06 | 0.05 | 0.05 | 0.05 | 0.05 | 0.05 | 0.05 | 0.05 |
| F1.73.9 | 0.09 | 0.05 | 0.05 | 0.05 | 0.05 | 0.05 | 0.04 | 0.05 |
| F1.78.24 | 0.06 | 0.05 | 0.05 | 0.05 | 0.05 | 0.05 | 0.05 | 0.05 |
| F1.83.6 | 0.07 | 0.06 | 0.05 | 0.05 | 0.05 | 0.05 | 0.04 | 0.05 |
| F1.92.17 | 0.10 | 0.06 | 0.05 | 0.06 | 0.05 | 0.05 | 0.05 | 0.05 |
| F1.110.24 | 0.06 | 0.07 | 0.04 | 0.05 | 0.05 | 0.05 | 0.04 | 0.05 |
| F1.111.11 | 0.15 | 0.06 | 0.06 | 0.06 | 0.05 | 0.06 | 0.06 | 0.07 |
| F1.115.8 | 0.10 | 0.05 | 0.08 | 0.05 | 0.04 | 0.05 | 0.05 | 0.06 |
| F1.118.4 | 0.06 | 0.05 | 0.04 | 0.04 | 0.04 | 0.04 | 0.05 | 0.06 |
| F1.120.16 | 0.08 | 0.06 | 0.04 | 0.04 | 0.04 | 0.04 | 0.05 | 0.06 |
| F1.136.7 | 0.08 | 0.04 | 0.04 | 0.04 | 0.04 | 0.04 | 0.05 | 0.06 |
| F1.145.22 | 0.11 | 0.05 | 0.04 | 0.04 | 0.04 | 0.04 | 0.04 | 0.06 |
| F1.3.9 | 0.13 | 0.06 | 0.05 | 0.05 | 0.05 | 0.05 | 0.07 | 0.07 |
| F1.9.19 | 0.08 | 0.06 | 0.06 | 0.04 | 0.05 | 0.07 | 0.05 | 0.06 |
| F1.62.9 | 0.13 | 0.07 | 0.05 | 0.05 | 0.05 | 0.05 | 0.05 | 0.06 |
| F1.153.1 | 0.11 | 0.05 | 0.04 | 0.05 | 0.04 | 0.05 | 0.06 | 0.07 |
| F2.55.11 | 2.85 | 2.51 | 2.41 | 1.34 | 0.26 | 0.07 | 0.05 | 0.06 |
| F2.154.1 | 2.73 | 2.42 | 2.29 | 1.13 | 0.20 | 0.06 | 0.05 | 0.06 |
| F2.169.2 | 2.73 | 2.38 | 2.36 | 1.25 | 0.24 | 0.06 | 0.05 | 0.07 |
| F2.23.8 | 2.51 | 2.42 | 2.12 | 1.38 | 0.30 | 0.10 | 0.07 | 0.05 |
| F2.39.2 | 2.58 | 2.38 | 2.21 | 1.35 | 0.32 | 0.09 | 0.05 | 0.06 |
| F2.92.1 | 2.41 | 2.28 | 1.92 | 0.85 | 0.15 | 0.07 | 0.05 | 0.05 |
| F2.152.3 | 2.63 | 2.43 | 2.18 | 1.53 | 0.35 | 0.09 | 0.05 | 0.05 |
| F3-38.7 | 3.22 | 2.83 | 2.57 | 1.01 | 0.19 | 0.06 | 0.05 | 0.07 |
| F3-54.12 | 3.03 | 2.80 | 2.64 | 1.63 | 0.33 | 0.09 | 0.07 | 0.07 |
| F3-81.19 | 2.94 | 2.88 | 1.21 | 0.19 | 0.05 | 0.05 | 0.05 | 0.07 |
| F4-13.7 | 0.06 | 0.04 | 0.03 | 0.03 | 0.04 | 0.08 | 0.05 | 0.04 |
| F4-18.4 | 0.06 | 0.03 | 0.03 | 0.03 | 0.04 | 0.04 | 0.04 | 0.04 |
| F4-21.16 | 0.08 | 0.04 | 0.03 | 0.03 | 0.04 | 0.04 | 0.04 | 0.04 |
| F4-26.1 | 0.12 | 0.04 | 0.28 | 0.08 | 0.16 | 0.19 | 0.05 | 0.38 |
| Y035 | 2.70 | 2.50 | 2.20 | 0.94 | 0.17 | 0.05 | 0.05 | 0.06 |
| T2-23 | 0.67 | 0.14 | 0.06 | 0.05 | 0.05 | 0.05 | 0.05 | 0.06 |
| hIgG1 | 0.14 | 0.07 | 0.06 | 0.06 | 0.05 | 0.05 | 0.05 | 0.07 |

### Example 8 Humanization Design of GPC3 Antibodies

By comparing the IMGT database (http://imgt.cines.fr) for germline genes from heavy chain/light chain variable regions of human antibodies with the Molecular Operating Environment (MOE) software, germline genes, with high homology with murine antibodies, from heavy chain and light chain variable regions were respectively selected as templates, and CDRs of murine antibodies were respectively grafted into corresponding human to form a templates variable region sequence of FR1-CDR1-FR2-CDR2-FR3-CDR3-FR4. Back mutations and/or hotspot mutations were performed as needed. The antibody sequences and CDR sequences in this example were numbered according to the Kabat numbering scheme.

### 8.1 Humanization of Antibody F2.169.2

### 8.1.1 Selection of Germline Sequence for F2.169.2

With IGKV2-40*01 and IGKJ2*01 as humanized light chain templates, and IGHV3-72*01 and IGHJ1*01 as humanized heavy chain templates for the murine antibody F2.169.2, CDRs of the murine antibody F2.169.2 were respectively grafted into corresponding humanized templates to obtain a humanized antibody GPC3-hAb001 of F2.169.2. A variable region sequence of the humanized antibody is shown as follows:

GPC3-hAb001 HCVR (VH-CDR graft, IGHV3-72*01):
(SEQ ID NO: 594)
EVQLVESGGGLVQPGGSLRLSCAASGFTFSTNAMNWVRQAPG
KGLEWVGRIRSKSNNYATYYADSVKDRFTISRDDSKNSLYLQ
MNSLKTEDTAVYYCARDGYYVLFAYWGQGTLVTVSS.
GPC3-hAb001 LCVR (VL-CDR graft, IGKV2-40*01):
(SEQ ID NO: 595)
DIVMTQTPLSLPVTPGEPASISCRFSQSIVNSNGNTYLEWYL
QKPGQSPQLLIYKVSNRFSGVPDRFSGSGSGTDFTLKISRVE
AEDVGVYYCFQGSHVPFTFGQGTKLEIK.

### 8.1.2 Design of Back Mutations and Hotspot Mutations for F2.169.2 Humanized Antibodies

Key amino acids in FR sequences of F2.169.2 humanized antibodies were back-mutated as needed to ensure the original affinity. In view of a high-risk modifiable site NG on a light chain of F2.169.2, amino acid mutations were performed on the NG by means of computational simulation based on the antibody structure to eliminate modification risks. The detailed mutation design is shown in Table 25 (back mutations were performed in a natural numbering order).

TABLE 25
Design of back mutations and hotspot mutations
for F2.169.2 humanized antibodies

| VL | | VH | |
|---|---|---|---|
| L1 | Graft(IGKV2-40*01) + | H1 | Graft(IGHV3-72*01) + T28S, |
| | Y41F | | S30N, A99V |
| L1a | Graft(IGKV2-40*01) + | H2 | Graft(IGHV3-72*01) + |
| | Y41F + N33D | | T28S, S30N, N79S, A99V |
| L1b | Graft(IGKV2-40*01) + | H1a | Graft(IGHV3-72*01) + T28S, |
| | Y41F + G34A | | S30N, A99V + D68G |
| FR4 | IGKJ2*01 | FR4 | IGHJ1*01 |

### 8.1.3 Combinations of Sequences of F2.169.2 Humanized Antibodies

The designs of back mutations and hotspot mutations for the F2.169.2 humanized antibodies in Table 25 were combined to eventually obtain multiple F2.169.2 humanized antibodies (see Table 26 for details).

TABLE 26
Corresponding amino acid sequences
of F2.169.2 humanized antibodies

VL

| VH | L1 | L1a | L1b |
|---|---|---|---|
| H1 | hAb001H1L1 | hAb001H1L1a | hAb001H1L1b |
| H2 | hAb001H2L1 | hAb001H2L1a | hAb001H2L1b |
| H1a | hAb001H1aL1 | hAb001H1aL1a | hAb001H1aL1b |

Amino acid sequences of humanized heavy chain/light chain variable regions are shown in Table 27:

TABLE 27
Amino acid sequences of back-mutated
variable regions of F2.169.2
humanized antibodies

| VL/ | Sequence | |
|---|---|---|
| VH | No. | Amino acid sequence |
| L1 | SEQ ID | DIVMTQTPLSLPVTPGEPASISCRFSQSIVNS |
| | NO: 596 | NGNTYLEWFLQKPGQSPQLLIYKVSNRFSGVP |
| | | DRFSGSGSGTDFTLKISRVEAEDVGVYYCFQG |
| | | SHVPFTFGQGTKLEIK |
| L1a | SEQ ID | DIVMTQTPLSLPVTPGEPASISCRFSQSIVNS |
| | NO: 597 | DGNTYLEWFLQKPGQSPQLLIYKVSNRFSGVP |
| | | DRFSGSGSGTDFTLKISRVEAEDVGVYYCFQG |
| | | SHVPFTFGQGTKLEIK |
| L1b | SEQ ID | DIVMTQTPLSLPVTPGEPASISCRFSQSIVNS |
| | NO: 598 | NANTYLEWFLQKPGQSPQLLIYKVSNRFSGVP |
| | | DRFSGSGSGTDFTLKISRVEAEDVGVYYCFQG |
| | | SHVPFTFGQGTKLEIK |
| H1 | SEQ ID | EVQLVESGGGLVQPGGSLRLSCAASGFSFNTN |
| | NO: 599 | AMNWVRQAPGKGLEWVGRIRSKSNNYATYYAD |
| | | SVKDRFTISRDDSKNSLYLQMNSLKTEDTAVY |
| | | YCVRDGYYVLFAYWGQGTLVTVSS |
| H2 | SEQ ID | EVQLVESGGGLVQPGGSLRLSCAASGFSFNTN |
| | NO: 600 | AMNWVRQAPGKGLEWVGRIRSKSNNYATYYAD |
| | | SVKDRFTISRDDSKSSLYLQMNSLKTEDTAVY |
| | | YCVRDGYYVLFAYWGQGTLVTVSS |
| H1a | SEQ ID | EVQLVESGGGLVQPGGSLRLSCAASGFSFNTN |
| | NO: 601 | AMNWVRQAPGKGLEWVGRIRSKSNNYATYYAD |
| | | SVKGRFTISRDDSKNSLYLQMNSLKTEDTAVY |
| | | YCVRDGYYVLFAYWGQGTLVTVSS |

According to the Kabat numbering scheme, the analysis results for CDR sequences of heavy chain/light chain variable regions of the humanized antibodies are shown in Table 28:

TABLE 28
Kabat analysis results for CDR sequences of
heavy chain/light chain variable regions
of F2.169.2 humanized antibodies

| VL/VH | CDR1 | CDR2 | CDR3 |
|---|---|---|---|
| L1 | RFSQSIVN | KVSNRFS | FQGSHVPFT |
| | SNGNTYLE | SEQ ID | SEQ ID |
| | SEQ ID | NO: 508 | NO: 509 |
| | NO: 507 | | |
| L1a | RFSQSIVNS | KVSNRFS | FQGSHVPFT |
| | DGNTYLE | SEQ ID | SEQ ID |
| | SEQ ID | NO: 508 | NO: 509 |
| | NO: 602 | | |
| L1b | RFSQSIVN | KVSNRFS | FQGSHVPFT |
| | SNANTYLE | SEQ ID | SEQ ID |
| | SEQ ID | NO: 508 | NO: 509 |
| | NO: 603 | | |
| H1/ | TNAMN | RIRSKSNNYA | DGYYVLFAY |
| H2 | SEQ ID | TYYADSVKD | SEQ ID |
| | NO: 267 | SEQ ID | NO: 269 |
| | | NO: 268 | |
| H1a | TNAMN | RIRSKSNNYA | DGYYVLFAY |
| | SEQ ID | TYYADSVKG | |
| | | SEQ ID | SEQ ID |
| | NO: 267 | NO: 604 | NO: 269 |

### 8.2 Humanization of Antibody F2.154.1

### 8.2.1 Selection of Germline Sequence for F2.154.1

With IGKV2-40*01 and IGKJ2*01 as humanized light chain templates, and IGHV1-69*02 and IGHJ1*01 as humanized heavy chain templates for the murine antibody F2.154.1, CDRs of the murine antibody F2.154.1 were respectively grafted into corresponding humanized templates to obtain a humanized antibody GPC3-hAb003L of F2.154.1. A variable region sequence of the humanized antibody is shown as follows:

GPC3-hAb003L HCVR (VH-CDR graft, IGHV1-69*02):
(SEQ ID NO: 605)
EVQLVQSGAEVKKPGSSVKVSCKASGGTFSDYEMHWVRQ
APGQGLEWMGTIDPETGNTAYTQKFMDRVTITADKSTST
AYMELSSLRSEDTAVYYCARGYSFTYWGQGTLVTVSS.
GPC3-hAb003L LCVR (VL-CDR graft, IGKV2-40*01):
(SEQ ID NO: 606)
DIVMTQTPLSLPVTPGEPASISCRSSQNIVHSNGNTYLQ
WYLQKPGQSPQLLIYKVSNRFSGVPDRFSGSGSGTDFTL
KISRVEAEDVGVYYCFQGSHVPFTFGQGTKLEIK.

### 8.2.2 Design of Back Mutations and Hotspot Mutations for F2.154.1 Humanized Antibodies

Key amino acids in FR sequences of F2.154.1 humanized antibodies were back-mutated as needed to ensure the original affinity. In view of a high-risk modifiable site NG on a light chain of F2.154.1, amino acid mutations were performed on the NG by means of computational simulation based on the antibody structure to eliminate modification risks. The detailed mutation design is shown in Table 29 (back mutations were performed in a natural numbering order).

TABLE 29
Design of back mutations and hotspot mutations
for F2.154.1 humanized antibodies

| VL | | VH | |
|---|---|---|---|
| L1 | Graft(IGKV2-40*01) + | H1 | Graft(IGHV1-69*02) + |
| | Y54F | | G27Y, A97T |
| L1a | Graft(IGKV2-40*01) + | H2 | Graft(IGHV1-69*02) + |
| | Y54F + N33D | | G27Y, T28R, S30T, A97T |
| L1b | Graft(IGKV2-40*01) + | H3 | Graft(IGHV1-69*02) + |
| | Y54F + G34R | | G27Y, T28R, S30T, I70L, A97T |
| FR4 | IGKJ2*01 | FR4 | IGHJ1*01 |

### 8.2.3 Combinations of Sequences of F2.154.1 Humanized Antibodies

The designs of back mutations and hotspot mutations for the F2.154.1 humanized antibodies in Table 29 were combined to eventually obtain multiple F2.154.1 humanized antibodies (see Table 30 for details).

TABLE 30
Corresponding amino acid sequences
of F2.154.1 humanized antibodies

VL

| VH | L1 | L1a | L1b |
|---|---|---|---|
| H1 | hAb003LH1L1 | hAb003LH1L1a | hAb003LH1L1b |
| H2 | hAb003LH2L1 | hAb003LH2L1a | hAb003LH2L1b |
| H3 | hAb003LH3L1 | hAb003LH3L1a | hAb003LH3L1b |

Amino acid sequences of humanized heavy chain/light chain variable regions are shown in Table 31:

TABLE 31
Amino acid sequences of back-mutated variable
regions of F2.154.1 humanized antibodies

| VL/ | Sequence | |
|---|---|---|
| VH | No. | Amino acid sequence |
| L1 | SEQ ID | DIVMTQTPLSLPVTPGEPASISCRSSQNIVHSN |
| | NO: 607 | GNTYLQWYLQKPGQSPQLLIFKVSNRFSGVPDR |
| | | FSGSGSGTDFTLKISRVEAEDVGVYYCFQGSH |
| | | VPFTFGQGTKLEIK |
| L1a | SEQ ID | DIVMTQTPLSLPVTPGEPASISCRSSQNIVHSD |
| | NO: 608 | GNTYLQWYLQKPGQSPQLLIFKVSNRFSGVPDR |
| | | FSGSGSGTDFTLKISRVEAEDVGVYYCFQGSH |
| | | VPFTFGQGTKLEIK |
| L1b | SEQ ID | DIVMTQTPLSLPVTPGEPASISCRSSQNIVHSN |
| | NO: 609 | RNTYLQWYLQKPGQSPQLLIFKVSNRFSGVPDR |
| | | FSGSGSGTDFTLKISRVEAEDVGVYYCFQGSH |
| | | VPFTFGQGTKLEIK |
| H1 | SEQ ID | EVQLVQSGAEVKKPGSSVKVSCKASGYTFSD |
| | NO: 610 | YEMHWVRQAPGQGLEWMGTIDPETGNTAYTQ |
| | | KFMDRVTITADKSTSTAYMELSSLRSEDTAV |
| | | YYCTRGYSFTYWGQGTLVTVSS |
| H2 | SEQ ID | EVQLVQSGAEVKKPGSSVKVSCKASGYRFTD |
| | NO: 611 | YEMHWVRQAPGQGLEWMGTIDPETGNTAYTQ |
| | | KFMDRVTITADKSTSTAYMELSSLRSEDTAV |
| | | YYCTRGYSFTYWGQGTLVTVSS |
| H3 | SEQ ID | EVQLVQSGAEVKKPGSSVKVSCKASGYRFTD |
| | NO: 612 | YEMHWVRQAPGQGLEWMGTIDPETGNTAYTQ |
| | | KFMDRVTLTADKSTSTAYMELSSLRSEDTA |
| | | VYYCTRGYSFTYWGQGTLVTVSS |

According to the Kabat numbering scheme, the analysis results for CDR sequences of heavy chain/light chain variable regions of the humanized antibodies are shown in Table 32:

TABLE 32
Kabat analysis results for CDR sequences
of heavy chain/light chain variable
regions of F2.154.1 humanized antibodies

| VL/VH | CDR1 | CDR2 | CDR3 |
|---|---|---|---|
| L1 | RSSQNIVH | KVSNRFS | FQGSHVPFT |
| | SNGNTYLQ | SEQ ID | SEQ ID |
| | SEQ ID | NO: 502 | NO: 503 |
| | NO: 501 | | |
| L1a | RSSQNIVH | KVSNRFS | FQGSHVPFT |
| | SDGNTYLQ | SEQ ID | SEQ ID |
| | SEQ ID | NO: 502 | NO: 503 |
| | NO: 613 | | |
| L1b | RSSQNIVH | KVSNRFS | FQGSHVPFT |
| | SNRNTYLQ | SEQ ID | SEQ ID |
| | SEQ ID | NO: 502 | NO: 503 |
| | NO: 614 | | |
| H1/ | DYEMH | TIDPETGN | GYSFTY |
| H2/ | SEQ ID | TAYTQKFMD | SEQ ID |
| H3 | NO: 258 | SEQ ID | NO: 260 |
| | | NO: 259 | |

### 8.3 Humanization of Antibody F3.54.12

### 8.3.1 Selection of Germline Sequence for F3.54.12

With IGKV2-40*01 and IGKJ4*01 as humanized light chain templates, and IGHV1-18*01 and IGHJ6*01 as humanized heavy chain templates for the murine antibody F3.54.12, CDRs of the murine antibody F3.54.12 were respectively grafted into corresponding humanized templates to obtain a humanized antibody GPC3-hAb005L of F3.54.12. A variable region sequence of the humanized antibody is shown as follows:

GPC3-hAb005L HCVR (VH-CDR graft, IGHV1-18*01):
(SEQ ID NO: 615)
EVQLVQSGAEVKKPGASVKVSCKASGYTFTDYEMHWVRQAPG
QGLEWMGAIDPETGNTAYIQKFKGRVTMTTDTSTSTAYMELR
SLRSDDTAVYYCARFYSYSHWGQGTTVTVSS.
GPC3-hAb005L LCVR (VL-CDR graft, IGKV2-40*01):
(SEQ ID NO: 616)
DIVMTQTPLSLPVTPGEPASISCRSSQSLVHSNGNTYLHWYL
QKPGQSPQLLIYKVSNRFSGVPDRFSGSGSGTDFTLKISRVE
AEDVGVYYCSQNTHVPPTFGGGTKVEIK.

### 8.3.2 Design of Back Mutations and Hotspot Mutations for F3.54.12 Humanized Antibodies

Key amino acids in FR sequences of F3.54.12 humanized antibodies were back-mutated as needed to ensure the original affinity. In view of a high-risk modifiable site NG on a light chain of F3.54.12, amino acid mutations were performed on the NG by means of computational simulation based on the antibody structure to eliminate modification risks. The detailed mutation design is shown in Table 33 (back mutations were performed in a natural numbering order).

TABLE 33
Design of back mutations and hotspot mutations
for F3.54.12 humanized antibodies

| VL | | VH | |
|---|---|---|---|
| L1 | Graft(IGKV2-40*01) | H1 | Graft(IGHV1-18*01) + |
| | | | T74K, A97T |
| L1a | Graft(IGKV2-40*01) + | H2 | Graft(IGHV1-18*01) + |
| | N33D | | M70L, T74K, A97T |
| L1b | Graft(IGKV2-40*01) + | H3 | Graft(IGHV1-18*01) + |
| | G34R | | M48I, V68A, M70L, |
| | | | T74K, A97T |
| FR4 | IGKJ4*01 | FR4 | IGHJ6*01 |

### 8.3.3 Combinations of Sequences of F3.54.12 Humanized Antibodies

The designs of back mutations and hotspot mutations for the F3.54.12 humanized antibodies in Table 33 were combined to eventually obtain multiple F3.54.12 humanized antibodies (see Table 34 for details).

TABLE 34
Corresponding amino acid sequences
of F3.54.12 humanized antibodies

VL

| VH | L1 | L1a | L1b |
|---|---|---|---|
| H1 | hAb005LH1L1 | hAb005LH1L1a | hAb005LH1L1b |
| H2 | hAb005LH2L1 | hAb005LH2L1a | hAb005LH2L1b |
| H3 | hAb005LH3L1 | hAb005LH3L1a | hAb005LH3L1b |

Amino acid sequences of humanized heavy chain/light chain variable regions are shown in Table 35:

TABLE 35
Amino acid sequences of back-mutated
variable regions of F3.54.12
humanized antibodies

| | Se- | |
|---|---|---|
| VL/ | quence | |
| VH | No. | Amino acid sequence |
| L1 | SEQ ID | DIVMTQTPLSLPVTPGEPASISCRSSQSLVHSN |
| | NO: 617 | GNTYLHWYLQKPGQSPQLLIYKVSNRFSGVPD |
| | | RFSGSGSGTDFTLKISRVEAEDVGVYYCSQNT |
| | | HVPPTFGGGTKVEIK |
| L1a | SEQ ID | DIVMTQTPLSLPVTPGEPASISCRSSQSLVHSD |
| | NO: 618 | GNTYLHWYLQKPGQSPQLLIYKVSNRFSGVPD |
| | | RFSGSGSGTDFTLKISRVEAEDVGVYYCSQNT |
| | | HVPPTFGGGTKVEIK |
| L1b | SEQ ID | DIVMTQTPLSLPVTPGEPASISCRSSQSLVHSN |
| | NO: 619 | RNTYLHWYLQKPGQSPQLLIYKVSNRFSGVPDR |
| | | FSGSGSGTDFTLKISRVEAEDVGVYYCSQNTH |
| | | VPPTFGGGTKVEIK |
| H1 | SEQ ID | EVQLVQSGAEVKKPGASVKVSCKASGYTFTD |
| | NO: 620 | YEMHWVRQAPGQGLEWMGAIDPETGNTAYI |
| | | QKFKGRVTMTTDKSTSTAYMELRSLRSDDTA |
| | | VYYCTRFYSYSHWGQGTTVTVSS |
| H2 | SEQ ID | EVQLVQSGAEVKKPGASVKVSCKASGYTFTD |
| | NO: 621 | YEMHWVRQAPGQGLEWMGAIDPETGNTAYI |
| | | QKFKGRVTLTTDKSTSTAYMELRSLRSDDTAV |
| | | YYCTRFYSYSHWGQGTTVTVSS |
| H3 | SEQ ID | EVQLVQSGAEVKKPGASVKVSCKASGYTFTD |
| | NO: 622 | YEMHWVRQAPGQGLEWIGAIDPETGNTAYIQ |
| | | KFKGRATLTTDKSTSTAYMELRSLRSDDTAVY |
| | | YCTRFYSYSHWGQGTTVTVSS |

According to the Kabat numbering scheme, the analysis results for CDR sequences of heavy chain/light chain variable regions of the humanized antibodies are shown in Table 36:

TABLE 36
Kabat analysis results for CDR sequences
of heavy chain/light chain variable
regions of F3.54.12 humanized antibodies

| VL/VH | CDR1 | CDR2 | CDR3 |
|---|---|---|---|
| L1 | RSSQSLVH | KVSNRFS | SQNTH |
| | SNGNTYLH | SEQ ID | VPPT |
| | SEQ ID | NO: 538 | SEQ ID |
| | NO: 537 | | NO: 539 |
| L1a | RSSQSLVH | KVSNRFS | SQNTH |
| | SDGNTYLH | SEQ ID | VPPT |
| | SEQ ID | NO: 538 | SEQ ID |
| | NO: 623 | | NO: 539 |
| L1b | RSSQSLVH | KVSNRFS | SQNTH |
| | SNRNTYLH | SEQ ID | VPPT |
| | SEQ ID | NO: 538 | SEQ ID |
| | NO: 624 | | NO: 539 |
| H1/2/3 | DYEMH | AIDPETG | FYSYSH |
| | SEQ ID | NTAYIQK | SEQ ID |
| | NO: 312 | FKG | NO: 314 |
| | | SEQ ID | |
| | | NO: 313 | |

### 8.4 Humanization of Antibody F3.38.7

### 8.4.1 Selection of Germline Sequence for F3.38.7

With IGKV2-40*01 and IGKJ2*01 as humanized light chain templates, and IGHV1-69*02 and IGHJ1*01 as humanized heavy chain templates for the murine antibody F3.38.7, CDRs of the murine antibody F3.38.7 were respectively grafted into corresponding humanized templates to obtain a humanized antibody GPC3-hAb006L of F3.38.7. A variable region sequence of the humanized antibody is shown as follows:

GPC3-hAb006L HCVR (VH-CDR graft, IGHV1-69*02):
(SEQ ID NO: 625)
EVQLVQSGAEVKKPGSSVKVSCKASGGTFSDYEIHWVRQAPGQ
GLEWMGAIDPKTGNTAYNQKFMGRVTITADKSTSTAYMELSSL
RSEDTAVYYCARYFSFAYWGQGTLVTVSS.
GPC3-hAb006L LCVR (VL-CDR graft, IGKV2-40*01):
(SEQ ID NO: 626)
DIVMTQTPLSLPVTPGEPASISCRSSQTFVHSNGNTYLQWYL
QKPGQSPQLLIYKVSNRFSGVPDRESGSGSGTDFTLKISRVE
AEDVGVYYCFQGSHVPYTFGQGTKLEIK.

### 8.4.2 Design of Back Mutations and Hotspot Mutations for F3.38.7 Humanized Antibodies

Key amino acids in FR sequences of F3.38.7 humanized antibodies were back-mutated as needed to ensure the original affinity. In view of a high-risk modifiable site NG on a light chain of F3.38.7, amino acid mutations were performed on the NG by means of computational simulation based on the antibody structure to eliminate modification risks. The detailed mutation design is shown in Table 37 (back mutations were performed in a natural numbering order).

TABLE 37
Design of back mutations and hotspot mutations
for F3.38.7 humanized antibodies

| VL | | VH | |
|---|---|---|---|
| L1 | Graft(IGKV2-40*01) | H1 | Graft(IGHV1-69*02) + |
| | | | G27Y, A97T |
| L1a | Graft(IGKV2-40*01) + | H2 | Graft(IGHV1-69*02) + |
| | N33D | | G27Y, S30T, A97T |
| L1b | Graft(IGKV2-40*01) + | H3 | Graft(IGHV1-69*02) + |
| | G34A | | G27Y, S30T, I70L, A97T |
| L1c | Graft(IGKV2-40*01) + | FR4 | IGHJ1*01 |
| | G34R | | |
| L1d | Graft(IGKV2-40*01) + | | |
| | G34S | | |
| L1e | Graft(IGKV2-40*01) + | | |
| | G34V | | |
| FR4 | IGKJ2*01 | | |

### 8.4.3 Combinations of Sequences of F3.38.7 Humanized Antibodies

The designs of back mutations and hotspot mutations for the F3.38.7 humanized antibodies in Table 37 were combined to eventually obtain multiple F3.38.7 humanized antibodies (see Table 38 for details).

TABLE 38
Corresponding amino acid sequences of F3.38.7 humanized antibodies

| | VL | | | | | |
|---|---|---|---|---|---|---|
| VH | L1 | L1a | L1b | L1c | L1d | L1e |
| H1 | hAb006LH1L1 | hAb006LH1L1a | hAb006LH1L1b | — | — | — |
| H2 | hAb006LH2L1 | hAb006LH2L1a | hAb006LH2L1b | — | — | — |
| H3 | hAb006LH3L1 | hAb006LH3L1a | hAb006LH3L1b | hAb006LH3L1c | hAb006LH3L1d | hAb006LH3L1e |

Amino acid sequences of humanized heavy chain/light chain variable regions are shown in Table 39:

TABLE 39
Amino acid sequences of back-mutated variable regions of F3.38.7
humanized antibodies

| VL/VH | Sequence No. | Amino acid sequence |
|---|---|---|
| L1 | SEQ ID NO: 627 | DIVMTQTPLSLPVTPGEPASISCRSSQTFVHSNG |
| | | NTYLQWYLQKPGQSPQLLIYKVSNRFSGVPDR |
| | | FSGSGSGTDFTLKISRVEAEDVGVYYCFQGSHV |
| | | PYTFGQGTKLEIK |
| L1a | SEQ ID NO: 628 | DIVMTQTPLSLPVTPGEPASISCRSSQTFVHSDG |
| | | NTYLQWYLQKPGQSPQLLIYKVSNRFSGVPDR |
| | | FSGSGSGTDFTLKISRVEAEDVGVYYCFQGSHV |
| | | PYTFGQGTKLEIK |
| L1b | SEQ ID NO: 629 | DIVMTQTPLSLPVTPGEPASISCRSSQTFVHSNA |
| | | NTYLQWYLQKPGQSPQLLIYKVSNRFSGVPDR |
| | | FSGSGSGTDFTLKISRVEAEDVGVYYCFQGSHV |
| | | PYTFGQGTKLEIK |
| L1c | SEQ ID NO: 630 | DIVMTQTPLSLPVTPGEPASISCRSSQTFVHSNR |
| | | NTYLQWYLQKPGQSPQLLIYKVSNRFSGVPDR |
| | | FSGSGSGTDFTLKISRVEAEDVGVYYCFQGSHV |
| | | PYTFGQGTKLEIK |
| L1d | SEQ ID NO: 631 | DIVMTQTPLSLPVTPGEPASISCRSSQTFVHSNS |
| | | NTYLQWYLQKPGQSPQLLIYKVSNRFSGVPDR |
| | | FSGSGSGTDFTLKISRVEAEDVGVYYCFQGSHV |
| | | PYTFGQGTKLEIK |
| L1e | SEQ ID NO: 632 | DIVMTQTPLSLPVTPGEPASISCRSSQTFVHSNV |
| | | NTYLQWYLQKPGQSPQLLIYKVSNRFSGVPDR |
| | | FSGSGSGTDFTLKISRVEAEDVGVYYCFQGSHV |
| | | PYTFGQGTKLEIK |
| H1 | SEQ ID NO: 633 | EVQLVQSGAEVKKPGSSVKVSCKASGYTFSDY |
| | | EIHWVRQAPGQGLEWMGAIDPKTGNTAYNQK |
| | | FMGRVTITADKSTSTAYMELSSLRSEDTAVYY |
| | | CTRYFSFAYWGQGTLVTVSS |
| H2 | SEQ ID NO: 634 | EVQLVQSGAEVKKPGSSVKVSCKASGYTFTDY |
| | | EIHWVRQAPGQGLEWMGAIDPKTGNTAYNQK |
| | | FMGRVTITADKSTSTAYMELSSLRSEDTAVYY |
| | | CTRYFSFAYWGQGTLVTVSS |
| H3 | SEQ ID NO: 635 | EVQLVQSGAEVKKPGSSVKVSCKASGYTFTDY |
| | | EIHWVRQAPGQGLEWMGAIDPKTGNTAYNQK |
| | | FMGRVTLTADKSTSTAYMELSSLRSEDTAVYY |
| | | CTRYFSFAYWGQGTLVTVSS |

According to the Kabat numbering scheme, the analysis results for CDR sequences of heavy chain/light chain variable regions of the humanized antibodies are shown in Table 40:

TABLE 40
Kabat analysis results for CDR sequences of heavy chain/light chain variable
regions of F3.38.7 humanized antibodies

| VL/VH | CDR1 | CDR2 | CDR3 |
|---|---|---|---|
| L1 | RSSQTFVHSNGNTYLQ | KVSNRFS | FQGSHVPYT |
| | SEQ ID NO: 531 | SEQ ID NO: 532 | SEQ ID NO: 533 |
| L1a | RSSQTFVHSDGNTYLQ | KVSNRFS | FQGSHVPYT |
| | SEQ ID NO: 636 | SEQ ID NO: 532 | SEQ ID NO: 533 |
| L1b | RSSQTFVHSNANTYLQ | KVSNRFS | FQGSHVPYT |
| | SEQ ID NO: 637 | SEQ ID NO: 532 | SEQ ID NO: 533 |
| L1c | RSSQTFVHSNRNTYLQ | KVSNRFS | FQGSHVPYT |
| | SEQ ID NO: 638 | SEQ ID NO: 532 | SEQ ID NO: 533 |
| L1d | RSSQTFVHSNSNTYLQ | KVSNRFS | FQGSHVPYT |
| | SEQ ID NO: 639 | SEQ ID NO: 532 | SEQ ID NO: 533 |
| L1e | RSSQTFVHSNVNTYLQ | KVSNRFS | FQGSHVPYT |
| | SEQ ID NO: 640 | SEQ ID NO: 532 | SEQ ID NO: 533 |
| H1/2/3 | DYEIH | AIDPKTGNTAYNQ | YFSFAY |
| | SEQ ID NO: 303 | KFMG | SEQ ID NO: 305 |
| | | SEQ ID NO: 304 | |

### 8.5 Humanization of Antibody F3.81.19

### 8.5.1 Selection of Germline Sequence for F3.81.19

With IGKV2-40*01 and IGKJ2*01 as humanized light chain templates, and IGHV1-69*02 and IGHJ1*01 as humanized heavy chain templates for the murine antibody F3.81.19, CDRs of the murine antibody F3.81.19 were respectively grafted into corresponding humanized templates to obtain a humanized antibody GPC3-hAb007L of F3.81.19. A variable region sequence of the humanized antibody is shown as follows:

GPC3-hAb007L HCVR (VH-CDR graft, IGHV1-69*02):
(SEQ ID NO: 641)
EVQLVQSGAEVKKPGSSVKVSCKASGGTFSDYEMHWVRQA
PGQGLEWMGTIDPETGNTAYNQKFKGRVTITADKSTSTAY
MELSSLRSEDTAVYYCARPYSLTYWGQGTLVTVSS.
GPC3-hAb007L LCVR (VL-CDR graft, IGKV2-40*01):
(SEQ ID NO: 642)
DIVMTQTPLSLPVTPGEPASISCRSSQSFVHSNGDTYLQ
WYLQKPGQSPQLLIYKVSNRFSGVPDRFSGSGSGTDFTL
KISRVEAEDVGVYYCFQGSHVPYTFGQGTKLEIK.

### 8.5.2 Design of Back Mutations and Hotspot Mutations for F3.81.19 Humanized Antibodies

Key amino acids in FR sequences of F3.81.19 humanized antibodies were back-mutated as needed to ensure the original affinity. In view of a high-risk modifiable site NG on a light chain of F3.81.19, amino acid mutations were performed on the NG by means of computational simulation based on the antibody structure to eliminate modification risks. The detailed mutation design is shown in Table 41 (back mutations were performed in a natural numbering order).

TABLE 41
Design of back mutations and hotspot mutations
for F3.81.19 humanized antibodies

| VL | | VH | |
|---|---|---|---|
| L1 | Graft(IGKV2-40*01) | H1 | Graft(IGHV1-69*02) + |
| | | | G27Y, A97T |
| L1a | Graft(IGKV2-40*01) + | H2 | Graft(IGHV1-69*02) + |
| | N33D | | G27Y, S30T, A97T |
| L1b | Graft(IGKV2-40*01) + | H3 | Graft(IGHV1-69*02) + |
| | G34R | | G27Y, S30T, I70L, A97T |
| L1c | Graft(IGKV2-40*01) + | FR4 | IGHJ1*01 |
| | G34A | | |
| L1d | Graft(IGKV2-40*01) + | | |
| | G34S | | |
| L1e | Graft(IGKV2-40*01) + | | |
| | G34V | | |
| FR4 | IGKJ2*01 | | |

### 8.5.3 Combinations of Sequences of F3.81.19 Humanized Antibodies

The designs of back mutations and hotspot mutations for the F3.81.19 humanized antibodies in Table 41 were combined to eventually obtain multiple F3.81.19 humanized antibodies (see Table 42 for details).

TABLE 42
Corresponding amino acid sequences of F3.81.19 humanized antibodies

| | VL | | | | | |
|---|---|---|---|---|---|---|
| VH | L1 | L1a | L1b | L1c | L1d | L1e |
| H1 | hAb007LH1L1 | hAb007LH1L1a | hAb007LH1L1b | hAb007L H1L1c | hAb007L H1L1d | hAb007L H1L1e |
| H2 | hAb007LH2L1 | hAb007LH2L1a | hAb007LH2L1b | — | — | — |
| H3 | hAb007LH3L1 | hAb007LH3L1a | hAb007LH3L1b | — | — | — |

Amino acid sequences of humanized heavy chain/light chain variable regions are shown in Table 43:

TABLE 43
Amino acid sequences of back-mutated variable regions of F3.81.19
humanized antibodies

| VL/VH | Sequence No. | Amino acid sequence |
|---|---|---|
| L1 | SEQ ID NO: 643 | DIVMTQTPLSLPVTPGEPASISCRSSQSFVHSNG |
| | | DTYLQWYLQKPGQSPQLLIYKVSNRFSGVPDR |
| | | FSGSGSGTDFTLKISRVEAEDVGVYYCFQGSH |
| | | VPYTFGQGTKLEIK |
| L1a | SEQ ID NO: 644 | DIVMTQTPLSLPVTPGEPASISCRSSQSFVHSDG |
| | | DTYLQWYLQKPGQSPQLLIYKVSNRFSGVPDR |
| | | FSGSGSGTDFTLKISRVEAEDVGVYYCFQGSH |
| | | VPYTFGQGTKLEIK |
| L1b | SEQ ID NO: 645 | DIVMTQTPLSLPVTPGEPASISCRSSQSFVHSNR |
| | | DTYLQWYLQKPGQSPQLLIYKVSNRFSGVPDR |
| | | FSGSGSGTDFTLKISRVEAEDVGVYYCFQGSH |
| | | VPYTFGQGTKLEIK |
| L1c | SEQ ID NO: 646 | DIVMTQTPLSLPVTPGEPASISCRSSQSFVHSNA |
| | | DTYLQWYLQKPGQSPQLLIYKVSNRFSGVPDR |
| | | FSGSGSGTDFTLKISRVEAEDVGVYYCFQGSH |
| | | VPYTFGQGTKLEIK |
| L1d | SEQ ID NO: 647 | DIVMTQTPLSLPVTPGEPASISCRSSQSFVHSNS |
| | | DTYLQWYLQKPGQSPQLLIYKVSNRFSGVPDR |
| | | FSGSGSGTDFTLKISRVEAEDVGVYYCFQGSH |
| | | VPYTFGQGTKLEIK |
| L1e | SEQ ID NO: 648 | DIVMTQTPLSLPVTPGEPASISCRSSQSFVHSNV |
| | | DTYLQWYLQKPGQSPQLLIYKVSNRFSGVPDR |
| | | FSGSGSGTDFTLKISRVEAEDVGVYYCFQGSH |
| | | VPYTFGQGTKLEIK |
| H1 | SEQ ID NO: 649 | EVQLVQSGAEVKKPGSSVKVSCKASGYTFSD |
| | | YEMHWVRQAPGQGLEWMGTIDPETGNTAYN |
| | | QKFKGRVTITADKSTSTAYMELSSLRSEDTAV |
| | | YYCTRPYSLTYWGQGTLVTVSS |
| H2 | SEQ ID NO: 650 | EVQLVQSGAEVKKPGSSVKVSCKASGYTFTD |
| | | YEMHWVRQAPGQGLEWMGTIDPETGNTAYN |
| | | QKFKGRVTITADKSTSTAYMELSSLRSEDTAV |
| | | YYCTRPYSLTYWGQGTLVTVSS |
| H3 | SEQ ID NO: 651 | EVQLVQSGAEVKKPGSSVKVSCKASGYTFTD |
| | | YEMHWVRQAPGQGLEWMGTIDPETGNTAYN |
| | | QKFKGRVTLTADKSTSTAYMELSSLRSEDTAV |
| | | YYCTRPYSLTYWGQGTLVTVSS |

According to the Kabat numbering scheme, the analysis results for CDR sequences of heavy chain/light chain variable regions of the humanized antibodies are shown in Table 44:

TABLE 44
Kabat analysis results for CDR sequences of heavy chain/light chain variable
regions of F3.81.19 humanized antibodies

| VL/VH | CDR1 | CDR2 | CDR3 |
|---|---|---|---|
| L1 | RSSQSFVHSNGDTYLQ | KVSNRFS | FQGSHVPYT |
| | SEQ ID NO: 543 | SEQ ID NO: 544 | SEQ ID NO: 545 |
| L1a | RSSQSFVHSDGDTYLQ | KVSNRFS | FQGSHVPYT |
| | SEQ ID NO: 652 | SEQ ID NO: 544 | SEQ ID NO: 545 |
| L1b | RSSQSFVHSNRDTYLQ | KVSNRFS | FQGSHVPYT |
| | SEQ ID NO: 653 | SEQ ID NO: 544 | SEQ ID NO: 545 |
| L1c | RSSQSFVHSNADTYLQ | KVSNRFS | FQGSHVPYT |
| | SEQ ID NO: 654 | SEQ ID NO: 544 | SEQ ID NO: 545 |
| L1d | RSSQSFVHSNSDTYLQ | KVSNRFS | FQGSHVPYT |
| | SEQ ID NO: 655 | SEQ ID NO: 544 | SEQ ID NO: 545 |
| L1e | RSSQSFVHSNVDTYLQ | KVSNRFS | FQGSHVPYT |
| | SEQ ID NO: 656 | SEQ ID NO: 544 | SEQ ID NO: 545 |
| H1/2/3 | DYEMH | TIDPETGNTAYNQ | PYSLTY |
| | SEQ ID NO: 321 | KFKG | SEQ ID NO: 323 |
| | | SEQ ID NO: 322 | |

### 8.6 Construction and Expression Purification of GPC3 Humanized Full-Length Antibodies

PCR primers were designed to construct VH/VL gene fragments for various humanized antibodies, and then the gene fragments were homologously recombined with vectors to construct full-length expression vectors for the humanized antibodies. The humanized antibodies were expressed in the form of human IgG1. After plasmid construction, Expi293F cells were transiently transfected, and after 7 days, centrifugation was performed to collect supernatants, and the antibodies were purified by the purification method described in Example 1.

### Example 9 Identification of GPC3 Humanized Antibodies

### (A) Assay for Binding of GPC3 Humanized Antibodies to Human GPC3 Protein by Enzyme-Linked Immunosorbent Assay (ELISA)

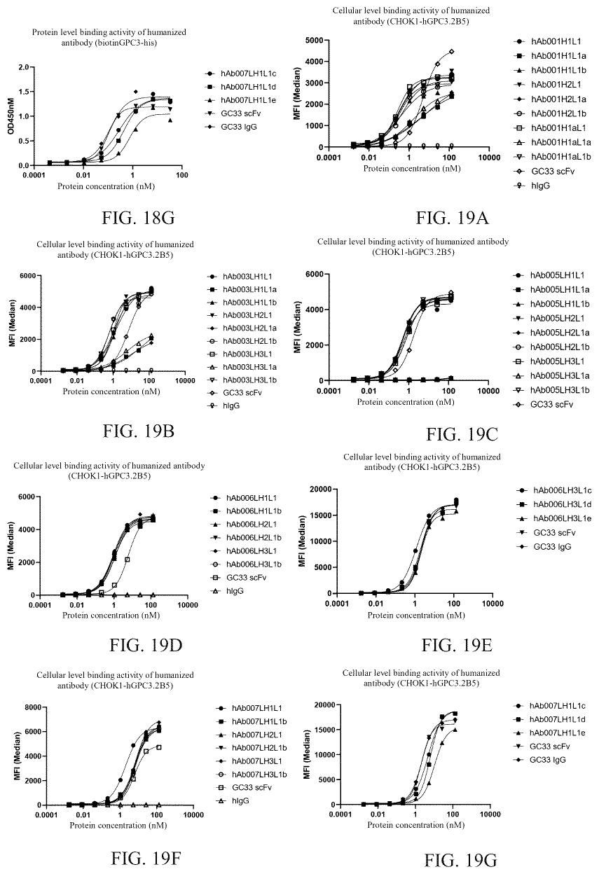

The obtained humanized antibodies were subjected to ELISA and data analysis according to the method described in Example 1 (A). An ELISA plate reader (Multimode Plate Reader, EnSight, purchased from Perkin Elmer) was used to read OD_{450 nm} values, with results for binding activity of GPC3 humanized antibodies with biotinylated labeled (Dojindo, Cat. No. LK03) human GPC3 protein (produced in-house) as shown in FIG. 18A to FIG. 18G and Table 45. The results showed that most of the humanized antibodies bound well to the human GPC3 protein. The data in the table are OD_{450 nm} values.

TABLE 45
Assay results for binding reactions of humanized
antibodies with human GPC3 protein by ELISA

| Antibody | Antibody concentration (nM) | | | | | | | |
|---|---|---|---|---|---|---|---|---|
| name | 66.66 | 13.33 | 2.67 | 0.53 | 0.11 | 0.02 | 0.004 | 0.0008 |
| hAb001H1L1 | 2.74 | 2.722 | 2.665 | 2.636 | 2 | 0.926 | 0.341 | 0.162 |
| hAb001H1L1a | 2.629 | 2.596 | 2.5 | 2.393 | 1.427 | 0.477 | 0.157 | 0.114 |
| hAb001H2L1 | 2.602 | 2.58 | 2.601 | 2.504 | 1.97 | 0.895 | 0.324 | 0.169 |
| hAb001H2L1a | 2.579 | 2.57 | 2.418 | 2.275 | 1.527 | 0.437 | 0.141 | 0.096 |
| hAb001H2L1b | 2.669 | 2.526 | 2.52 | 2.464 | 1.941 | 0.787 | 0.287 | 0.154 |
| hAb001H1aL1 | 2.673 | 2.543 | 2.423 | 2.331 | 1.85 | 0.874 | 0.327 | 0.184 |
| hAb001H1aL1b | 2.757 | 2.717 | 2.616 | 2.5 | 1.933 | 0.809 | 0.298 | 0.174 |
| GC33 scFv | 2.401 | 2.374 | 2.395 | 2.26 | 1.537 | 0.62 | 0.217 | 0.112 |
| hAb003LH1L1 | 2.488 | 2.568 | 2.472 | 2.502 | 2.154 | 1.185 | 0.427 | 0.16 |
| hAb003LH1L1a | 2.45 | 2.205 | 2.267 | 1.969 | 1.122 | 0.334 | 0.115 | 0.082 |
| hAb003LH1L1b | 2.654 | 2.437 | 2.498 | 2.352 | 2.095 | 1.115 | 0.371 | 0.147 |
| hAb003LH2L1 | 2.543 | 2.533 | 2.47 | 2.428 | 1.983 | 1.032 | 0.376 | 0.151 |
| hAb003LH2L1a | 2.344 | 2.183 | 2.144 | 1.9 | 1.178 | 0.423 | 0.146 | 0.088 |
| hAb003LH2L1b | 2.596 | 2.501 | 2.389 | 2.44 | 1.971 | 1.048 | 0.372 | 0.171 |
| hAb003LH3L1 | 2.393 | 2.299 | 2.559 | 2.259 | 1.89 | 0.915 | 0.297 | 0.137 |
| hAb003LH3L1a | 2.475 | 2.151 | 2.132 | 2.039 | 1.303 | 0.423 | 0.128 | 0.086 |
| hAb003LH3L1b | 2.613 | 2.586 | 2.443 | 2.536 | 2.002 | 1.079 | 0.381 | 0.157 |
| GC33 scFv | 2.693 | 2.445 | 2.373 | 2.254 | 1.845 | 0.767 | 0.266 | 0.137 |
| hAb005LH1L1 | 1.169 | 1.098 | 1.167 | 1.101 | 0.84 | 0.442 | 0.202 | 0.078 |
| hAb005LH1L1a | 0.558 | 0.208 | 0.113 | 0.056 | 0.057 | 0.051 | 0.05 | 0.053 |
| hAb005LH1L1b | 1.134 | 1.037 | 1.164 | 1.036 | 0.812 | 0.45 | 0.172 | 0.075 |
| hAb005LH2L1 | 1.158 | 1.119 | 1.122 | 1.011 | 0.812 | 0.464 | 0.185 | 0.083 |
| hAb005LH2L1a | 0.589 | 0.249 | 0.148 | 0.066 | 0.048 | 0.053 | 0.049 | 0.049 |
| hAb005LH2L1b | 1.161 | 1.073 | 1.039 | 1.052 | 0.856 | 0.43 | 0.194 | 0.074 |
| hAb005LH3L1 | 1.122 | 1.067 | 1.105 | 0.923 | 0.715 | 0.441 | 0.162 | 0.079 |
| hAb005LH3L1a | 0.402 | 0.153 | 0.101 | 0.058 | 0.05 | 0.049 | 0.049 | 0.047 |
| hAb005LH3L1b | 1.042 | 1.033 | 1.098 | 0.952 | 0.838 | 0.426 | 0.169 | 0.074 |
| GC33 scFv | 1.094 | 1.071 | 1.118 | 1.063 | 0.623 | 0.335 | 0.108 | 0.07 |
| hAb006LH1L1 | 2.429 | 2.417 | 2.362 | 2.393 | 1.899 | 1.055 | 0.365 | 0.117 |
| hAb006LH1L1a | 2.101 | 1.922 | 1.491 | 0.904 | 0.268 | 0.101 | 0.078 | 0.076 |
| hAb006LH1L1b | 2.568 | 2.469 | 2.455 | 2.404 | 1.707 | 0.841 | 0.288 | 0.15 |
| hAb006LH2L1 | 2.28 | 2.286 | 2.398 | 2.362 | 1.831 | 1.001 | 0.629 | 0.192 |
| hAb006LH2L1a | 1.919 | 1.735 | 1.372 | 0.584 | 0.139 | 0.076 | 0.072 | 0.073 |
| hAb006LH2L1b | 2.366 | 2.4 | 2.552 | 2.414 | 1.907 | 0.872 | 0.316 | 0.16 |
| hAb006LH3L1 | 2.329 | 2.252 | 2.199 | 2.06 | 1.645 | 0.819 | 0.287 | 0.154 |
| hAb006LH3L1a | 2.073 | 1.826 | 1.479 | 0.909 | 0.207 | 0.087 | 0.072 | 0.076 |
| hAb006LH3L1b | 2.504 | 2.363 | 2.415 | 2.362 | 1.926 | 0.878 | 0.309 | 0.155 |
| GC33 scFv | 2.297 | 2.33 | 2.322 | 2.236 | 1.639 | 0.775 | 0.286 | 0.164 |
| hAb007LH1L1 | 2.431 | 2.376 | 2.417 | 2.138 | 1.357 | 0.496 | 0.17 | 0.109 |
| hAb007LH1L1a | 2.218 | 2.197 | 2.098 | 1.575 | 0.634 | 0.218 | 0.112 | 0.087 |
| hAb007LH1L1b | 2.461 | 2.385 | 2.339 | 1.979 | 1.071 | 0.341 | 0.137 | 0.099 |
| hAb007LH2L1 | 2.319 | 2.33 | 2.262 | 2.258 | 1.72 | 0.654 | 0.233 | 0.121 |
| hAb007LH2L1a | 2.086 | 2.118 | 1.952 | 1.291 | 0.491 | 0.173 | 0.099 | 0.092 |
| hAb007LH2L1b | 2.252 | 2.329 | 2.2 | 1.845 | 0.924 | 0.297 | 0.133 | 0.099 |
| hAb007LH3L1 | 2.19 | 2.157 | 2.108 | 1.776 | 0.959 | 0.323 | 0.133 | 0.097 |
| hAb007LH3L1a | 2.033 | 2.037 | 1.782 | 1.036 | 0.353 | 0.14 | 0.088 | 0.083 |
| hAb007LH3L1b | 2.286 | 2.252 | 2.209 | 1.797 | 0.784 | 0.225 | 0.112 | 0.088 |
| GC33 scFv | 2.254 | 2.289 | 2.351 | 2.266 | 1.626 | 0.766 | 0.275 | 0.145 |
| | Antibody concentration (nM) | | | | | | | |
| | 34.72 | 6.94 | 1.39 | 0.28 | 0.056 | 0.011 | 0.002 | 0.0004 |
| hAb006LH3L1c | 1.362 | 1.436 | 1.462 | 1.297 | 0.756 | 0.265 | 0.102 | 0.071 |
| hAb006LH3L1d | 1.45 | 1.326 | 1.61 | 1.111 | 0.59 | 0.228 | 0.096 | 0.058 |
| hAb006LH3L1e | 1.322 | 1.438 | 1.361 | 1.228 | 0.592 | 0.187 | 0.085 | 0.06 |
| hAb007LH1L1c | 1.288 | 1.452 | 1.091 | 0.718 | 0.25 | 0.098 | 0.064 | 0.052 |
| hAb007LH1L1d | 1.314 | 1.342 | 1.103 | 0.503 | 0.16 | 0.101 | 0.057 | 0.059 |
| hAb007LH1L1e | 0.921 | 1.171 | 0.732 | 0.248 | 0.104 | 0.058 | 0.05 | 0.049 |
| GC33 scFv | 1.139 | 1.202 | 1.209 | 1.018 | 0.339 | 0.106 | 0.067 | 0.051 |
| GC33 IgG | 1.305 | 1.364 | 1.5 | 0.993 | 0.442 | 0.127 | 0.067 | 0.054 |

### (B) Assay for Binding of GPC3 Humanized Antibodies to Cells Expressing GPC3 Protein and Cells not Expressing GPC3 Protein (Negative Cells) by Flow Cytometry (FACS)

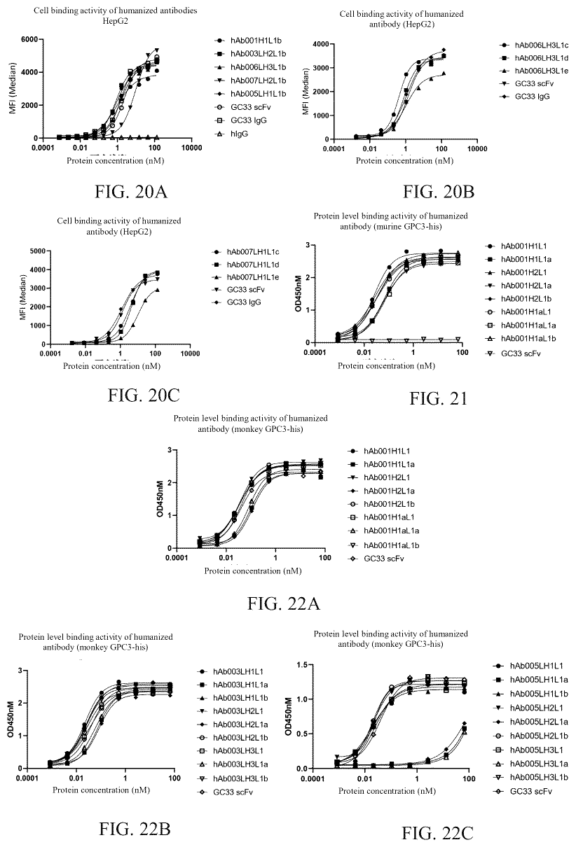

The desired cells were expanded to a logarithmic growth phase in a T-75 cell culture flask, the medium was removed by pipetting, and the cells were washed twice with a PBS buffer, and digested with trypsin. Then a complete medium was added to stop the digestion, and the cells were blown into a single cell suspension. After counting, the cells were centrifuged, and the cell pellet was resuspended with an FACS buffer (PBS+2% fetal bovine serum) to 2×10⁶ cells/mL. The cell resuspension solution was added to a 96-well FACS reaction plate at 50 μl/well, and a humanized antibody sample to be tested was added at 50 μl/well for incubation at 4° C. for 1 h. After the plate was centrifuged and washed 3 times with a PBS buffer, a goat anti-human IgG H+L antibody (Jackson, Cat. No. 109605088) was added at 50 μl/well for incubation on ice for 1 h. After the plate was centrifuged and washed 3 times with a PBS buffer, 100 μl of the resulting solution was assayed and analyzed by FACS (FACS Canto™, purchased from BD Biosciences). Data analysis was performed by software (FlowJo) to obtain the mean fluorescence intensity (MFI) of the cells. Then, analysis was performed by software (GraphPad Prism8), data were fitted, and EC₅₀ values were calculated. The results showed that most of the humanized antibodies could bind to CHO-K1-human GPC3 cells (Table 46 and FIG. 19A to FIG. 19G), but did not bind to CHO-K1 cells; and Table 47 and FIG. 20A to FIG. 20C show that most of the humanized antibodies could bind to HepG2 cells. hIgG was a negative control.

TABLE 46
Assay results for binding reactions of humanized
antibodies with CHO-K1-human GPC3 cells by FACS

| | CHOK1-hGPC3.2B5 | |
|---|---|---|
| Antibody | Maximum mean fluorescence intensity | Ec50 |
| name | Max MFI | (nM) |
| hAb001H1L1 | 3405 | 0.313 |
| hAb001H1L1a | 2362 | 3.655 |
| hAb001H1L1b | 3155 | 0.399 |
| hAb001H2L1 | 3553 | 0.359 |
| hAb001H2L1a | 2549 | 6.172 |
| hAb001H2L1b | 3374 | 0.455 |
| hAb001H1aL1 | 3273 | 0.269 |
| hAb001H1aL1a | 2526 | 1.634 |
| hAb001H1aL1b | 3070 | 0.325 |
| GC33 scFv | 4465 | 5.006 |
| hIgG | 24.6 | Negative |
| hAb003LH1L1 | 5216 | 1.418 |
| hAb003LH1L1a | 2066 | 45.080 |
| hAb003LH1L1b | 5013 | 1.200 |
| hAb003LH2L1 | 5137 | 1.066 |
| hAb003LH2L1a | 1774 | 11.550 |
| hAb003LH2L1b | 4998 | 0.584 |
| hAb003LH3L1 | 5044 | 1.085 |
| hAb003LH3L1a | 2243 | 6.024 |
| hAb003LH3L1b | 4983 | 0.647 |
| GC33 scFv | 4819 | 7.130 |
| hIgG | 43.8 | Negative |
| hAb005LH1L1 | 4496 | 0.563 |
| hAb005LH1L1a | 122 | 314.900 |
| hAb005LH1L1b | 4612 | 0.491 |
| hAb005LH2L1 | 4540 | 0.736 |
| hAb005LH2L1a | 153 | 118.400 |
| hAb005LH2L1b | 4772 | 0.540 |
| hAb005LH3L1 | 4704 | 0.734 |
| hAb005LH3L1a | 136 | 132.900 |
| hAb005LH3L1b | 4740 | 0.558 |
| GC33 scFv | 4964 | 1.745 |
| hAb006LH1L1 | 4775 | 0.906 |
| hAb006LH1L1b | 4631 | 1.096 |
| hAb006LH2L1 | 4878 | 1.018 |
| hAb006LH2L1b | 4789 | 1.142 |
| hAb006LH3L1 | 4922 | 0.929 |
| hAb006LH3L1b | 4674 | 0.970 |
| GC33 scFv | 4589 | 5.933 |
| hIgG | 21.4 | Negative |
| hAb007LH1L1 | 6436 | 2.023 |
| hAb007LH1L1b | 6074 | 6.466 |
| hAb007LH2L1 | 6186 | 6.556 |
| hAb007LH2L1b | 6037 | 6.157 |
| hAb007LH3L1 | 6757 | 6.980 |
| hAb007LH3L1b | 6243 | 7.327 |
| GC33 scFv | 4717 | 6.254 |
| hIgG | 22.5 | Negative |
| hAb006LH3L1c | 17973 | 1.160 |
| hAb006LH3L1d | 17598 | 1.977 |
| hAb006LH3L1e | 15754 | 2.058 |
| hAb007LH1L1c | 18228 | 4.609 |
| hAb007LH1L1d | 18227 | 6.280 |
| hAb007LH1L1e | 14979 | 10.400 |
| GC33 scFv | 16705 | 2.042 |
| GC33 IgG | 17041 | 2.193 |

TABLE 47
Assay results for binding reactions of humanized
antibodies with HepG2 cells by FACS

| | HepG2 | |
|---|---|---|
| Antibody | Maximum mean fluorescence intensity | Ec50 |
| name | Max MFI | (nM) |
| hAb001H1L1b | 4087 | 0.918 |
| hAb003LH2L1b | 4616 | 0.840 |
| hAb006LH3L1b | 4609 | 1.492 |
| hAb007LH2L1b | 5322 | 9.533 |
| hAb005LH1L1b | 4804 | 1.009 |
| GC33 scFv | 4933 | 2.421 |
| GC33 IgG | 4722 | 1.564 |
| hIgG | 58.8 | Negative |
| hAb006LH3L1c | 3523 | 0.417 |
| hAb006LH3L1d | 3495 | 0.772 |
| hAb006LH3L1e | 2778 | 1.014 |
| hAb007LH1L1c | 3766 | 3.667 |
| hAb007LH1L1d | 3832 | 4.388 |
| hAb007LH1L1e | 2914 | 10.06 |
| GC33 scFv | 3480 | 1.363 |
| GC33 IgG | 3754 | 1.208 |

### Example 10 Assay for Cross-Binding Activity of GPC3 Humanized Antibodies

### (A) Assay for Binding of Humanized Antibodies to Monkey GPC3 Protein and Murine GPC3 Protein by ELISA

A monkey GPC3-His protein (purchased from Acro, Cat. No. GP3-C5225) and a murine GPC3-his protein (purchased from Sino Biological, Cat. No. 50989-M08B) were subjected to ELISA and data analysis according to the method described in Example 1 (A). The ELISA results for the humanized antibodies and the murine GPC3 protein are shown in FIG. 21 and Table 48. The results showed that antibodies of hAb001 series bound well to the murine GPC3 protein, while the remaining antibodies did not bind to the murine GPC3 protein. The data in the table are OD_{450 nm} values.

TABLE 48
Assay results for binding reactions of humanized
antibodies with mouse GPC3 protein by ELISA

| Antibody | Antibody concentration (nM) | | | | | | | |
|---|---|---|---|---|---|---|---|---|
| name | 66.66 | 13.33 | 2.67 | 0.53 | 0.11 | 0.02 | 0.004 | 0.0008 |
| hAb001H1L1 | 2.637 | 2.823 | 2.688 | 2.802 | 2.315 | 1.32 | 0.48 | 0.239 |
| hAb001H1L1a | 2.583 | 2.566 | 2.493 | 2.365 | 1.655 | 0.657 | 0.356 | 0.117 |
| hAb001H2L1 | 2.618 | 2.621 | 2.593 | 2.479 | 2.105 | 1.269 | 0.524 | 0.277 |
| hAb001H2L1a | 2.481 | 2.407 | 2.35 | 2.186 | 1.59 | 0.664 | 0.191 | 0.094 |
| hAb001H2L1b | 2.644 | 2.585 | 2.445 | 2.384 | 2.064 | 1.094 | 0.407 | 0.199 |
| hAb001H1aL1 | 2.678 | 2.63 | 2.518 | 2.447 | 1.953 | 1.154 | 0.44 | 0.255 |
| hAb001H1aL1a | 2.452 | 2.443 | 2.488 | 2.287 | 1.489 | 0.67 | 0.206 | 0.092 |
| hAb001H1aL1b | 2.758 | 2.729 | 2.681 | 2.522 | 2.054 | 1.146 | 0.346 | 0.185 |
| GC33 scFv | 0.095 | 0.078 | 0.107 | 0.051 | 0.059 | 0.052 | 0.059 | 0.065 |

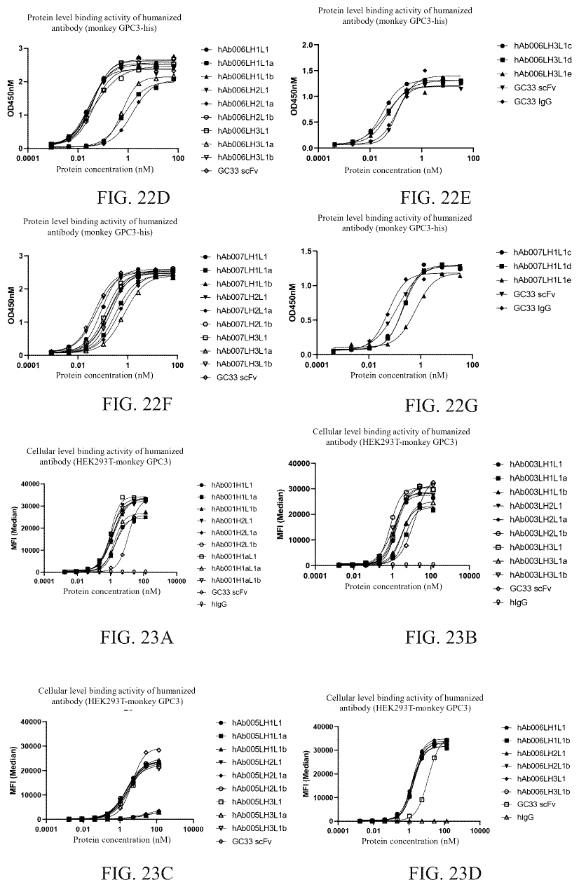

The ELISA results for the humanized antibodies and the monkey GPC3 protein are shown in FIG. 22A to FIG. 22G and Table 49. The results showed that most of the humanized antibodies bound to the monkey GPC3 protein.

TABLE 49
Assay results for binding reactions of humanized
antibodies with monkey GPC3 protein by ELISA

| Antibody | Antibody concentration (nM) | | | | | | | |
|---|---|---|---|---|---|---|---|---|
| name | 66.66 | 13.33 | 2.67 | 0.53 | 0.11 | 0.02 | 0.004 | 0.0008 |
| hAb001H1L1 | 2.55 | 2.551 | 2.544 | 2.459 | 1.956 | 1.078 | 0.373 | 0.198 |
| hAb001H1L1a | 2.161 | 2.369 | 2.265 | 2.082 | 1.23 | 0.39 | 0.211 | 0.068 |
| hAb001H2L1 | 2.679 | 2.644 | 2.593 | 2.467 | 2.114 | 1.048 | 0.4 | 0.304 |
| hAb001H2L1a | 2.224 | 2.341 | 2.25 | 2.024 | 1.13 | 0.34 | 0.094 | 0.063 |
| hAb001H2L1b | 2.577 | 2.471 | 2.453 | 2.538 | 1.933 | 0.865 | 0.317 | 0.143 |
| hAb001H1aL1 | 2.525 | 2.522 | 2.568 | 2.36 | 1.996 | 1.03 | 0.379 | 0.201 |
| hAb001H1aL1a | 2.341 | 2.418 | 2.441 | 2.193 | 1.437 | 0.437 | 0.132 | 0.073 |
| hAb001H1aL1b | 2.588 | 2.536 | 2.394 | 2.392 | 1.992 | 1.025 | 0.347 | 0.144 |
| GC33 scFv | 2.343 | 2.204 | 2.403 | 2.237 | 1.76 | 0.838 | 0.265 | 0.118 |
| hAb003LH1L1 | 2.583 | 2.615 | 2.582 | 2.653 | 2.314 | 1.338 | 0.467 | 0.188 |
| hAb003LH1L1a | 2.416 | 2.351 | 2.29 | 2.196 | 1.48 | 0.483 | 0.148 | 0.087 |
| hAb003LH1L1b | 2.551 | 2.639 | 2.58 | 2.461 | 2.184 | 1.221 | 0.418 | 0.192 |
| hAb003LH2L1 | 2.513 | 2.438 | 2.572 | 2.579 | 2.164 | 1.215 | 0.413 | 0.175 |
| hAb003LH2L1a | 2.232 | 2.34 | 2.171 | 2.118 | 1.368 | 0.475 | 0.16 | 0.15 |
| hAb003LH2L1b | 2.35 | 2.272 | 2.36 | 2.297 | 1.956 | 1.104 | 0.386 | 0.185 |
| hAb003LH3L1 | 2.526 | 2.364 | 2.357 | 2.22 | 1.847 | 0.963 | 0.357 | 0.16 |
| hAb003LH3L1a | 2.389 | 2.297 | 2.385 | 2.206 | 1.592 | 0.633 | 0.202 | 0.089 |
| hAb003LH3L1b | 2.533 | 2.401 | 2.399 | 2.502 | 2.104 | 1.168 | 0.432 | 0.165 |
| GC33 scFv | 2.487 | 2.358 | 2.452 | 2.44 | 1.866 | 0.881 | 0.304 | 0.144 |
| hAb005LH1L1 | 1.1 | 1.216 | 1.21 | 1.158 | 0.94 | 0.594 | 0.103 | 0.069 |
| hAb005LH1L1a | 0.578 | 0.195 | 0.111 | 0.053 | 0.066 | 0.039 | 0.045 | 0.06 |
| hAb005LH1L1b | 1.161 | 1.157 | 1.108 | 1.095 | 0.949 | 0.664 | 0.215 | 0.107 |
| hAb005LH2L1 | 1.202 | 1.151 | 1.238 | 1.276 | 1.132 | 0.649 | 0.222 | 0.164 |
| hAb005LH2L1a | 0.652 | 0.276 | 0.136 | 0.056 | 0.042 | 0.041 | 0.04 | 0.099 |
| hAb005LH2L1b | 1.232 | 1.235 | 1.324 | 1.248 | 1.174 | 0.687 | 0.232 | 0.09 |
| hAb005LH3L1 | 1.236 | 1.129 | 1.27 | 1.146 | 0.997 | 0.609 | 0.226 | 0.088 |
| hAb005LH3L1a | 0.527 | 0.161 | 0.098 | 0.042 | 0.038 | 0.039 | 0.041 | 0.04 |
| hAb005LH3L1b | 1.176 | 1.273 | 1.327 | 1.275 | 1.113 | 0.663 | 0.237 | 0.088 |
| GC33 scFv | 1.283 | 1.297 | 1.29 | 1.312 | 0.986 | 0.487 | 0.171 | 0.072 |
| hAb006LH1L1 | 2.476 | 2.561 | 2.433 | 2.497 | 2.015 | 1.1 | 0.369 | 0.121 |
| hAb006LH1L1a | 2.078 | 1.821 | 1.628 | 0.917 | 0.227 | 0.078 | 0.058 | 0.05 |
| hAb006LH1L1b | 2.771 | 2.504 | 2.748 | 2.487 | 2.127 | 0.877 | 0.309 | 0.111 |
| hAb006LH2L1 | 2.387 | 2.227 | 2.457 | 2.376 | 2.014 | 1.057 | 0.358 | 0.129 |
| hAb006LH2L1a | 2.062 | 1.837 | 1.392 | 0.549 | 0.137 | 0.065 | 0.055 | 0.047 |
| hAb006LH2L1b | 2.685 | 2.462 | 2.704 | 2.567 | 2.096 | 0.924 | 0.34 | 0.128 |
| hAb006LH3L1 | 2.525 | 2.439 | 2.356 | 2.1 | 1.745 | 0.911 | 0.294 | 0.116 |
| hAb006LH3L1a | 2.176 | 2.09 | 1.947 | 1.049 | 0.226 | 0.08 | 0.057 | 0.044 |
| hAb006LH3L1b | 2.671 | 2.38 | 2.548 | 2.564 | 1.988 | 1.015 | 0.306 | 0.125 |
| GC33 scFv | 2.326 | 2.309 | 2.443 | 2.413 | 1.758 | 0.806 | 0.253 | 0.101 |
| hAb007LH1L1 | 2.556 | 2.541 | 2.596 | 2.427 | 1.464 | 0.491 | 0.122 | 0.085 |
| hAb007LH1L1a | 2.357 | 2.439 | 2.207 | 1.573 | 0.605 | 0.197 | 0.097 | 0.067 |
| hAb007LH1L1b | 2.535 | 2.37 | 2.389 | 1.973 | 0.958 | 0.305 | 0.128 | 0.071 |
| hAb007LH2L1 | 2.574 | 2.468 | 2.473 | 2.308 | 1.739 | 0.707 | 0.253 | 0.101 |
| hAb007LH2L1a | 2.447 | 2.388 | 2.097 | 1.407 | 0.468 | 0.182 | 0.093 | 0.065 |
| hAb007LH2L1b | 2.613 | 2.555 | 2.345 | 2.087 | 0.967 | 0.32 | 0.127 | 0.08 |
| hAb007LH3L1 | 2.55 | 2.388 | 2.39 | 2.17 | 1.123 | 0.368 | 0.198 | 0.079 |
| hAb007LH3L1a | 2.341 | 2.371 | 1.892 | 1.079 | 0.337 | 0.135 | 0.079 | 0.069 |
| hAb007LH3L1b | 2.576 | 2.473 | 2.371 | 1.95 | 0.801 | 0.252 | 0.103 | 0.069 |
| GC33 scFv | 2.404 | 2.583 | 2.499 | 2.484 | 1.805 | 0.829 | 0.257 | 0.119 |
| | Antibody concentration (nM) | | | | | | | |
| | 34.72 | 6.94 | 1.39 | 0.28 | 0.056 | 0.011 | 0.002 | 0.0004 |
| hAb006LH3L1c | 1.262 | 1.335 | 1.319 | 1.239 | 0.876 | 0.332 | 0.122 | 0.071 |
| hAb006LH3L1d | 1.3 | 1.288 | 1.282 | 1.104 | 0.719 | 0.308 | 0.121 | 0.068 |
| hAb006LH3L1e | 1.262 | 1.219 | 1.075 | 1.139 | 0.67 | 0.24 | 0.114 | 0.066 |
| GC33 scFv | 1.139 | 1.202 | 1.209 | 1.018 | 0.339 | 0.106 | 0.067 | 0.051 |
| GC33 IgG | 1.305 | 1.364 | 1.5 | 0.993 | 0.442 | 0.127 | 0.067 | 0.054 |
| hAb007LH1L1c | 1.248 | 1.264 | 1.303 | 0.744 | 0.268 | 0.103 | 0.069 | 0.05 |
| hAb007LH1L1d | 1.245 | 1.304 | 1.198 | 0.752 | 0.235 | 0.09 | 0.078 | 0.053 |
| hAb007LH1L1e | 1.14 | 1.168 | 0.848 | 0.399 | 0.148 | 0.14 | 0.148 | 0.043 |
| GC33 scFv | 1.277 | 1.282 | 1.217 | 0.871 | 0.444 | 0.177 | 0.075 | 0.048 |
| GC33 IgG | 1.233 | 1.167 | 1.092 | 1.092 | 0.582 | 0.196 | 0.083 | 0.052 |

### (B) Assay for Binding of Humanized Antibodies to Cells Expressing Monkey GPC3 by FACS

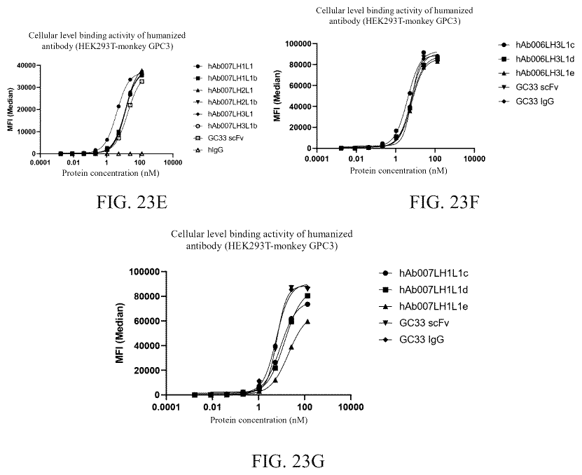

HEK293T-monkey GPC3 cells were subjected to FACS assay and data analysis according to the method described in Example 4 (B). The analysis results are shown in Table 50 and FIG. 23A to FIG. 23G. The results showed that most of the humanized antibodies bound to HEK293T-monkey-GPC3 cells.

TABLE 50
Assay results for binding reactions of humanized
antibodies with HEK293T-monkey GPC3 cells by FACS

| | 293T-cynoGPC3 | |
|---|---|---|
| Antibody | Maximum mean fluorescence intensity | Ec50 |
| name | Max MFI | (nM) |
| hAb001H1L1 | 33451 | 1.329 |
| hAb001H1L1a | 24884 | 1.867 |
| hAb001H1L1b | 32934 | 1.542 |
| hAb001H2L1 | 33260 | 1.077 |
| hAb001H2L1a | 26875 | 2.028 |
| hAb001H2L1b | 33137 | 1.410 |
| hAb001H1aL1 | 33989 | 1.005 |
| hAb001H1aL1a | 27176 | 1.683 |
| hAb001H1aL1b | 32614 | 0.960 |
| GC33 scFv | 32015 | 12.260 |
| hIgG | 66.3 | Negative |
| hAb003LH1L1 | 28230 | 1.560 |
| hAb003LH1L1a | 22647 | 4.767 |
| hAb003LH1L1b | 28412 | 1.460 |
| hAb003LH2L1 | 29572 | 1.321 |
| hAb003LH2L1a | 23208 | 3.092 |
| hAb003LH2L1b | 31087 | 0.793 |
| hAb003LH3L1 | 30860 | 1.576 |
| hAb003LH3L1a | 24518 | 3.271 |
| hAb003LH3L1b | 31266 | 1.284 |
| GC33 scFv | 32485 | 14.670 |
| hIgG | 66.3 | Negative |
| hAb005LH1L1 | 23363 | 2.171 |
| hAb005LH1L1a | 2907 | 71.640 |
| hAb005LH1L1b | 24304 | 2.617 |
| hAb005LH2L1 | 23360 | 2.882 |
| hAb005LH2L1a | 3494 | 32.830 |
| hAb005LH2L1b | 22846 | 2.422 |
| hAb005LH3L1 | 22247 | 3.094 |
| hAb005LH3L1a | 2915 | 111.900 |
| hAb005LH3L1b | 22057 | 2.367 |
| GC33 scFv | 28313 | 4.600 |
| hAb006LH1L1 | 33504 | 1.535 |
| hAb006LH1L1b | 31847 | 1.729 |
| hAb006LH2L1 | 32846 | 1.650 |
| hAb006LH2L1b | 31454 | 1.828 |
| hAb006LH3L1 | 34573 | 1.701 |
| hAb006LH3L1b | 32540 | 1.778 |
| GC33 scFv | 34233 | 11.900 |
| hIgG | 47.7 | Negative |
| hAb007LH1L1 | 36305 | 4.100 |
| hAb007LH1L1b | 35376 | 12.440 |
| hAb007LH2L1 | 37755 | 14.780 |
| hAb007LH2L1b | 35793 | 13.250 |
| hAb007LH3L1 | 37382 | 13.620 |
| hAb007LH3L1b | 35926 | 13.710 |
| GC33 scFv | 32645 | 18.360 |
| hIgG | 39.5 | Negative |
| hAb006LH3L1c | 91573 | 4.058 |
| hAb006LH3L1d | 84845 | 6.163 |
| hAb006LH3L1e | 82861 | 6.536 |
| hAb007LH1L1c | 73562 | 8.799 |
| hAb007LH1L1d | 80437 | 13.56 |
| hAb007LH1L1e | 59675 | 19.92 |
| GC33 scFv | 87052 | 6.29 |
| GC33 IgG | 86985 | 5.957 |

### Example 11 Assay for Affinity of GPC3 Humanized Antibodies

Anti-GPC3 humanized antibodies were captured using a Protein A chip (GE Healthcare; 29-127-558). The sample and running buffer was HBS-EP+ (10 mM HEPES, 150 mM NaCl, 3 mM EDTA, 0.05% surfactant P20) (GE Healthcare; BR-1006-69). The flow cell was set at 25° C. The sample block was set at 16° C. Both were pretreated with the running buffer. In each cycle, first, the antibody to be tested was captured using the Protein A chip, and then a single concentration of GPC3 antigen protein was injected. The association and dissociation processes of the antibody with the antigen protein were recorded, and finally, the chip was regenerated using Glycine pH 1.5 (GE Healthcare; BR-1003-54). The association was determined by injecting different concentrations of human GPC3-His in the solution and maintaining for 240 s, wherein the flow rate was 30 μL/min, and the protein was diluted at a 1:1 dilution ratio from 200 nM to obtain 5 concentrations in total. The dissociation phase was monitored for up to 600 s and triggered by switching from the sample solution to the running buffer. The surface was regenerated by washing with 10 mM glycine solution (pH 1.5) at a flow rate of 30 μL/min for 30 s. The difference in bulk refractive index was corrected by subtracting the responses obtained from the goat anti-human Fc surface. Blank injections (double reference) were also subtracted. To calculate the apparent KD and other kinetic parameters, the Langmuir 1:1 model was used. The association rate (Kₐ), dissociation rate (K_{d}), and binding affinity (KD) of humanized antibodies with the human GPC3 protein are shown in Table 51.

TABLE 51
Binding affinity of humanized antibodies to human GPC3 protein

| Antibody name | Ka (1/Ms) | Kd (1/s) | KD (M) |
|---|---|---|---|
| hAb001H1L1b | 4.74E+07 | 9.37E−01 | 1.98E−08 |
| hAb001H2L1b | 5.26E+05 | 1.01E−02 | 1.92E−08 |
| hAb001H1aL1 | 2.60E+06 | 7.01E−02 | 2..70E−08 |
| hAb001H1aL1b | 1.53E+05 | 1.67E−02 | 1.09E−07 |
| hAb003LH1L1 | 2.34E+05 | 4.53E−04 | 1.93E−09 |
| hAb003LH1L1b | 2.46E+05 | 4.15E−04 | 1.68E−09 |
| hAb003LH2L1b | 2.40E+05 | 5.01E−04 | 2.08E−09 |
| hAb005LH1L1b | 2.72E+05 | 2.31E−03 | 8.49E−09 |
| hAb005LH2L1b | 2.71E+05 | 2.13E−03 | 7.85E−09 |
| hAb006LH1L1b | 1.68E+05 | 1.25E−03 | 7.41E−09 |
| hAb006LH2L1 | 1.58E+05 | 6.78E−04 | 4.30E−09 |
| hAb006LH2L1b | 1.38E+05 | 1.28E−03 | 9.28E−09 |
| hAb006LH3L1b | 1.62E+05 | 1.36E−03 | 8.40E−09 |
| hAb006LH3L1c | 3.19E+05 | 1.00E−03 | 3.15E−09 |
| hAb007LH1L1 | 1.09E+05 | 1.14E−04 | 1.05E−09 |
| hAb007LH1L1b | 1.03E+05 | 2.28E−04 | 2.21E−09 |
| hAb007LH2L1b | 8.80E+04 | 3.15E−04 | 3.58E−09 |
| hAb007LH1L1c | 9.27E+04 | 3.97E−04 | 4.28E−09 |
| GC33 scFv | 1.74E+05 | 2.77E−04 | 1.92E−08 |

The monoclonal antibody specifically recognizing glypican-3 and use thereof provided in the present invention are described in detail above. The principle and implementations of the present invention are described with specific examples herein. The foregoing examples are merely intended to help understand the method and core idea of the present invention. It should be pointed out that those skilled in the art can further make several improvements and modifications to the present invention, without departing from the principle of the present invention; moreover, these improvements and modifications also fall within the protection scope of the claims of the present invention.

1. An anti-glypican-3 (GPC3) antibody or antigen-binding moiety, wherein the antibody or antigen-binding moiety comprises heavy chain CDRs having CDR1-VH, CDR2-VH and CDR3-VH, wherein the CDR1-VH, the CDR2-VH and the CDR3-VH have any sequence selected from the following or a sequence combination having 1, 2, 3 or more amino acid insertions, deletions and/or substitutions compared with the sequence, and preferably, the substitutions are conservative amino acid substitutions:
(1) the CDR1-VH may be selected from SEQ ID NO: 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168, 171, 174, 177, 180, 183, 186, 189, 192, 195, 198, 201, 204, 207, 210, 213, 216, 219, 222, 225, 228, 231, 234, 237, 240, 243, 246, 249, 252, 255, 258, 261, 264, 267, 270, 273, 276, 279, 282, 285, 288, 291, 294, 297, 300, 303, 306, 309, 312, 315, 318, 321, 324, 327, 330, 333, 336, 339, 342, 345, 348, 351, 354, 357, 360, 363, 366, 369 or 372;
(2) the CDR2-VH may be selected from SEQ ID NO: 70, 73, 76, 79, 82, 85, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, 217, 220, 223, 226, 229, 232, 235, 238, 241, 244, 247, 250, 253, 256, 259, 262, 265, 268, 271, 274, 277, 280, 283, 286, 289, 292, 295, 298, 301, 304, 307, 310, 313, 316, 319, 322, 325, 328, 331, 334, 337, 340, 343, 346, 349, 352, 355, 358, 361, 364, 367, 370, 373 or 604;
(3) the CDR3-VH may be selected from SEQ ID NO: 71, 74, 77, 80, 83, 86, 89, 92, 95, 98, 101, 104, 107, 110, 113, 116, 119, 122, 125, 128, 131, 134, 137, 140, 143, 146, 149, 152, 155, 158, 161, 164, 167, 170, 173, 176, 179, 182, 185, 188, 191, 194, 197, 200, 203, 206, 209, 212, 215, 218, 221, 224, 227, 230, 233, 236, 239, 242, 245, 248, 251, 254, 257, 260, 263, 266, 269, 272, 275, 278, 281, 284, 287, 290, 293, 296, 299, 302, 305, 308, 311, 314, 317, 320, 323, 326, 329, 332, 335, 338, 341, 344, 347, 350, 353, 356, 359, 362, 365, 368, 371 or 374;
and/or, light chain CDRs having CDR1-VL, CDR2-VL and CDR3-VL, wherein the CDR1-VL, the CDR2-VL and the CDR3-VL have any sequence selected from the following or a sequence combination having 1, 2, 3 or more amino acid insertions, deletions and/or substitutions compared with the sequence, and preferably, the substitutions are conservative amino acid substitutions:
(4) the CDR1-VL may be selected from SEQ ID NO: 375, 378, 381, 384, 387, 390, 393, 396, 399, 402, 405, 408, 411, 414, 417, 420, 423, 426, 429, 432, 435, 438, 441, 444, 447, 450, 453, 456, 459, 462, 465, 468, 471, 474, 477, 480, 483, 486, 489, 492, 495, 498, 501, 504, 507, 510, 513, 516, 519, 522, 525, 528, 531, 534, 537, 540, 543, 546, 549, 552, 555, 558, 561, 564, 567, 570, 573, 576, 602, 603, 613, 614, 623, 624, 636, 637, 638, 639, 640, 652, 653, 654, 655 or 656;
(5) the CDR2-VL may be selected from SEQ ID NO: 376, 379, 382, 385, 388, 391, 394, 397, 400, 403, 406, 409, 412, 415, 418, 421, 424, 427, 430, 433, 436, 439, 442, 445, 448, 451, 454, 457, 460, 463, 466, 469, 472, 475, 478, 481, 484, 487, 490, 493, 496, 499, 502, 505, 508, 511, 514, 517, 520, 523, 526, 529, 532, 535, 538, 541, 544, 547, 550, 553, 556, 559, 562, 565, 568, 571, 574 or 577; and
(6) the CDR3-VL may be selected from SEQ ID NO: 377, 380, 383, 386, 389, 392, 395, 398, 401, 404, 407, 410, 413, 416, 419, 422, 425, 428, 431, 434, 437, 440, 443, 446, 449, 452, 455, 458, 461, 464, 467, 470, 473, 476, 479, 482, 485, 488, 491, 494, 497, 500, 503, 506, 509, 512, 515, 518, 521, 524, 527, 530, 533, 536, 539, 542, 545, 548, 551, 554, 557, 560, 563, 566, 569, 572, 575 or 578.

2. The antibody or antigen-binding moiety according to claim 1, wherein the CDR1-VH, the CDR2-VH and the CDR3-VH are selected from any sequence combination of the following VH1-VH102 or sequence combinations having 1, 2, 3 or more amino acid insertions, deletions and/or substitutions compared with the sequence combination, and preferably, the substitutions are conservative amino acid substitutions:

| | SEQ ID NO: | | |
|---|---|---|---|
| No. | CDR1-VH | CDR2-VH | CDR3-VH |
| VH1 | 69 | 70 | 71 |
| VH2 | 72 | 73 | 74 |
| VH3 | 75 | 76 | 77 |
| VH4 | 78 | 79 | 80 |
| VH5 | 81 | 82 | 83 |
| VH6 | 84 | 85 | 86 |
| VH7 | 87 | 88 | 89 |
| VH8 | 90 | 91 | 92 |
| VH9 | 93 | 94 | 95 |
| VH10 | 96 | 97 | 98 |
| VH11 | 99 | 100 | 101 |
| VH12 | 102 | 103 | 104 |
| VH13 | 105 | 106 | 107 |
| VH14 | 108 | 109 | 110 |
| VH15 | 111 | 112 | 113 |
| VH16 | 114 | 115 | 116 |
| VH17 | 117 | 118 | 119 |
| VH18 | 120 | 121 | 122 |
| VH19 | 123 | 124 | 125 |
| VH20 | 126 | 127 | 128 |
| VH21 | 129 | 130 | 131 |
| VH22 | 132 | 133 | 134 |
| VH23 | 135 | 136 | 137 |
| VH24 | 138 | 139 | 140 |
| VH25 | 141 | 142 | 143 |
| VH26 | 144 | 145 | 146 |
| VH27 | 147 | 148 | 149 |
| VH28 | 150 | 151 | 152 |
| VH29 | 153 | 154 | 155 |
| VH30 | 156 | 157 | 158 |
| VH31 | 159 | 160 | 161 |
| VH32 | 162 | 163 | 164 |
| VH33 | 165 | 166 | 167 |
| VH34 | 168 | 169 | 170 |
| VH35 | 171 | 172 | 173 |
| VH36 | 174 | 175 | 176 |
| VH37 | 177 | 178 | 179 |
| VH38 | 180 | 181 | 182 |
| VH39 | 183 | 184 | 185 |
| VH40 | 186 | 187 | 188 |
| VH41 | 189 | 190 | 191 |
| VH42 | 192 | 193 | 194 |
| VH43 | 195 | 196 | 197 |
| VH44 | 198 | 199 | 200 |
| VH45 | 201 | 202 | 203 |
| VH46 | 204 | 205 | 206 |
| VH47 | 207 | 208 | 209 |
| VH48 | 210 | 211 | 212 |
| VH49 | 213 | 214 | 215 |
| VH50 | 216 | 217 | 218 |
| VH51 | 219 | 220 | 221 |
| VH52 | 222 | 223 | 224 |
| VH53 | 225 | 226 | 227 |
| VH54 | 228 | 229 | 230 |
| VH55 | 231 | 232 | 233 |
| VH56 | 234 | 235 | 236 |
| VH57 | 237 | 238 | 239 |
| VH58 | 240 | 241 | 242 |
| VH59 | 243 | 244 | 245 |
| VH60 | 246 | 247 | 248 |
| VH61 | 249 | 250 | 251 |
| VH62 | 252 | 253 | 254 |
| VH63 | 255 | 256 | 257 |
| VH64 | 258 | 259 | 260 |
| VH65 | 261 | 262 | 263 |
| VH66 | 264 | 265 | 266 |
| VH67 | 267 | 268 | 269 |
| VH68 | 270 | 271 | 272 |
| VH69 | 273 | 274 | 275 |
| VH70 | 276 | 277 | 278 |
| VH71 | 279 | 280 | 281 |
| VH72 | 282 | 283 | 284 |
| VH73 | 285 | 286 | 287 |
| VH74 | 288 | 289 | 290 |
| VH75 | 291 | 292 | 293 |
| VH76 | 294 | 295 | 296 |
| VH77 | 297 | 298 | 299 |
| VH78 | 300 | 301 | 302 |
| VH79 | 303 | 304 | 305 |
| VH80 | 306 | 307 | 308 |
| VH81 | 309 | 310 | 311 |
| VH82 | 312 | 313 | 314 |
| VH83 | 315 | 316 | 317 |
| VH84 | 318 | 319 | 320 |
| VH85 | 321 | 322 | 323 |
| VH86 | 324 | 325 | 326 |
| VH87 | 327 | 328 | 329 |
| VH88 | 330 | 331 | 332 |
| VH89 | 333 | 334 | 335 |
| VH90 | 336 | 337 | 338 |
| VH91 | 339 | 340 | 341 |
| VH92 | 342 | 343 | 344 |
| VH93 | 345 | 346 | 347 |
| VH94 | 348 | 349 | 350 |
| VH95 | 351 | 352 | 353 |
| VH96 | 354 | 355 | 356 |
| VH97 | 357 | 358 | 359 |
| VH98 | 360 | 361 | 362 |
| VH99 | 363 | 364 | 365 |
| VH100 | 366 | 367 | 368 |
| VH101 | 369 | 370 | 371 |
| VH102 | 372 | 373 | 374 |
| VH103 | 267 | 604 | 269 |

the CDR1-VL, the CDR2-VL and the CDR3-VL are selected from any sequence combination of the following VL1-VL68 or sequence combinations having 1, 2, 3 or more amino acid insertions, deletions and/or substitutions compared with the sequence combination, and preferably, the substitutions are conservative amino acid substitutions:

| | SEQ ID NO: | | |
|---|---|---|---|
| No. | CDR1-VL | CDR2-VL | CDR3-VL |
| VL1 | 375 | 376 | 377 |
| VL2 | 378 | 379 | 380 |
| VL3 | 381 | 382 | 383 |
| VL4 | 384 | 385 | 386 |
| VL5 | 387 | 388 | 389 |
| VL6 | 390 | 391 | 392 |
| VL7 | 393 | 394 | 395 |
| VL8 | 396 | 397 | 398 |
| VL9 | 399 | 400 | 401 |
| VL10 | 402 | 403 | 404 |
| VL11 | 405 | 406 | 407 |
| VL12 | 408 | 409 | 410 |
| VL13 | 411 | 412 | 413 |
| VL14 | 414 | 415 | 416 |
| VL15 | 417 | 418 | 419 |
| VL16 | 420 | 421 | 422 |
| VL17 | 423 | 424 | 425 |
| VL18 | 426 | 427 | 428 |
| VL19 | 429 | 430 | 431 |
| VL20 | 432 | 433 | 434 |
| VL21 | 435 | 436 | 437 |
| VL22 | 438 | 439 | 440 |
| VL23 | 441 | 442 | 443 |
| VL24 | 444 | 445 | 446 |
| VL25 | 447 | 448 | 449 |
| VL26 | 450 | 451 | 452 |
| VL27 | 453 | 454 | 455 |
| VL28 | 456 | 457 | 458 |
| VL29 | 459 | 460 | 461 |
| VL30 | 462 | 463 | 464 |
| VL31 | 465 | 466 | 467 |
| VL32 | 468 | 469 | 470 |
| VL33 | 471 | 472 | 473 |
| VL34 | 474 | 475 | 476 |
| VL35 | 477 | 478 | 479 |
| VL36 | 480 | 481 | 482 |
| VL37 | 483 | 484 | 485 |
| VL38 | 486 | 487 | 488 |
| VL39 | 489 | 490 | 491 |
| VL40 | 492 | 493 | 494 |
| VL41 | 495 | 496 | 497 |
| VL42 | 498 | 499 | 500 |
| VL43 | 501 | 502 | 503 |
| VL44 | 504 | 505 | 506 |
| VL45 | 507 | 508 | 509 |
| VL46 | 510 | 511 | 512 |
| VL47 | 513 | 514 | 515 |
| VL48 | 516 | 517 | 518 |
| VL49 | 519 | 520 | 521 |
| VL50 | 522 | 523 | 524 |
| VL51 | 525 | 526 | 527 |
| VL52 | 528 | 529 | 530 |
| VL53 | 531 | 532 | 533 |
| VL54 | 534 | 535 | 536 |
| VL55 | 537 | 538 | 539 |
| VL56 | 540 | 541 | 542 |
| VL57 | 543 | 544 | 545 |
| VL58 | 546 | 547 | 548 |
| VL59 | 549 | 550 | 551 |
| VL60 | 552 | 553 | 554 |
| VL61 | 555 | 556 | 557 |
| VL62 | 558 | 559 | 560 |
| VL63 | 561 | 562 | 563 |
| VL64 | 564 | 565 | 566 |
| VL65 | 567 | 568 | 569 |
| VL66 | 570 | 571 | 572 |
| VL67 | 573 | 574 | 575 |
| VL68 | 576 | 577 | 578 |
| VL69 | 602 | 508 | 509 |
| VL70 | 603 | 508 | 509 |
| VL71 | 613 | 502 | 503 |
| VL72 | 614 | 502 | 503 |
| VL73 | 623 | 538 | 539 |
| VL74 | 624 | 538 | 539 |
| VL75 | 636 | 532 | 533 |
| VL76 | 637 | 532 | 533 |
| VL77 | 638 | 532 | 533 |
| VL78 | 639 | 532 | 533 |
| VL79 | 640 | 532 | 533 |
| VL80 | 652 | 544 | 545 |
| VL81 | 653 | 544 | 545 |
| VL82 | 654 | 544 | 545 |
| VL83 | 655 | 544 | 545 |
| VL84 | 656 | 544 | 545 |

3. The antibody or antigen-binding moiety according to claim 2, wherein the antibody or antigen-binding moiety comprises a combination of heavy chain CDRs and light chain CDRs selected from: VH1+VL1, VH2+VL1, VH3+VL2, VH4+VL3, VH5+VL3, VH6+VL4, VH7+VL5, VH8+VL5, VH9+VL6, VH10+VL7, VH11+VL7, VH12+VL8, VH13+VL9, VH14+VL9, VH15+VL10, VH16+VL11, VH17+VL11, VH18+VL12, VH19+VL13, VH20+VL13, VH21+VL14, VH22+VL15, VH23+VL15, VH24+VL16, VH25+VL17, VH26+VL17, VH27+VL18, VH28+VL19, VH29+VL19, VH30+VL20, VH31+VL21, VH32+VL21, VH33+VL22, VH34+VL23, VH35+VL23, VH36+VL24, VH37+VL25, VH38+VL25, VH39+VL26, VH40+VL27, VH41+VL27, VH42+VL28, VH43+VL29, VH44+VL29, VH45+VL30, VH46+VL31, VH47+VL31, VH48+VL32, VH49+VL33, VH50+VL33, VH51+VL34, VH52+VL35, VH53+VL35, VH54+VL36, VH55+VL37, VH56+VL37, VH57+VL38, VH58+VL39, VH59+VL39, VH60+VL40, VH61+VL41, VH62+VL41, VH63+VL42, VH64+VL43, VH65+VL43, VH66+VL44, VH67+VL45, VH68+VL45, VH69+VL46, VH70+VL47, VH71+VL47, VH72+VL48, VH73+VL49, VH74+VL49, VH75+VL50, VH76+VL51, VH77+VL51, VH78+VL52, VH79+VL53, VH80+VL53, VH81+VL54, VH82+VL55, VH83+VL55, VH84+VL56, VH85+VL57, VH86+VL57, VH87+VL58, VH88+VL59, VH89+VL59, VH90+VL60, VH91+VL61, VH92+VL61, VH93+VL62, VH94+VL63, VH95+VL63, VH96+VL64, VH97+VL65, VH98+VL65, VH99+VL66, VH100+VL67, VH101+VL67, VH102+VL68, VH103+VL45, VH103+VL69, VH103+VL70, VH67+VL69, VH67+VL70, VH64+VL71, VH64+VL72, VH82+VL73, VH82+VL74, VH79+VL75, VH79+VL76, VH79+VL77, VH79+VL78, VH79+VL79, VH85+VL80, VH85+VL81, VH85+VL82, VH85+VL83, VH85+VL84, and a combination of CDRs having 1, 2, 3 or more amino acid insertions, deletions and/or substitutions compared with a sequence of the combination of heavy chain CDRs and light chain CDRs, and preferably, the substitutions are conservative amino acid substitutions.

4. The antibody or antigen-binding moiety according to claim 1, comprising a sequence having at least 80%, 85%, 90%, 91%, 92%, 93%, 94%, 95%, 96%, 97%, 98%, 99% or 100% identity to the CDR1, the CDR2, and/or the CDR3, respectively.

5. An anti-glypican-3 (GPC3) antibody or antigen-binding moiety, wherein the antibody or antigen-binding moiety comprises: (1) a heavy chain variable region set forth in any one of SEQ ID NOs: 1-34, 594, 599-601, 605, 610-612, 615, 620-622, 625, 633-635, 641, and 649-651; or a sequence having at least 80%, 85%, 90%, 91%, 92%, 93%, 94%, 95%, 96%, 97%, 98%, 99% or 100% identity to the sequence set forth in any one of SEQ ID NOs: 1-34, 594, 599-601, 605, 610-612, 615, 620-622, 625, 633-635, 641, and 649-651; or a sequence having at most 20, 19, 18, 17, 16, 15, 14, 13, 12, 11, 10, 9, 8, 7, 6, 5, 4, 3, 2 or 1 mutation compared with the sequence set forth in any one of SEQ ID NOs: 1-34, 594, 599-601, 605, 610-612, 615, 620-622, 625, 633-635, 641, and 649-651; and the mutation may be selected from an insertion, a deletion and/or a substitution, and preferably, the substitution is a conservative amino acid substitution;
and/or, (2) a light chain variable region set forth in any one of SEQ ID NOs: 35-68, 595-598, 606-609, 616-619, 626-632, and 642-648; or a sequence having at least 80%, 85%, 90%, 91%, 92%, 93%, 94%, 95%, 96%, 97%, 98%, 99% or 100% identity to the sequence set forth in any one of SEQ ID NOs: 35-68, 595-597, 606-609, 616-619, 626-632, and 642-648; or a sequence having at most 20, 19, 18, 17, 16, 15, 14, 13, 12, 11, 10, 9, 8, 7, 6, 5, 4, 3, 2 or 1 mutation compared with the sequence set forth in any one of SEQ ID NOs: 35-68, 595-597, 606-609, 616-619, 626-632, and 642-648; and the mutation may be selected from an insertion, a deletion and/or a substitution, and preferably, the substitution is a conservative amino acid substitution.

6. The antibody or antigen-binding moiety according to claim 5, wherein the antibody or antigen-binding moiety comprises: (1) the heavy chain variable region and the light chain variable region have sequences set forth in SEQ ID NO: 1 and SEQ ID NO: 35, respectively;
(2) the heavy chain variable region and the light chain variable region have sequences set forth in SEQ ID NO: 2 and SEQ ID NO: 36, respectively;
(3) the heavy chain variable region and the light chain variable region have sequences set forth in SEQ ID NO: 3 and SEQ ID NO: 37, respectively;
(4) the heavy chain variable region and the light chain variable region have sequences set forth in SEQ ID NO: 4 and SEQ ID NO: 38, respectively;
(5) the heavy chain variable region and the light chain variable region have sequences set forth in SEQ ID NO: 5 and SEQ ID NO: 39, respectively;
(6) the heavy chain variable region and the light chain variable region have sequences set forth in SEQ ID NO: 6 and SEQ ID NO: 40, respectively;
(7) the heavy chain variable region and the light chain variable region have sequences set forth in SEQ ID NO: 7 and SEQ ID NO: 41, respectively;
(8) the heavy chain variable region and the light chain variable region have sequences set forth in SEQ ID NO: 8 and SEQ ID NO: 42, respectively;
(9) the heavy chain variable region and the light chain variable region have sequences set forth in SEQ ID NO: 9 and SEQ ID NO: 43, respectively;
(10) the heavy chain variable region and the light chain variable region have sequences set forth in SEQ ID NO: 10 and SEQ ID NO: 44, respectively;
(11) the heavy chain variable region and the light chain variable region have sequences set forth in SEQ ID NO: 11 and SEQ ID NO: 45, respectively;
(12) the heavy chain variable region and the light chain variable region have sequences set forth in SEQ ID NO: 12 and SEQ ID NO: 46, respectively;
(13) the heavy chain variable region and the light chain variable region have sequences set forth in SEQ ID NO: 13 and SEQ ID NO: 47, respectively;
(14) the heavy chain variable region and the light chain variable region have sequences set forth in SEQ ID NO: 14 and SEQ ID NO: 48, respectively;
(15) the heavy chain variable region and the light chain variable region have sequences set forth in SEQ ID NO: 15 and SEQ ID NO: 49, respectively;
(16) the heavy chain variable region and the light chain variable region have sequences set forth in SEQ ID NO: 16 and SEQ ID NO: 50, respectively;
(17) the heavy chain variable region and the light chain variable region have sequences set forth in SEQ ID NO: 17 and SEQ ID NO: 51, respectively;
(18) the heavy chain variable region and the light chain variable region have sequences set forth in SEQ ID NO: 18 and SEQ ID NO: 52, respectively;
(19) the heavy chain variable region and the light chain variable region have sequences set forth in SEQ ID NO: 19 and SEQ ID NO: 53, respectively;
(20) the heavy chain variable region and the light chain variable region have sequences set forth in SEQ ID NO: 20 and SEQ ID NO: 54, respectively;
(21) the heavy chain variable region and the light chain variable region have sequences set forth in SEQ ID NO: 21 and SEQ ID NO: 55, respectively;
(22) the heavy chain variable region and the light chain variable region have sequences set forth in SEQ ID NO: 22 and SEQ ID NO: 56, respectively;
(23) the heavy chain variable region and the light chain variable region have sequences set forth in SEQ ID NO: 23 and SEQ ID NO: 57, respectively;
(24) the heavy chain variable region and the light chain variable region have sequences set forth in SEQ ID NO: 24 and SEQ ID NO: 58, respectively;
(25) the heavy chain variable region and the light chain variable region have sequences set forth in SEQ ID NO: 25 and SEQ ID NO: 59, respectively;
(26) the heavy chain variable region and the light chain variable region have sequences set forth in SEQ ID NO: 26 and SEQ ID NO: 60, respectively;
(27) the heavy chain variable region and the light chain variable region have sequences set forth in SEQ ID NO: 27 and SEQ ID NO: 61, respectively;
(28) the heavy chain variable region and the light chain variable region have sequences set forth in SEQ ID NO: 28 and SEQ ID NO: 62, respectively;
(29) the heavy chain variable region and the light chain variable region have sequences set forth in SEQ ID NO: 29 and SEQ ID NO: 63, respectively;
(30) the heavy chain variable region and the light chain variable region have sequences set forth in SEQ ID NO: 30 and SEQ ID NO: 64, respectively;
(31) the heavy chain variable region and the light chain variable region have sequences set forth in SEQ ID NO: 31 and SEQ ID NO: 65, respectively;
(32) the heavy chain variable region and the light chain variable region have sequences set forth in SEQ ID NO: 32 and SEQ ID NO: 66, respectively;
(33) the heavy chain variable region and the light chain variable region have sequences set forth in SEQ ID NO: 33 and SEQ ID NO: 67, respectively;
(34) the heavy chain variable region and the light chain variable region have sequences set forth in SEQ ID NO: 34 and SEQ ID NO: 68, respectively;
(35) the heavy chain variable region and the light chain variable region have sequences set forth in SEQ ID NO: 594 and SEQ ID NO: 595, respectively;
(36) the heavy chain variable region and the light chain variable region have sequences set forth in SEQ ID NO: 599 and SEQ ID NOs: 596-598, respectively;
(37) the heavy chain variable region and the light chain variable region have sequences set forth in SEQ ID NO: 600 and SEQ ID NOs: 596-598, respectively;
(38) the heavy chain variable region and the light chain variable region have sequences set forth in SEQ ID NO: 601 and SEQ ID NOs: 596-598, respectively;
(39) the heavy chain variable region and the light chain variable region have sequences set forth in SEQ ID NO: 605 and SEQ ID NO: 606, respectively;
(40) the heavy chain variable region and the light chain variable region have sequences set forth in SEQ ID NO: 610 and SEQ ID NOs: 607-609, respectively;
(41) the heavy chain variable region and the light chain variable region have sequences set forth in SEQ ID NO: 611 and SEQ ID NOs: 607-609, respectively;
(42) the heavy chain variable region and the light chain variable region have sequences set forth in SEQ ID NO: 612 and SEQ ID NOs: 607-609, respectively;
(43) the heavy chain variable region and the light chain variable region have sequences set forth in SEQ ID NO: 615 and SEQ ID NO: 616, respectively;
(44) the heavy chain variable region and the light chain variable region have sequences set forth in SEQ ID NO: 620 and SEQ ID NOs: 617-619, respectively;
(45) the heavy chain variable region and the light chain variable region have sequences set forth in SEQ ID NO: 621 and SEQ ID NOs: 617-619, respectively;
(46) the heavy chain variable region and the light chain variable region have sequences set forth in SEQ ID NO: 622 and SEQ ID NOs: 617-619, respectively;
(47) the heavy chain variable region and the light chain variable region have sequences set forth in SEQ ID NO: 625 and SEQ ID NO: 626, respectively;
(48) the heavy chain variable region and the light chain variable region have sequences set forth in SEQ ID NO: 633 and SEQ ID NOs: 627-629, respectively;
(49) the heavy chain variable region and the light chain variable region have sequences set forth in SEQ ID NO: 634 and SEQ ID NOs: 627-629, respectively;
(50) the heavy chain variable region and the light chain variable region have sequences set forth in SEQ ID NO: 635 and SEQ ID NOs: 627-632, respectively;
(51) the heavy chain variable region and the light chain variable region have sequences set forth in SEQ ID NO: 641 and SEQ ID NO: 642, respectively;
(52) the heavy chain variable region and the light chain variable region have sequences set forth in SEQ ID NO: 649 and SEQ ID NOs: 643-648, respectively;
(53) the heavy chain variable region and the light chain variable region have sequences set forth in SEQ ID NO: 650 and SEQ ID NOs: 643-645, respectively;
(54) the heavy chain variable region and the light chain variable region have sequences set forth in SEQ ID NO: 651 and SEQ ID NOs: 643-645, respectively;
(55) the heavy chain variable region and the light chain variable region have sequences having at least 70%, 75%, 80%, 85%, 90%, 95%, 96%, 97%, 98%, 99% or more identity to the sequences set forth in (1) to (54) above, respectively.

7. The antibody or antigen-binding moiety according to claim 1, wherein the antibody or antigen-binding moiety specifically binds to a human, monkey, and/or murine GPC3 protein; and preferably, the antibody or antigen-binding moiety binds to human, monkey, and/or murine GPC3 with a dissociation constant (KD) not greater than 1.00E-7 M, 1.00E-8 M, 2.00E-8 M, 3.00E-8 M, 4.00E-8 M, 5.00E-8 M, 6.00E-8 M, 7.00E-8 M, 8.00E-8 M, 9.00E-8 M, 1.00E-9 M, 2.00E-9 M, 3.00E-9 M, 4.00E-9 M, 5.00E-9 M, 6.00E-9 M, 7.00E-9 M, 8.00E-9 M, 9.00E-9 M or 1.00E-10 M.

8. The antibody or antigen-binding moiety according to claim 1, wherein the antibody or antigen-binding moiety is selected from full-length antibodies, VH single domain antibodies, Fab fragments, Fab′ fragments, F(ab)′2 fragments, Fd fragments, Fv fragments, complementarity determining region (CDR) fragments, single chain variable fragments (scFv), scFv2, disulfide stabilized variable fragments (dsFv), domain antibodies, bivalent single chain antibodies, single chain phage antibodies, bispecific diabodies, triabodies, tetrabodies, and minimal recognition units of antibodies; or
the antibody or antigen-binding moiety is a murine antibody, a humanized antibody, a fully human antibody, or a chimeric antibody.

9. (canceled)

10. The antibody or antigen-binding moiety according to claim 1, wherein the antibody or antigen-binding moiety is capable of specifically binding to a peptide having a sequence of amino acid residues 524-563 of glypican-3.

11. An immunoconjugate, wherein the immunoconjugate comprises the antibody or antigen-binding moiety according to claim 1 and an effector molecule; and preferably, the effector molecule is linked to the antibody or antigen-binding moiety; optionally, the effector molecule comprises a therapeutic agent or a marker; preferably, the therapeutic agent is selected from a drug, a toxin, a radioisotope, a chemotherapeutic drug, and an immunomodulator, and the marker is selected from an isotope, a fluorescent compound, a chemiluminescent compound, an enzyme, a metal ion, a radiocontrast medium, a paramagnetic ion, an ultrasound contrast agent, and a photosensitizer; and more preferably, the drug is vinblastine or daunomycin, and the toxin is Pseudomonas exotoxin, diphtheria toxin, alkaloids, methotrexate, anthracyclines (doxorubicin), taxanes, or a toxin compound; or
the immunoconjugate further comprises a linker for conjugating the effector molecule to the antibody, and the linker comprises but is not limited to hydrazones, thioethers, esters, disulfides, and peptide-containing linkers.

12. (canceled)

13. (canceled)

14. A chimeric antigen receptor (CAR), wherein the CAR comprises an extracellular antigen-binding domain, a transmembrane domain, and an intracellular signaling domain, and the extracellular antigen-binding domain comprises the antibody or the antigen-binding moiety according to claim 1.

15. An immunocompetent cell, wherein the immunocompetent cell expresses the chimeric antigen receptor according to claim 14 or comprises a nucleic acid molecule encoding the chimeric antigen receptor according to claim 14; and preferably, the immunocompetent cell is selected from: a T cell, a natural killer cell (NK cell), a natural killer T cell (NKT cell), a double negative T cell (DNT cell), a monocyte, a macrophage, a dendritic cell, and a mast cell, and the T cell is preferably selected from a cytotoxic T cell, a regulatory T cell, and a helper T cell.

16. A multispecific molecule, wherein the multispecific molecule comprises the antibody or antigen-binding moiety according to claim 1; and preferably, the multispecific molecule further comprises an antibody or antigen-binding moiety specifically binding to an antigen other than GPC3 or binding to a GPC3 epitope different from the antibody or antigen-binding fragment according to claim 1; optionally, the antigen other than GPC3 is an antigen on surface of a T cell, a B cell, a natural killer cell, a dendritic cell, a macrophage, a monocyte, or a neutrophil; and preferably, the antigen other than GPC3 is selected from: CD3, CD3γ, CD3δ, CD3ε, CD3ζ, CD16, CD16A, CD32B, PD-1, PD-2, PD-L1, VEGF, NKG2D, CD19, CD20, CD40, CD47, 4-1BB, CD137, EGFR, EGFRVIII, TNF-alpha, CD33, HER2, HER3, HAS, CD5, CD27, EphA2, EpCAM, MUC1, MUC16, CEA, Claudin18.2, a folate receptor, Claudin6, WT1, NY-ESO-1, MAGE3, ASGPR1, and CDH16; or
the multispecific molecule is a tandem scFv, a bifunctional antibody (Db), a single chain bifunctional antibody (scDb), a dual affinity retargeting (DART) antibody, F(ab′)2, a dual variable domain (DVD) antibody, a knobs-into-holes (KiH) antibody, a dock-and-lock (DNL) antibody, a chemically cross-linked antibody, a heteropolyantibody or a heteroconjugate antibody.

17. (canceled)

18. (canceled)

19. An isolated nucleic acid molecule, wherein the nucleic acid molecule encodes the antibody or antigen-binding moiety according to claim 1.

20. A vector, wherein the expression vector comprises the nucleic acid molecule according to claim 19.

21. A host cell, wherein the host cell comprises the nucleic acid molecule according to claim 19, and preferably, the host cell is a prokaryotic cell or a eukaryotic cell, comprising a bacteria (E. coli), a fungus (yeast), an insect cell, or a mammalian cell (CHO cell line or 293T cell line).

22. (canceled)

23. (canceled)

24. A pharmaceutical composition, comprising a therapeutically effective amount of the antibody or antigen-binding moiety according to claim 1, and a pharmaceutically acceptable carrier.

25. (canceled)

26. A method for treating a subject suffering from a GPC3-mediated tumor, comprising: administering to the subject a therapeutically effective amount of the antibody or antigen-binding moiety according to claim 1; wherein the tumor is preferably selected from diseases such as hepatocellular carcinoma, melanoma, ovarian clear cell carcinoma, hepatoblastoma, neuroblastoma, Wilms' tumor, small cell lung cancer, lung adenocarcinoma, gastric cancer, colon cancer, rectal cancer, cervical cancer, breast cancer, ovarian cancer, skin cancer, lymphoma, prostate cancer, pancreatic cancer, renal cancer, esophageal cancer, thyroid cancer, testicular cancer, bladder cancer, bronchogenic carcinoma, nasopharyngeal cancer, head and neck cancer, endometrial cancer, brain cancer, bone cancer, leukemia, malignant mesothelioma, and liposarcoma; and preferably, hepatocellular carcinoma.

27. (canceled)

28. A kit, comprising the antibody or antigen-binding moiety according to claim 1.

29. (canceled)

30. A method for detecting GPC3 expression in a biological sample, comprising: exposing a sample from a subject to the antibody or antigen-binding moiety according to claim 1, and detecting binding of the antibody or antigen-binding moiety to the sample.


SEQUENCE LISTING (656 sequences)

SEQ ID NO: 1 - protein, 118 aa, Artificial sequence
  Synthetic polypeptide
RVQLQQSGAELVKPGASVRMSCKASGYTFTAYSIEWMKQNLGKSLEWIGNFRPYNDDTKYNENFKGRATLTVEKSSATVYLELSRLTSDDSAVYYCARSGPTDYFDYWGQGTTLIVSS

SEQ ID NO: 2 - protein, 119 aa, Artificial sequence
  Synthetic polypeptide
EVQLQQSGPELVKPGASVKISCKTSGFLFTGHYMHWVKSSHGNILDWIGYIYPYNAISFYNQKFKGKATLTVDKSSNTAYMELRSLTSEDSAVYYCARGDYGNYVTAYWGQGTLVTVSA

SEQ ID NO: 3 - protein, 121 aa, Artificial sequence
  Synthetic polypeptide
EVQLVESGGDSVKPGGSLKLSCAASGFTFSSYGMSWVRQTPDKRLEWVATINSGGSYTYYQDSVKGRFTISRDNAKNTLYLQMSSLKSEDTAIYYCVRHHYGSSYDYFDYWGQGTTLTVSS

SEQ ID NO: 4 - protein, 121 aa, Artificial sequence
  Synthetic polypeptide
HVQLQQPGAELVKPGASVKLSCKASGYTFTSYWMHWVRQRPGQGLEWIEMIHPNSGSTNYNEKFKSKATLTVDKSSSTAYMQLSSLTSEDSAVYYCARGNYNSSLYAMDYWGQGTSVTVSS

SEQ ID NO: 5 - protein, 119 aa, Artificial sequence
  Synthetic polypeptide
QVQLQQSGAELARPGASVKLSCKASGYTFTSYGISWVKQITGQGLEWIGEIYPRSESTYYNEKFKGKATLTADKSSSTAYMELRSLTSEDSAVYFCARDYGNIYSFAYWGQGTLVTVSA

SEQ ID NO: 6 - protein, 117 aa, Artificial sequence
  Synthetic polypeptide
QVQLKQSGAELVRPGASVKLSCKASGYTFTDYYINWVKQRPGQGLEWIARIYPGSGNTYYNEKFKGKATLTAEKSSSTAYMQFSSLTSEDSAVYFCACSTYYTMDYWGQGTSVTVSS

SEQ ID NO: 7 - protein, 119 aa, Artificial sequence
  Synthetic polypeptide
QVQLQQSGAELVKPGASVKMSCKASGYTFTTYPIEWMKQNHGKSLEWIGNFHPYNDDTNYNVKFKGKATLTVEKSSSTVYLELSRLTSDDSALYYCARRREYYGSFDVWGTGTTVTVSS

SEQ ID NO: 8 - protein, 119 aa, Artificial sequence
  Synthetic polypeptide
QVQLQQSGPELVKPGASVKISCKASGYAFSSSWMNWVKQRPGKGLEWIGRIYPGDGDTNYNGRFKGKATLTVDKSSSTAYMQLSSLTSEDSAVYFCARGGVFTMALDHWGQGTTLTVSS

SEQ ID NO: 9 - protein, 119 aa, Artificial sequence
  Synthetic polypeptide
EVQLQQSGPELVKPGASVKMSCKASGYTFTDYNMHWVRQSHGKSLEWIGYINPNNGDTIYNQNFKGKATLTVNKSSSTAYMELRSLTSEDSAVYYCSRTITYYYSMDYWGPGTSVTVSS

SEQ ID NO: 10 - protein, 117 aa, Artificial sequence
  Synthetic polypeptide
EVKLVESGGGLVQPGGSLKLSCAASGFTFSDFYMYWVRQTPEKRLEWVAFISNGGGSTYYPDTVKGRFTISRDNVKNTLYLQMSRLKSEDTAMYYCARPDTSWFAYWGQGSLVTVSA

SEQ ID NO: 11 - protein, 117 aa, Artificial sequence
  Synthetic polypeptide
EVQLQQSGPELVKPGASVKISCKASGYTFTDYYMNWVKQSHGKSLEWIGDIYPNNGDTTYNQRFKGKATLTVDKSSSTAYMELRSLASEDSAVYYCACPYNNPFGYWGQGTLVTVSA

SEQ ID NO: 12 - protein, 119 aa, Artificial sequence
  Synthetic polypeptide
EVQLQQSGPELVKPGASVKMSCKASGYTFTEYYMHWVKQSHGKSLEWIGYIYPNNGGNGYNQKFKGKATLTVDKSSSTAYMGLRSLTSEDSAVYYCVRGYYSNWYFDVWGTGTTVTVSS

SEQ ID NO: 13 - protein, 119 aa, Artificial sequence
  Synthetic polypeptide
QVQLQQSGAELAKPGASVKLSCKASGYSFTSYWIHWVKQRPGQGLEWIGYINPSSGYTKYNQKFKDKATLTADKSSSTAYMQLSSLTYEDSAVYYCARFGSSSFYFDYWGQGTTLTVSS

SEQ ID NO: 14 - protein, 117 aa, Artificial sequence
  Synthetic polypeptide
EFQLQQSGPELVKPGASVKISCKASGYSFTDYNMNWVKQSNGKSLEWIGLIHPKFSVTSYNRKFKGKATLTVDQSSSTAYMQLNSLTSEDSAVYYCARWAGDYFDYWGQGTTLTVSS

SEQ ID NO: 15 - protein, 119 aa, Artificial sequence
  Synthetic polypeptide
EVQLQQSGPVMVKPGPSVKISCQTSGFTFTDYYIHWVKQSHGKSLEWIGLVFPYDGGTNYNQKFKDKATLTLDTSSSTAYMELNSLTSEDSAVYYCARAGGFSYALNYWGQGTSVTVSS

SEQ ID NO: 16 - protein, 119 aa, Artificial sequence
  Synthetic polypeptide
QVQLQQSGTDLARPGASVKLSCKASGYTFTSFGISWVKQRTGQGLEWIGEIYPRSGSPYYNEKFTGKATLTADKSSSTAYMELRSLTSEDSAVYFCAKDFGSSYVFDYWGQGTALTVSS

SEQ ID NO: 17 - protein, 117 aa, Artificial sequence
  Synthetic polypeptide
QVQLQQPGTELVKPGASVKLSCKASGYTFTSYWIHWVKQRPGQGLEWIGNINPPNAGTNYNEKFKSKATLTVDKSSSTAYMQLSSLTSEDSAVYYCARGRWLLHDYWGQGTTLTVSS

SEQ ID NO: 18 - protein, 118 aa, Artificial sequence
  Synthetic polypeptide
DVQLQESGPGLVKPSQSLSLTCSVTGYSITSGYYWNWIRQFPGNRLEWMAYISYDGINNYNPSLKNRISIARDTSTNQFFLKLNSVTSEDTATYYCATTTEVALFDHWGQGTTLTVSS

SEQ ID NO: 19 - protein, 121 aa, Artificial sequence
  Synthetic polypeptide
QVQLQQSDAELVKPGASVKISCKVSGYIFTDHTIHWMKQRPEQGLEWIGYIYPRDGSIKYNENFKGKATLTADKSSSATYMQLNSLTSEDSAVYFCARPIYYDYAYYFDYWGQGTTLTVSS

SEQ ID NO: 20 - protein, 123 aa, Artificial sequence
  Synthetic polypeptide
QIQLVQSGPELKKPGETVKISCKASGYTFTTAGMQWVQKMPGKGFKWIGWINTHSGEPQYAEDFKGRFAFSLETSASTAYLQISNLKNEDTATYFCARSFYYHGSSFFYFDYWGQGTTLTVSS

SEQ ID NO: 21 - protein, 121 aa, Artificial sequence
  Synthetic polypeptide
EVKLVESGGGLVKPGGSLKLSCAASGLTFSTCTMSWVRQTPAKRLEWVATISSGGGNTYYPDSVKGRFTISRDNARNTLYLQMSSLRSEDTAMYYCVRLYYDGSPYAMDYWGQGTSVTVSS

SEQ ID NO: 22 - protein, 115 aa, Artificial sequence
  Synthetic polypeptide
QVQLQQSGAELVRPGASVKLSCKASGYRFTDYEMHWVKQTPVHGLEWIGTIDPETGNTAYTQKFMDKATLTADKSSSTAYMELRSLTSEDSAVYYCTRGYSFTYWGQGTLATVSA

SEQ ID NO: 23 - protein, 120 aa, Artificial sequence
  Synthetic polypeptide
EVQLVETGGGLVQPKGSLKLSCAASGFSFNTNAMNWVRQAPGKGLEWVARIRSKSNNYATYYADSVKDRFTISRDDSQSMLYLQMNNLKTEDTAMYYCVRDGYYVLFAYWGQGTLVTVSA

SEQ ID NO: 24 - protein, 115 aa, Artificial sequence
  Synthetic polypeptide
QVQLQQSGAELVRPGASVKVSCKASGYTFTDYEMHWMKQTPVHGLEWIGANEPETGRSAYDQKFKGKATLTADKSSSTAYMELRSLTSEDSAVYYCTRFYSFAYWGQGTLVTVSA

SEQ ID NO: 25 - protein, 115 aa, Artificial sequence
  Synthetic polypeptide
QVQLQQSGAELVRPGASVKLSCKASGYIFSDYEIHWVKQTPVHGLECIGAVDPETGGTAYNQKFKGKATLTADKSSSTAYMELRSLTSEDSAVYFCTRFYSFAYWGQGTLVTVST

SEQ ID NO: 26 - protein, 116 aa, Artificial sequence
  Synthetic polypeptide
EVQLQQSGPELVKPGASVKMSCKASGYTFTDYYMTWVKQSHGKSLEWIGRVNPRNGDTTYNQKFEGRATLTLDKSLSTAFMQLNSLTSEDSAVYYCINSKGHFDYWGQGTTLTVSS

SEQ ID NO: 27 - protein, 115 aa, Artificial sequence
  Synthetic polypeptide
QVQLQQSGAELVRPGASVKLSCKASGYTFTDYEIHWVKQTPVLGLEWIGAIDPKTGNTAYNQKFMGKATLTADKSSTTAYMDLRSLTSEDSAVYYCTRYFSFAYWGQGTLVTVSA

SEQ ID NO: 28 - protein, 115 aa, Artificial sequence
  Synthetic polypeptide
QVQLQQSGAELVRPGASVKLSCKASGYTFTDYEMHWVKQTPVHGLEWIGAIDPETGNTAYIQKFKGKATLTTDKSSSTAYMELRSLTSEDSAVYYCTRFYSYSHWGQGTTLTVSS

SEQ ID NO: 29 - protein, 115 aa, Artificial sequence
  Synthetic polypeptide
QVQLQQSGAELVRPGASVKLSCKASGYTFTDYEMHWVKQTPVHGLEWIGTIDPETGNTAYNQKFKGKATLTADKSSSTAYMELRSLTSEDSAVYYCTRPYSLTYWGQGTLVTVSA

SEQ ID NO: 30 - protein, 122 aa, Artificial sequence
  Synthetic polypeptide
QVQLQQPGAELVKPGASVKLSCKASGYTFTNNWVHWVKQRPGQGLEWTGMIHPDSGSTIYNEKFQSKATLTVDKSSSTAYMQLSSLTSEDSAVYYCARGYYDSSSYYAMDYWGQGTSVTVSS

SEQ ID NO: 31 - protein, 120 aa, Artificial sequence
  Synthetic polypeptide
QVQLHQSGAEMAKPGASVNLSCKTSGFTFTDYGISWVKQRTRQGLEWIGDIYPRHINIYYNEKFKDKATLTADRSSSTAYMELRSMTSEDSAVYFCARRGTTGRGALDYWGQGTSLTVSS

SEQ ID NO: 32 - protein, 118 aa, Artificial sequence
  Synthetic polypeptide
DVQLQESGPGLVKPSQSLSLTCSVTGYSITSGYYWNWIRQFPGNKLEWMGYISYDVTVNYNPSLKNRISITRDTSKNQFFLKLNSVTTEDTATYYCATITSVASMDHWGQGTSVTVSS

SEQ ID NO: 33 - protein, 120 aa, Artificial sequence
  Synthetic polypeptide
QVQLKQSGPGLVQPSQSLSITCTVSGFSLTSYGVHWVRQSPGKGLEWLGVIWSSGNTDYNAVLISRLSIKKDDSKSQVFFKMNSPQVDDSAIYYCGRNGGWLLRGFMDFWGQGTSVTVSS

SEQ ID NO: 34 - protein, 115 aa, Artificial sequence
  Synthetic polypeptide
QVQLQQSGAELVRPGASVKLSCKASGYTFTDYEMHWVKQTPVHGLEWFGAIDPETGGTAYNQKFRAKATLTADKSSSTAYMELRSLTSEDSAVYYCTRFYSFAYWGQGTLVTVSA

SEQ ID NO: 35 - protein, 107 aa, Artificial sequence
  Synthetic polypeptide
DIQMTQSPASLSASVGETVTITCRASDNIYSYLAWYQQKQGQSPQLLVYNAKTLAEGVPSRFSGSGSVTQFSLKIISLQPEDFGTYYCQHHFGTPRTFGGGTKLEIK

SEQ ID NO: 36 - protein, 112 aa, Artificial sequence
  Synthetic polypeptide
DVLMTQTPLSLPVSLGDQASISCRSSQSIVHSSGNTYLEWYLQKPGQSPKLLIYKVSNRFSGVPDRFSGSGSGTDFTLKISRVEAEDLGVYYCFQGSHLPWTFGGGTKLDIK

SEQ ID NO: 37 - protein, 112 aa, Artificial sequence
  Synthetic polypeptide
DIVMTQAAPSVPVMPGESVSISCRSSKSLLHSNGNTHLYWFLQRPGQSPQLLIYRMSNLASGVPDRFSGSGSGTAFTLRISRVEAEDVGVYYCMQHLEYPLTFGAGTKLELK

SEQ ID NO: 38 - protein, 107 aa, Artificial sequence
  Synthetic polypeptide
DIQMTQTTSSLSASLGDRVTISCSASQGISNYLNWYQQKPDGTVKLLIYYASNLHSGVPSRFSGSGSGTAYSLTISNLEPEDIATYYCQQYSILPYTFGGGTKLEIK

SEQ ID NO: 39 - protein, 107 aa, Artificial sequence
  Synthetic polypeptide
DIQMTQSPASLSASVGETVTITCRTSENIYSYLAWYQQKQGKSPQLLVYNAKTLAEGVPSRFSGSGSGTQFSLKINSLQPEDFGSYYCQHHYGTPLTFGAGTKLELK

SEQ ID NO: 40 - protein, 108 aa, Artificial sequence
  Synthetic polypeptide
QIVLTQSPAIMSASLGERVTMTCTASSGVSSSYLHWYQQKPGSSPKLWIYTTSNLASGVPARFSGSGSGTSYSLTISSMEAEDAATYYCHQYHRSPYTFGGGTKLEIK

SEQ ID NO: 41 - protein, 113 aa, Artificial sequence
  Synthetic polypeptide
DIVMTQSPSSLAMSVGQKVTMSCKSSQSLLNRRNQKNYLAWYQQKPGQSPKLLVYFASTRESGVPDRFIGSGSGTDFTLTISSVQAADLADYFCQQHYSTPYTFGGRTKLEIK

SEQ ID NO: 42 - protein, 106 aa, Artificial sequence
  Synthetic polypeptide
DIQMTQSPSSLSASLGGKVTITCKASQDINKYIAWYQHKPGKGPRLLIHYTSTLQPGIPSRFSGSGSGRDYSFSISNLEPEDIATYYCLQYDNLFTFGGGTKLEIK

SEQ ID NO: 43 - protein, 106 aa, Artificial sequence
  Synthetic polypeptide
QIVLTQSPALMSASPGEKVTMTCSASSSVSYMYWYQQKPRSSPKPWIYLTSNLASGVPARFSGSGSGTSYSLTISSMEAEDAATYYCQQWSSNPLTFGAGTKLELK

SEQ ID NO: 44 - protein, 107 aa, Artificial sequence
  Synthetic polypeptide
DIQMTQTTSSLSASLGDRVTISCRASQDIDNYLNWYQQKPDGTVKVLISYTSRLHSGVPSRFSGSGSGTDYSLTISNLEQEDIATYFCQQGNTLPWTFGGGTKLEIK

SEQ ID NO: 45 - protein, 112 aa, Artificial sequence
  Synthetic polypeptide
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 46 - protein, 112 aa, Artificial sequence
  Synthetic polypeptide
DVLMTQTPLSLPVSLGDQASISCRSSQSIVHSNGKTYLEWYLQKPGQSPKLLIYKVSNRFSGVPDRFSGSGSGTDFTLKISRVEAEDLGVYYCFQGSHVPFTFGSGTKLEIK

SEQ ID NO: 47 - protein, 110 aa, Artificial sequence
  Synthetic polypeptide
DIVLTQSPASLAVSLGQRATISCRASQSVSSSSYSYMHWYQQKPGQPPKLLIKYASNLDSGVPARFSGSGSGTDFTLNIHPVEEEDTATYYCQHSWEIPAFGGGTKLEIK

SEQ ID NO: 48 - protein, 107 aa, Artificial sequence
  Synthetic polypeptide
DIVLTQSPATLSVTPGDSVSLSCRASQSISNNLHWYQQKSHESPRLLIKYASQSISGIPSRFSGSGSGTDFTLTINSVETEDFGMYFCQQSNSWPQTFGGGTKLEIK

SEQ ID NO: 49 - protein, 107 aa, Artificial sequence
  Synthetic polypeptide
DIQMTQSPSSLSASLGGKVTITCKASQDINNYIAWYQHKPGKGPRLLIHFTSTLQPGIPSRFSGSGSGRDYSFSISNLEPEDIATYYCLQYDNLLLTFGAGTKLELK

SEQ ID NO: 50 - protein, 107 aa, Artificial sequence
  Synthetic polypeptide
DIQMTQSPASLSASVGETVTITCRASENIYSYLTWYQQKQGKSPQLLVYNAKTLAEGVPSRFSGSGSGTQFSLKINSLQPEDFGTYYCQHHYGTPPTFGTGTKLDLK

SEQ ID NO: 51 - protein, 107 aa, Artificial sequence
  Synthetic polypeptide
DIQMTQSPASLSISVGETVTITCRASENIYSNLAWYQQKQGKSPQLLVYGTKNLAEGVPSRFSGSGSGTQYSLKINSLQSEDFGNYYCQHFWGTPYTFGGGTKLEIL

SEQ ID NO: 52 - protein, 112 aa, Artificial sequence
  Synthetic polypeptide
DVLLTQTPLSLPVSLGDQASISCRSSQNIVHGNGNTYLEWFLQKPGQSPKLLIYKVSNRFSGVPDRFSGSGSGTDFTLKISRVEAEDLGLYYCFQASHVPWTFGGGTKLEIK

SEQ ID NO: 53 - protein, 112 aa, Artificial sequence
  Synthetic polypeptide
DVVMTQSPLSLPVSLGDQASISCRSRQSLVHSNGNTYLHWYLQKPGQSPKVLIYKVSNRFSGVPDRFSGSGSGTDFTLKISRVEAEDLGVYFCSQSTHVPYTFGGGTKLEIK

SEQ ID NO: 54 - protein, 107 aa, Artificial sequence
  Synthetic polypeptide
DIVMTQSQKFMSTTVGDRVSITCKASQNVGTAVAWYQQKPGQSPKLLIYSASNRYTGVPDRFTGSGSGADFTLTISNMQSEDLADYFCQQYSRYPLTFGAGTKLELK

SEQ ID NO: 55 - protein, 107 aa, Artificial sequence
  Synthetic polypeptide
DIQMTQTTSSLSASLGDRVTISCRASQDISNHLNWYQQKPDGTVKLLIYYTSRLHSGVPSRFSGSGSGTDYSLTISNLEQEDIATYFCQQDSKPPPTFGSGTKLEIK

SEQ ID NO: 56 - protein, 112 aa, Artificial sequence
  Synthetic polypeptide
DVLMTQTPLSLPVSLGDQASISCRSSQNIVHSNGNTYLQWYLQKPGQSPKLLIFKVSNRFSGVPDRFSGSGSGTDFTLKISRVEAEDLGVYYCFQGSHVPFTFGTGTKLEIK

SEQ ID NO: 57 - protein, 112 aa, Artificial sequence
  Synthetic polypeptide
DVLMTQTPLSLPVSLGDQASISCRFSQSIVNSNGNTYLEWFLQKPGQSPKLLIYKVSNRFSGVPDRFSGSGSGTDFTLKISRVEAEDLGVYYCFQGSHVPFTFGTGTKLEIK

SEQ ID NO: 58 - protein, 112 aa, Artificial sequence
  Synthetic polypeptide
DVVMTQTPLSLPVSLGDQASISCRSSQGLVHNNGNTYLHWYLQKPGQSPKLLIYKVSNRFSGVPDRFSGSGSGTDFTLKISRVEAEDLGVYFCSQNTHVPPTFGGGTKLEIK

SEQ ID NO: 59 - protein, 112 aa, Artificial sequence
  Synthetic polypeptide
DVVMTQTPLSLPVSLGDQGSISCRSSQSLVHSNGNTYLQWYLQKPGQSPKLLIYKVSNRFSGVPDRFSGSGSGTDFTLKISRVEAEDLGVYFCCQSTHVLPTFGGGTKLEIK

SEQ ID NO: 60 - protein, 112 aa, Artificial sequence
  Synthetic polypeptide
DVLMTQTPLSLPVSLGDQASISCRSSQSIVHSNGNTYLEWFLQKPGQSPKSLIYKVSNRFSGVPDRFSGSGSGTDFTLKISRVEAEDLGVYYCFQGSHVPPTFGAGTKLELK

SEQ ID NO: 61 - protein, 112 aa, Artificial sequence
  Synthetic polypeptide
DVLMTQTPLSLPVSLGDQASISCRSSQTFVHSNGNTYLQWYLQKPGQSPKLLIYKVSNRFSGVPDRFSGSGSGTDFTLKISRVEAEDLGVYYCFQGSHVPYTFGSGTKLEIK

SEQ ID NO: 62 - protein, 112 aa, Artificial sequence
  Synthetic polypeptide
DVVMTQTPLSLPVSLGDQASISCRSSQSLVHSNGNTYLHWYLQKPGQSPKLLIYKVSNRFSGVPDRFSGSSSGTDFTLKISRVEAEDLGVYFCSQNTHVPPTFGGGTKLEIK

SEQ ID NO: 63 - protein, 112 aa, Artificial sequence
  Synthetic polypeptide
DVLLTQTPLSLPVSLGDQASISCRSSQSFVHSNGDTYLQWYLQKPGQSPKLLIYKVSNRFSGVPDRFSGSGSGTDFTLKISRVEAEDLGVYYCFQGSHVPYTFGSGTKLEIK

SEQ ID NO: 64 - protein, 107 aa, Artificial sequence
  Synthetic polypeptide
NIQMTQTTSSLSASLGDRVTISCSASQGIFNYLNWYQQKPDGTVKLLIYYTSSLHSGVPSRFSGSGSGTDYSLTISNLEPEDIATYYCQQYSKLPWTFGGGTKLEIK

SEQ ID NO: 65 - protein, 107 aa, Artificial sequence
  Synthetic polypeptide
DILLTQSPAILSVSPGERVSFSCRASQSIGTSIHWYQQRTNGSPRLLIKYAFESISGIPSRFGGSGSRTDFTLTINSVESEDIADYYCQQTYNWPYTFGGGTKLEIK

SEQ ID NO: 66 - protein, 112 aa, Artificial sequence
  Synthetic polypeptide
DILMTQTPLSLPVSLGDQASISCRSSQNIVHRNGNTYLEWYLQKPGQSPKLLLYKVSNRFSGVPDRFSGSGSGTDFTLKISRVEAEDLGIYYCFQGSHFPFTFGSGTKLEIK

SEQ ID NO: 67 - protein, 112 aa, Artificial sequence
  Synthetic polypeptide
DVVMTQTPLSLPVSLGDQASISCRSSQSLLHSNGNTYLHWYLQRSGQSPNLLIYKVSNRFSGVPDRFSGGGSGTDFTLKISRVEAEDLGIYFCSQSTHVPWTFGGGTKLEIK

SEQ ID NO: 68 - protein, 112 aa, Artificial sequence
  Synthetic polypeptide
DVVMTQTPLSLPVSLGDQASISCRSSQSLVHSNGNTYLQWYLQKPGQSPKLLIYKVSNRFSGVPDRFSGSGSGTDFTLKITRVEAEDLGVYFCSQSTHVPYTFGSGTKLEIK

SEQ ID NO: 69 - protein, 5 aa, Artificial sequence
  Synthetic polypeptide
AYSIE

SEQ ID NO: 70 - protein, 17 aa, Artificial sequence
  Synthetic polypeptide
NFRPYNDDTKYNENFKG

SEQ ID NO: 71 - protein, 9 aa, Artificial sequence
  Synthetic polypeptide
SGPTDYFDY

SEQ ID NO: 72 - protein, 7 aa, Artificial sequence
  Synthetic polypeptide
GYTFTAY

SEQ ID NO: 73 - protein, 6 aa, Artificial sequence
  Synthetic polypeptide
RPYNDD

SEQ ID NO: 74 - protein, 9 aa, Artificial sequence
  Synthetic polypeptide
SGPTDYFDY

SEQ ID NO: 75 - protein, 8 aa, Artificial sequence
  Synthetic polypeptide
GYTFTAYS

SEQ ID NO: 76 - protein, 8 aa, Artificial sequence
  Synthetic polypeptide
FRPYNDDT

SEQ ID NO: 77 - protein, 11 aa, Artificial sequence
  Synthetic polypeptide
ARSGPTDYFDY

SEQ ID NO: 78 - protein, 5 aa, Artificial sequence
  Synthetic polypeptide
GHYMH

SEQ ID NO: 79 - protein, 17 aa, Artificial sequence
  Synthetic polypeptide
YIYPYNAISFYNQKFKG

SEQ ID NO: 80 - protein, 10 aa, Artificial sequence
  Synthetic polypeptide
GDYGNYVTAY

SEQ ID NO: 81 - protein, 7 aa, Artificial sequence
  Synthetic polypeptide
GFLFTGH

SEQ ID NO: 82 - protein, 6 aa, Artificial sequence
  Synthetic polypeptide
YPYNAI

SEQ ID NO: 83 - protein, 10 aa, Artificial sequence
  Synthetic polypeptide
GDYGNYVTAY

SEQ ID NO: 84 - protein, 8 aa, Artificial sequence
  Synthetic polypeptide
GFLFTGHY

SEQ ID NO: 85 - protein, 8 aa, Artificial sequence
  Synthetic polypeptide
IYPYNAIS

SEQ ID NO: 86 - protein, 12 aa, Artificial sequence
  Synthetic polypeptide
ARGDYGNYVTAY

SEQ ID NO: 87 - protein, 5 aa, Artificial sequence
  Synthetic polypeptide
SYGMS

SEQ ID NO: 88 - protein, 17 aa, Artificial sequence
  Synthetic polypeptide
TINSGGSYTYYQDSVKG

SEQ ID NO: 89 - protein, 12 aa, Artificial sequence
  Synthetic polypeptide
HHYGSSYDYFDY

SEQ ID NO: 90 - protein, 7 aa, Artificial sequence
  Synthetic polypeptide
GFTFSSY

SEQ ID NO: 91 - protein, 6 aa, Artificial sequence
  Synthetic polypeptide
NSGGSY

SEQ ID NO: 92 - protein, 12 aa, Artificial sequence
  Synthetic polypeptide
HHYGSSYDYFDY

SEQ ID NO: 93 - protein, 8 aa, Artificial sequence
  Synthetic polypeptide
GFTFSSYG

SEQ ID NO: 94 - protein, 8 aa, Artificial sequence
  Synthetic polypeptide
INSGGSYT

SEQ ID NO: 95 - protein, 14 aa, Artificial sequence
  Synthetic polypeptide
VRHHYGSSYDYFDY

SEQ ID NO: 96 - protein, 5 aa, Artificial sequence
  Synthetic polypeptide
SYWMH

SEQ ID NO: 97 - protein, 17 aa, Artificial sequence
  Synthetic polypeptide
MIHPNSGSTNYNEKFKS

SEQ ID NO: 98 - protein, 12 aa, Artificial sequence
  Synthetic polypeptide
GNYNSSLYAMDY

SEQ ID NO: 99 - protein, 7 aa, Artificial sequence
  Synthetic polypeptide
GYTFTSY

SEQ ID NO: 100 - protein, 6 aa, Artificial sequence
  Synthetic polypeptide
HPNSGS

SEQ ID NO: 101 - protein, 12 aa, Artificial sequence
  Synthetic polypeptide
GNYNSSLYAMDY

SEQ ID NO: 102 - protein, 8 aa, Artificial sequence
  Synthetic polypeptide
GYTFTSYW

SEQ ID NO: 103 - protein, 8 aa, Artificial sequence
  Synthetic polypeptide
IHPNSGST

SEQ ID NO: 104 - protein, 14 aa, Artificial sequence
  Synthetic polypeptide
ARGNYNSSLYAMDY

SEQ ID NO: 105 - protein, 5 aa, Artificial sequence
  Synthetic polypeptide
SYGIS

SEQ ID NO: 106 - protein, 17 aa, Artificial sequence
  Synthetic polypeptide
EIYPRSESTYYNEKFKG

SEQ ID NO: 107 - protein, 10 aa, Artificial sequence
  Synthetic polypeptide
DYGNIYSFAY

SEQ ID NO: 108 - protein, 7 aa, Artificial sequence
  Synthetic polypeptide
GYTFTSY

SEQ ID NO: 109 - protein, 6 aa, Artificial sequence
  Synthetic polypeptide
YPRSES

SEQ ID NO: 110 - protein, 10 aa, Artificial sequence
  Synthetic polypeptide
DYGNIYSFAY

SEQ ID NO: 111 - protein, 8 aa, Artificial sequence
  Synthetic polypeptide
GYTFTSYG

SEQ ID NO: 112 - protein, 8 aa, Artificial sequence
  Synthetic polypeptide
IYPRSEST

SEQ ID NO: 113 - protein, 12 aa, Artificial sequence
  Synthetic polypeptide
ARDYGNIYSFAY

SEQ ID NO: 114 - protein, 5 aa, Artificial sequence
  Synthetic polypeptide
DYYIN

SEQ ID NO: 115 - protein, 17 aa, Artificial sequence
  Synthetic polypeptide
RIYPGSGNTYYNEKFKG

SEQ ID NO: 116 - protein, 8 aa, Artificial sequence
  Synthetic polypeptide
STYYTMDY

SEQ ID NO: 117 - protein, 7 aa, Artificial sequence
  Synthetic polypeptide
GYTFTDY

SEQ ID NO: 118 - protein, 6 aa, Artificial sequence
  Synthetic polypeptide
YPGSGN

SEQ ID NO: 119 - protein, 8 aa, Artificial sequence
  Synthetic polypeptide
STYYTMDY

SEQ ID NO: 120 - protein, 8 aa, Artificial sequence
  Synthetic polypeptide
GYTFTDYY

SEQ ID NO: 121 - protein, 8 aa, Artificial sequence
  Synthetic polypeptide
IYPGSGNT

SEQ ID NO: 122 - protein, 10 aa, Artificial sequence
  Synthetic polypeptide
ACSTYYTMDY

SEQ ID NO: 123 - protein, 5 aa, Artificial sequence
  Synthetic polypeptide
TYPIE

SEQ ID NO: 124 - protein, 17 aa, Artificial sequence
  Synthetic polypeptide
NFHPYNDDTNYNVKFKG

SEQ ID NO: 125 - protein, 10 aa, Artificial sequence
  Synthetic polypeptide
RREYYGSFDV

SEQ ID NO: 126 - protein, 7 aa, Artificial sequence
  Synthetic polypeptide
GYTFTTY

SEQ ID NO: 127 - protein, 6 aa, Artificial sequence
  Synthetic polypeptide
HPYNDD

SEQ ID NO: 128 - protein, 10 aa, Artificial sequence
  Synthetic polypeptide
RREYYGSFDV

SEQ ID NO: 129 - protein, 8 aa, Artificial sequence
  Synthetic polypeptide
GYTFTTYP

SEQ ID NO: 130 - protein, 8 aa, Artificial sequence
  Synthetic polypeptide
FHPYNDDT

SEQ ID NO: 131 - protein, 12 aa, Artificial sequence
  Synthetic polypeptide
ARRREYYGSFDV

SEQ ID NO: 132 - protein, 5 aa, Artificial sequence
  Synthetic polypeptide
SSWMN

SEQ ID NO: 133 - protein, 17 aa, Artificial sequence
  Synthetic polypeptide
RIYPGDGDTNYNGRFKG

SEQ ID NO: 134 - protein, 10 aa, Artificial sequence
  Synthetic polypeptide
GGVFTMALDH

SEQ ID NO: 135 - protein, 7 aa, Artificial sequence
  Synthetic polypeptide
GYAFSSS

SEQ ID NO: 136 - protein, 6 aa, Artificial sequence
  Synthetic polypeptide
YPGDGD

SEQ ID NO: 137 - protein, 10 aa, Artificial sequence
  Synthetic polypeptide
GGVFTMALDH

SEQ ID NO: 138 - protein, 8 aa, Artificial sequence
  Synthetic polypeptide
GYAFSSSW

SEQ ID NO: 139 - protein, 8 aa, Artificial sequence
  Synthetic polypeptide
IYPGDGDT

SEQ ID NO: 140 - protein, 12 aa, Artificial sequence
  Synthetic polypeptide
ARGGVFTMALDH

SEQ ID NO: 141 - protein, 5 aa, Artificial sequence
  Synthetic polypeptide
DYNMH

SEQ ID NO: 142 - protein, 17 aa, Artificial sequence
  Synthetic polypeptide
YINPNNGDTIYNQNFKG

SEQ ID NO: 143 - protein, 10 aa, Artificial sequence
  Synthetic polypeptide
TITYYYSMDY

SEQ ID NO: 144 - protein, 7 aa, Artificial sequence
  Synthetic polypeptide
GYTFTDY

SEQ ID NO: 145 - protein, 6 aa, Artificial sequence
  Synthetic polypeptide
NPNNGD

SEQ ID NO: 146 - protein, 10 aa, Artificial sequence
  Synthetic polypeptide
TITYYYSMDY

SEQ ID NO: 147 - protein, 8 aa, Artificial sequence
  Synthetic polypeptide
GYTFTDYN

SEQ ID NO: 148 - protein, 9 aa, Artificial sequence
  Synthetic polypeptide
IGYTFTDYT

SEQ ID NO: 149 - protein, 12 aa, Artificial sequence
  Synthetic polypeptide
SRTITYYYSMDY

SEQ ID NO: 150 - protein, 5 aa, Artificial sequence
  Synthetic polypeptide
DFYMY

SEQ ID NO: 151 - protein, 17 aa, Artificial sequence
  Synthetic polypeptide
FISNGGGSTYYPDTVKG

SEQ ID NO: 152 - protein, 8 aa, Artificial sequence
  Synthetic polypeptide
PDTSWFAY

SEQ ID NO: 153 - protein, 7 aa, Artificial sequence
  Synthetic polypeptide
GFTFSDF

SEQ ID NO: 154 - protein, 6 aa, Artificial sequence
  Synthetic polypeptide
SNGGGS

SEQ ID NO: 155 - protein, 8 aa, Artificial sequence
  Synthetic polypeptide
PDTSWFAY

SEQ ID NO: 156 - protein, 8 aa, Artificial sequence
  Synthetic polypeptide
GFTFSDFY

SEQ ID NO: 157 - protein, 8 aa, Artificial sequence
  Synthetic polypeptide
ISNGGGST

SEQ ID NO: 158 - protein, 10 aa, Artificial sequence
  Synthetic polypeptide
ARPDTSWFAY

SEQ ID NO: 159 - protein, 5 aa, Artificial sequence
  Synthetic polypeptide
DYYMN

SEQ ID NO: 160 - protein, 17 aa, Artificial sequence
  Synthetic polypeptide
DIYPNNGDTTYNQRFKG

SEQ ID NO: 161 - protein, 8 aa, Artificial sequence
  Synthetic polypeptide
PYNNPFGY

SEQ ID NO: 162 - protein, 7 aa, Artificial sequence
  Synthetic polypeptide
GYTFTDY

SEQ ID NO: 163 - protein, 6 aa, Artificial sequence
  Synthetic polypeptide
YPNNGD

SEQ ID NO: 164 - protein, 8 aa, Artificial sequence
  Synthetic polypeptide
PYNNPFGY

SEQ ID NO: 165 - protein, 8 aa, Artificial sequence
  Synthetic polypeptide
GYTFTDYY

SEQ ID NO: 166 - protein, 8 aa, Artificial sequence
  Synthetic polypeptide
IYPNNGDT

SEQ ID NO: 167 - protein, 10 aa, Artificial sequence
  Synthetic polypeptide
ACPYNNPFGY

SEQ ID NO: 168 - protein, 5 aa, Artificial sequence
  Synthetic polypeptide
EYYMH

SEQ ID NO: 169 - protein, 17 aa, Artificial sequence
  Synthetic polypeptide
YIYPNNGGNGYNQKFKG

SEQ ID NO: 170 - protein, 10 aa, Artificial sequence
  Synthetic polypeptide
GYYSNWYFDV

SEQ ID NO: 171 - protein, 7 aa, Artificial sequence
  Synthetic polypeptide
GYTFTEY

SEQ ID NO: 172 - protein, 6 aa, Artificial sequence
  Synthetic polypeptide
YPNNGG

SEQ ID NO: 173 - protein, 10 aa, Artificial sequence
  Synthetic polypeptide
GYYSNWYFDV

SEQ ID NO: 174 - protein, 8 aa, Artificial sequence
  Synthetic polypeptide
GYTFTEYY

SEQ ID NO: 175 - protein, 8 aa, Artificial sequence
  Synthetic polypeptide
IYPNNGGN

SEQ ID NO: 176 - protein, 12 aa, Artificial sequence
  Synthetic polypeptide
VRGYYSNWYFDV

SEQ ID NO: 177 - protein, 5 aa, Artificial sequence
  Synthetic polypeptide
SYWIH

SEQ ID NO: 178 - protein, 17 aa, Artificial sequence
  Synthetic polypeptide
YINPSSGYTKYNQKFKD

SEQ ID NO: 179 - protein, 10 aa, Artificial sequence
  Synthetic polypeptide
FGSSSFYFDY

SEQ ID NO: 180 - protein, 7 aa, Artificial sequence
  Synthetic polypeptide
GYSFTSY

SEQ ID NO: 181 - protein, 6 aa, Artificial sequence
  Synthetic polypeptide
NPSSGY

SEQ ID NO: 182 - protein, 10 aa, Artificial sequence
  Synthetic polypeptide
FGSSSFYFDY

SEQ ID NO: 183 - protein, 8 aa, Artificial sequence
  Synthetic polypeptide
GYSFTSYW

SEQ ID NO: 184 - protein, 8 aa, Artificial sequence
  Synthetic polypeptide
INPSSGYT

SEQ ID NO: 185 - protein, 12 aa, Artificial sequence
  Synthetic polypeptide
ARFGSSSFYFDY

SEQ ID NO: 186 - protein, 5 aa, Artificial sequence
  Synthetic polypeptide
DYNMN

SEQ ID NO: 187 - protein, 17 aa, Artificial sequence
  Synthetic polypeptide
LIHPKFSVTSYNRKFKG

SEQ ID NO: 188 - protein, 8 aa, Artificial sequence
  Synthetic polypeptide
WAGDYFDY

SEQ ID NO: 189 - protein, 7 aa, Artificial sequence
  Synthetic polypeptide
GYSFTDY

SEQ ID NO: 190 - protein, 6 aa, Artificial sequence
  Synthetic polypeptide
HPKFSV

SEQ ID NO: 191 - protein, 8 aa, Artificial sequence
  Synthetic polypeptide
WAGDYFDY

SEQ ID NO: 192 - protein, 8 aa, Artificial sequence
  Synthetic polypeptide
GYSFTDYN

SEQ ID NO: 193 - protein, 8 aa, Artificial sequence
  Synthetic polypeptide
IHPKFSVT

SEQ ID NO: 194 - protein, 10 aa, Artificial sequence
  Synthetic polypeptide
ARWAGDYFDY

SEQ ID NO: 195 - protein, 5 aa, Artificial sequence
  Synthetic polypeptide
DYYIH

SEQ ID NO: 196 - protein, 17 aa, Artificial sequence
  Synthetic polypeptide
LVFPYDGGTNYNQKFKD

SEQ ID NO: 197 - protein, 10 aa, Artificial sequence
  Synthetic polypeptide
AGGFSYALNY

SEQ ID NO: 198 - protein, 7 aa, Artificial sequence
  Synthetic polypeptide
GFTFTDY

SEQ ID NO: 199 - protein, 6 aa, Artificial sequence
  Synthetic polypeptide
FPYDGG

SEQ ID NO: 200 - protein, 10 aa, Artificial sequence
  Synthetic polypeptide
AGGFSYALNY

SEQ ID NO: 201 - protein, 8 aa, Artificial sequence
  Synthetic polypeptide
GFTFTDYY

SEQ ID NO: 202 - protein, 8 aa, Artificial sequence
  Synthetic polypeptide
VFPYDGGT

SEQ ID NO: 203 - protein, 12 aa, Artificial sequence
  Synthetic polypeptide
ARAGGFSYALNY

SEQ ID NO: 204 - protein, 5 aa, Artificial sequence
  Synthetic polypeptide
SFGIS

SEQ ID NO: 205 - protein, 17 aa, Artificial sequence
  Synthetic polypeptide
EIYPRSGSPYYNEKFTG

SEQ ID NO: 206 - protein, 10 aa, Artificial sequence
  Synthetic polypeptide
DFGSSYVFDY

SEQ ID NO: 207 - protein, 7 aa, Artificial sequence
  Synthetic polypeptide
GYTFTSF

SEQ ID NO: 208 - protein, 6 aa, Artificial sequence
  Synthetic polypeptide
YPRSGS

SEQ ID NO: 209 - protein, 10 aa, Artificial sequence
  Synthetic polypeptide
DFGSSYVFDY

SEQ ID NO: 210 - protein, 8 aa, Artificial sequence
  Synthetic polypeptide
GYTFTSFG

SEQ ID NO: 211 - protein, 8 aa, Artificial sequence
  Synthetic polypeptide
IYPRSGSP

SEQ ID NO: 212 - protein, 12 aa, Artificial sequence
  Synthetic polypeptide
AKDFGSSYVFDY

SEQ ID NO: 213 - protein, 5 aa, Artificial sequence
  Synthetic polypeptide
SYWIH

SEQ ID NO: 214 - protein, 17 aa, Artificial sequence
  Synthetic polypeptide
NINPPNAGTNYNEKFKS

SEQ ID NO: 215 - protein, 8 aa, Artificial sequence
  Synthetic polypeptide
GRWLLHDY

SEQ ID NO: 216 - protein, 7 aa, Artificial sequence
  Synthetic polypeptide
GYTFTSY

SEQ ID NO: 217 - protein, 6 aa, Artificial sequence
  Synthetic polypeptide
NPPNAG

SEQ ID NO: 218 - protein, 8 aa, Artificial sequence
  Synthetic polypeptide
GRWLLHDY

SEQ ID NO: 219 - protein, 8 aa, Artificial sequence
  Synthetic polypeptide
GYTFTSYW

SEQ ID NO: 220 - protein, 8 aa, Artificial sequence
  Synthetic polypeptide
INPPNAGT

SEQ ID NO: 221 - protein, 10 aa, Artificial sequence
  Synthetic polypeptide
ARGRWLLHDY

SEQ ID NO: 222 - protein, 6 aa, Artificial sequence
  Synthetic polypeptide
SGYYWN

SEQ ID NO: 223 - protein, 16 aa, Artificial sequence
  Synthetic polypeptide
YISYDGINNYNPSLKN

SEQ ID NO: 224 - protein, 9 aa, Artificial sequence
  Synthetic polypeptide
TTEVALFDH

SEQ ID NO: 225 - protein, 8 aa, Artificial sequence
  Synthetic polypeptide
GYSITSGY

SEQ ID NO: 226 - protein, 5 aa, Artificial sequence
  Synthetic polypeptide
SYDGI

SEQ ID NO: 227 - protein, 9 aa, Artificial sequence
  Synthetic polypeptide
TTEVALFDH

SEQ ID NO: 228 - protein, 9 aa, Artificial sequence
  Synthetic polypeptide
GYSITSGYY

SEQ ID NO: 229 - protein, 7 aa, Artificial sequence
  Synthetic polypeptide
ISYDGIN

SEQ ID NO: 230 - protein, 11 aa, Artificial sequence
  Synthetic polypeptide
ATTTEVALFDH

SEQ ID NO: 231 - protein, 5 aa, Artificial sequence
  Synthetic polypeptide
DHTIH

SEQ ID NO: 232 - protein, 17 aa, Artificial sequence
  Synthetic polypeptide
YIYPRDGSIKYNENFKG

SEQ ID NO: 233 - protein, 12 aa, Artificial sequence
  Synthetic polypeptide
PIYYDYAYYFDY

SEQ ID NO: 234 - protein, 7 aa, Artificial sequence
  Synthetic polypeptide
GYIFTDH

SEQ ID NO: 235 - protein, 6 aa, Artificial sequence
  Synthetic polypeptide
YPRDGS

SEQ ID NO: 236 - protein, 12 aa, Artificial sequence
  Synthetic polypeptide
PIYYDYAYYFDY

SEQ ID NO: 237 - protein, 8 aa, Artificial sequence
  Synthetic polypeptide
GYIFTDHT

SEQ ID NO: 238 - protein, 8 aa, Artificial sequence
  Synthetic polypeptide
IYPRDGSI

SEQ ID NO: 239 - protein, 14 aa, Artificial sequence
  Synthetic polypeptide
ARPIYYDYAYYFDY

SEQ ID NO: 240 - protein, 5 aa, Artificial sequence
  Synthetic polypeptide
TAGMQ

SEQ ID NO: 241 - protein, 17 aa, Artificial sequence
  Synthetic polypeptide
WINTHSGEPQYAEDFKG

SEQ ID NO: 242 - protein, 14 aa, Artificial sequence
  Synthetic polypeptide
SFYYHGSSFFYFDY

SEQ ID NO: 243 - protein, 7 aa, Artificial sequence
  Synthetic polypeptide
GYTFTTA

SEQ ID NO: 244 - protein, 6 aa, Artificial sequence
  Synthetic polypeptide
NTHSGE

SEQ ID NO: 245 - protein, 14 aa, Artificial sequence
  Synthetic polypeptide
SFYYHGSSFFYFDY

SEQ ID NO: 246 - protein, 8 aa, Artificial sequence
  Synthetic polypeptide
GYTFTTAG

SEQ ID NO: 247 - protein, 8 aa, Artificial sequence
  Synthetic polypeptide
INTHSGEP

SEQ ID NO: 248 - protein, 16 aa, Artificial sequence
  Synthetic polypeptide
ARSFYYHGSSFFYFDY

SEQ ID NO: 249 - protein, 5 aa, Artificial sequence
  Synthetic polypeptide
TCTMS

SEQ ID NO: 250 - protein, 17 aa, Artificial sequence
  Synthetic polypeptide
TISSGGGNTYYPDSVKG

SEQ ID NO: 251 - protein, 12 aa, Artificial sequence
  Synthetic polypeptide
LYYDGSPYAMDY

SEQ ID NO: 252 - protein, 7 aa, Artificial sequence
  Synthetic polypeptide
GLTFSTC

SEQ ID NO: 253 - protein, 6 aa, Artificial sequence
  Synthetic polypeptide
SSGGGN

SEQ ID NO: 254 - protein, 12 aa, Artificial sequence
  Synthetic polypeptide
LYYDGSPYAMDY

SEQ ID NO: 255 - protein, 8 aa, Artificial sequence
  Synthetic polypeptide
GLTFSTCT

SEQ ID NO: 256 - protein, 8 aa, Artificial sequence
  Synthetic polypeptide
ISSGGGNT

SEQ ID NO: 257 - protein, 14 aa, Artificial sequence
  Synthetic polypeptide
VRLYYDGSPYAMDY

SEQ ID NO: 258 - protein, 5 aa, Artificial sequence
  Synthetic polypeptide
DYEMH

SEQ ID NO: 259 - protein, 17 aa, Artificial sequence
  Synthetic polypeptide
TIDPETGNTAYTQKFMD

SEQ ID NO: 260 - protein, 6 aa, Artificial sequence
  Synthetic polypeptide
GYSFTY

SEQ ID NO: 261 - protein, 7 aa, Artificial sequence
  Synthetic polypeptide
GYRFTDY

SEQ ID NO: 262 - protein, 6 aa, Artificial sequence
  Synthetic polypeptide
DPETGN

SEQ ID NO: 263 - protein, 6 aa, Artificial sequence
  Synthetic polypeptide
GYSFTY

SEQ ID NO: 264 - protein, 8 aa, Artificial sequence
  Synthetic polypeptide
GYRFTDYE

SEQ ID NO: 265 - protein, 8 aa, Artificial sequence
  Synthetic polypeptide
IDPETGNT

SEQ ID NO: 266 - protein, 8 aa, Artificial sequence
  Synthetic polypeptide
TRGYSFTY

SEQ ID NO: 267 - protein, 5 aa, Artificial sequence
  Synthetic polypeptide
TNAMN

SEQ ID NO: 268 - protein, 19 aa, Artificial sequence
  Synthetic polypeptide
RIRSKSNNYATYYADSVKD

SEQ ID NO: 269 - protein, 9 aa, Artificial sequence
  Synthetic polypeptide
DGYYVLFAY

SEQ ID NO: 270 - protein, 7 aa, Artificial sequence
  Synthetic polypeptide
GFSFNTN

SEQ ID NO: 271 - protein, 8 aa, Artificial sequence
  Synthetic polypeptide
RSKSNNYA

SEQ ID NO: 272 - protein, 9 aa, Artificial sequence
  Synthetic polypeptide
DGYYVLFAY

SEQ ID NO: 273 - protein, 8 aa, Artificial sequence
  Synthetic polypeptide
GFSFNTNA

SEQ ID NO: 274 - protein, 10 aa, Artificial sequence
  Synthetic polypeptide
IRSKSNNYAT

SEQ ID NO: 275 - protein, 11 aa, Artificial sequence
  Synthetic polypeptide
VRDGYYVLFAY

SEQ ID NO: 276 - protein, 5 aa, Artificial sequence
  Synthetic polypeptide
DYEMH

SEQ ID NO: 277 - protein, 17 aa, Artificial sequence
  Synthetic polypeptide
ANEPETGRSAYDQKFKG

SEQ ID NO: 278 - protein, 6 aa, Artificial sequence
  Synthetic polypeptide
FYSFAY

SEQ ID NO: 279 - protein, 7 aa, Artificial sequence
  Synthetic polypeptide
GYTFTDY

SEQ ID NO: 280 - protein, 6 aa, Artificial sequence
  Synthetic polypeptide
EPETGR

SEQ ID NO: 281 - protein, 6 aa, Artificial sequence
  Synthetic polypeptide
FYSFAY

SEQ ID NO: 282 - protein, 8 aa, Artificial sequence
  Synthetic polypeptide
GYTFTDYE

SEQ ID NO: 283 - protein, 8 aa, Artificial sequence
  Synthetic polypeptide
NEPETGRS

SEQ ID NO: 284 - protein, 8 aa, Artificial sequence
  Synthetic polypeptide
TRFYSFAY

SEQ ID NO: 285 - protein, 5 aa, Artificial sequence
  Synthetic polypeptide
DYEIH

SEQ ID NO: 286 - protein, 17 aa, Artificial sequence
  Synthetic polypeptide
AVDPETGGTAYNQKFKG

SEQ ID NO: 287 - protein, 6 aa, Artificial sequence
  Synthetic polypeptide
FYSFAY

SEQ ID NO: 288 - protein, 7 aa, Artificial sequence
  Synthetic polypeptide
GYIFSDY

SEQ ID NO: 289 - protein, 6 aa, Artificial sequence
  Synthetic polypeptide
DPETGG

SEQ ID NO: 290 - protein, 6 aa, Artificial sequence
  Synthetic polypeptide
FYSFAY

SEQ ID NO: 291 - protein, 8 aa, Artificial sequence
  Synthetic polypeptide
GYIFSDYE

SEQ ID NO: 292 - protein, 8 aa, Artificial sequence
  Synthetic polypeptide
VDPETGGT

SEQ ID NO: 293 - protein, 8 aa, Artificial sequence
  Synthetic polypeptide
TRFYSFAY

SEQ ID NO: 294 - protein, 5 aa, Artificial sequence
  Synthetic polypeptide
DYYMT

SEQ ID NO: 295 - protein, 17 aa, Artificial sequence
  Synthetic polypeptide
RVNPRNGDTTYNQKFEG

SEQ ID NO: 296 - protein, 7 aa, Artificial sequence
  Synthetic polypeptide
SKGHFDY

SEQ ID NO: 297 - protein, 7 aa, Artificial sequence
  Synthetic polypeptide
GYTFTDY

SEQ ID NO: 298 - protein, 6 aa, Artificial sequence
  Synthetic polypeptide
NPRNGD

SEQ ID NO: 299 - protein, 7 aa, Artificial sequence
  Synthetic polypeptide
SKGHFDY

SEQ ID NO: 300 - protein, 8 aa, Artificial sequence
  Synthetic polypeptide
GYTFTDYY

SEQ ID NO: 301 - protein, 8 aa, Artificial sequence
  Synthetic polypeptide
VNPRNGDT

SEQ ID NO: 302 - protein, 9 aa, Artificial sequence
  Synthetic polypeptide
INSKGHFDY

SEQ ID NO: 303 - protein, 5 aa, Artificial sequence
  Synthetic polypeptide
DYEIH

SEQ ID NO: 304 - protein, 17 aa, Artificial sequence
  Synthetic polypeptide
AIDPKTGNTAYNQKFMG

SEQ ID NO: 305 - protein, 6 aa, Artificial sequence
  Synthetic polypeptide
YFSFAY

SEQ ID NO: 306 - protein, 7 aa, Artificial sequence
  Synthetic polypeptide
GYTFTDY

SEQ ID NO: 307 - protein, 6 aa, Artificial sequence
  Synthetic polypeptide
DPKTGN

SEQ ID NO: 308 - protein, 6 aa, Artificial sequence
  Synthetic polypeptide
YFSFAY

SEQ ID NO: 309 - protein, 8 aa, Artificial sequence
  Synthetic polypeptide
GYTFTDYE

SEQ ID NO: 310 - protein, 8 aa, Artificial sequence
  Synthetic polypeptide
IDPKTGNT

SEQ ID NO: 311 - protein, 8 aa, Artificial sequence
  Synthetic polypeptide
TRYFSFAY

SEQ ID NO: 312 - protein, 5 aa, Artificial sequence
  Synthetic polypeptide
DYEMH

SEQ ID NO: 313 - protein, 17 aa, Artificial sequence
  Synthetic polypeptide
AIDPETGNTAYIQKFKG

SEQ ID NO: 314 - protein, 6 aa, Artificial sequence
  Synthetic polypeptide
FYSYSH

SEQ ID NO: 315 - protein, 7 aa, Artificial sequence
  Synthetic polypeptide
GYTFTDY

SEQ ID NO: 316 - protein, 6 aa, Artificial sequence
  Synthetic polypeptide
DPETGN

SEQ ID NO: 317 - protein, 6 aa, Artificial sequence
  Synthetic polypeptide
FYSYSH

SEQ ID NO: 318 - protein, 8 aa, Artificial sequence
  Synthetic polypeptide
GYTFTDYE

SEQ ID NO: 319 - protein, 8 aa, Artificial sequence
  Synthetic polypeptide
IDPETGNT

SEQ ID NO: 320 - protein, 8 aa, Artificial sequence
  Synthetic polypeptide
TRFYSYSH

SEQ ID NO: 321 - protein, 5 aa, Artificial sequence
  Synthetic polypeptide
DYEMH

SEQ ID NO: 322 - protein, 17 aa, Artificial sequence
  Synthetic polypeptide
TIDPETGNTAYNQKFKG

SEQ ID NO: 323 - protein, 6 aa, Artificial sequence
  Synthetic polypeptide
PYSLTY

SEQ ID NO: 324 - protein, 7 aa, Artificial sequence
  Synthetic polypeptide
GYTFTDY

SEQ ID NO: 325 - protein, 6 aa, Artificial sequence
  Synthetic polypeptide
DPETGN

SEQ ID NO: 326 - protein, 6 aa, Artificial sequence
  Synthetic polypeptide
PYSLTY

SEQ ID NO: 327 - protein, 8 aa, Artificial sequence
  Synthetic polypeptide
GYTFTDYE

SEQ ID NO: 328 - protein, 8 aa, Artificial sequence
  Synthetic polypeptide
IDPETGNT

SEQ ID NO: 329 - protein, 8 aa, Artificial sequence
  Synthetic polypeptide
TRPYSLTY

SEQ ID NO: 330 - protein, 5 aa, Artificial sequence
  Synthetic polypeptide
NNWVH

SEQ ID NO: 331 - protein, 17 aa, Artificial sequence
  Synthetic polypeptide
MIHPDSGSTIYNEKFQS

SEQ ID NO: 332 - protein, 13 aa, Artificial sequence
  Synthetic polypeptide
GYYDSSSYYAMDY

SEQ ID NO: 333 - protein, 7 aa, Artificial sequence
  Synthetic polypeptide
GYTFTNN

SEQ ID NO: 334 - protein, 6 aa, Artificial sequence
  Synthetic polypeptide
HPDSGS

SEQ ID NO: 335 - protein, 13 aa, Artificial sequence
  Synthetic polypeptide
GYYDSSSYYAMDY

SEQ ID NO: 336 - protein, 8 aa, Artificial sequence
  Synthetic polypeptide
GYTFTNNW

SEQ ID NO: 337 - protein, 8 aa, Artificial sequence
  Synthetic polypeptide
IHPDSGST

SEQ ID NO: 338 - protein, 15 aa, Artificial sequence
  Synthetic polypeptide
ARGYYDSSSYYAMDY

SEQ ID NO: 339 - protein, 5 aa, Artificial sequence
  Synthetic polypeptide
DYGIS

SEQ ID NO: 340 - protein, 17 aa, Artificial sequence
  Synthetic polypeptide
DIYPRHINIYYNEKFKD

SEQ ID NO: 341 - protein, 11 aa, Artificial sequence
  Synthetic polypeptide
RGTTGRGALDY

SEQ ID NO: 342 - protein, 7 aa, Artificial sequence
  Synthetic polypeptide
GFTFTDY

SEQ ID NO: 343 - protein, 6 aa, Artificial sequence
  Synthetic polypeptide
YPRHIN

SEQ ID NO: 344 - protein, 11 aa, Artificial sequence
  Synthetic polypeptide
RGTTGRGALDY

SEQ ID NO: 345 - protein, 8 aa, Artificial sequence
  Synthetic polypeptide
GFTFTDYG

SEQ ID NO: 346 - protein, 8 aa, Artificial sequence
  Synthetic polypeptide
IYPRHINI

SEQ ID NO: 347 - protein, 13 aa, Artificial sequence
  Synthetic polypeptide
ARRGTTGRGALDY

SEQ ID NO: 348 - protein, 6 aa, Artificial sequence
  Synthetic polypeptide
SGYYWN

SEQ ID NO: 349 - protein, 16 aa, Artificial sequence
  Synthetic polypeptide
YISYDVTVNYNPSLKN

SEQ ID NO: 350 - protein, 9 aa, Artificial sequence
  Synthetic polypeptide
ITSVASMDH

SEQ ID NO: 351 - protein, 8 aa, Artificial sequence
  Synthetic polypeptide
GYSITSGY

SEQ ID NO: 352 - protein, 5 aa, Artificial sequence
  Synthetic polypeptide
SYDVT

SEQ ID NO: 353 - protein, 9 aa, Artificial sequence
  Synthetic polypeptide
ITSVASMDH

SEQ ID NO: 354 - protein, 9 aa, Artificial sequence
  Synthetic polypeptide
GYSITSGYY

SEQ ID NO: 355 - protein, 7 aa, Artificial sequence
  Synthetic polypeptide
ISYDVTV

SEQ ID NO: 356 - protein, 11 aa, Artificial sequence
  Synthetic polypeptide
ATITSVASMDH

SEQ ID NO: 357 - protein, 5 aa, Artificial sequence
  Synthetic polypeptide
SYGVH

SEQ ID NO: 358 - protein, 16 aa, Artificial sequence
  Synthetic polypeptide
VIWSSGNTDYNAVLIS

SEQ ID NO: 359 - protein, 12 aa, Artificial sequence
  Synthetic polypeptide
NGGWLLRGFMDF

SEQ ID NO: 360 - protein, 7 aa, Artificial sequence
  Synthetic polypeptide
GFSLTSY

SEQ ID NO: 361 - protein, 5 aa, Artificial sequence
  Synthetic polypeptide
WSSGN

SEQ ID NO: 362 - protein, 12 aa, Artificial sequence
  Synthetic polypeptide
NGGWLLRGFMDF

SEQ ID NO: 363 - protein, 8 aa, Artificial sequence
  Synthetic polypeptide
GFSLTSYG

SEQ ID NO: 364 - protein, 7 aa, Artificial sequence
  Synthetic polypeptide
IWSSGNT

SEQ ID NO: 365 - protein, 14 aa, Artificial sequence
  Synthetic polypeptide
GRNGGWLLRGFMDF

SEQ ID NO: 366 - protein, 5 aa, Artificial sequence
  Synthetic polypeptide
DYEMH

SEQ ID NO: 367 - protein, 17 aa, Artificial sequence
  Synthetic polypeptide
AIDPETGGTAYNQKFRA

SEQ ID NO: 368 - protein, 6 aa, Artificial sequence
  Synthetic polypeptide
FYSFAY

SEQ ID NO: 369 - protein, 7 aa, Artificial sequence
  Synthetic polypeptide
GYTFTDY

SEQ ID NO: 370 - protein, 6 aa, Artificial sequence
  Synthetic polypeptide
DPETGG

SEQ ID NO: 371 - protein, 6 aa, Artificial sequence
  Synthetic polypeptide
FYSFAY

SEQ ID NO: 372 - protein, 8 aa, Artificial sequence
  Synthetic polypeptide
GYTFTDYE

SEQ ID NO: 373 - protein, 8 aa, Artificial sequence
  Synthetic polypeptide
IDPETGGT

SEQ ID NO: 374 - protein, 8 aa, Artificial sequence
  Synthetic polypeptide
TRFYSFAY

SEQ ID NO: 375 - protein, 11 aa, Artificial sequence
  Synthetic polypeptide
RASDNIYSYLA

SEQ ID NO: 376 - protein, 7 aa, Artificial sequence
  Synthetic polypeptide
NAKTLAE

SEQ ID NO: 377 - protein, 9 aa, Artificial sequence
  Synthetic polypeptide
QHHFGTPRT

SEQ ID NO: 378 - protein, 6 aa, Artificial sequence
  Synthetic polypeptide
DNIYSY

SEQ ID NO: 379 - protein, 3 aa, Artificial sequence
  Synthetic polypeptide
NAK

SEQ ID NO: 380 - protein, 9 aa, Artificial sequence
  Synthetic polypeptide
QHHFGTPRT

SEQ ID NO: 381 - protein, 16 aa, Artificial sequence
  Synthetic polypeptide
RSSQSIVHSSGNTYLE

SEQ ID NO: 382 - protein, 7 aa, Artificial sequence
  Synthetic polypeptide
KVSNRFS

SEQ ID NO: 383 - protein, 9 aa, Artificial sequence
  Synthetic polypeptide
FQGSHLPWT

SEQ ID NO: 384 - protein, 11 aa, Artificial sequence
  Synthetic polypeptide
QSIVHSSGNTY

SEQ ID NO: 385 - protein, 3 aa, Artificial sequence
  Synthetic polypeptide
KVS

SEQ ID NO: 386 - protein, 9 aa, Artificial sequence
  Synthetic polypeptide
FQGSHLPWT

SEQ ID NO: 387 - protein, 16 aa, Artificial sequence
  Synthetic polypeptide
RSSKSLLHSNGNTHLY

SEQ ID NO: 388 - protein, 7 aa, Artificial sequence
  Synthetic polypeptide
RMSNLAS

SEQ ID NO: 389 - protein, 9 aa, Artificial sequence
  Synthetic polypeptide
MQHLEYPLT

SEQ ID NO: 390 - protein, 11 aa, Artificial sequence
  Synthetic polypeptide
KSLLHSNGNTH

SEQ ID NO: 391 - protein, 3 aa, Artificial sequence
  Synthetic polypeptide
RMS

SEQ ID NO: 392 - protein, 9 aa, Artificial sequence
  Synthetic polypeptide
MQHLEYPLT

SEQ ID NO: 393 - protein, 11 aa, Artificial sequence
  Synthetic polypeptide
SASQGISNYLN

SEQ ID NO: 394 - protein, 7 aa, Artificial sequence
  Synthetic polypeptide
YASNLHS

SEQ ID NO: 395 - protein, 9 aa, Artificial sequence
  Synthetic polypeptide
QQYSILPYT

SEQ ID NO: 396 - protein, 6 aa, Artificial sequence
  Synthetic polypeptide
QGISNY

SEQ ID NO: 397 - protein, 3 aa, Artificial sequence
  Synthetic polypeptide
YAS

SEQ ID NO: 398 - protein, 9 aa, Artificial sequence
  Synthetic polypeptide
QQYSILPYT

SEQ ID NO: 399 - protein, 11 aa, Artificial sequence
  Synthetic polypeptide
RTSENIYSYLA

SEQ ID NO: 400 - protein, 7 aa, Artificial sequence
  Synthetic polypeptide
NAKTLAE

SEQ ID NO: 401 - protein, 9 aa, Artificial sequence
  Synthetic polypeptide
QHHYGTPLT

SEQ ID NO: 402 - protein, 6 aa, Artificial sequence
  Synthetic polypeptide
ENIYSY

SEQ ID NO: 403 - protein, 3 aa, Artificial sequence
  Synthetic polypeptide
NAK

SEQ ID NO: 404 - protein, 9 aa, Artificial sequence
  Synthetic polypeptide
QHHYGTPLT

SEQ ID NO: 405 - protein, 12 aa, Artificial sequence
  Synthetic polypeptide
TASSGVSSSYLH

SEQ ID NO: 406 - protein, 7 aa, Artificial sequence
  Synthetic polypeptide
TTSNLAS

SEQ ID NO: 407 - protein, 9 aa, Artificial sequence
  Synthetic polypeptide
HQYHRSPYT

SEQ ID NO: 408 - protein, 7 aa, Artificial sequence
  Synthetic polypeptide
SGVSSSY

SEQ ID NO: 409 - protein, 3 aa, Artificial sequence
  Synthetic polypeptide
TTS

SEQ ID NO: 410 - protein, 9 aa, Artificial sequence
  Synthetic polypeptide
HQYHRSPYT

SEQ ID NO: 411 - protein, 17 aa, Artificial sequence
  Synthetic polypeptide
KSSQSLLNRRNQKNYLA

SEQ ID NO: 412 - protein, 7 aa, Artificial sequence
  Synthetic polypeptide
FASTRES

SEQ ID NO: 413 - protein, 9 aa, Artificial sequence
  Synthetic polypeptide
QQHYSTPYT

SEQ ID NO: 414 - protein, 12 aa, Artificial sequence
  Synthetic polypeptide
QSLLNRRNQKNY

SEQ ID NO: 415 - protein, 3 aa, Artificial sequence
  Synthetic polypeptide
FAS

SEQ ID NO: 416 - protein, 9 aa, Artificial sequence
  Synthetic polypeptide
QQHYSTPYT

SEQ ID NO: 417 - protein, 11 aa, Artificial sequence
  Synthetic polypeptide
KASQDINKYIA

SEQ ID NO: 418 - protein, 7 aa, Artificial sequence
  Synthetic polypeptide
YTSTLQP

SEQ ID NO: 419 - protein, 8 aa, Artificial sequence
  Synthetic polypeptide
LQYDNLFT

SEQ ID NO: 420 - protein, 6 aa, Artificial sequence
  Synthetic polypeptide
QDINKY

SEQ ID NO: 421 - protein, 3 aa, Artificial sequence
  Synthetic polypeptide
YTS

SEQ ID NO: 422 - protein, 8 aa, Artificial sequence
  Synthetic polypeptide
LQYDNLFT

SEQ ID NO: 423 - protein, 10 aa, Artificial sequence
  Synthetic polypeptide
SASSSVSYMY

SEQ ID NO: 424 - protein, 7 aa, Artificial sequence
  Synthetic polypeptide
LTSNLAS

SEQ ID NO: 425 - protein, 9 aa, Artificial sequence
  Synthetic polypeptide
QQWSSNPLT

SEQ ID NO: 426 - protein, 5 aa, Artificial sequence
  Synthetic polypeptide
SSVSY

SEQ ID NO: 427 - protein, 3 aa, Artificial sequence
  Synthetic polypeptide
LTS

SEQ ID NO: 428 - protein, 9 aa, Artificial sequence
  Synthetic polypeptide
QQWSSNPLT

SEQ ID NO: 429 - protein, 11 aa, Artificial sequence
  Synthetic polypeptide
RASQDIDNYLN

SEQ ID NO: 430 - protein, 7 aa, Artificial sequence
  Synthetic polypeptide
YTSRLHS

SEQ ID NO: 431 - protein, 9 aa, Artificial sequence
  Synthetic polypeptide
QQGNTLPWT

SEQ ID NO: 432 - protein, 6 aa, Artificial sequence
  Synthetic polypeptide
QDIDNY

SEQ ID NO: 433 - protein, 3 aa, Artificial sequence
  Synthetic polypeptide
YTS

SEQ ID NO: 434 - protein, 9 aa, Artificial sequence
  Synthetic polypeptide
QQGNTLPWT

SEQ ID NO: 435 - protein, 16 aa, Artificial sequence
  Synthetic polypeptide
RSSQSLVHSNGNTYLH

SEQ ID NO: 436 - protein, 7 aa, Artificial sequence
  Synthetic polypeptide
KVSNRFS

SEQ ID NO: 437 - protein, 9 aa, Artificial sequence
  Synthetic polypeptide
SQSTHVPWT

SEQ ID NO: 438 - protein, 11 aa, Artificial sequence
  Synthetic polypeptide
QSIVHSNGKTY

SEQ ID NO: 439 - protein, 3 aa, Artificial sequence
  Synthetic polypeptide
KVS

SEQ ID NO: 440 - protein, 9 aa, Artificial sequence
  Synthetic polypeptide
SQSTHVPWT

SEQ ID NO: 441 - protein, 16 aa, Artificial sequence
  Synthetic polypeptide
RSSQSIVHSNGKTYLE

SEQ ID NO: 442 - protein, 7 aa, Artificial sequence
  Synthetic polypeptide
KVSNRFS

SEQ ID NO: 443 - protein, 9 aa, Artificial sequence
  Synthetic polypeptide
FQGSHVPFT

SEQ ID NO: 444 - protein, 11 aa, Artificial sequence
  Synthetic polypeptide
QSIVHSNGKTY

SEQ ID NO: 445 - protein, 3 aa, Artificial sequence
  Synthetic polypeptide
KVS

SEQ ID NO: 446 - protein, 9 aa, Artificial sequence
  Synthetic polypeptide
FQGSHVPFT

SEQ ID NO: 447 - protein, 15 aa, Artificial sequence
  Synthetic polypeptide
RASQSVSSSSYSYMH

SEQ ID NO: 448 - protein, 7 aa, Artificial sequence
  Synthetic polypeptide
YASNLDS

SEQ ID NO: 449 - protein, 8 aa, Artificial sequence
  Synthetic polypeptide
QHSWEIPA

SEQ ID NO: 450 - protein, 10 aa, Artificial sequence
  Synthetic polypeptide
QSVSSSSYSY

SEQ ID NO: 451 - protein, 3 aa, Artificial sequence
  Synthetic polypeptide
YAS

SEQ ID NO: 452 - protein, 8 aa, Artificial sequence
  Synthetic polypeptide
QHSWEIPA

SEQ ID NO: 453 - protein, 11 aa, Artificial sequence
  Synthetic polypeptide
RASQSISNNLH

SEQ ID NO: 454 - protein, 7 aa, Artificial sequence
  Synthetic polypeptide
YASQSIS

SEQ ID NO: 455 - protein, 9 aa, Artificial sequence
  Synthetic polypeptide
QQSNSWPQT

SEQ ID NO: 456 - protein, 6 aa, Artificial sequence
  Synthetic polypeptide
QSISNN

SEQ ID NO: 457 - protein, 3 aa, Artificial sequence
  Synthetic polypeptide
YAS

SEQ ID NO: 458 - protein, 9 aa, Artificial sequence
  Synthetic polypeptide
QQSNSWPQT

SEQ ID NO: 459 - protein, 11 aa, Artificial sequence
  Synthetic polypeptide
KASQDINNYIA

SEQ ID NO: 460 - protein, 7 aa, Artificial sequence
  Synthetic polypeptide
FTSTLQP

SEQ ID NO: 461 - protein, 9 aa, Artificial sequence
  Synthetic polypeptide
LQYDNLLLT

SEQ ID NO: 462 - protein, 6 aa, Artificial sequence
  Synthetic polypeptide
QDINNY

SEQ ID NO: 463 - protein, 3 aa, Artificial sequence
  Synthetic polypeptide
FTS

SEQ ID NO: 464 - protein, 9 aa, Artificial sequence
  Synthetic polypeptide
LQYDNLLLT

SEQ ID NO: 465 - protein, 11 aa, Artificial sequence
  Synthetic polypeptide
RASENIYSYLT

SEQ ID NO: 466 - protein, 7 aa, Artificial sequence
  Synthetic polypeptide
NAKTLAE

SEQ ID NO: 467 - protein, 9 aa, Artificial sequence
  Synthetic polypeptide
QHHYGTPPT

SEQ ID NO: 468 - protein, 6 aa, Artificial sequence
  Synthetic polypeptide
ENIYSY

SEQ ID NO: 469 - protein, 3 aa, Artificial sequence
  Synthetic polypeptide
NAK

SEQ ID NO: 470 - protein, 9 aa, Artificial sequence
  Synthetic polypeptide
QHHYGTPPT

SEQ ID NO: 471 - protein, 11 aa, Artificial sequence
  Synthetic polypeptide
RASENIYSNLA

SEQ ID NO: 472 - protein, 7 aa, Artificial sequence
  Synthetic polypeptide
GTKNLAE

SEQ ID NO: 473 - protein, 9 aa, Artificial sequence
  Synthetic polypeptide
QHFWGTPYT

SEQ ID NO: 474 - protein, 6 aa, Artificial sequence
  Synthetic polypeptide
ENIYSN

SEQ ID NO: 475 - protein, 3 aa, Artificial sequence
  Synthetic polypeptide
GTK

SEQ ID NO: 476 - protein, 9 aa, Artificial sequence
  Synthetic polypeptide
QHFWGTPYT

SEQ ID NO: 477 - protein, 16 aa, Artificial sequence
  Synthetic polypeptide
RSSQNIVHGNGNTYLE

SEQ ID NO: 478 - protein, 7 aa, Artificial sequence
  Synthetic polypeptide
KVSNRFS

SEQ ID NO: 479 - protein, 9 aa, Artificial sequence
  Synthetic polypeptide
FQASHVPWT

SEQ ID NO: 480 - protein, 11 aa, Artificial sequence
  Synthetic polypeptide
QNIVHGNGNTY

SEQ ID NO: 481 - protein, 3 aa, Artificial sequence
  Synthetic polypeptide
KVS

SEQ ID NO: 482 - protein, 9 aa, Artificial sequence
  Synthetic polypeptide
FQASHVPWT

SEQ ID NO: 483 - protein, 16 aa, Artificial sequence
  Synthetic polypeptide
RSRQSLVHSNGNTYLH

SEQ ID NO: 484 - protein, 7 aa, Artificial sequence
  Synthetic polypeptide
KVSNRFS

SEQ ID NO: 485 - protein, 9 aa, Artificial sequence
  Synthetic polypeptide
SQSTHVPYT

SEQ ID NO: 486 - protein, 11 aa, Artificial sequence
  Synthetic polypeptide
QSLVHSNGNTY

SEQ ID NO: 487 - protein, 3 aa, Artificial sequence
  Synthetic polypeptide
KVS

SEQ ID NO: 488 - protein, 9 aa, Artificial sequence
  Synthetic polypeptide
SQSTHVPYT

SEQ ID NO: 489 - protein, 11 aa, Artificial sequence
  Synthetic polypeptide
KASQNVGTAVA

SEQ ID NO: 490 - protein, 7 aa, Artificial sequence
  Synthetic polypeptide
SASNRYT

SEQ ID NO: 491 - protein, 9 aa, Artificial sequence
  Synthetic polypeptide
QQYSRYPLT

SEQ ID NO: 492 - protein, 6 aa, Artificial sequence
  Synthetic polypeptide
QNVGTA

SEQ ID NO: 493 - protein, 3 aa, Artificial sequence
  Synthetic polypeptide
SAS

SEQ ID NO: 494 - protein, 9 aa, Artificial sequence
  Synthetic polypeptide
QQYSRYPLT

SEQ ID NO: 495 - protein, 11 aa, Artificial sequence
  Synthetic polypeptide
RASQDISNHLN

SEQ ID NO: 496 - protein, 7 aa, Artificial sequence
  Synthetic polypeptide
YTSRLHS

SEQ ID NO: 497 - protein, 9 aa, Artificial sequence
  Synthetic polypeptide
QQDSKPPPT

SEQ ID NO: 498 - protein, 6 aa, Artificial sequence
  Synthetic polypeptide
QDISNH

SEQ ID NO: 499 - protein, 3 aa, Artificial sequence
  Synthetic polypeptide
YTS

SEQ ID NO: 500 - protein, 9 aa, Artificial sequence
  Synthetic polypeptide
QQDSKPPPT

SEQ ID NO: 501 - protein, 16 aa, Artificial sequence
  Synthetic polypeptide
RSSQNIVHSNGNTYLQ

SEQ ID NO: 502 - protein, 7 aa, Artificial sequence
  Synthetic polypeptide
KVSNRFS

SEQ ID NO: 503 - protein, 9 aa, Artificial sequence
  Synthetic polypeptide
FQGSHVPFT

SEQ ID NO: 504 - protein, 11 aa, Artificial sequence
  Synthetic polypeptide
QNIVHSNGNTY

SEQ ID NO: 505 - protein, 3 aa, Artificial sequence
  Synthetic polypeptide
KVS

SEQ ID NO: 506 - protein, 9 aa, Artificial sequence
  Synthetic polypeptide
FQGSHVPFT

SEQ ID NO: 507 - protein, 16 aa, Artificial sequence
  Synthetic polypeptide
RFSQSIVNSNGNTYLE

SEQ ID NO: 508 - protein, 7 aa, Artificial sequence
  Synthetic polypeptide
KVSNRFS

SEQ ID NO: 509 - protein, 9 aa, Artificial sequence
  Synthetic polypeptide
FQGSHVPFT

SEQ ID NO: 510 - protein, 11 aa, Artificial sequence
  Synthetic polypeptide
QSIVNSNGNTY

SEQ ID NO: 511 - protein, 3 aa, Artificial sequence
  Synthetic polypeptide
KVS

SEQ ID NO: 512 - protein, 9 aa, Artificial sequence
  Synthetic polypeptide
FQGSHVPFT

SEQ ID NO: 513 - protein, 16 aa, Artificial sequence
  Synthetic polypeptide
RSSQGLVHNNGNTYLH

SEQ ID NO: 514 - protein, 7 aa, Artificial sequence
  Synthetic polypeptide
KVSNRFS

SEQ ID NO: 515 - protein, 9 aa, Artificial sequence
  Synthetic polypeptide
SQNTHVPPT

SEQ ID NO: 516 - protein, 11 aa, Artificial sequence
  Synthetic polypeptide
QGLVHNNGNTY

SEQ ID NO: 517 - protein, 3 aa, Artificial sequence
  Synthetic polypeptide
KVS

SEQ ID NO: 518 - protein, 9 aa, Artificial sequence
  Synthetic polypeptide
SQNTHVPPT

SEQ ID NO: 519 - protein, 16 aa, Artificial sequence
  Synthetic polypeptide
RSSQSLVHSNGNTYLQ

SEQ ID NO: 520 - protein, 7 aa, Artificial sequence
  Synthetic polypeptide
KVSNRFS

SEQ ID NO: 521 - protein, 9 aa, Artificial sequence
  Synthetic polypeptide
CQSTHVLPT

SEQ ID NO: 522 - protein, 11 aa, Artificial sequence
  Synthetic polypeptide
QSLVHSNGNTY

SEQ ID NO: 523 - protein, 3 aa, Artificial sequence
  Synthetic polypeptide
KVS

SEQ ID NO: 524 - protein, 9 aa, Artificial sequence
  Synthetic polypeptide
CQSTHVLPT

SEQ ID NO: 525 - protein, 16 aa, Artificial sequence
  Synthetic polypeptide
RSSQSIVHSNGNTYLE

SEQ ID NO: 526 - protein, 7 aa, Artificial sequence
  Synthetic polypeptide
KVSNRFS

SEQ ID NO: 527 - protein, 9 aa, Artificial sequence
  Synthetic polypeptide
FQGSHVPPT

SEQ ID NO: 528 - protein, 11 aa, Artificial sequence
  Synthetic polypeptide
QSIVHSNGNTY

SEQ ID NO: 529 - protein, 3 aa, Artificial sequence
  Synthetic polypeptide
KVS

SEQ ID NO: 530 - protein, 9 aa, Artificial sequence
  Synthetic polypeptide
FQGSHVPPT

SEQ ID NO: 531 - protein, 16 aa, Artificial sequence
  Synthetic polypeptide
RSSQTFVHSNGNTYLQ

SEQ ID NO: 532 - protein, 7 aa, Artificial sequence
  Synthetic polypeptide
KVSNRFS

SEQ ID NO: 533 - protein, 9 aa, Artificial sequence
  Synthetic polypeptide
FQGSHVPYT

SEQ ID NO: 534 - protein, 11 aa, Artificial sequence
  Synthetic polypeptide
QTFVHSNGNTY

SEQ ID NO: 535 - protein, 3 aa, Artificial sequence
  Synthetic polypeptide
KVS

SEQ ID NO: 536 - protein, 9 aa, Artificial sequence
  Synthetic polypeptide
FQGSHVPYT

SEQ ID NO: 537 - protein, 16 aa, Artificial sequence
  Synthetic polypeptide
RSSQSLVHSNGNTYLH

SEQ ID NO: 538 - protein, 7 aa, Artificial sequence
  Synthetic polypeptide
KVSNRFS

SEQ ID NO: 539 - protein, 9 aa, Artificial sequence
  Synthetic polypeptide
SQNTHVPPT

SEQ ID NO: 540 - protein, 11 aa, Artificial sequence
  Synthetic polypeptide
QSLVHSNGNTY

SEQ ID NO: 541 - protein, 3 aa, Artificial sequence
  Synthetic polypeptide
KVS

SEQ ID NO: 542 - protein, 9 aa, Artificial sequence
  Synthetic polypeptide
SQNTHVPPT

SEQ ID NO: 543 - protein, 16 aa, Artificial sequence
  Synthetic polypeptide
RSSQSFVHSNGDTYLQ

SEQ ID NO: 544 - protein, 7 aa, Artificial sequence
  Synthetic polypeptide
KVSNRFS

SEQ ID NO: 545 - protein, 9 aa, Artificial sequence
  Synthetic polypeptide
FQGSHVPYT

SEQ ID NO: 546 - protein, 11 aa, Artificial sequence
  Synthetic polypeptide
QSFVHSNGDTY

SEQ ID NO: 547 - protein, 3 aa, Artificial sequence
  Synthetic polypeptide
KVS

SEQ ID NO: 548 - protein, 9 aa, Artificial sequence
  Synthetic polypeptide
FQGSHVPYT

SEQ ID NO: 549 - protein, 11 aa, Artificial sequence
  Synthetic polypeptide
SASQGIFNYLN

SEQ ID NO: 550 - protein, 7 aa, Artificial sequence
  Synthetic polypeptide
YTSSLHS

SEQ ID NO: 551 - protein, 9 aa, Artificial sequence
  Synthetic polypeptide
QQYSKLPWT

SEQ ID NO: 552 - protein, 6 aa, Artificial sequence
  Synthetic polypeptide
QGIFNY

SEQ ID NO: 553 - protein, 3 aa, Artificial sequence
  Synthetic polypeptide
YTS

SEQ ID NO: 554 - protein, 9 aa, Artificial sequence
  Synthetic polypeptide
QQYSKLPWT

SEQ ID NO: 555 - protein, 11 aa, Artificial sequence
  Synthetic polypeptide
RASQSIGTSIH

SEQ ID NO: 556 - protein, 7 aa, Artificial sequence
  Synthetic polypeptide
YAFESIS

SEQ ID NO: 557 - protein, 9 aa, Artificial sequence
  Synthetic polypeptide
QQTYNWPYT

SEQ ID NO: 558 - protein, 6 aa, Artificial sequence
  Synthetic polypeptide
QSIGTS

SEQ ID NO: 559 - protein, 3 aa, Artificial sequence
  Synthetic polypeptide
YAF

SEQ ID NO: 560 - protein, 9 aa, Artificial sequence
  Synthetic polypeptide
QQTYNWPYT

SEQ ID NO: 561 - protein, 16 aa, Artificial sequence
  Synthetic polypeptide
RSSQNIVHRNGNTYLE

SEQ ID NO: 562 - protein, 6 aa, Artificial sequence
  Synthetic polypeptide
KVSNRF

SEQ ID NO: 563 - protein, 9 aa, Artificial sequence
  Synthetic polypeptide
FQGSHFPFT

SEQ ID NO: 564 - protein, 11 aa, Artificial sequence
  Synthetic polypeptide
QNIVHRNGNTY

SEQ ID NO: 565 - protein, 3 aa, Artificial sequence
  Synthetic polypeptide
KVS

SEQ ID NO: 566 - protein, 9 aa, Artificial sequence
  Synthetic polypeptide
FQGSHFPFT

SEQ ID NO: 567 - protein, 16 aa, Artificial sequence
  Synthetic polypeptide
RSSQSLLHSNGNTYLH

SEQ ID NO: 568 - protein, 7 aa, Artificial sequence
  Synthetic polypeptide
KVSNRFS

SEQ ID NO: 569 - protein, 9 aa, Artificial sequence
  Synthetic polypeptide
SQSTHVPWT

SEQ ID NO: 570 - protein, 11 aa, Artificial sequence
  Synthetic polypeptide
QSLLHSNGNTY

SEQ ID NO: 571 - protein, 3 aa, Artificial sequence
  Synthetic polypeptide
KVS

SEQ ID NO: 572 - protein, 9 aa, Artificial sequence
  Synthetic polypeptide
SQSTHVPWT

SEQ ID NO: 573 - protein, 16 aa, Artificial sequence
  Synthetic polypeptide
RSSQSLVHSNGNTYLQ

SEQ ID NO: 574 - protein, 7 aa, Artificial sequence
  Synthetic polypeptide
KVSNRFS

SEQ ID NO: 575 - protein, 9 aa, Artificial sequence
  Synthetic polypeptide
SQSTHVPYT

SEQ ID NO: 576 - protein, 11 aa, Artificial sequence
  Synthetic polypeptide
QSLVHSNGNTY

SEQ ID NO: 577 - protein, 3 aa, Artificial sequence
  Synthetic polypeptide
KVS

SEQ ID NO: 578 - protein, 9 aa, Artificial sequence
  Synthetic polypeptide
SQSTHVPYT

SEQ ID NO: 579 - protein, 115 aa, Artificial sequence
  Synthetic polypeptide
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSA

SEQ ID NO: 580 - protein, 113 aa, Artificial sequence
  Synthetic polypeptide
DIVMTQTPLSLPVITGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKR

SEQ ID NO: 581 - protein, 475 aa, Artificial sequence
  Synthetic polypeptide
DIVMTQTPLSLPVITGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRGGGGSGGGGSGGGGSEVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 582 - protein, 121 aa, Artificial sequence
  Synthetic polypeptide
QVQLQESGGGLVQPGRSLRLSCAASGFTFSSYAMHWVRQAPGKGLEWVSAISSSGRSTYYADSVEGRFTISRDNSKNTLYLQMNSLRAEDTAVYYCAKDRRGSHADALNVWGQGTLVTVSS

SEQ ID NO: 583 - protein, 111 aa, Artificial sequence
  Synthetic polypeptide
QSALTQPPSASGSPGQSVTISCTGTSSDVGGYNYVSWYQQYPGKAPKLLIYGNSNRPSGVPDRFSGSKSGTSASLAITGLQAEDGADYYCQSYDSSLRVVFGGGTKVTVLG

SEQ ID NO: 584 - protein, 479 aa, Artificial sequence
  Synthetic polypeptide
QSALTQPPSASGSPGQSVTISCTGTSSDVGGYNYVSWYQQYPGKAPKLLIYGNSNRPSGVPDRFSGSKSGTSASLAITGLQAEDGADYYCQSYDSSLRVVFGGGTKVTVLGGGGGSGGGGSGGGGSQVQLQESGGGLVQPGRSLRLSCAASGFTFSSYAMHWVRQAPGKGLEWVSAISSSGRSTYYADSVEGRFTISRDNSKNTLYLQMNSLRAEDTAVYYCAKDRRGSHADALNVWGQGTLVTVSSEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 585 - protein, 40 aa, Artificial sequence
  Synthetic polypeptide
AELAYDLDVDDAPGNSQQATPKDNEISTFHNLGNVHSPLK

SEQ ID NO: 586 - protein, 44 aa, Artificial sequence
  Synthetic polypeptide
AELAYDLDVDDAPGNSQQATPKDNEISTFHNLGNVHSPLKCKLH

SEQ ID NO: 587 - protein, 330 aa, Artificial sequence
  Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 588 - protein, 107 aa, Artificial sequence [sequence omitted]

SEQ ID NO: 589 - protein, 115 aa, Artificial sequence
  Synthetic polypeptide
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 590 - protein, 112 aa, Artificial sequence
  Synthetic polypeptide
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 591 - protein, 474 aa, Artificial sequence
  Synthetic polypeptide
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 592 - protein, 445 aa, Artificial sequence
  Synthetic polypeptide
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 593 - protein, 219 aa, Artificial sequence
  Synthetic polypeptide
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 594 - protein, 120 aa, Artificial sequence
  Synthetic polypeptide
EVQLVESGGGLVQPGGSLRLSCAASGFTFSTNAMNWVRQAPGKGLEWVGRIRSKSNNYATYYADSVKDRFTISRDDSKNSLYLQMNSLKTEDTAVYYCARDGYYVLFAYWGQGTLVTVSS

SEQ ID NO: 595 - protein, 112 aa, Artificial sequence
  Synthetic polypeptide
DIVMTQTPLSLPVTPGEPASISCRFSQSIVNSNGNTYLEWYLQKPGQSPQLLIYKVSNRFSGVPDRFSGSGSGTDFTLKISRVEAEDVGVYYCFQGSHVPFTFGQGTKLEIK

SEQ ID NO: 596 - protein, 112 aa, Artificial sequence
  Synthetic polypeptide
DIVMTQTPLSLPVTPGEPASISCRFSQSIVNSNGNTYLEWFLQKPGQSPQLLIYKVSNRFSGVPDRFSGSGSGTDFTLKISRVEAEDVGVYYCFQGSHVPFTFGQGTKLEIK

SEQ ID NO: 597 - protein, 112 aa, Artificial sequence
  Synthetic polypeptide
DIVMTQTPLSLPVTPGEPASISCRFSQSIVNSDGNTYLEWFLQKPGQSPQLLIYKVSNRFSGVPDRFSGSGSGTDFTLKISRVEAEDVGVYYCFQGSHVPFTFGQGTKLEIK

SEQ ID NO: 598 - protein, 112 aa, Artificial sequence
  Synthetic polypeptide
DIVMTQTPLSLPVTPGEPASISCRFSQSIVNSNANTYLEWFLQKPGQSPQLLIYKVSNRFSGVPDRFSGSGSGTDFTLKISRVEAEDVGVYYCFQGSHVPFTFGQGTKLEIK

SEQ ID NO: 599 - protein, 120 aa, Artificial sequence
  Synthetic polypeptide
EVQLVESGGGLVQPGGSLRLSCAASGFSFNTNAMNWVRQAPGKGLEWVGRIRSKSNNYATYYADSVKDRFTISRDDSKNSLYLQMNSLKTEDTAVYYCVRDGYYVLFAYWGQGTLVTVSS

SEQ ID NO: 600 - protein, 120 aa, Artificial sequence
  Synthetic polypeptide
EVQLVESGGGLVQPGGSLRLSCAASGFSFNTNAMNWVRQAPGKGLEWVGRIRSKSNNYATYYADSVKDRFTISRDDSKSSLYLQMNSLKTEDTAVYYCVRDGYYVLFAYWGQGTLVTVSS

SEQ ID NO: 601 - protein, 120 aa, Artificial sequence
  Synthetic polypeptide
EVQLVESGGGLVQPGGSLRLSCAASGFSFNTNAMNWVRQAPGKGLEWVGRIRSKSNNYATYYADSVKGRFTISRDDSKNSLYLQMNSLKTEDTAVYYCVRDGYYVLFAYWGQGTLVTVSS

SEQ ID NO: 602 - protein, 16 aa, Artificial sequence
  Synthetic polypeptide
RFSQSIVNSDGNTYLE

SEQ ID NO: 603 - protein, 16 aa, Artificial sequence
  Synthetic polypeptide
RFSQSIVNSNANTYLE

SEQ ID NO: 604 - protein, 19 aa, Artificial sequence
  Synthetic polypeptide
RIRSKSNNYATYYADSVKG

SEQ ID NO: 605 - protein, 115 aa, Artificial sequence
  Synthetic polypeptide
EVQLVQSGAEVKKPGSSVKVSCKASGGTFSDYEMHWVRQAPGQGLEWMGTIDPETGNTAYTQKFMDRVTITADKSTSTAYMELSSLRSEDTAVYYCARGYSFTYWGQGTLVTVSS

SEQ ID NO: 606 - protein, 112 aa, Artificial sequence
  Synthetic polypeptide
DIVMTQTPLSLPVTPGEPASISCRSSQNIVHSNGNTYLQWYLQKPGQSPQLLIYKVSNRFSGVPDRFSGSGSGTDFTLKISRVEAEDVGVYYCFQGSHVPFTFGQGTKLEIK

SEQ ID NO: 607 - protein, 112 aa, Artificial sequence
  Synthetic polypeptide
DIVMTQTPLSLPVTPGEPASISCRSSQNIVHSNGNTYLQWYLQKPGQSPQLLIFKVSNRFSGVPDRFSGSGSGTDFTLKISRVEAEDVGVYYCFQGSHVPFTFGQGTKLEIK

SEQ ID NO: 608 - protein, 112 aa, Artificial sequence
  Synthetic polypeptide
DIVMTQTPLSLPVTPGEPASISCRSSQNIVHSDGNTYLQWYLQKPGQSPQLLIFKVSNRFSGVPDRFSGSGSGTDFTLKISRVEAEDVGVYYCFQGSHVPFTFGQGTKLEIK

SEQ ID NO: 609 - protein, 112 aa, Artificial sequence
  Synthetic polypeptide
DIVMTQTPLSLPVTPGEPASISCRSSQNIVHSNRNTYLQWYLQKPGQSPQLLIFKVSNRFSGVPDRFSGSGSGTDFTLKISRVEAEDVGVYYCFQGSHVPFTFGQGTKLEIK

SEQ ID NO: 610 - protein, 115 aa, Artificial sequence
  Synthetic polypeptide
EVQLVQSGAEVKKPGSSVKVSCKASGYTFSDYEMHWVRQAPGQGLEWMGTIDPETGNTAYTQKFMDRVTITADKSTSTAYMELSSLRSEDTAVYYCTRGYSFTYWGQGTLVTVSS

SEQ ID NO: 611 - protein, 115 aa, Artificial sequence
  Synthetic polypeptide
EVQLVQSGAEVKKPGSSVKVSCKASGYRFTDYEMHWVRQAPGQGLEWMGTIDPETGNTAYTQKFMDRVTITADKSTSTAYMELSSLRSEDTAVYYCTRGYSFTYWGQGTLVTVSS

SEQ ID NO: 612 - protein, 115 aa, Artificial sequence
  Synthetic polypeptide
EVQLVQSGAEVKKPGSSVKVSCKASGYRFTDYEMHWVRQAPGQGLEWMGTIDPETGNTAYTQKFMDRVTLTADKSTSTAYMELSSLRSEDTAVYYCTRGYSFTYWGQGTLVTVSS

SEQ ID NO: 613 - protein, 16 aa, Artificial sequence
  Synthetic polypeptide
RSSQNIVHSDGNTYLQ

SEQ ID NO: 614 - protein, 16 aa, Artificial sequence
  Synthetic polypeptide
RSSQNIVHSNRNTYLQ

SEQ ID NO: 615 - protein, 115 aa, Artificial sequence
  Synthetic polypeptide
EVQLVQSGAEVKKPGASVKVSCKASGYTFTDYEMHWVRQAPGQGLEWMGAIDPETGNTAYIQKFKGRVTMTTDTSTSTAYMELRSLRSDDTAVYYCARFYSYSHWGQGTTVTVSS

SEQ ID NO: 616 - protein, 112 aa, Artificial sequence
  Synthetic polypeptide
DIVMTQTPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGGGTKVEIK

SEQ ID NO: 617 - protein, 112 aa, Artificial sequence; identical to SEQ ID NO: 616
  Synthetic polypeptide

SEQ ID NO: 618 - protein, 112 aa, Artificial sequence
  Synthetic polypeptide
DIVMTQTPLSLPVTPGEPASISCRSSQSLVHSDGNTYLHWYLQKPGQSPQLLIYKVSNRFSGVPDRFSGSGSGTDFTLKISRVEAEDVGVYYCSQNTHVPPTFGGGTKVEIK

SEQ ID NO: 619 - protein, 112 aa, Artificial sequence
  Synthetic polypeptide
DIVMTQTPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGGGTKVEIK

SEQ ID NO: 620 - protein, 115 aa, Artificial sequence
  Synthetic polypeptide
EVQLVQSGAEVKKPGASVKVSCKASGYTFTDYEMHWVRQAPGQGLEWMGAIDPETGNTAYIQKFKGRVTMTTDKSTSTAYMELRSLRSDDTAVYYCTRFYSYSHWGQGTTVTVSS

SEQ ID NO: 621 - protein, 115 aa, Artificial sequence
  Synthetic polypeptide
EVQLVQSGAEVKKPGASVKVSCKASGYTFTDYEMHWVRQAPGQGLEWMGAIDPETGNTAYIQKFKGRVTLTTDKSTSTAYMELRSLRSDDTAVYYCTRFYSYSHWGQGTTVTVSS

SEQ ID NO: 622 - protein, 115 aa, Artificial sequence
  Synthetic polypeptide
EVQLVQSGAEVKKPGASVKVSCKASGYTFTDYEMHWVRQAPGQGLEWIGAIDPETGNTAYIQKFKGRATLTTDKSTSTAYMELRSLRSDDTAVYYCTRFYSYSHWGQGTTVTVSS

SEQ ID NO: 623 - protein, 16 aa, Artificial sequence
  Synthetic polypeptide
RSSQSLVHSDGNTYLH

SEQ ID NO: 624 - protein, 16 aa, Artificial sequence
  Synthetic polypeptide
RSSQSLVHSNRNTYLH

SEQ ID NO: 625 - protein, 115 aa, Artificial sequence
  Synthetic polypeptide
EVQLVQSGAEVKKPGSSVKVSCKASGGTFSDYEIHWVRQAPGQGLEWMGAIDPKTGNTAYNQKFMGRVTITADKSTSTAYMELSSLRSEDTAVYYCARYFSFAYWGQGTLVTVSS

SEQ ID NO: 626 - protein, 112 aa, Artificial sequence
  Synthetic polypeptide
DIVMTQTPLSLPVTPGEPASISCRSSQTFVHSNGNTYLQWYLQKPGQSPQLLIYKVSNRFSGVPDRFSGSGSGTDFTLKISRVEAEDVGVYYCFQGSHVPYTFGQGTKLEIK

SEQ ID NO: 627 - protein, 112 aa, Artificial sequence; identical to SEQ ID NO: 626
  Synthetic polypeptide

SEQ ID NO: 628 - protein, 112 aa, Artificial sequence
  Synthetic polypeptide
DIVMTQTPLSLPVTPGEPASISCRSSQTFVHSDGNTYLQWYLQKPGQSPQLLIYKVSNRFSGVPDRFSGSGSGTDFTLKISRVEAEDVGVYYCFQGSHVPYTFGQGTKLEIK

SEQ ID NO: 629 - protein, 112 aa, Artificial sequence
  Synthetic polypeptide
DIVMTQTPLSLPVTPGEPASISCRSSQTFVHSNANTYLQWYLQKPGQSPQLLIYKVSNRFSGVPDRFSGSGSGTDFTLKISRVEAEDVGVYYCFQGSHVPYTFGQGTKLEIK

SEQ ID NO: 630 - protein, 112 aa, Artificial sequence
  Synthetic polypeptide
DIVMTQTPLSLPVTPGEPASISCRSSQTFVHSNRNTYLQWYLQKPGQSPQLLIYKVSNRFSGVPDRFSGSGSGTDFTLKISRVEAEDVGVYYCFQGSHVPYTFGQGTKLEIK

SEQ ID NO: 631 - protein, 112 aa, Artificial sequence
  Synthetic polypeptide
DIVMTQTPLSLPVTPGEPASISCRSSQTFVHSNSNTYLQWYLQKPGQSPQLLIYKVSNRFSGVPDRFSGSGSGTDFTLKISRVEAEDVGVYYCFQGSHVPYTFGQGTKLEIK

SEQ ID NO: 632 - protein, 112 aa, Artificial sequence
  Synthetic polypeptide
DIVMTQTPLSLPVTPGEPASISCRSSQTFVHSNVNTYLQWYLQKPGQSPQLLIYKVSNRFSGVPDRFSGSGSGTDFTLKISRVEAEDVGVYYCFQGSHVPYTFGQGTKLEIK

SEQ ID NO: 633 - protein, 115 aa, Artificial sequence
  Synthetic polypeptide
EVQLVQSGAEVKKPGSSVKVSCKASGYTFSDYEIHWVRQAPGQGLEWMGAIDPKTGNTAYNQKFMGRVTITADKSTSTAYMELSSLRSEDTAVYYCTRYFSFAYWGQGTLVTVSS

SEQ ID NO: 634 - protein, 115 aa, Artificial sequence
  Synthetic polypeptide
EVQLVQSGAEVKKPGSSVKVSCKASGYTFTDYEIHWVRQAPGQGLEWMGAIDPKTGNTAYNQKFMGRVTITADKSTSTAYMELSSLRSEDTAVYYCTRYFSFAYWGQGTLVTVSS

SEQ ID NO: 635 - protein, 115 aa, Artificial sequence
  Synthetic polypeptide
EVQLVQSGAEVKKPGSSVKVSCKASGYTFTDYEIHWVRQAPGQGLEWMGAIDPKTGNTAYNQKFMGRVTLTADKSTSTAYMELSSLRSEDTAVYYCTRYFSFAYWGQGTLVTVSS

SEQ ID NO: 636 - protein, 16 aa, Artificial sequence
  Synthetic polypeptide
RSSQTFVHSDGNTYLQ

SEQ ID NO: 637 - protein, 16 aa, Artificial sequence
  Synthetic polypeptide
RSSQTFVHSNANTYLQ

SEQ ID NO: 638 - protein, 16 aa, Artificial sequence
  Synthetic polypeptide
RSSQTFVHSNRNTYLQ

SEQ ID NO: 639 - protein, 16 aa, Artificial sequence
  Synthetic polypeptide
RSSQTFVHSNSNTYLQ

SEQ ID NO: 640 - protein, 16 aa, Artificial sequence
  Synthetic polypeptide
RSSQTFVHSNVNTYLQ

SEQ ID NO: 641 - protein, 115 aa, Artificial sequence
  Synthetic polypeptide
EVQLVQSGAEVKKPGSSVKVSCKASGGTFSDYEMHWVRQAPGQGLEWMGTIDPETGNTAYNQKFKGRVTITADKSTSTAYMELSSLRSEDTAVYYCARPYSLTYWGQGTLVTVSS

SEQ ID NO: 642 - protein, 112 aa, Artificial sequence
  Synthetic polypeptide
DIVMTQTPLSLPVTPGEPASISCRSSQSFVHSNGDTYLQWYLQKPGQSPQLLIYKVSNRFSGVPDRFSGSGSGTDFTLKISRVEAEDVGVYYCFQGSHVPYTFGQGTKLEIK

SEQ ID NO: 643 - protein, 112 aa, Artificial sequence; identical to SEQ ID NO: 642
  Synthetic polypeptide

SEQ ID NO: 644 - protein, 112 aa, Artificial sequence
  Synthetic polypeptide
DIVMTQTPLSLPVTPGEPASISCRSSQSFVHSDGDTYLQWYLQKPGQSPQLLIYKVSNRFSGVPDRFSGSGSGTDFTLKISRVEAEDVGVYYCFQGSHVPYTFGQGTKLEIK

SEQ ID NO: 645 - protein, 112 aa, Artificial sequence
  Synthetic polypeptide
DIVMTQTPLSLPVTPGEPASISCRSSQSFVHSNRDTYLQWYLQKPGQSPQLLIYKVSNRFSGVPDRFSGSGSGTDFTLKISRVEAEDVGVYYCFQGSHVPYTFGQGTKLEIK

SEQ ID NO: 646 - protein, 112 aa, Artificial sequence
  Synthetic polypeptide
DIVMTQTPLSLPVTPGEPASISCRSSQSFVHSNADTYLQWYLQKPGQSPQLLIYKVSNRFSGVPDRFSGSGSGTDFTLKISRVEAEDVGVYYCFQGSHVPYTFGQGTKLEIK

SEQ ID NO: 647 - protein, 112 aa, Artificial sequence
  Synthetic polypeptide
DIVMTQTPLSLPVTPGEPASISCRSSQSFVHSNSDTYLQWYLQKPGQSPQLLIYKVSNRFSGVPDRFSGSGSGTDFTLKISRVEAEDVGVYYCFQGSHVPYTFGQGTKLEIK

SEQ ID NO: 648 - protein, 112 aa, Artificial sequence
  Synthetic polypeptide
DIVMTQTPLSLPVTPGEPASISCRSSQSFVHSNVDTYLQWYLQKPGQSPQLLIYKVSNRFSGVPDRFSGSGSGTDFTLKISRVEAEDVGVYYCFQGSHVPYTFGQGTKLEIK

SEQ ID NO: 649 - protein, 115 aa, Artificial sequence
  Synthetic polypeptide
EVQLVQSGAEVKKPGSSVKVSCKASGYTFSDYEMHWVRQAPGQGLEWMGTIDPETGNTAYNQKFKGRVTITADKSTSTAYMELSSLRSEDTAVYYCTRPYSLTYWGQGTLVTVSS

SEQ ID NO: 650 - protein, 115 aa, Artificial sequence
  Synthetic polypeptide
EVQLVQSGAEVKKPGSSVKVSCKASGYTFTDYEMHWVRQAPGQGLEWMGTIDPETGNTAYNQKFKGRVTITADKSTSTAYMELSSLRSEDTAVYYCTRPYSLTYWGQGTLVTVSS

SEQ ID NO: 651 - protein, 115 aa, Artificial sequence
  Synthetic polypeptide
EVQLVQSGAEVKKPGSSVKVSCKASGYTFTDYEMHWVRQAPGQGLEWMGTIDPETGNTAYNQKFKGRVTLTADKSTSTAYMELSSLRSEDTAVYYCTRPYSLTYWGQGTLVTVSS

SEQ ID NO: 652 - protein, 16 aa, Artificial sequence
  Synthetic polypeptide
RSSQSFVHSDGDTYLQ

SEQ ID NO: 653 - protein, 16 aa, Artificial sequence
  Synthetic polypeptide
RSSQSFVHSNRDTYLQ

SEQ ID NO: 654 - protein, 16 aa, Artificial sequence
  Synthetic polypeptide
RSSQSFVHSNADTYLQ

SEQ ID NO: 655 - protein, 16 aa, Artificial sequence
  Synthetic polypeptide
RSSQSFVHSNSDTYLQ

SEQ ID NO: 656 - protein, 16 aa, Artificial sequence
  Synthetic polypeptide
RSSQSFVHSNVDTYLQ